(19) United States
(12) Patent Application Publication
(10) Pub. No.: US 2011/0263441 A1
(43) Pub. Date: Oct. 27, 2011

(54) COMPOSITIONS, KITS, AND METHODS FOR IDENTIFICATION, ASSESSMENT, PREVENTION, AND THERAPY OF HEPATIC DISORDERS

(76) Inventors: Todd R. Golub, Newton, MA (US); Yujin Hoshida, Brookline, MA (US); Bryan C. Fuchs, Quincy, MA (US); Kenneth K. Tanabe, Brookline, MA (US); Josep M. Llovet Bayer, Barcelona (ES); Augusto Villanueva, Barcelona (ES)

(21) Appl. No.: 13/124,487

(22) PCT Filed: Oct. 15, 2009
(86) PCT No.: PCT/US2009/060859

§ 371 Date: Jul. 15, 2011

Related U.S. Application Data
(60) Provisional application No. 61/196,110, filed on Oct. 15, 2008.

(51) Int. Cl. C40B 30/00; G01N 33/566; C40B 40/10; C12Q 1/34; C12Q 1/48; C40B 40/06; C12Q 1/68; C12Q 1/26

(52) U.S. Cl. 506/7; 435/6.11; 435/7.92; 435/25; 435/18; 435/15; 435/7.24; 436/501; 506/16; 506/18

(57) ABSTRACT

The invention relates to compositions, kits, and methods for detecting, characterizing, preventing, and treating hepatic disorders such as hepatocellular carcinoma and/or cirrhosis. A variety of informative biomarkers corresponding thereto, are provided, wherein alterations in expression relative to a control is correlated with the presence of a hepatic disorder, likelihood of survival from a hepatic disorder, and likelihood of recurrence of a hepatic disorder.

### CROSS-REFERENCE TO RELATED APPLICATIONS

This application claims the benefit of priority to U.S. Provisional Application No. 61/196,110, filed on Oct. 15, 2008, the contents of which are hereby incorporated in their entirety.

### GOVERNMENT FUNDING

Work described herein was supported, at least in part, by the National Cancer Institute (grant number 5U54CA112962-03). The government may therefore have certain rights to this invention.

### BACKGROUND OF THE INVENTION

Liver disease is increasing in incidence, morbidity and mortality because of the lack of effective preventive, diagnostic, and prognostic measures, as well as the absence of specific treatments. Therapy is largely symptomatic or supportive for fatty liver, hepatitis, cirrhosis, hepatocellular cancer and metabolic disorders which are the common diseases of the liver. For example, hepatocellular carcinoma (HCC), a malignant tumor of the liver, is the third leading cause of cancer-related death in the world, and its incidence is increasing in Europe and the US. HCC is now the leading cause of death among cirrhotic patients and accounts for 80% to 90% of all liver cancers. In developing countries, hepatocellular carcinoma often comes to medical attention when the tumors are at an advanced stage and curative therapies are of limited benefit. In developed countries, however, at-risk populations of patients (e.g., those who are infected with hepatitis virus and have cirrhosis) are often under close surveillance; as a result, hepatocellular carcinoma is usually detected when the tumors are small and treatment is more likely to be successful (Llovet, J. M., et al. (2003) Lancet 362, 1907-1917; Llovet, J. M., et al. (2008) J. Hepatol. 48, S20-S37). Liver cancer has been treated with, for example, hepatectomy, percutaneous local therapy (e.g., radiofrequency ablation therapy or ethanol injection therapy), transcatheter hepatic arterial embolization (TAE), continuous arterial infusion chemotherapy, or radiation therapy. Nevertheless, recurrences eventually occur in most patients (Llovet, J. M., et al. (2003) Lancet 362, 1907-1917; Llovet, J. M., et al. (2008) J. Hepatol. 48, S20-S37). Studies suggest that chemopreventive strategies suppress recurrence and prolong survival (Llovet, J. M., et al. (2003) Lancet 362, 1907-1917; Ikeda, K., et al. (2000) Hepatology 32, 228-232; Muto, Y., et al. (1996) N. Engl. J. Med. 334, 1561-1567; Takayama, T. et al. (2000) Lancet 356, 802-807 [Erratum, (2000) Lancet 356, 1690]; Lau, W. Y., et al. (1999) Lancet 353:797-801. One may wish to treat patients at greatest risk for recurrence. Several methods have been used to predict survival among patients with hepatocellular carcinoma, including the enumeration of anatomical and histopathological attributes (e.g., tumor multinodularity and vascular invasion), but these have become less useful as hepatocellular carcinoma is increasingly diagnosed at earlier stages.

In addition, liver cirrhosis represents the terminal stage of many chronic liver diseases, and is estimated to affect up to 1% of population (Schuppan and Afdhal, (2008) Lancet 371, 838-851). Cirrhosis-related mortality is high, with deaths attributable either to portal hypertension-associated complications such as gastrointestinal varices, or to hepatocellular carcinoma which occurs in nearly one third of patients with cirrhosis (Llovet J. M., et al. (2003) Lancet 362, 1907-1917). Even after surgical excision or percutaneous ablation of initial hepatocellular carcinomas, most patients develop subsequent de novo tumors due to carcinogenic microenvironment in the cirrhotic liver, called the “field effect” (Sherman, M., et al. (2008) N. Engl. J. Med. 359, 2045-2047). While survival of patients with cirrhosis is diminished compared to the general population, it is clear that the clinical course can be highly variable. Some patients have a rapidly deteriorating course, whereas others have minimally progressive disease, surviving decades (Schuppan and Afdhal, (2008) Lancet 371, 838-851). This clinical course is in part predictable using a composite measure of liver function known as Child-Pugh staging, wherein Child-Pugh Class A have a 100% 1-year survival rate, compared for example, to a rate of 45% for Child-Pugh Class C patients (Schuppan and Afdhal, (2008) Lancet 371, 838-851). However, the majority of newly diagnosed patients are Class A, and for these patients particularly, additional prognostic biomarkers are lacking. Although a number of chemopreventive strategies are being explored as a means of abrogating the lethal complications of cirrhosis, including hepatocellular carcinoma, such approaches, including anti-inflammatory and anti-fibrotic therapies, have met with variable success, are accompanied by significant toxicity, and are expensive ((Schuppan and Afdhal, (2008) Lancet 371, 838-851; Webster, D. P., et al. (2009) Lancet Infect. Dis. 9, 108-117; Di Bisceglie, A. M., et al. (2008) N. Engl. J. Med. 359, 2429-2441).

A technical challenge facing the use of gene expression profiling to predict the outcome of hepatic disorders has been the lack of suitable specimens from patients. Current methods of genome-wide expression profiling require frozen tissue for analysis, whereas tissue banks with clinical outcome data generally have formalin fixed, paraffin embedded specimens. Even today the vast majority of specimens are formalin-fixed; the collection of frozen tissues has yet to become routine clinical practice.

Thus, there is a pressing need for new biomarkers to effectively prevent, diagnose, prognose, and treat subjects at risk for developing hepatic disorders (e.g., hepatocellular carcinoma and/or cirrhosis), such that early intervention and intense surveillance might be focused on the population most likely to benefit. A new method for genome-wide expression profiling of tissues, including formalin-fixed, paraffin-embedded tissues, is described herein. The method may be applied to the analysis of the clinical outcome of hepatic disorders (e.g., hepatocellular carcinoma and/or cirrhosis), including novel methods for prognosing subjects to stratify those with increased risk of multi-centric recurrence from among early stage cancer patients, including hepatocellular carcinoma patients.

### SUMMARY OF THE INVENTION

The present invention features, at least in part, a method for determining if a subject is at risk for developing a hepatic disorder, comprising comparing the level of expression of a marker or a plurality of markers in a subject sample and the level of expression of the marker or plurality of markers in a control sample, wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample is an indication that the subject is at risk for developing the hepatic disorder. These markers were identified using a novel gene-expression profiling assay involving fixed embedded tissue, an innovative complementary DNA-mediated annealing, selection, extension, and ligation (DASL) assay, and a novel microarray.

In one embodiment, the marker or plurality of markers have increased expression relative to a control. In another embodiment, the marker or plurality of markers have decreased expression relative to a control. In still another embodiment, at least one marker has increased expression and at least one marker has decreased expression relative to a control.

In another embodiment, the hepatic disorder is liver cancer (e.g., HCC) and/or cirrhosis. In still another embodiment, the marker or plurality of markers comprise a transcribed polynucleotide or portion thereof. In yet another embodiment, the marker or plurality of markers corresponds to a secreted protein.

In one aspect, the level of expression of the marker or plurality of markers in the samples is assessed by detecting the presence of a marker protein in the samples e.g., the presence of the marker protein is detected using a reagent which specifically binds with the protein, e.g., reagents selected from the group consisting of an antibody, an antibody derivative, and an antibody fragment. In another aspect, the level of expression of the marker or plurality of markers in the samples is assessed by detecting the presence in the sample of a transcribed polynucleotide or portion thereof, corresponding to a nucleic acid marker (e.g., mRNA or a cDNA). In one embodiment, detecting a transcribed polynucleotide comprises amplifying the transcribed polynucleotide. In another embodiment, the level of expression of the marker or plurality of markers in the samples is assessed by detecting the presence in the sample of a transcribed polynucleotide which anneals with a nucleic acid marker or a portion thereof under stringent hybridization conditions.

In other embodiments, the level of expression of the marker or plurality of markers in the subject sample differs from the level of expression of the marker in the control sample by a factor of at least about 2 or at least about 5. In another embodiment, the level of expression of the marker or plurality of markers is determined using oligonucleotide microarrays. In still another embodiment, the level of expression of the marker or plurality of markers is determined using a complementary DNA-mediated annealing, selection, extension, and ligation assay. In yet another embodiment, the subject has undergone tumor resection. In another embodiment, the subject sample is obtained from non-tumor liver tissue or tissue surrounding a resected tumor and can be selected from the group consisting of fresh tissue, fresh frozen tissue, needle biopsy tissue, and fixed embedded (e.g., formalin-fixed, paraffin-embedded) tissue.

The present invention also features a method for determining the likelihood of survival of a subject having a hepatic disorder comprising comparing the level of expression of a marker or a plurality of markers in a subject sample and the level of expression of the marker or plurality of markers in a control sample, wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample indicates the likelihood of survival of the subject.

The present invention further features a method of predicting the likelihood of recurrence of a hepatic disorder (e.g., multi-centric recurrence of a liver cancer and/or cirrhosis) in a subject comprising comparing the level of expression of a marker or a plurality of markers in a subject sample and the level of expression of the marker or plurality of markers in a control sample, wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample predicts the likelihood of recurrence (e.g., multi-centric recurrence of a liver cancer and/or cirrhosis) of the hepatic disorder in the subject.

In one embodiment, the method further comprises the step of recommending a treatment (e.g., adjuvant or neoadjuvant treatment) for the subject based on the likelihood multi-centric recurrence of a hepatic disorder.

The present invention also features a method of classifying a tissue sample according to the predicted treatment outcome comprising comparing the level of expression of a marker or a plurality of markers in the tissue sample and the level of expression of the marker or plurality of markers in a control sample, wherein the marker or plurality of markers are selected from the group consisting of the markers listed in Table 2A, Table 2B, Table 13A, Table 13B, and FIG. 19 and the difference between the level of expression of the marker or plurality of markers in the tissue sample and the control sample classifies the tissue sample according to the predicted treatment outcome.

In one embodiment, the predicted treatment outcome is the likelihood of survival of an individual having a hepatic disorder or at risk for developing a hepatic disorder. In another embodiment, the predicted treatment outcome is the likelihood of centric recurrence, including multi-centric recurrence, of a hepatic disorder in an individual having a hepatic disorder or at risk for developing a hepatic disorder. In yet another embodiment, the predicted treatment outcome is the likely timing of centric recurrence, including multi-centric recurrence, of a hepatic disorder in an individual having a hepatic disorder or at risk for developing a hepatic disorder.

The present invention further features a method of assessing the efficacy of a hepatic disorder therapy in a subject, the method comprising comparing the level of expression of a marker or a plurality of markers in a first sample obtained from the subject and the level of expression of the marker or plurality of markers in a second sample obtained from the subject following provision of a portion of the therapy, wherein the marker or plurality of markers are selected from the group consisting of the markers listed in Table 2A, Table 2B, Table 13A, Table 13B, and FIG. 19 and a significant difference between the level of expression of the marker or plurality of markers indicates the efficacy of the hepatic disorder therapy.

The present invention also features a method of identifying an agent or compound for use in modulating development of a hepatic disorder, said method comprising the steps of providing a sample, contacting the sample with a candidate compound, and detecting an increase or decrease in expression of a marker or a plurality of markers selected from the group consisting of the markers in Table 2A, Table 2B, Table 13A, Table 13B, and FIG. 19 relative to a control, wherein an agent or compound that increases or decreases the expression of said marker or plurality of markers relative to the control is an agent or compound for use in modulating development of the hepatic disorder.

The present invention also features a method of assessing the efficacy of an agent or test compound for modulating development of a hepatic disorder, said method comprising the steps of providing a cell or cell lysate sample, contacting the cell or cell lysate sample with a candidate compound, and detecting an increase or decrease in expression of a marker or a plurality of markers selected from the group consisting of the markers in Table 2A, Table 2B, Table 13A, Table 13B, and FIG. 19 relative to a control wherein an agent or test compound that increases or decreases the expression of the marker or plurality of markers relative to the control is a compound for use in modulating development of the hepatic disorder.

The present invention further features a method of assessing whether a subject is afflicted with a hepatic disorder, the method comprising comparing the level of expression of a marker or a plurality of markers in a subject sample and the level of expression of the marker or plurality of markers in a control sample, wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample is an indication that the subject is afflicted with the hepatic disorder.

The present invention also features a method of monitoring the effect of erlotinib administered to a subject for preventing a hepatic disorder, the method comprising comparing the level of expression of a marker or a plurality of markers in a subject sample; and the level of expression of the marker or plurality of markers in a control sample, wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample is an indication that the hepatic disorder is being prevented by the erlotinib administration to the subject.

The present invention also features a diagnostic array comprising a solid support and a plurality of diagnostic agents coupled to the solid support, wherein each of the agents is used to assay the expression level of a marker or a plurality of markers is selected from the group consisting of the markers listed in Table 2A, Table 2B, Table 13A, Table 13B, and FIG. 19.

In one embodiment, each of the diagnostic agents of the diagnostic array is an oligonucleotide probe or an antibody.

The present invention also provides several kits. In one embodiment, a kit is provided for assessing whether a subject is afflicted with a hepatic disorder, the kit comprising reagents for assessing expression of a marker or a plurality of markers selected from the group consisting of the markers listed in Table 2A, Table 2B, Table 13A, Table 13B, and FIG. 19, and instructions for use. In another embodiment, a kit is provided comprising the diagnostic arrays of the present invention and instructions for use. In still another embodiment, a kit is provided for assessing the presence of cells having or indicative of a hepatic disorder, the kit comprising at least one nucleic acid probe wherein the probe or probes specifically bind with transcribed polynucleotides corresponding to a marker or a plurality of markers selected from the group consisting of the markers listed in Table 2A, Table 2B, Table 13A, Table 13B, and FIG. 19. In yet another embodiment, a kit is provided for assessing the presence of cells having or indicative of a hepatic disorder, the kit comprising at least one antibody, wherein the antibody or antibodies specifically bind with a marker or a plurality of markers selected from the group consisting of the markers listed in Table 2A, Table 2B, Table 13A, Table 13B, and FIG. 19. In another embodiment, a kit is provided for assessing the suitability of one or more test compounds for treating a hepatic disorder in a subject, the kit comprising one or more test compounds and a reagent for assessing expression of a marker or a plurality of markers selected from the group consisting of the markers listed in Table 2A, Table 2B, Table 13A, Table 13B, and FIG. 19.

### BRIEF DESCRIPTION OF THE DRAWINGS

FIG. 1 depicts the effect of missing gene expression signals by reducing the number of probes for each gene in the DASL assay. FIG. 1A depicts missing signals by reducing the number of probes assigned for each gene. Left panel shows expression levels of 502 cancer-related genes (Cancer Panel, Illumina) computed as average of 3 independent probes for each gene. Right panel shows signals falling below the level of negative control probes (black bars) by randomly picking a single probe from the 3 probes representing each gene. FIG. 1B depicts hierarchical clustering using 5 datasets generated by randomly picking 1 probe from the 3 probes. FIG. 1C depicts a comparison of rank of top HCC marker genes (top and bottom 20 genes) between 1-probe and 3-probe datasets.

FIG. 2 depicts statistical predictions including leave-one-out cross validation-based survival prediction using FFPE HCC tissues (FIG. 2A) and previously reported survival-predictive signature (Lee, et al. Hepatology 2004; 40:667) recapitulated in the dataset (left panel) without association with survival (right panel) (FIG. 2B).

FIG. 3 depicts statistical prediction including leave-one-out cross validation-based survival prediction using publicly available gene expression dataset of fresh frozen HCC tissues (n=67, NCBI Gene Expression Omnibus dataset accession # GSE9843) (FIG. 3A) and previously reported survival-predictive signature (Lee, et al. Hepatology 2004; 40:667) recapitulated in the dataset (left panel) without association with survival (right panel) (FIG. 3B).

FIG. 4 depicts smoothed tumor recurrence hazard over time after surgery for training (FIG. 4A) and validation (FIG. 4B) sets. There is no peak of early recurrence in training set.

FIG. 5 depicts survival curves according to the grade of hepatitis activity (based on Batts and Ludwig. Am J Surg Pathol 1995; 19:1409) in the training set.

FIG. 6 depicts overall recurrence curves in the validation set according to the prediction made by the late recurrence-predictive signature (132 genes, FIG. 6A), the overall recurrence-predictive signature (174 genes, FIG. 6B) and the correlation between survival- and late recurrence-predictive signatures (FIG. 6C): genes on microarray were rank-ordered according to their correlation with survival time, and subset of late recurrence signature genes associated with higher (upper panel) or lower (lower panel) risk of late recurrence was separately evaluated for its overrepresentation on poor survival or good survival side in the rank-ordered gene list, respectively, using Gene Set Enrichment Analysis (p<0.001). Early recurrences (<2 years following resection) are censored in the analysis of late recurrence. Red and blue lines indicate prediction of higher and lower risk of late/overall recurrence, respectively.

FIG. 7 depicts assessment of clonality between primary and recurrent tumors. FIG. 7A shows how many homozygous loci in the primary tumors appear to be heterozygous in paired recurrent tumors. FIG. 7B shows how many heterozygous loci in the primary tumors appear to be homozygous in paired recurrent tumors. DLBCL: diffuse large B-cell lymphoma.

FIG. 8 depicts gene expression signals in genome-wide microarray datasets profiling panels of multiple human tissue types. FIG. 8A shows a panel of cancer tissues (PNAS 2001; 98:1514) and FIG. 8B shows a panel of normal tissues (PNAS 2004; 101; 606). Red color indicates “present” (i.e., expressed) genes.

FIG. 9 depicts the selection process for 6,000 transcriptionally informative genes in the DASL assay. FIG. 9A: shows that in each of previously generated 24 microarray datasets, coefficient of variation (CV) was calculated for each gene and summarized on to the list of NCBI RefSeq ID. FIG. 9B shows that the top 6,000 genes cover 70-90% of genes in microarray-based signatures (375 gene sets) and literature-based molecular pathways (450 gene sets) collected in Molecular Signature Database (MSigDB). FIG. 9C shows the age of FFPE blocks and % P-call in 10 prostate cancer samples. Red arrow head indicates samples fixed 24 years before RNA extraction; blue arrow head indicates a sample fixed 7 years before RNA extraction.

FIG. 10 depicts quality assessment of DASL profile based on the proportion of “present” (i.e., expressed) genes (% P-call) in the training set. Correlation coefficient of each array to the “median” array was plotted against % P-call for tumor (FIG. 10A) and adjacent liver (FIG. 10B) profiles from the training set. For each tissue type, quality threshold was defined as a % P-call where the correlation starts to drop. Vertical lines in graph indicate % P-call threshold of 65% and 70% for tumor and liver profiles, respectively. The same quality threshold was applied to the profiles from validation set.

FIG. 11 depicts a comparison of gene expression fold change between intact and FFPE-RNA.

FIG. 12 depicts a prediction of prostate cancer using the DASL profile of marker genes defined by a meta-analysis of published 7 frozen sample-based microarray datasets.

FIG. 13 depicts the survival signature in a publicly available independent dataset of fresh frozen non-tumor liver tissues (n=10).

FIG. 14 depicts survival curves for three geographic sites in the validation set: US (n=88, median follow-up 2.4 years), Spain (n=45, median follow-up 3.1 years), and Italy (n=92, median follow-up 1.9 years). FIG. 14A shows overall survival and FIG. 14B shows survival curves according to the survival prediction. Red lines indicate poor survival prediction; blue lines indicate good survival prediction.

FIG. 15 depicts the design of the study. In the training set, tumor tissue and liver tissue adjacent to the tumor were profiled separately, and each was used to generate an outcome model. The model based on adjacent liver tissue was validated with the use of an independent validation set.

FIG. 16 depicts survival signatures and survival curves in the training set. Survival curves are shown for survival according to the association of the gene signature with survival, based on leave-one-out cross-validation testing (FIG. 16A) for overall survival according to the level of expression of the 186 signature genes (FIG. 16B). FIG. 16C shows the expression pattern of the survival signature (comprising 186 genes). The 20 genes most closely associated with a poor prognosis are listed on the left, and the 20 most closely associated with a good prognosis on the right. Red indicates high expression; blue indicates low expression, and FIG. 16D shows representative photomicrographs of sections of liver tissue adjacent to tumor that were profiled in this study; there were no histologic correlates with survival. Staining was with hematoxylin and eosin.

FIG. 17 depicts survival signatures and survival curves in the validation set. FIG. 17A shows the expression pattern of the 186-gene survival signature. Red indicates a poor prognosis; blue indicates a good prognosis. Survival curves are shown for overall survival according to the level of expression of the 186 signature genes among all 225 patients whose tissue samples constituted the validation set (FIG. 17B) and among the 168 patients with a longer duration of follow-up (treated no later than 2004) (FIG. 17C). FIG. 17D shows the probability of late recurrence according to the level of expression of the late recurrence gene signature.

FIG. 18 depicts hazard ratios for poor survival and late recurrence in selected subgroups of patients in the validation set. The hazard ratio was for poor survival among patients with the poor-prognosis gene signature (FIG. 18A) or for late recurrence (FIG. 18B) among patients with the late-recurrence gene signature, as compared with those without the signature. BCLC denotes Barcelona Clinic Liver Cancer staging system, which ranks hepatocellular carcinoma in five stages, ranging from 0 (very early stage) to D (terminal stage).

FIG. 19 depicts the probe identification, gene identification, gene symbol, gene description, and primer sequence information regarding the DASL platform.

FIG. 20 depicts a schematic of the study design in which fine needle liver biopsy specimens collected from a prospectively followed patient cohort were subjected to whole-genome gene-expression profiling.

FIG. 21 depicts survival curves for overall survival according to the level of expression of the 186-gene survival signature among all of the 276 patients.

FIG. 22 depicts hazard ratios for overall survival, hepatic decompensation, and hepatocellular carcinoma development in selected subgroups of patients. The hazard ratio was for poor survival (FIG. 22A), hepatic decompensation (FIG. 22B), or hepatocellular carcinoma development (FIG. 22C) among patients with the poor-prognosis gene signature as compared with those without the signature.

FIG. 23 depicts an estimation of survival benefit of chemopreventive therapy according to signature-based prediction. FIG. 23A shows a Markov model of survival based on survival curves presented in FIG. 21. FIG. 23B shows life years gained by chemopreventive therapy in which the vertical line in the graph indicates the hazard ratio achieved by interferon therapy reported by Nishiguchi, S. et al. (2001) Lancet 357, 196-197.

FIG. 24 depicts the correlation between prognosis and induced cirrhosis.

FIG. 25 depicts the correlation between prognosis and cancer-preventive effects of erlotinib.

### BRIEF DESCRIPTION OF THE TABLES

Table 1 depicts univariate Cox regression of clinical variables for patient survival in the training set.

Table 2 depicts survival signature genes (genes correlated with poor survival in Table 2A and genes correlated with good survival in Table 2B) defined in adjacent liver tissue as defined in the training set.

Table 3 depicts functional annotation of survival signature by gene set enrichment analysis in the training set.

Table 4 depicts gene expression-based survival prediction and histological inflammation of the liver in the training set.

Table 5 depicts univariate Cox regression analysis of clinical risk factors in the validation set.

Table 6 depicts multivariate Cox regression subgroup analysis in the validation set.

Table 7 depicts clonality analysis of paired primary and recurrent HCC (Table 7A) and clonality analysis of paired primary and recurrent/metastatic non-HCC tumors (Table 7B).

Table 8 depicts datasets used to select transcriptionally informative genes.

Table 9 depicts concordance in gene expression change (DHL4 vs. Hela cell lines) between intact and FFPE-RNA in the DASL assay.

Table 10 depicts leave-one-out cross-validation error rates for outcome prediction using HCC tissue data in the training set.

Table 11 depicts characteristics of patients in the training set and in the validation set at the time of surgery.

Table 12 depicts associations of gene-expression signatures and clinical variables with late recurrence or overall survival, from multivariate analysis of validation set.

Table 13 depicts late-recurrence signature genes (genes correlated with higher late-recurrence in Table 13A and genes correlated with lower late-recurrence in Table 13B) defined in adjacent liver tissue as defined in the training set.

Table 14 depicts a summary of clinical characteristics for the patient cohort at the time of enrollment.

Table 15 depicts associations of a 186-gene survival signature and clinical variables with clinical outcome (univariate analysis).

Table 16 depicts associations of a 186-gene survival signature and clinical variables with clinical outcome (multivariate analysis).

Table 17 depicts associations of a 186-gene survival signature and clinical variables with clinical outcome in Child-Pugh class A and Hepatitis C infection (multivariate subgroup analysis).

Table 18 depicts associations of a 186-gene survival signature with non-cancer-related death (multivariate analysis).

Table 19 depicts gene sets associated with clinical outcome (high risk of hepatocellular carcinoma development, Table 19A; low risk of hepatocellular carcinoma development, Table 19B; poor survival, Table 19C, and good survival, Table 19D) by gene set enrichment analysis.

Table 20 depicts associations of a 186-gene survival signature and clinical variables with overall survival (multivariate analysis), age, esophageal/gastric varices, and albumin.

Table 21 depicts associations of a 186-gene survival signature and clinical variables with overall survival (multivariate analysis) and MELD score.

Table 22 depicts associations of a 186-gene survival signature and hepatitis C-related clinical variables with clinical outcome (univariate and multivariate analysis).

Table 23 depicts associations of a 186-gene survival signature and clinical variables with ascites or gastrointestinal bleeding (multivariate analysis).

Table 24 depicts associations of a 186-gene survival signature and hepatocellular carcinoma development according to Baveno IV stage (multivariate subgroup analysis).

### DETAILED DESCRIPTION OF THE INVENTION

The present invention is based, at least in part, on methods and compositions related to a novel gene-expression profiling assay involving fixed embedded tissue, an innovative complementary DNA-mediated annealing, selection, extension, and ligation (DASL) assay, and a novel microarray. Furthermore, the present invention is based, in part, on informative genes useful in applications related to treatment, diagnosis, and prognosis of the clinical outcome of hepatic disorders (e.g., hepatocellular carcinoma and/or cirrhosis), including novel methods for prognosing subjects to stratify those with increased risk of multi-centric recurrence from among early stage cancer patients, including hepatocellular carcinoma patients.

Various aspects of the invention are described in further detail in the following subsections.

### I. DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “hepatic disorder” and/or “liver disorder” and/or a related phrase refers to conditions related to the liver, such as alcoholic cirrhosis, alpha-1 antitypsin deficiency, autoimmune cirrhosis, cryptogenic cirrhosis, fulminant hepatitis, hepatitis B and C, and steatohepatitis, biliary tract disorders, cystic fibrosis, primary biliary cirrhosis, sclerosing cholangitis, biliary obstruction, and cancer (e.g., hepatic carcinoma). Other well-known hepatic disorders can be found in the prior art, e.g., Wiesner, R. H, Current Indications, Contra Indications and Timing for Liver Transplantation (1996), in Transplantation of the Liver, Saunders (publ.); Busuttil, R. W. and Klintmalm, G. B. (eds.) Chapter 6; Klein, A. W., (1998) Partial Hypertension: The Role of Liver Transplantation, Musby (publ.) in Current Surgical Therapy 6.sup.th Ed. Cameron, J. (ed) for more specific disclosure relating to relevant hepatic disorders.

The terms “tumor” or “cancer” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, gastrointestinal cancers, e.g., colorectal, anal, esophageal, gallbladder, gastric, liver, pancreatic, and small intestine cancers, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like.

The term “hepatocellular cancer” as used herein, is meant to include primary malignancies of the liver. “Multi-centric recurrence” or “late recurrence” refers to subsequent liver tumor development after removal of an earlier liver tumor. In particular, it may include de novo tumor development owing to a diseased liver even after complete removal of an early stage tumor.

The term “altered amount” of a marker or “altered level” of a marker refers to increased or decreased copy number of a marker or chromosomal region and/or increased or decreased expression level of a particular marker gene or genes in an experimental sample, as compared to the expression level or copy number of the marker in a control sample. The term “altered amount” of a marker also includes an increased or decreased protein level of a marker in an experimental sample, as compared to the protein level of the marker in a control sample. In addition, the term “altered amount” of a marker also includes an increased or decreased nucleic acid level of a marker, e.g., a messenger RNA or microRNA in a sample, e.g., an experimental sample, as compared to the nucleic acid level of the marker in a control sample.

The amount of a marker, e.g., expression or copy number of a marker, or protein level of a marker, in a subject or sample is “significantly” higher or lower than that of a control, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject or sample can be considered “significantly” higher or lower than that of a control if the amount is at least about two, and preferably at least about three, four, or five times, higher or lower, respectively, than the normal amount of the marker.

The “copy number of a gene” or the “copy number of a marker” refers to the number of DNA sequences in a cell encoding a particular gene product. Generally, for a given gene, a mammal has two copies of each gene. The copy number can be increased, however, by gene amplification or duplication, or reduced by deletion.

The “normal” copy number of a marker or “normal” level of expression of a marker is the level of expression or copy number of the marker in a biological sample, e.g., a sample containing tissue, whole blood, serum, plasma, buccal scrape, saliva, cerebrospinal fluid, urine, stool, and bone marrow, from a subject, e.g., a human, not afflicted with cancer.

The term “altered level of expression” of a marker refers to an expression level of a marker in a test sample e.g., a sample derived from a patient suffering from cancer, that is greater or less than the standard error of the assay employed to assess expression or copy number, and is preferably at least twice, and more preferably three, four, five or ten or more times the expression level or copy number of the marker in a control sample (e.g., sample from a healthy subjects not having the associated disease) and preferably, the average expression level or copy number of the marker in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subjects not having the associated disease) and preferably, the average expression level or copy number of the marker in several control samples.

An “overexpression” or “significantly higher level of expression or copy number” of a marker refers to an expression level or copy number in a test sample that is greater than the standard error of the assay employed to assess expression or copy number, and is preferably at least twice, and more preferably three, four, five or ten or more times the expression level or copy number of the marker in a control sample (e.g., sample from a healthy subject not afflicted with cancer) and preferably, the average expression level or copy number of the marker in several control samples.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) and preferably, the average expression level or copy number of the marker in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker or altered interaction with transcriptional activators or inhibitors, or altered methylation status.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

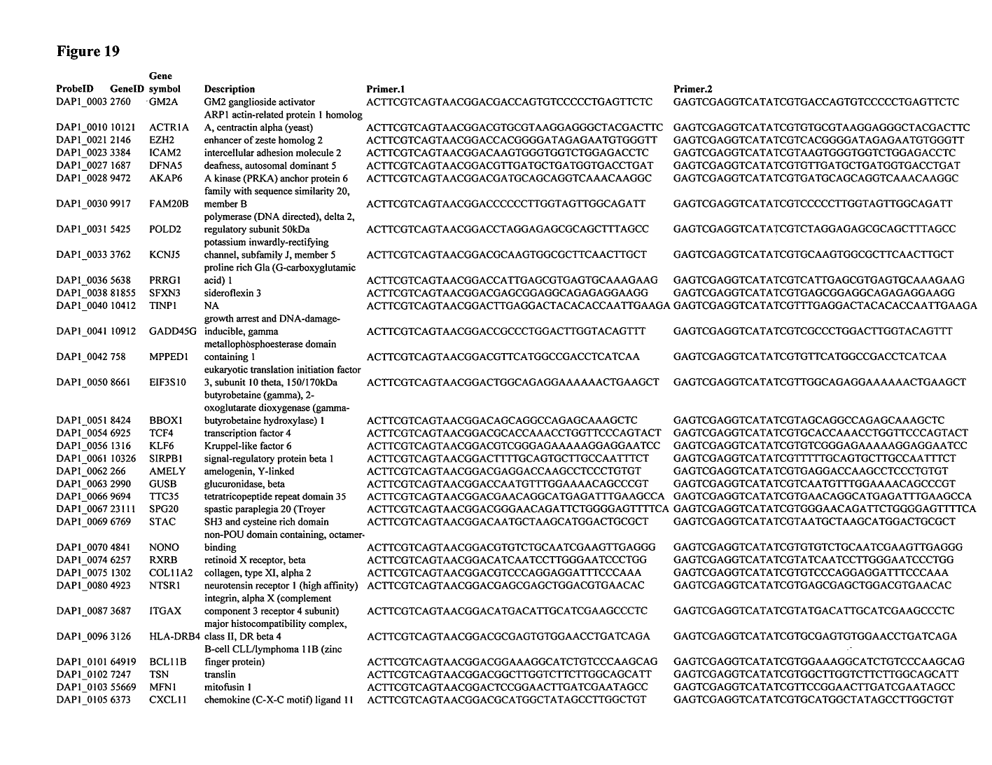

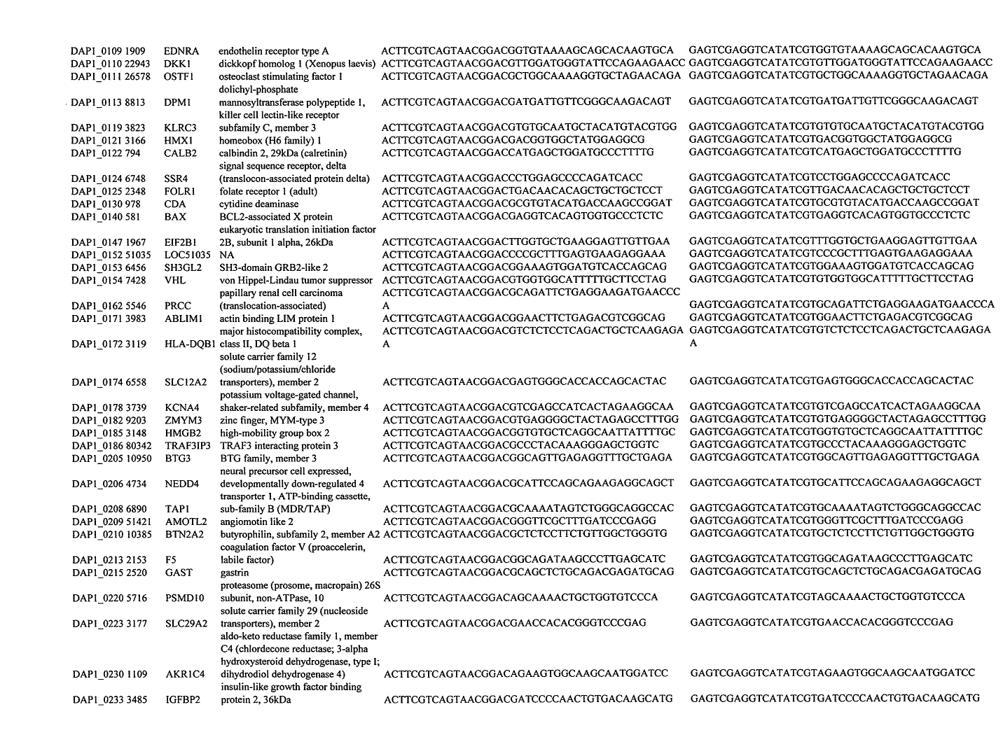

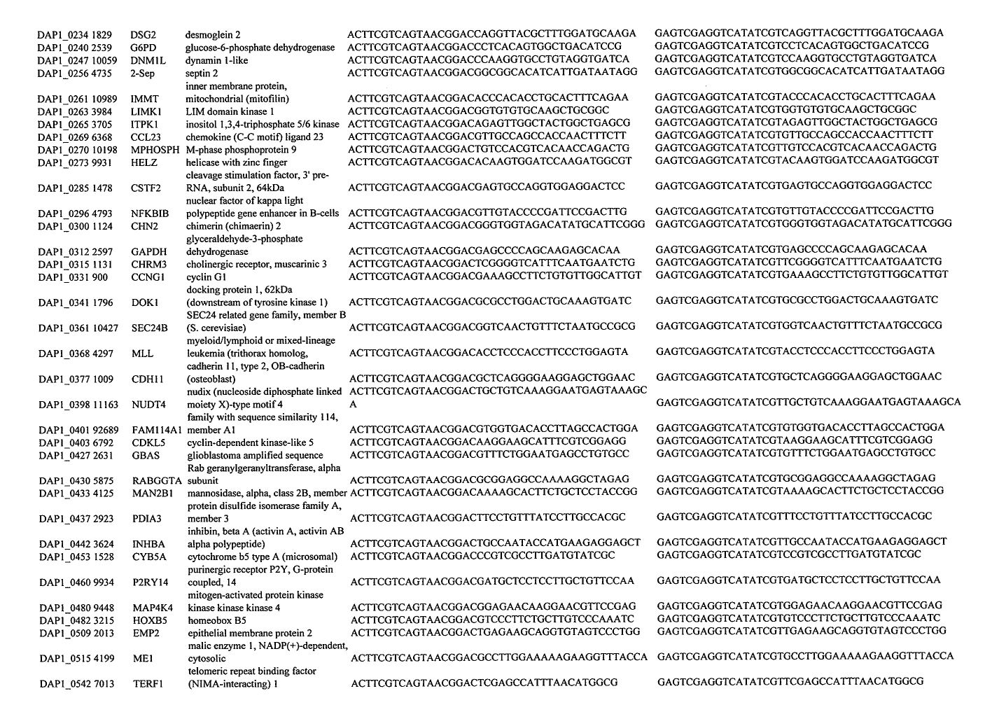

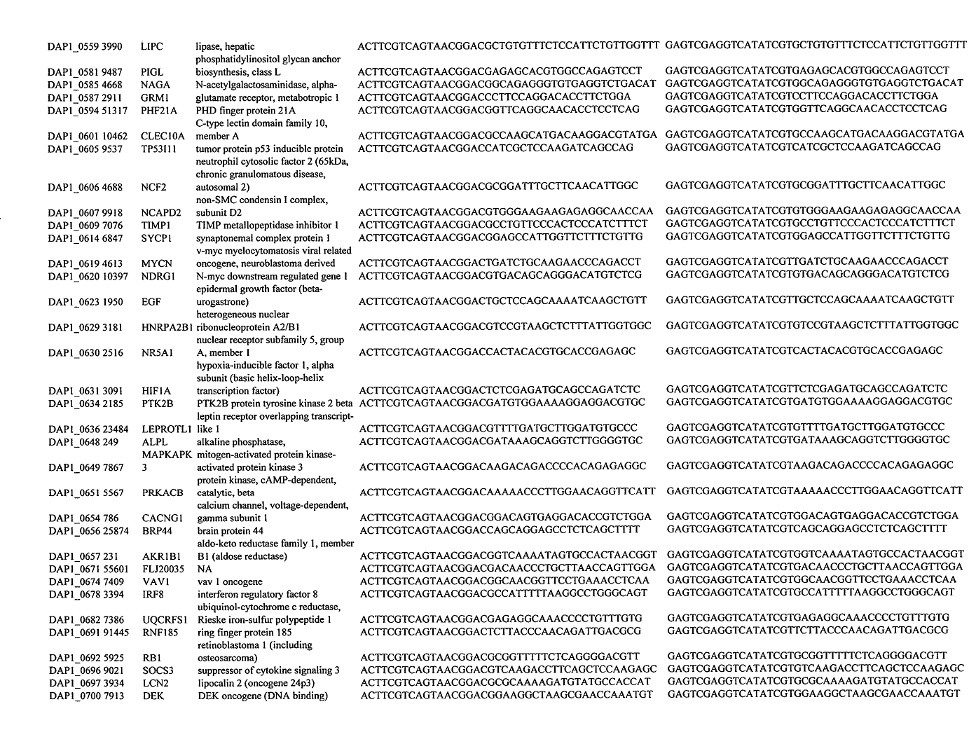

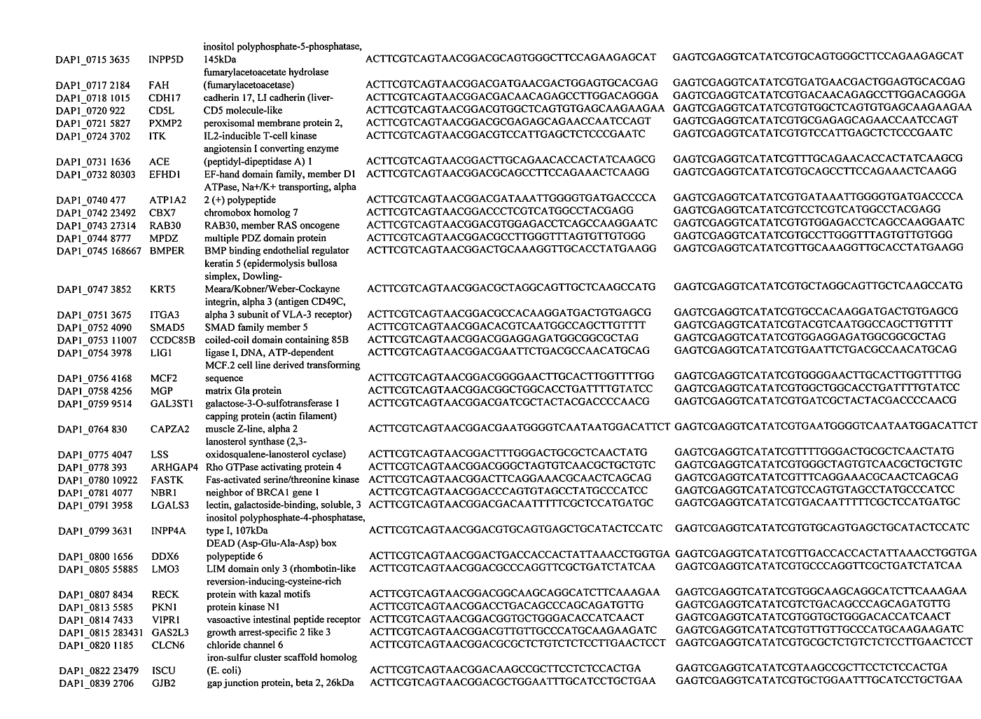

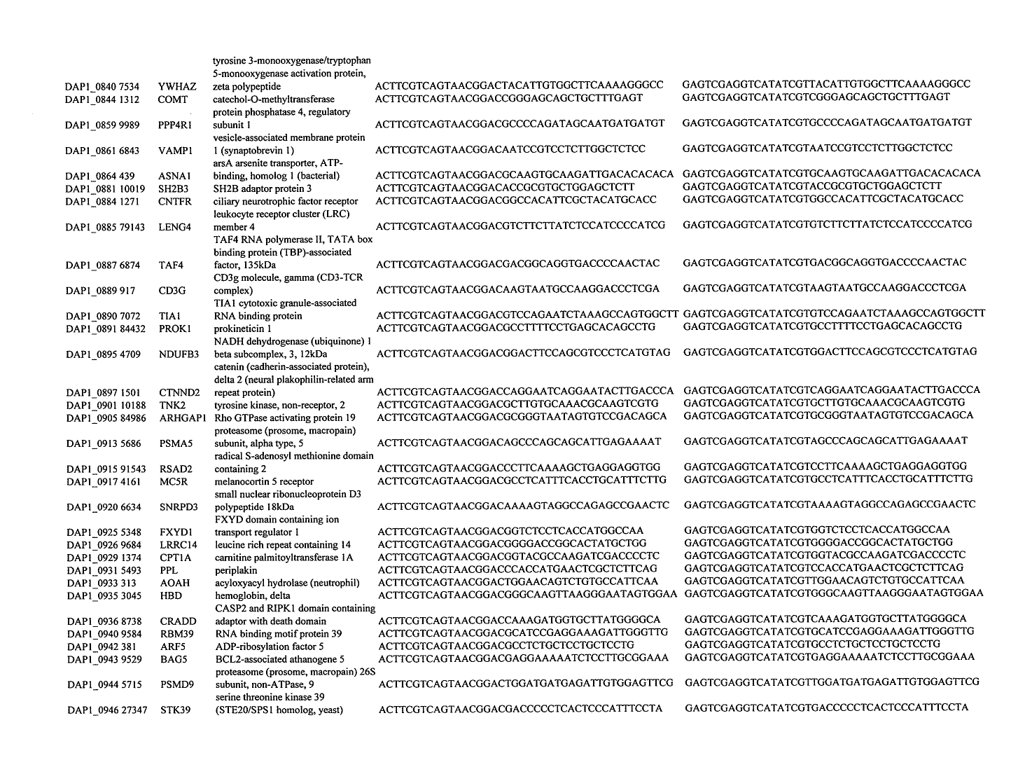

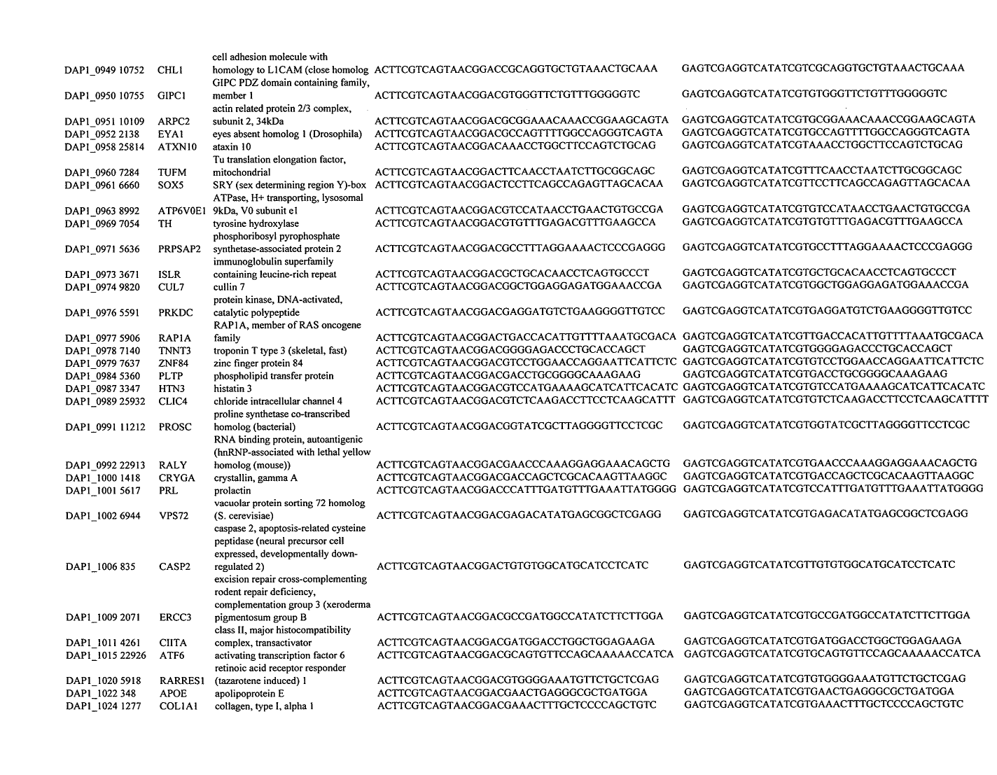

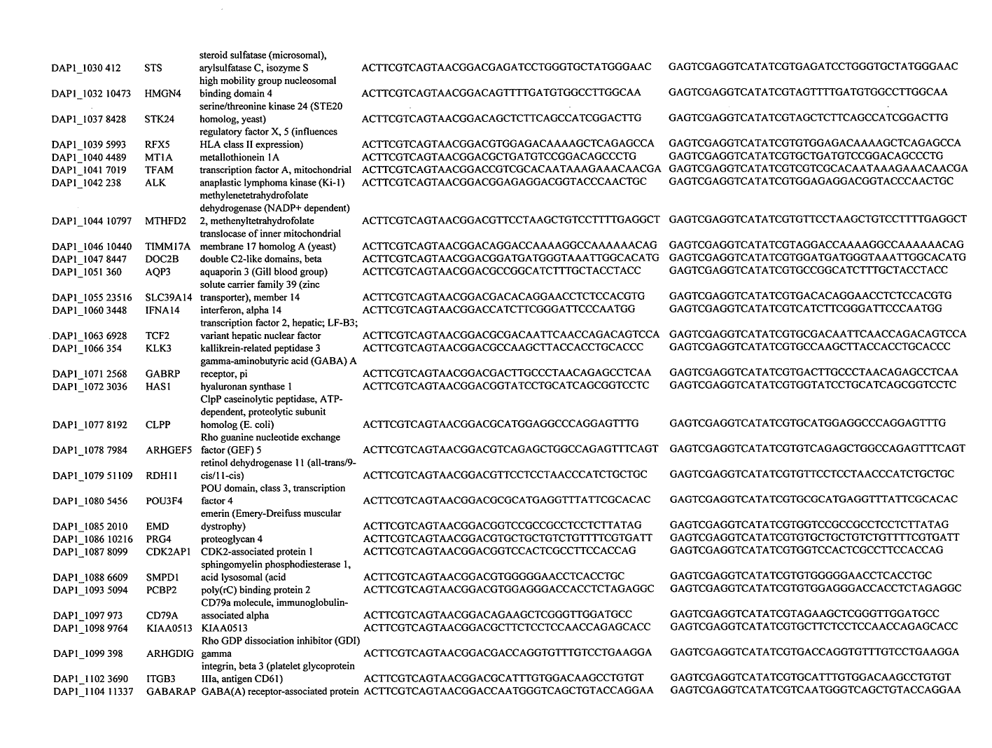

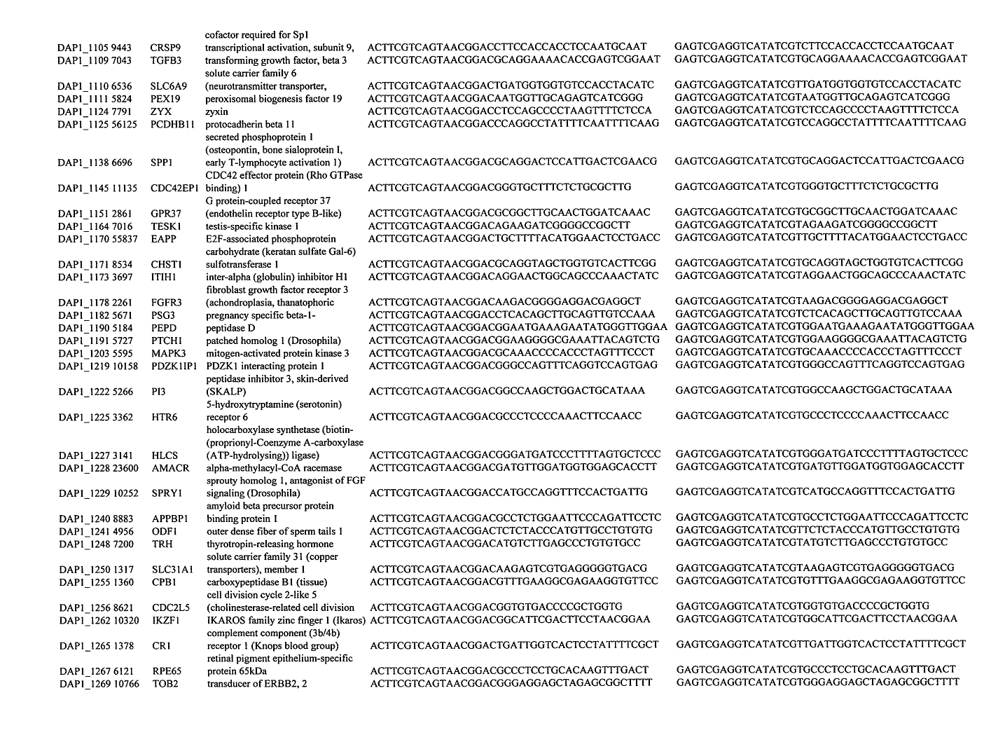

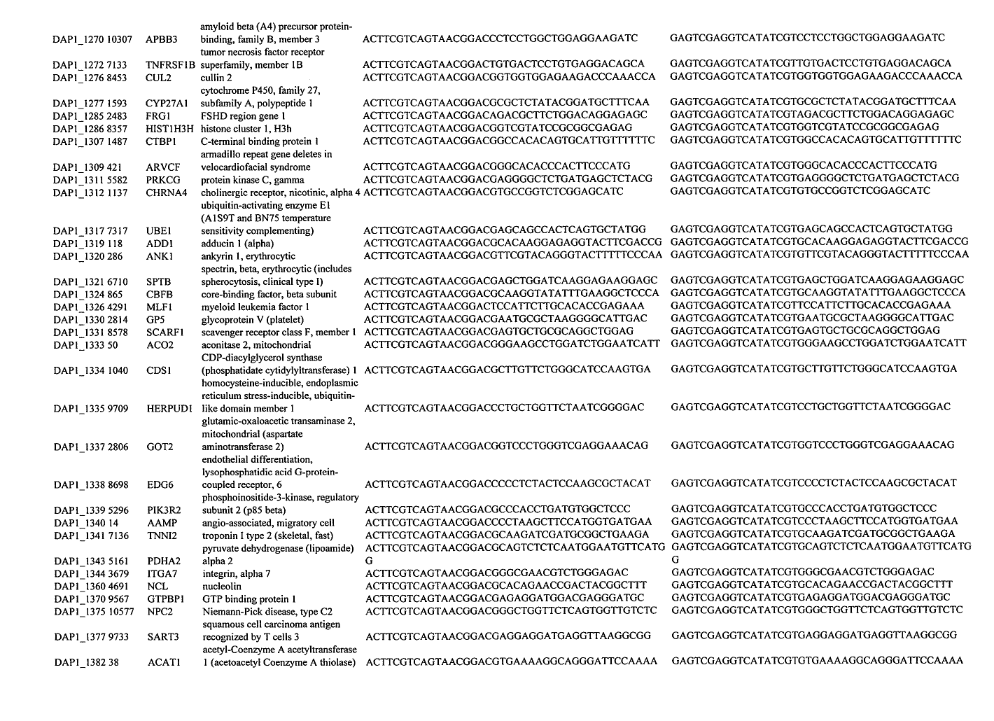

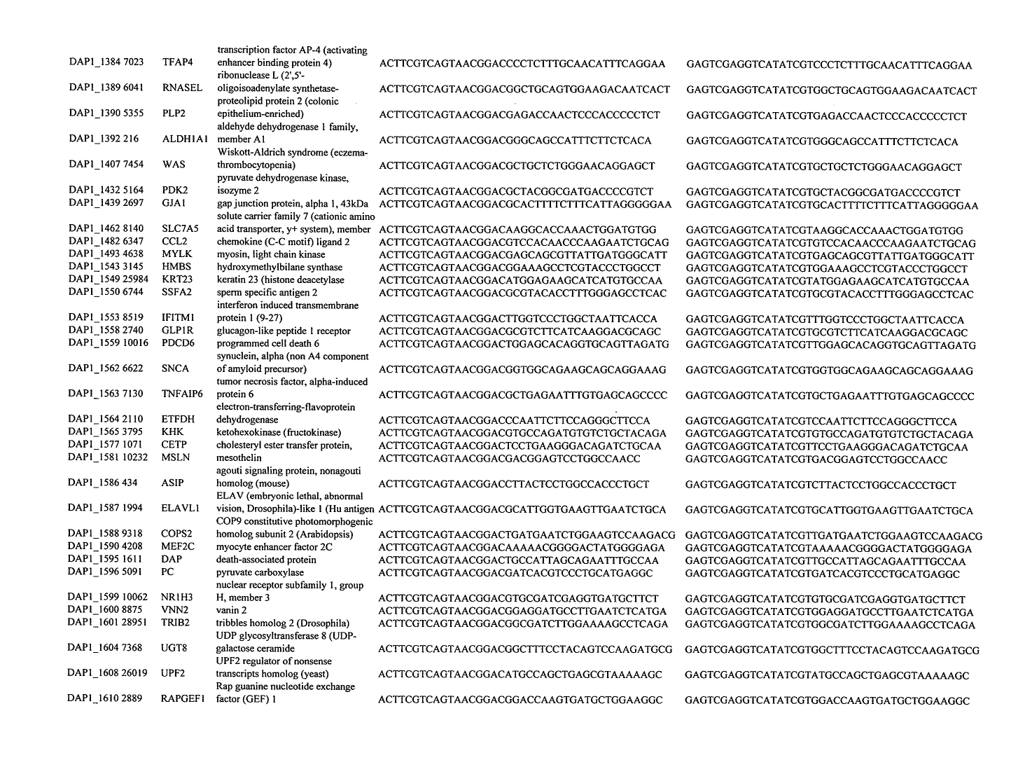

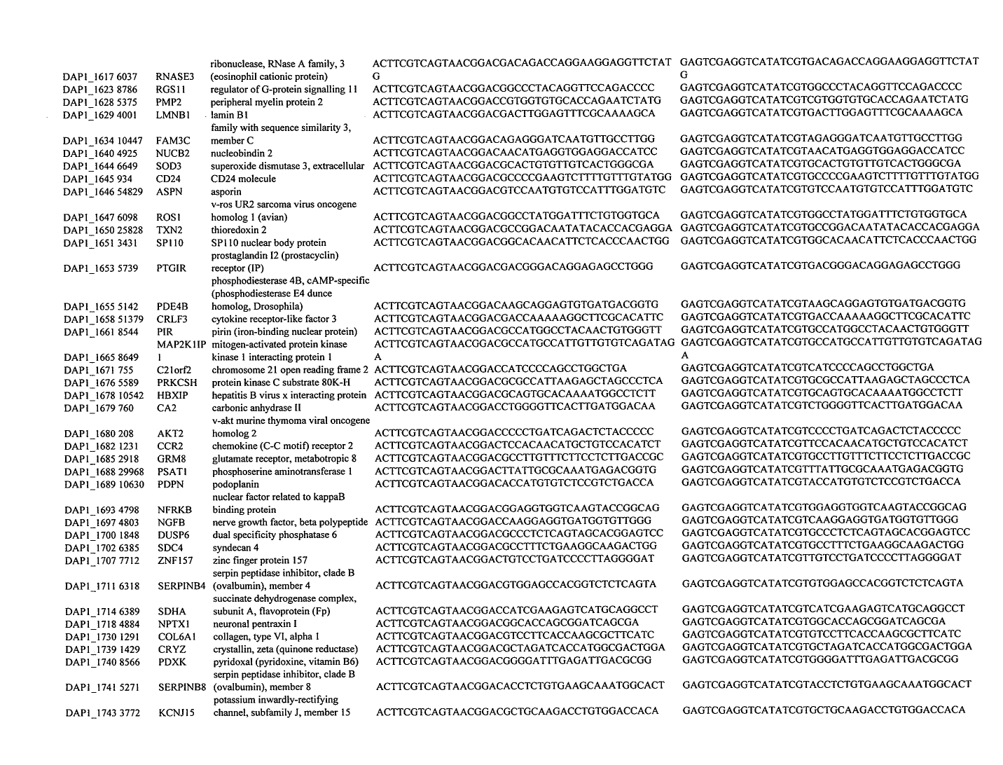

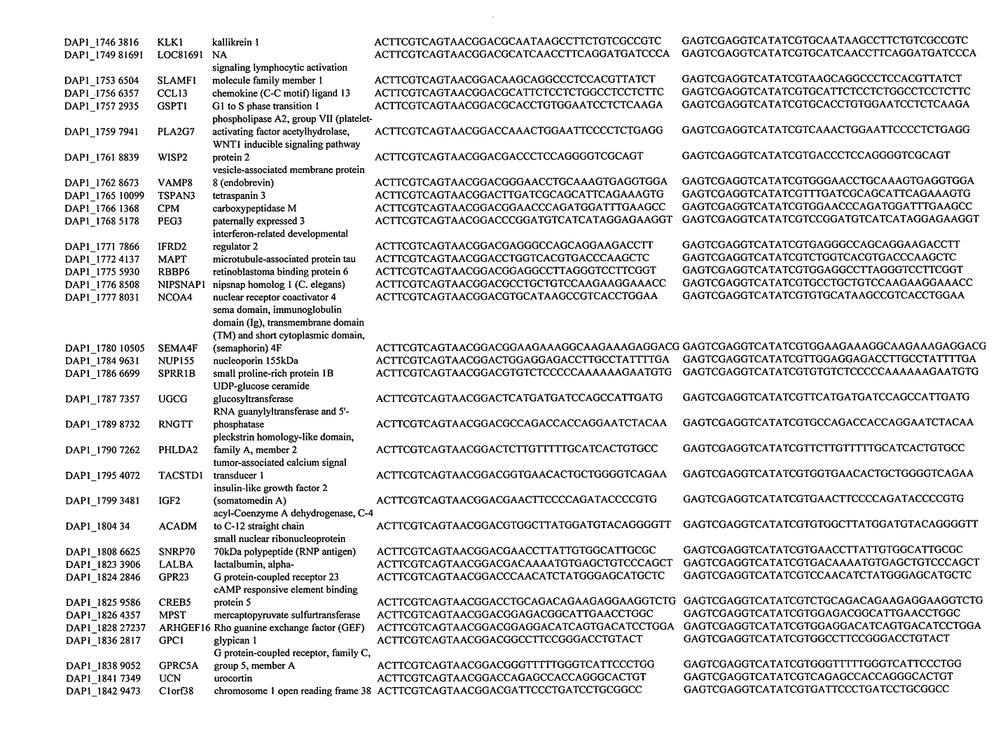

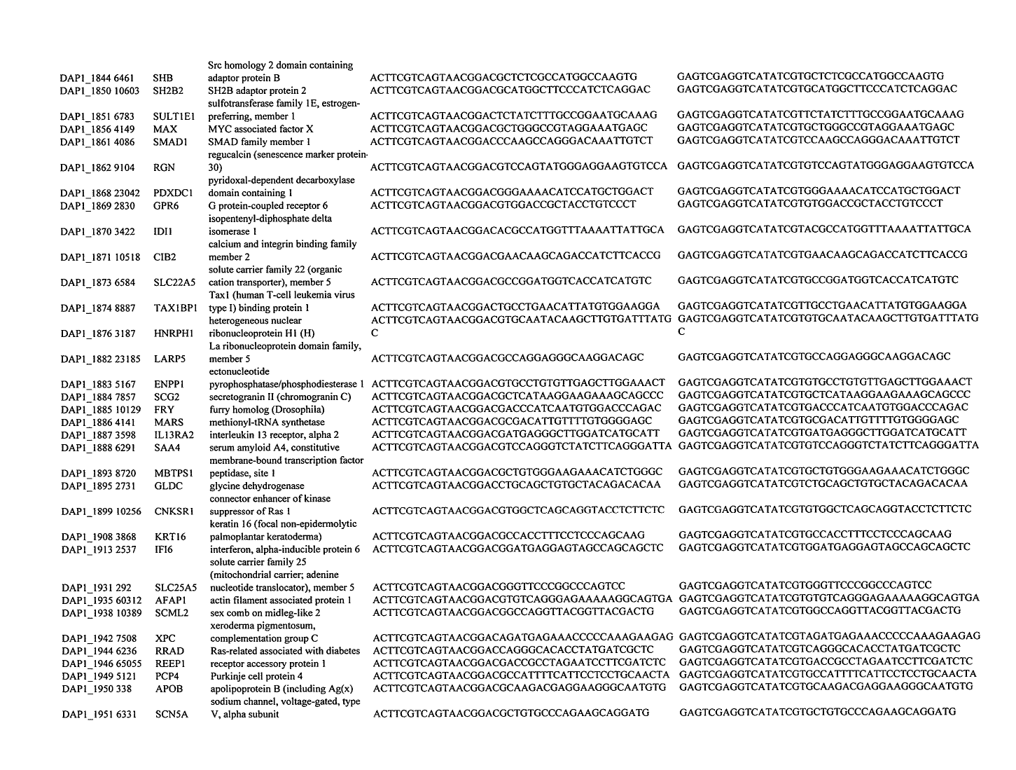

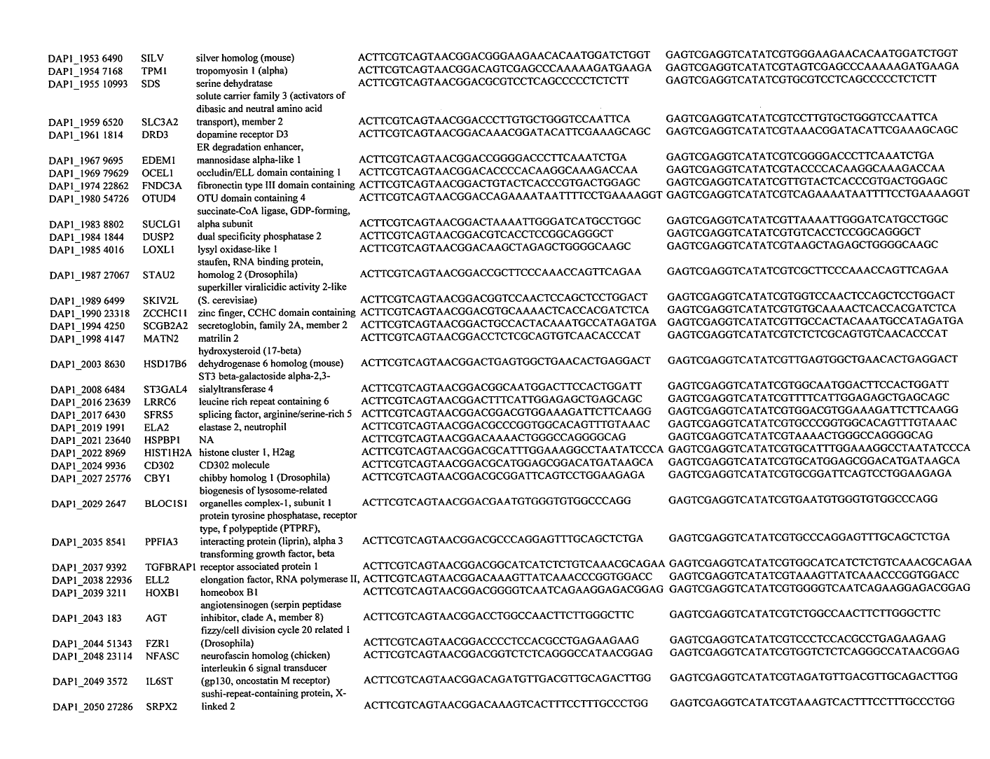

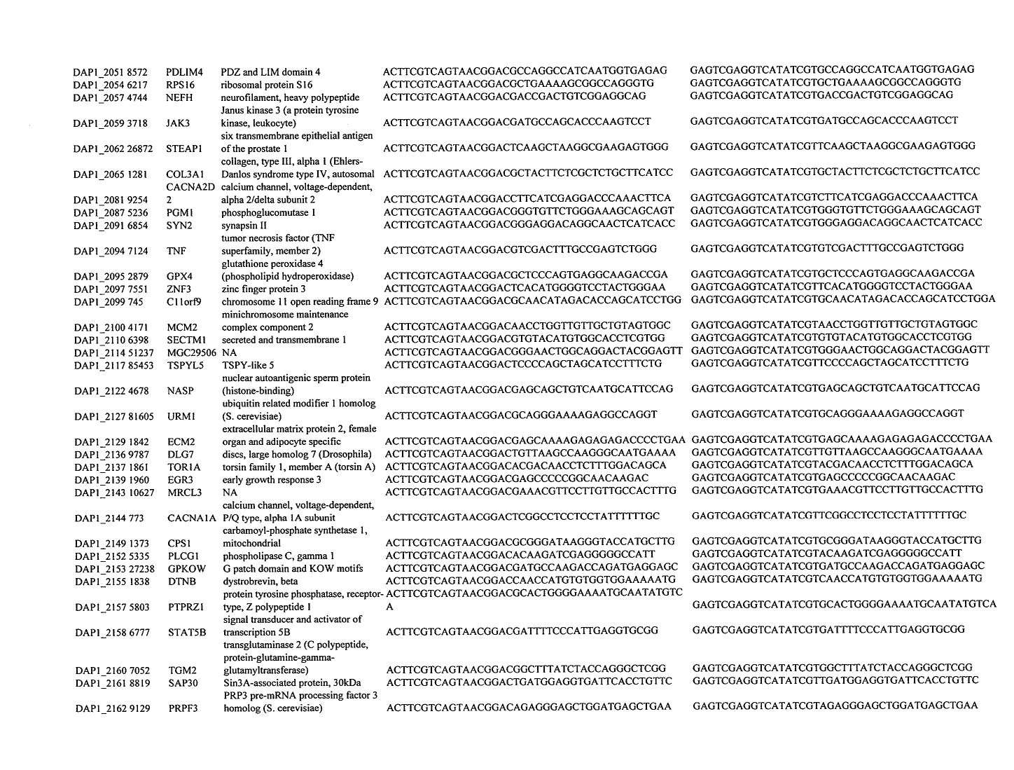

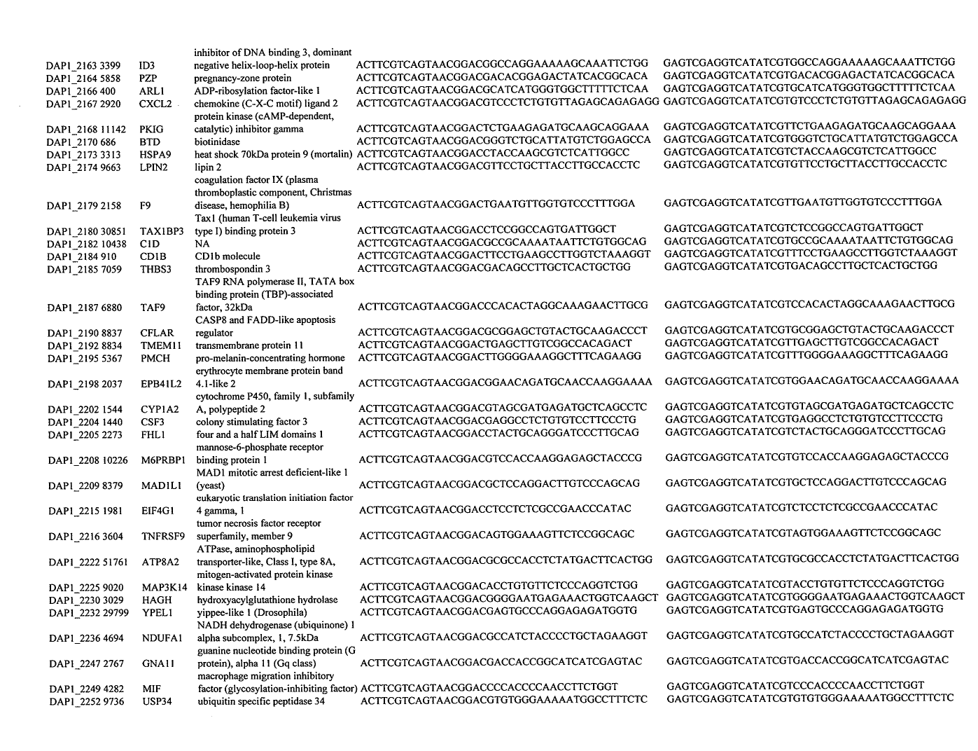

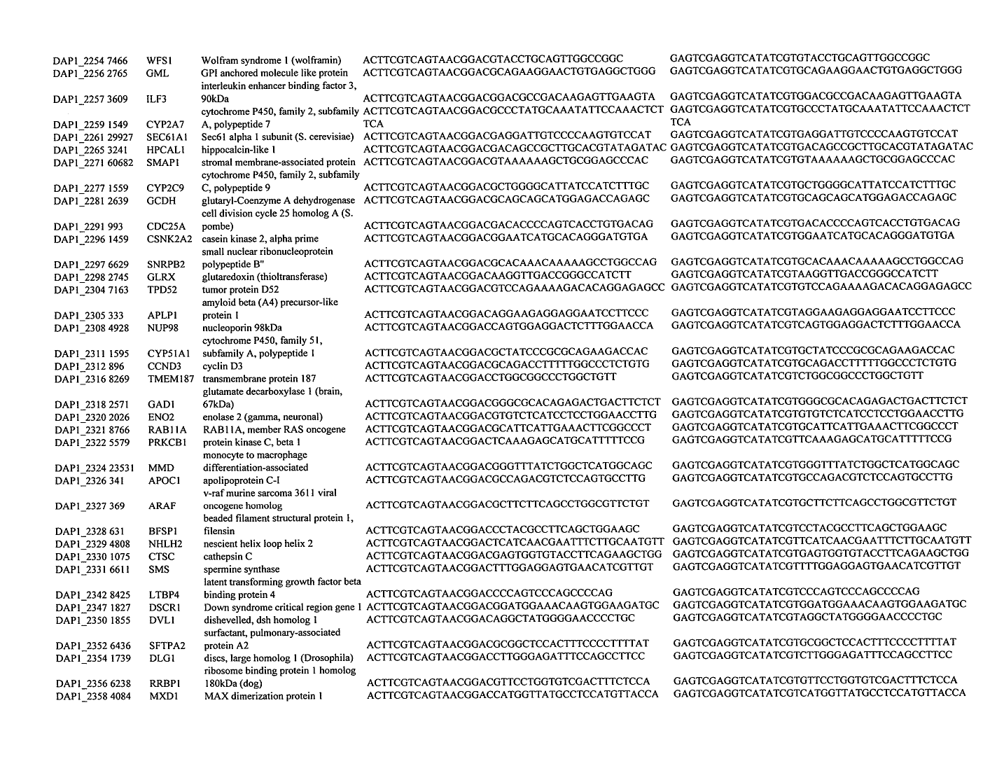

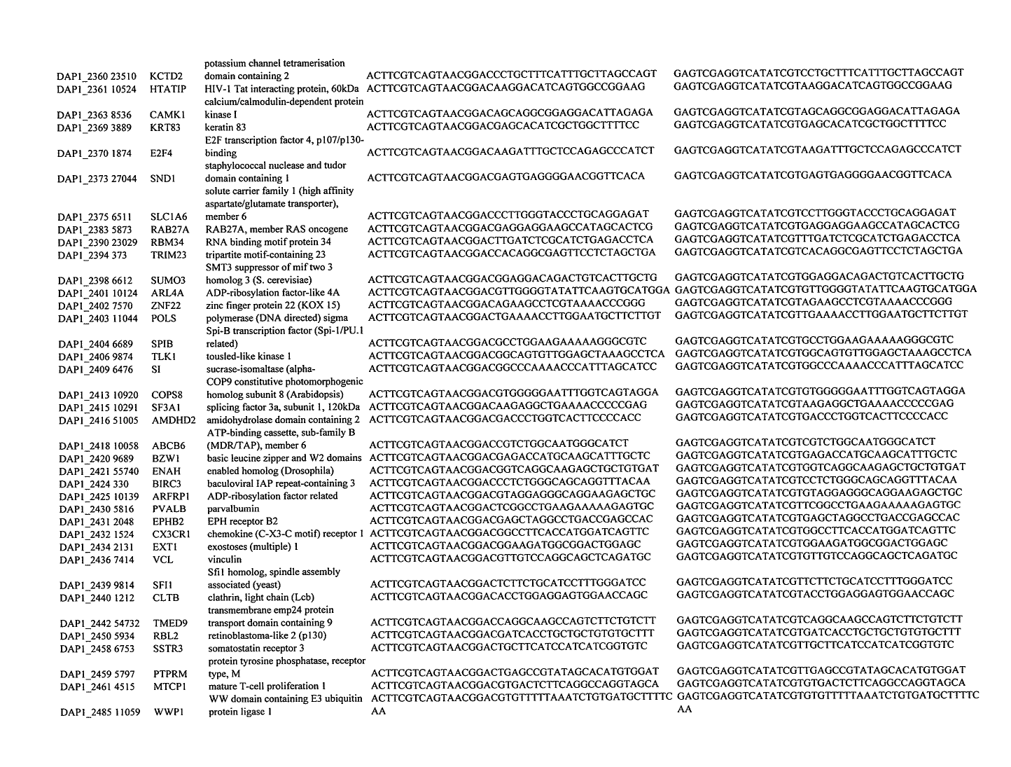

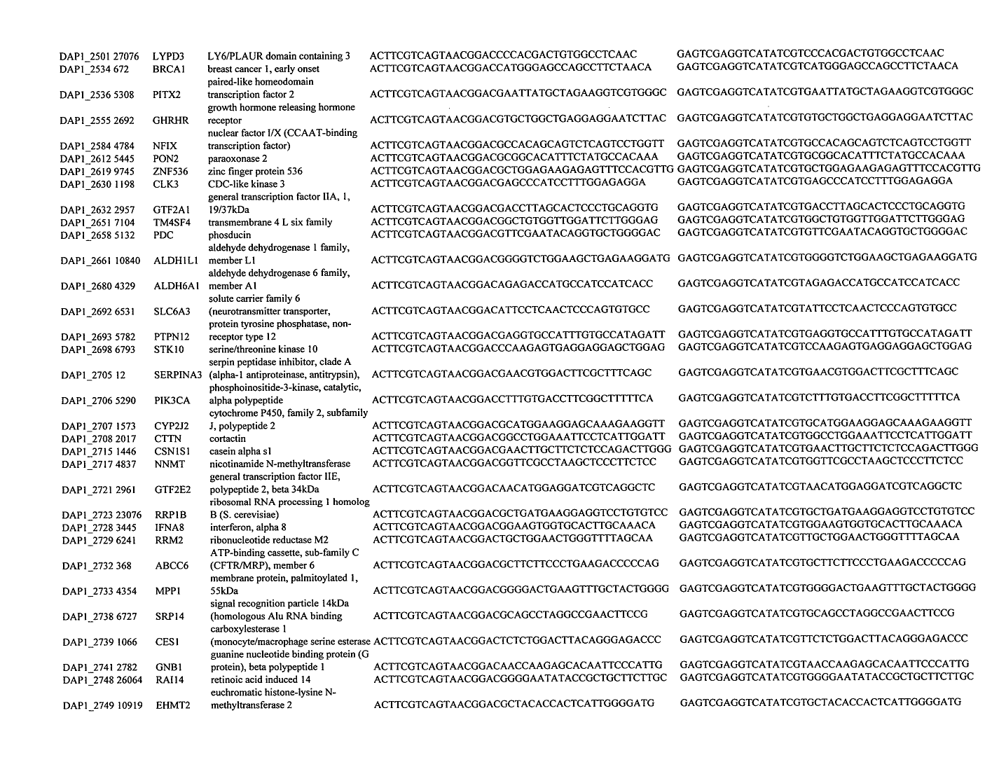

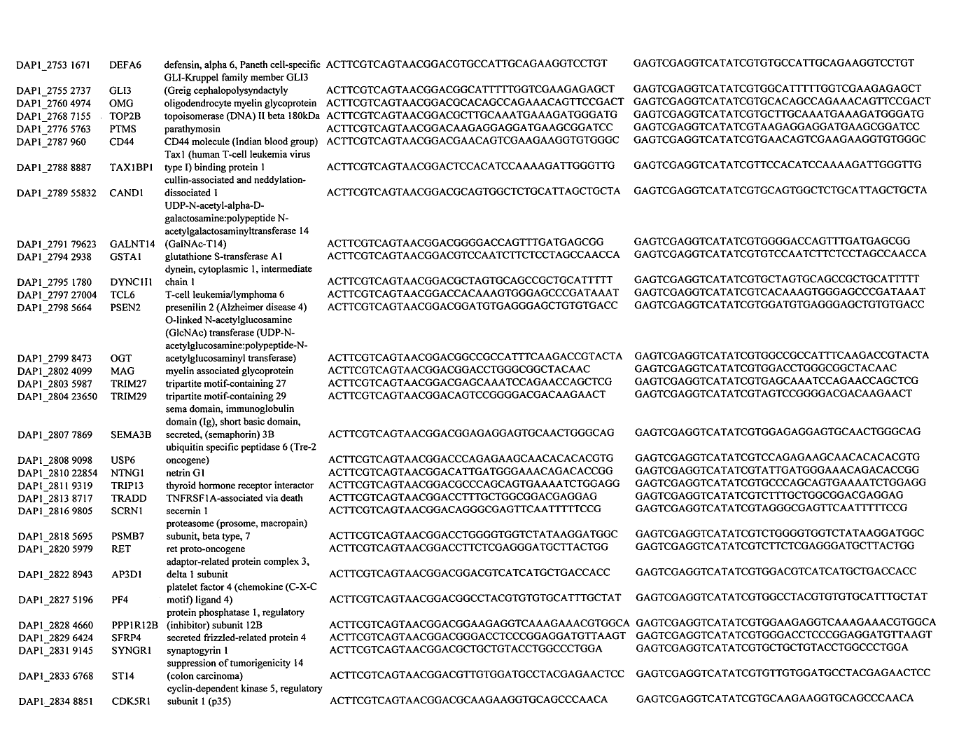

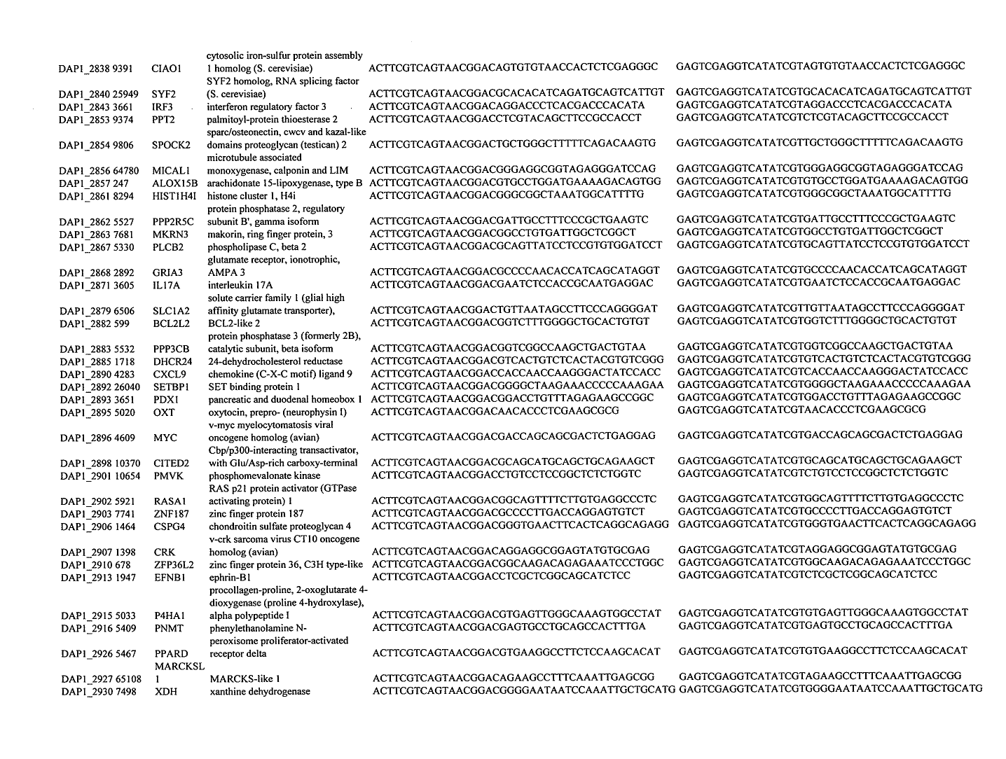

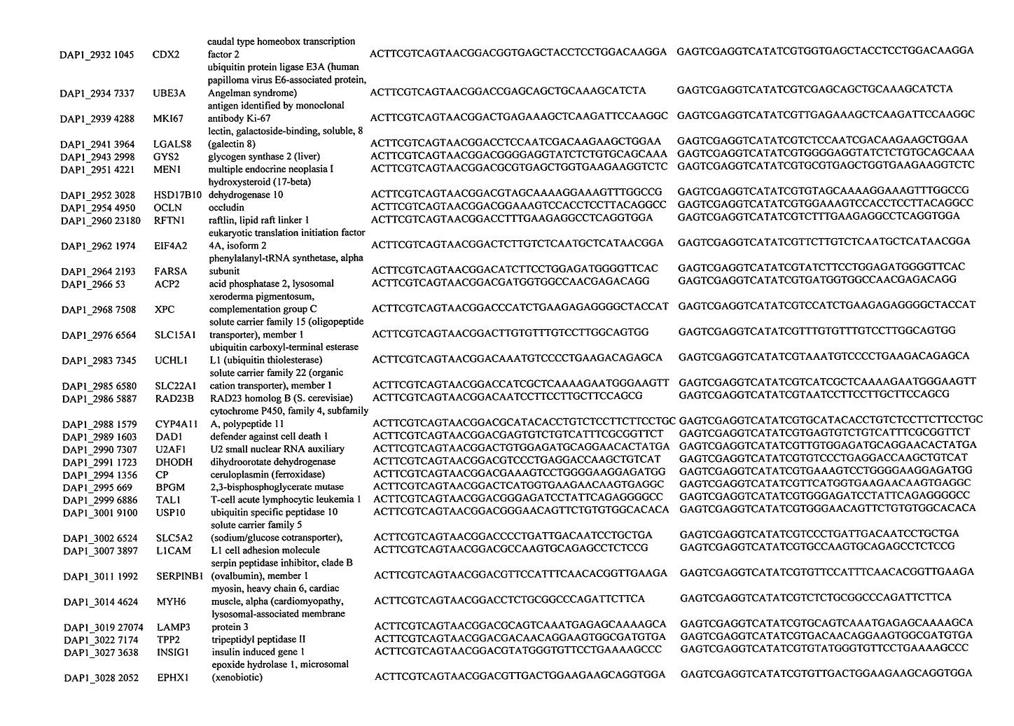

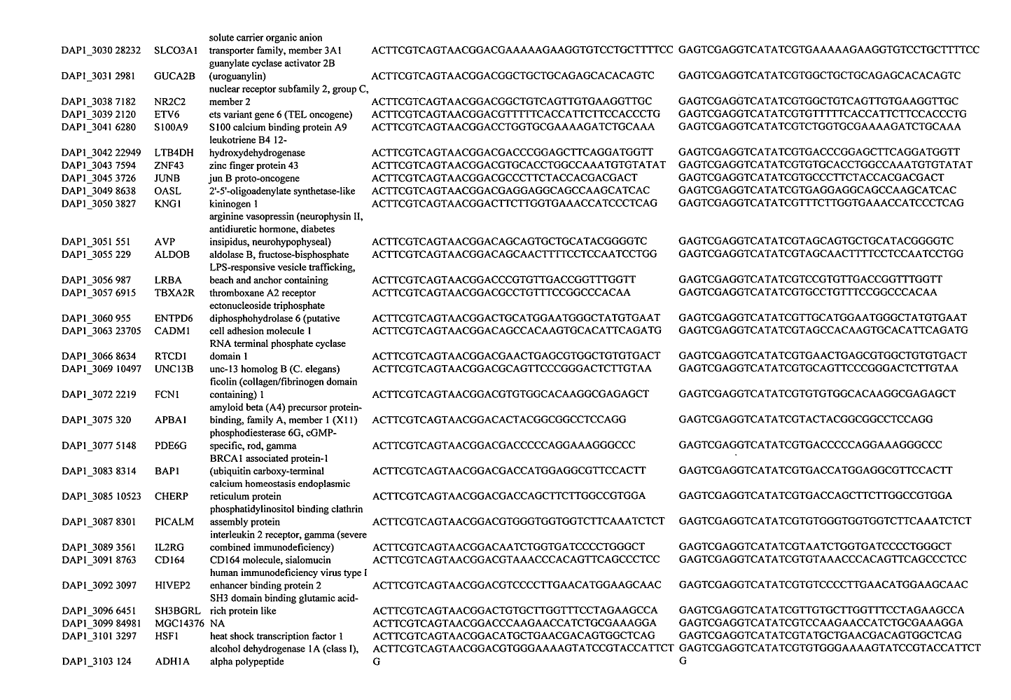

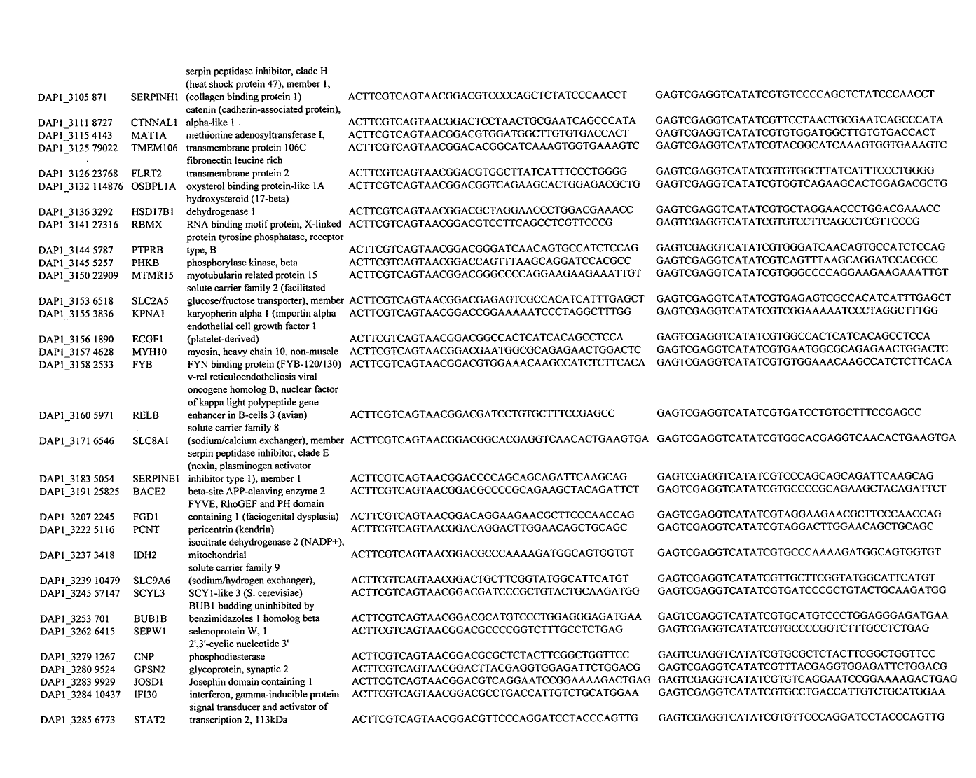

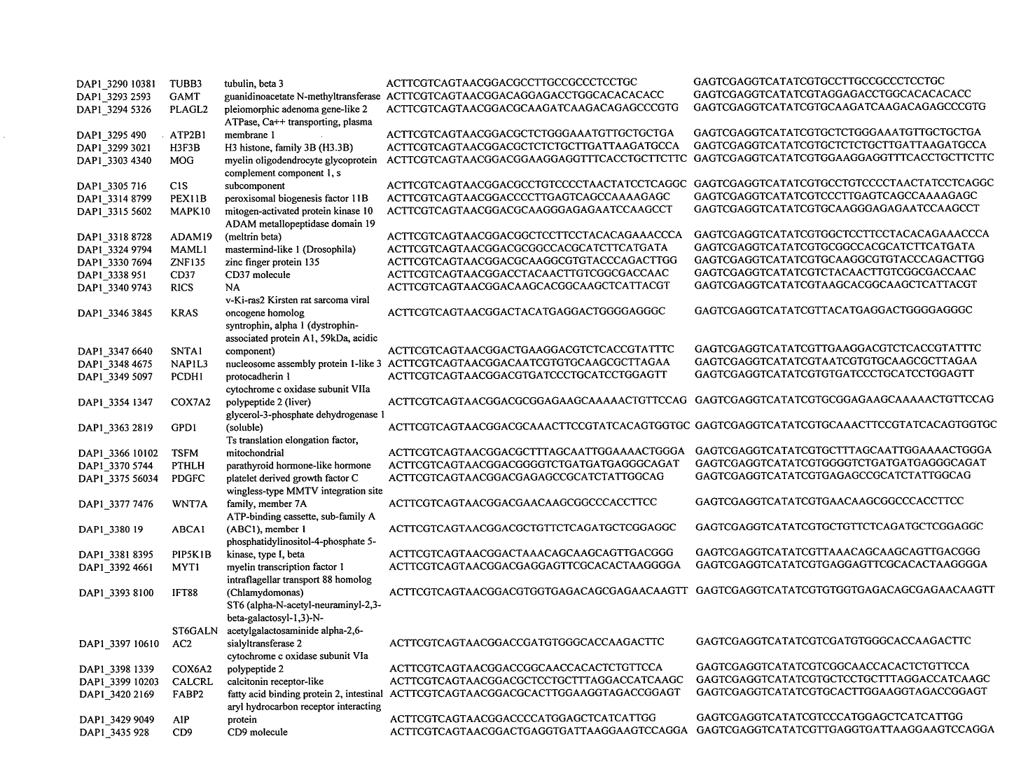

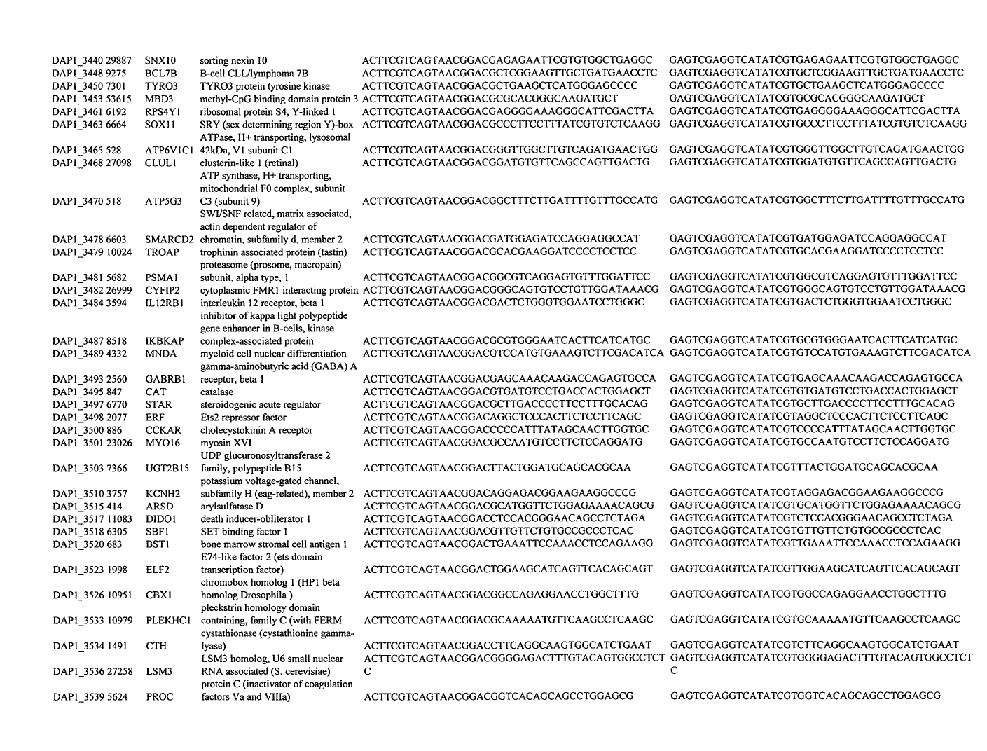

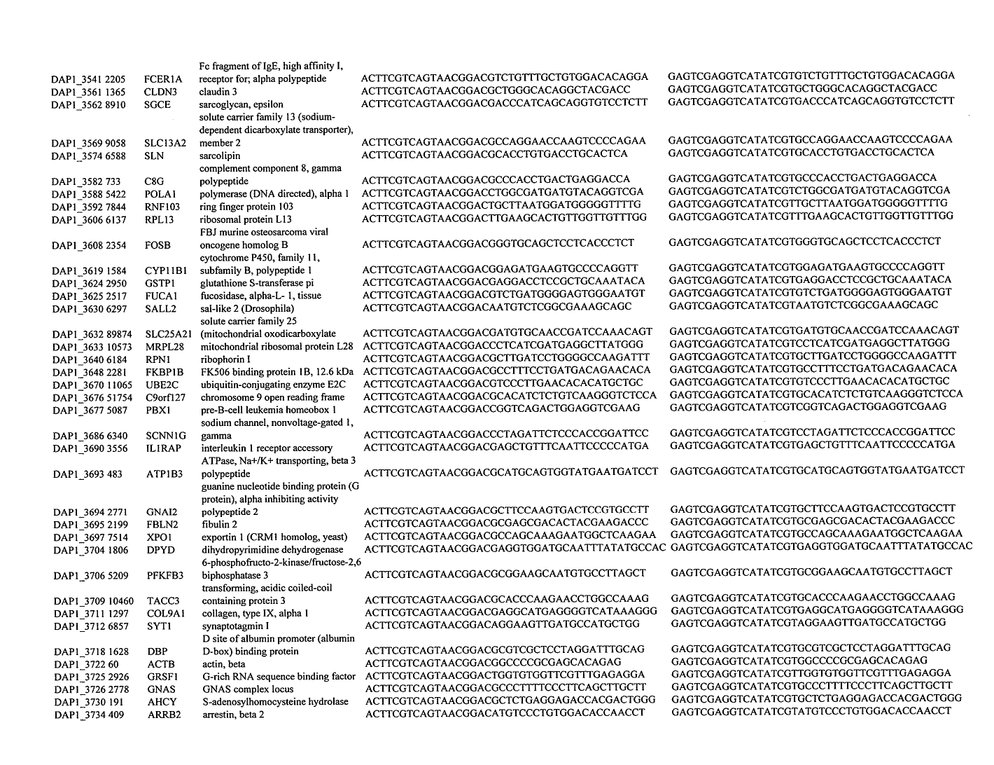

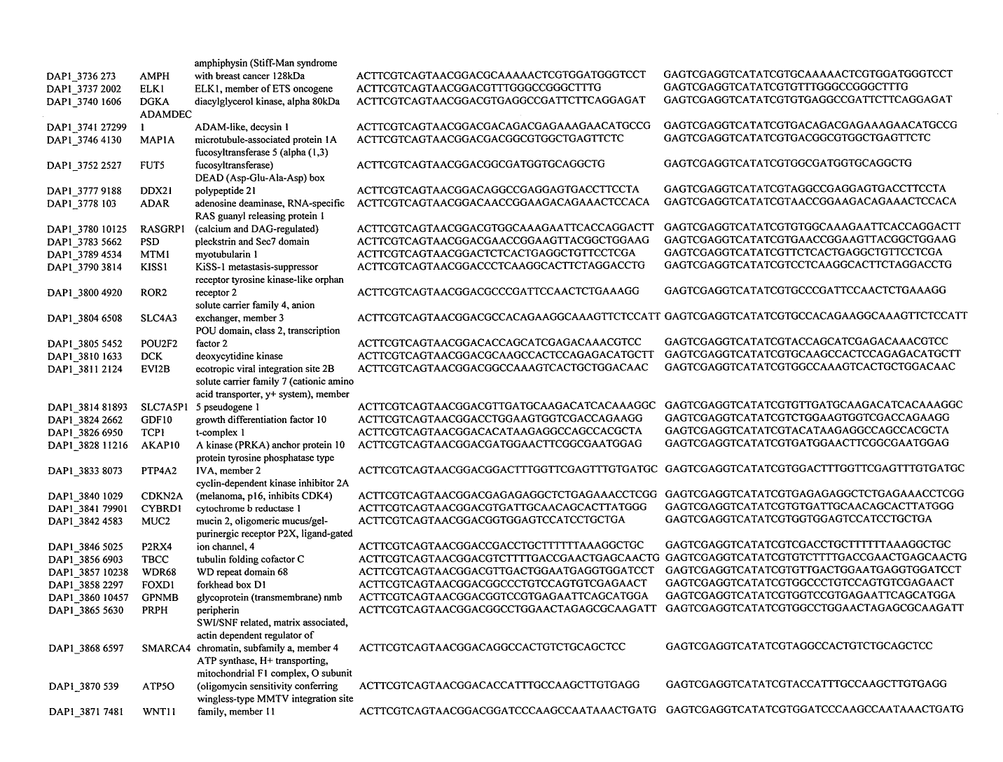

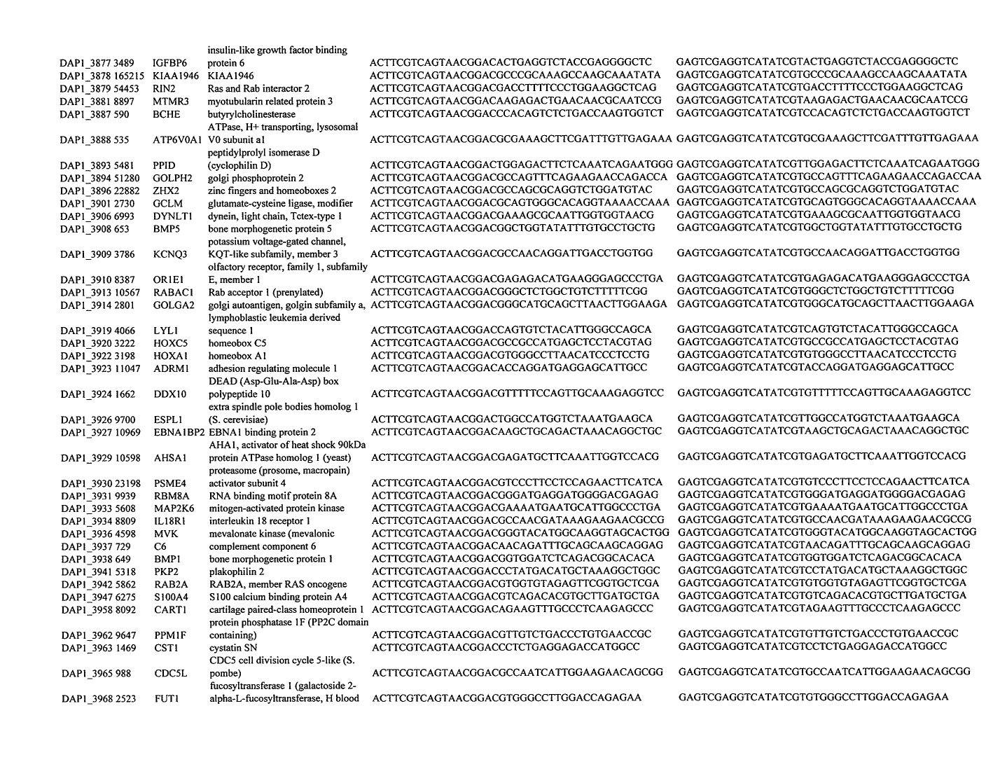

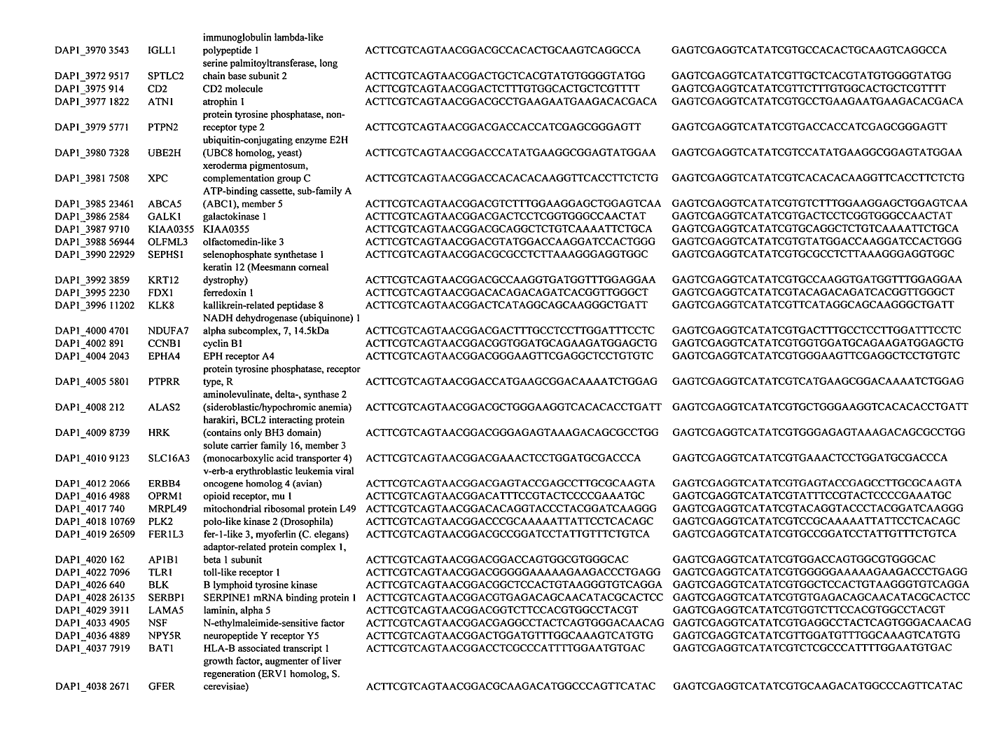

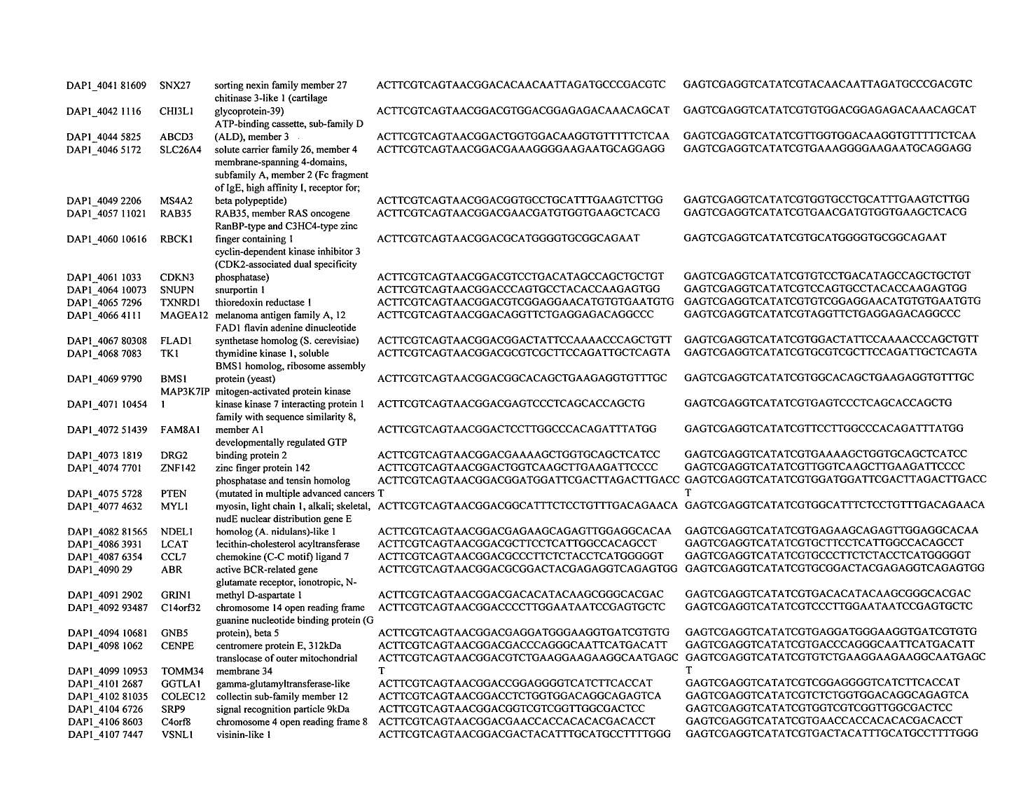

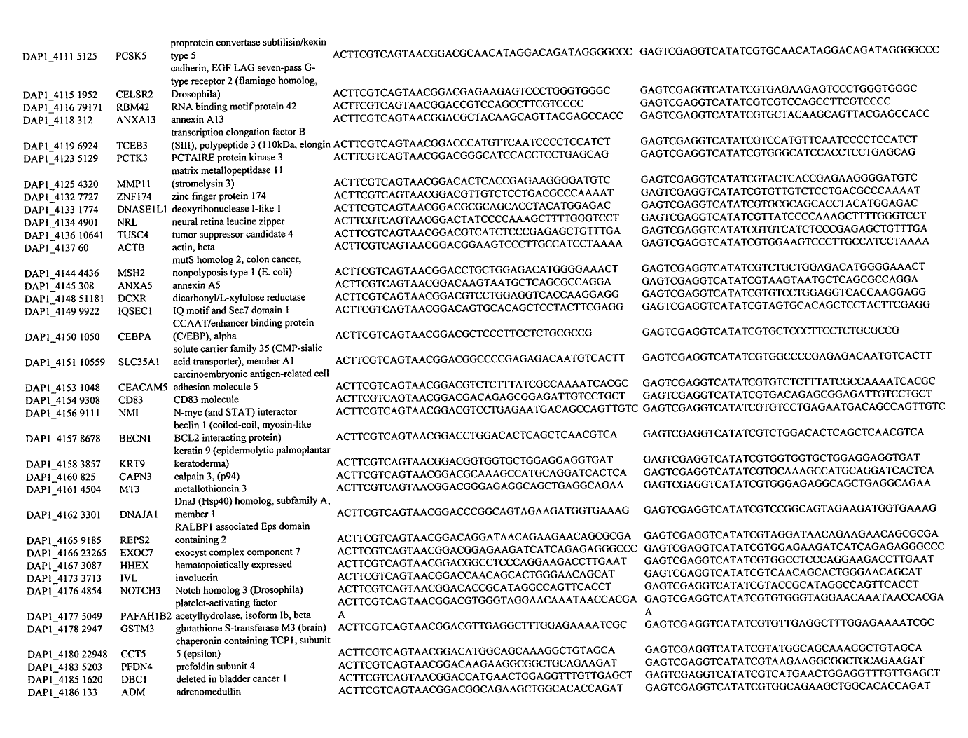

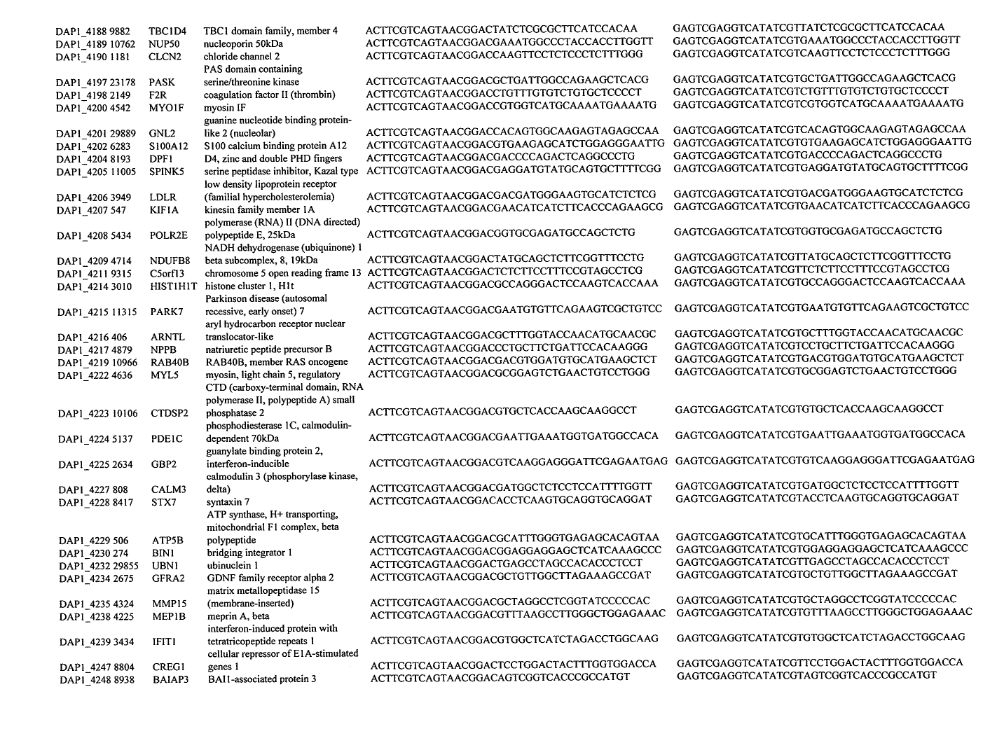

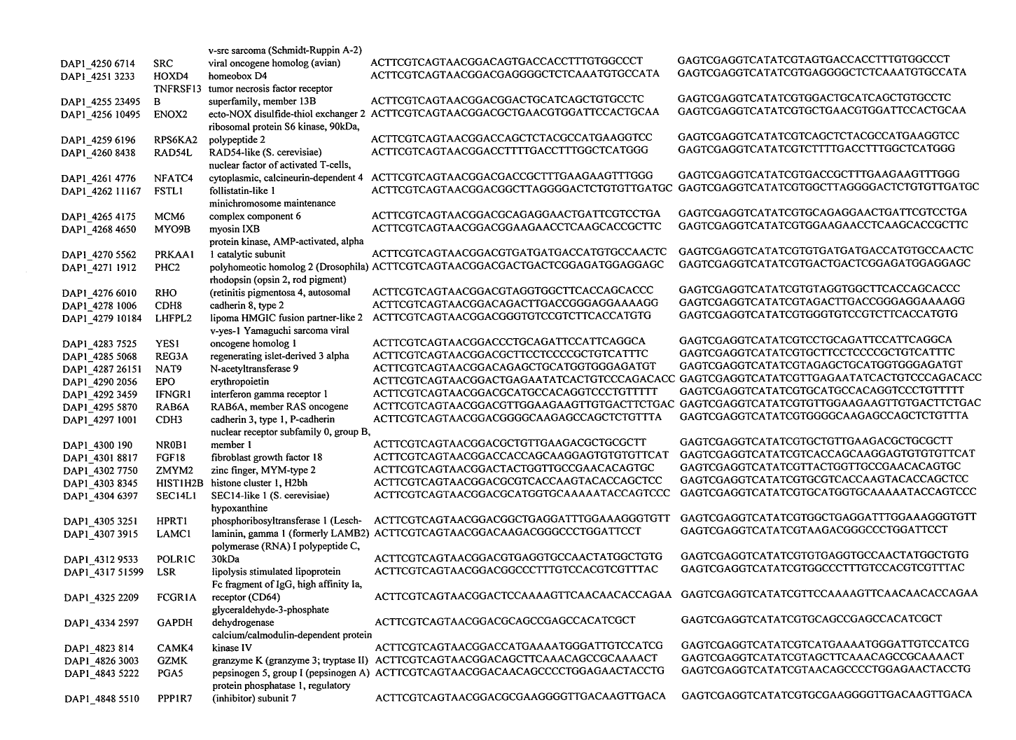

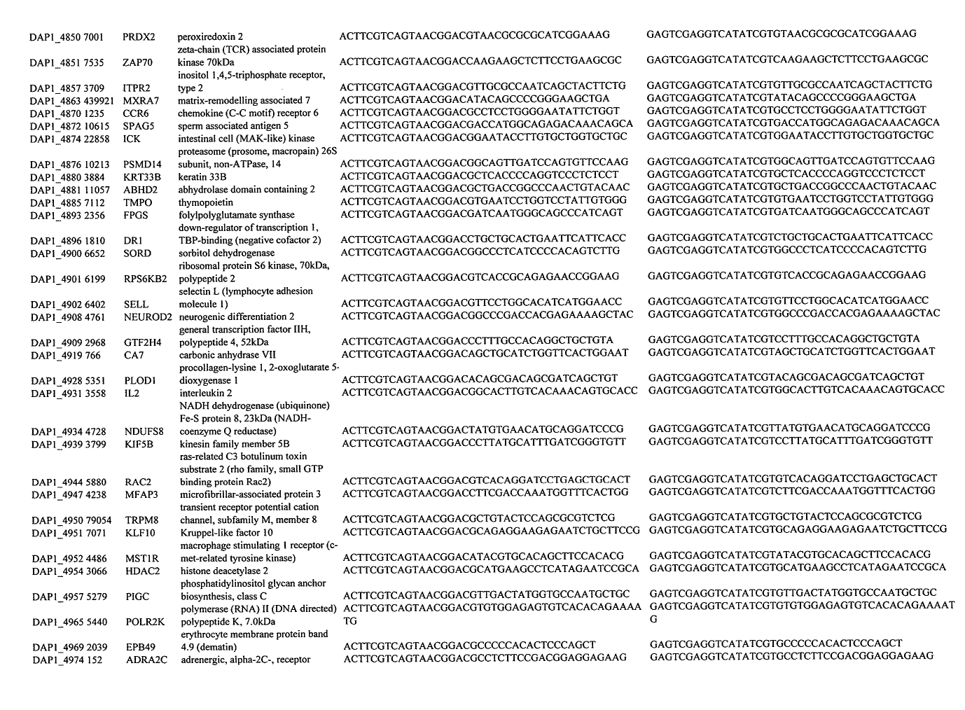

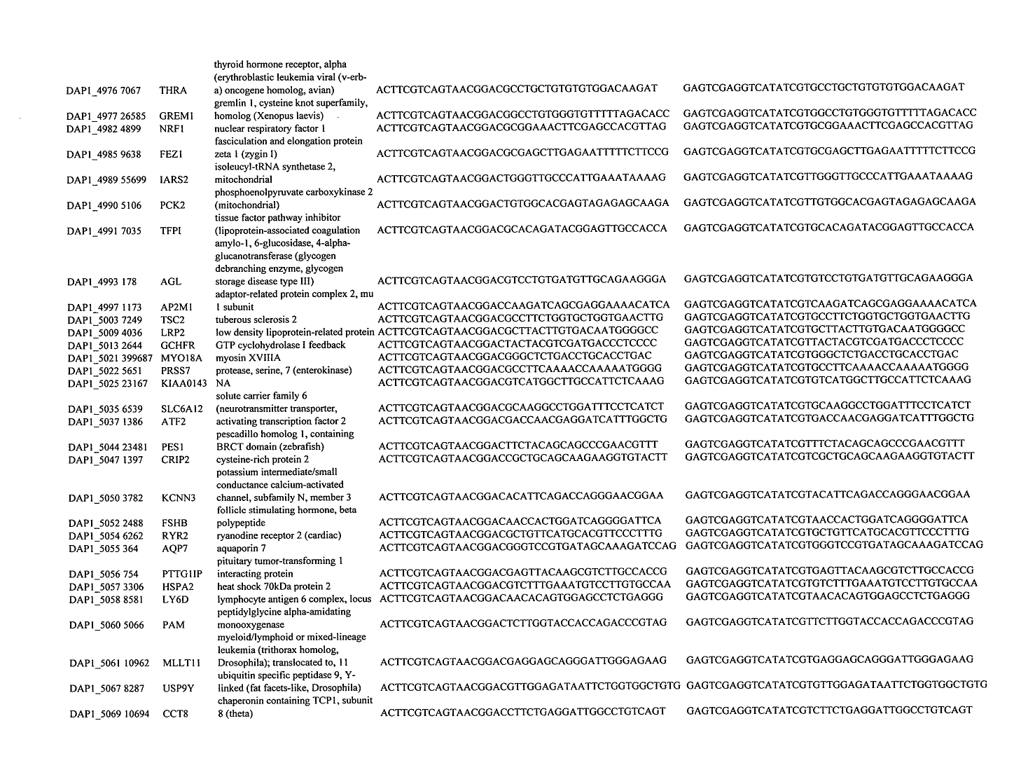

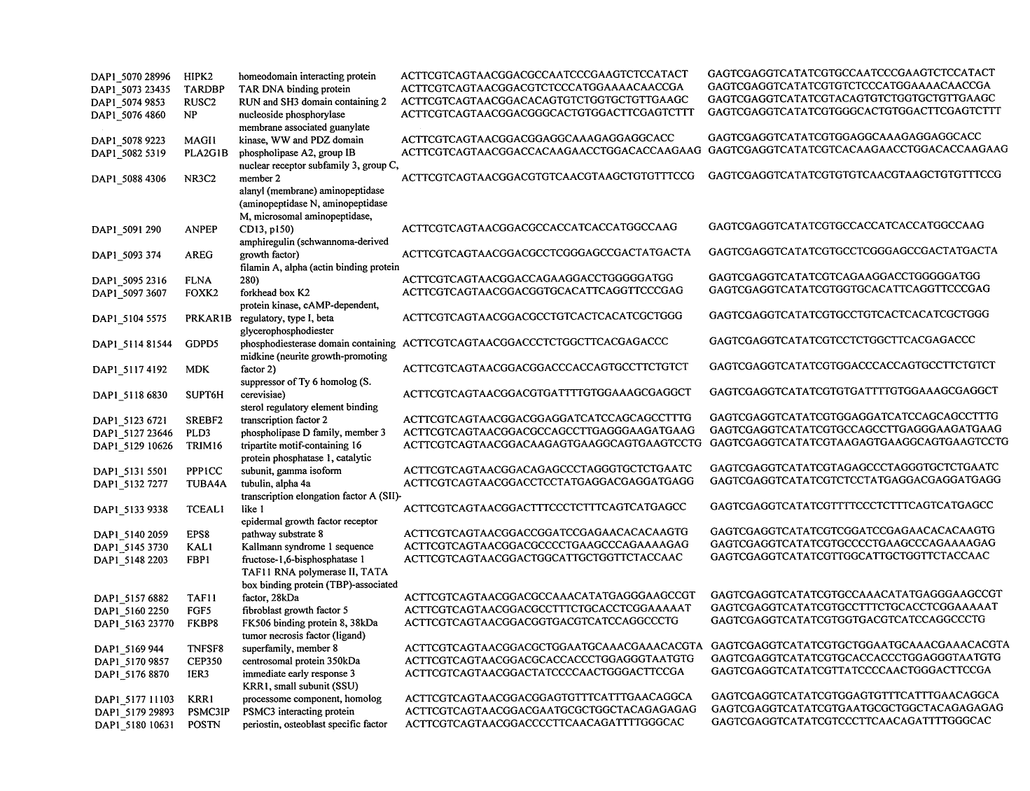

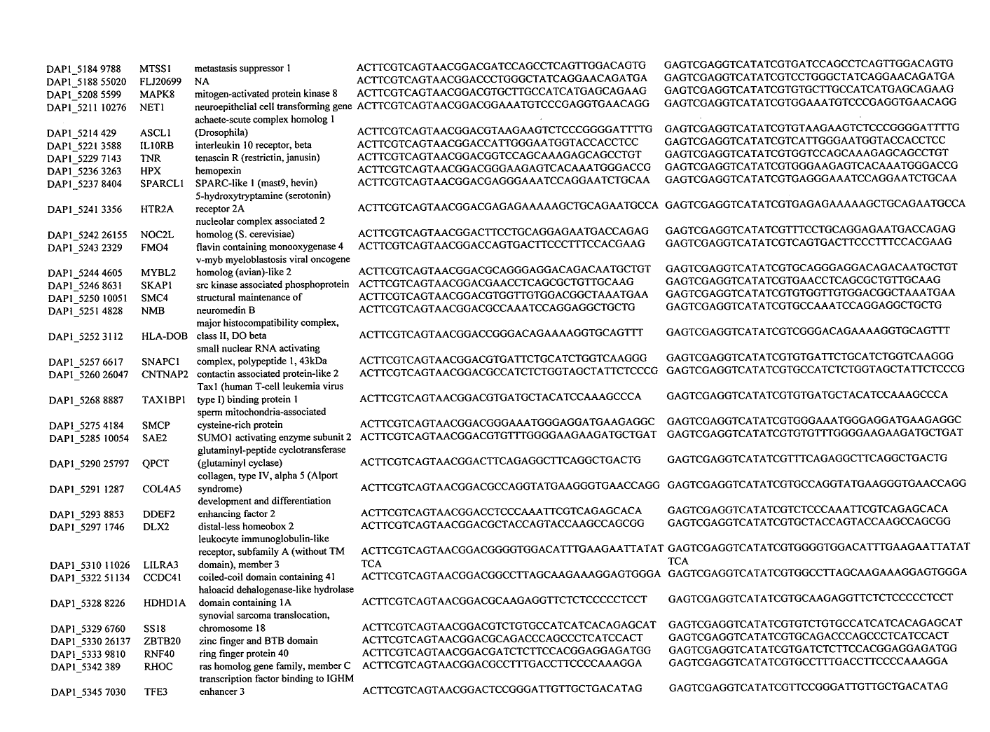

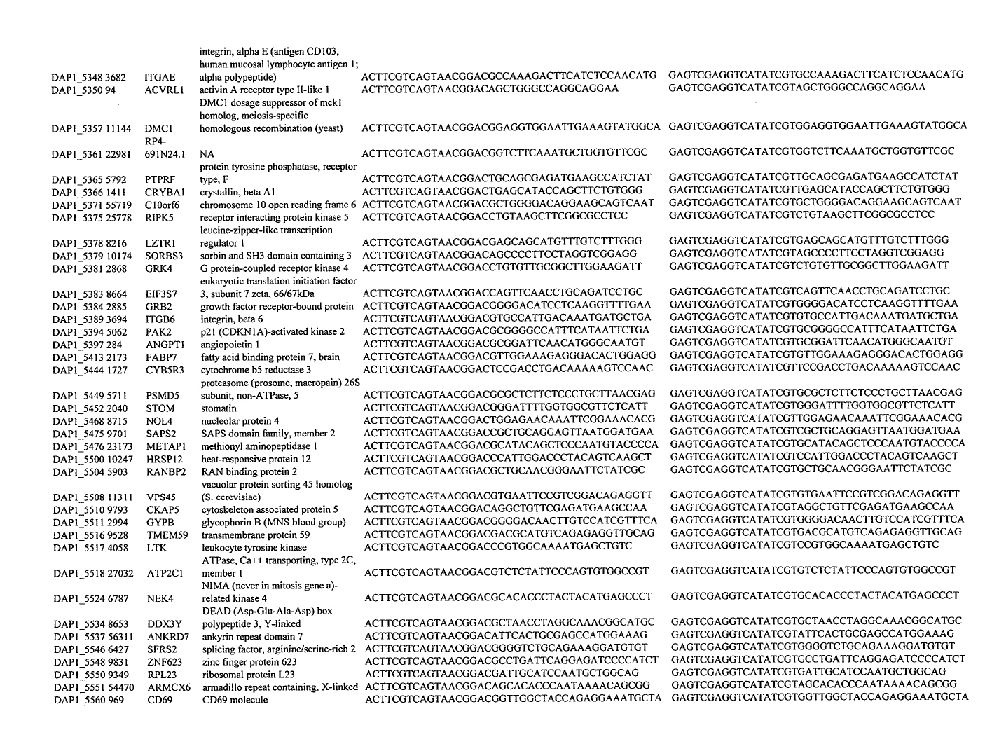

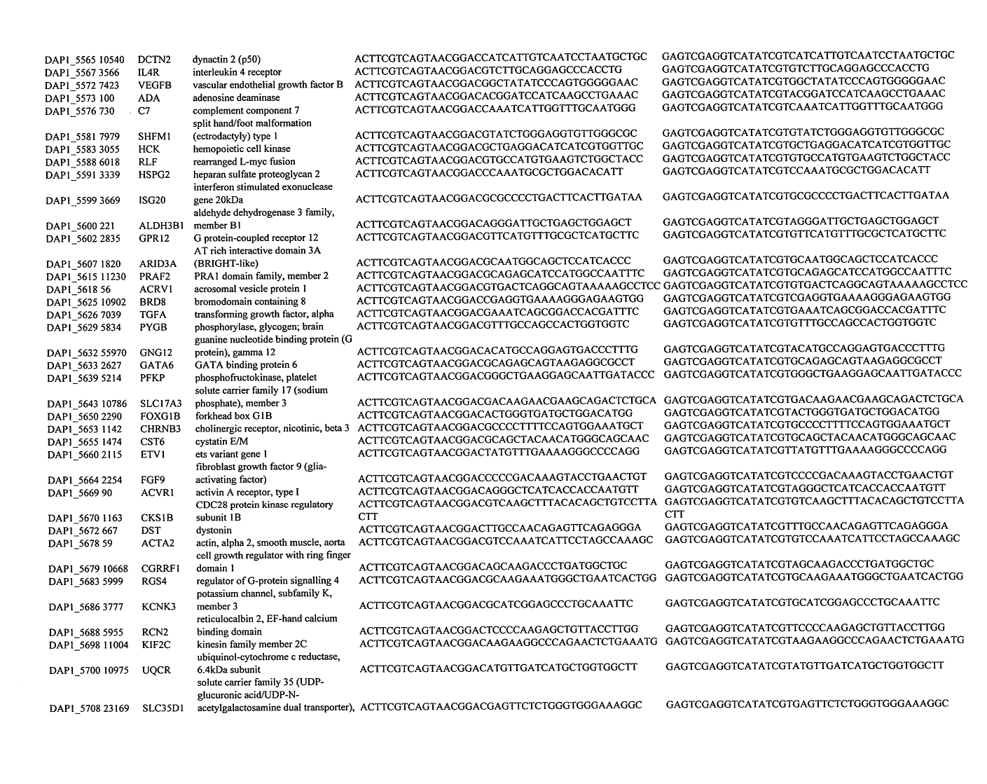

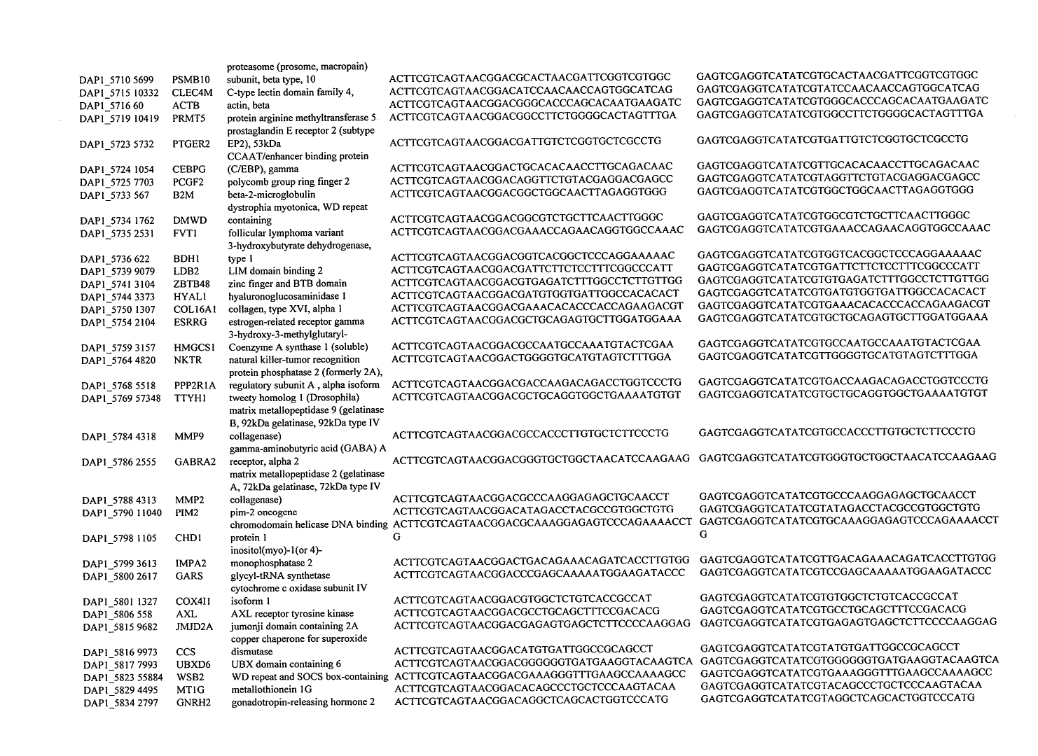

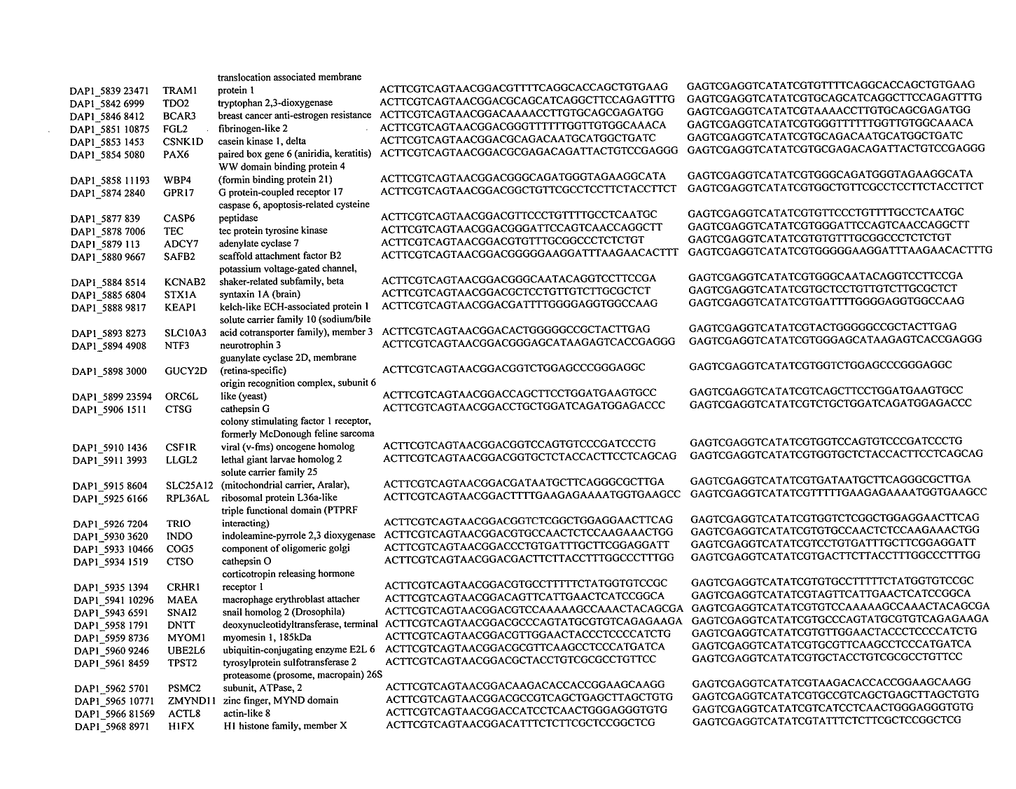

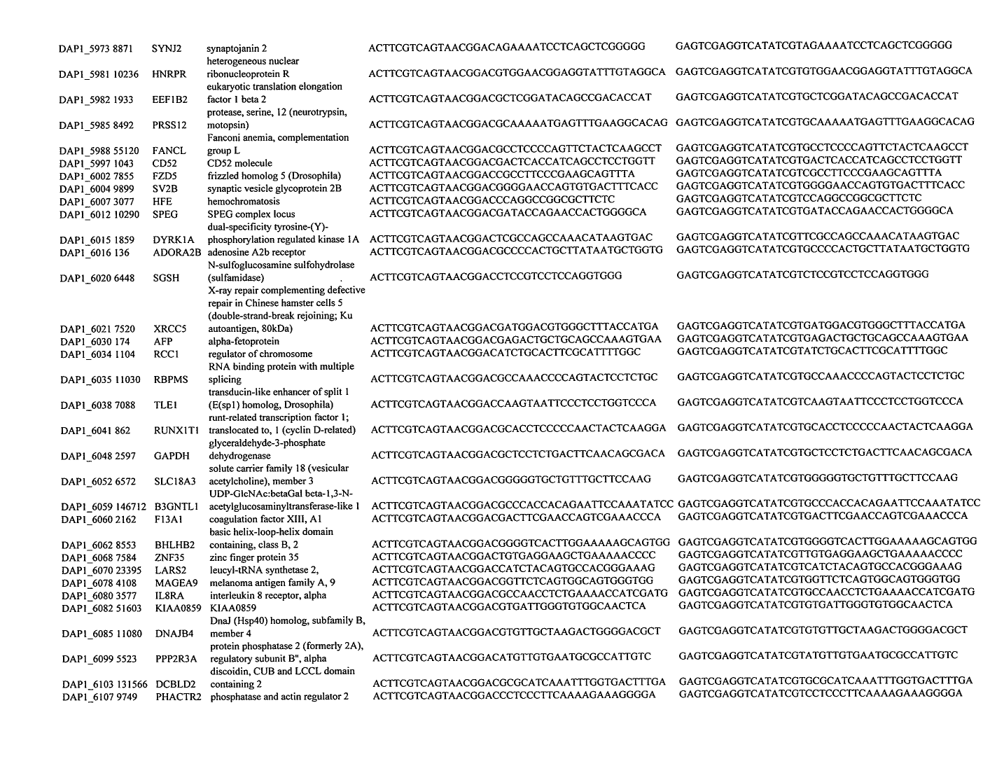

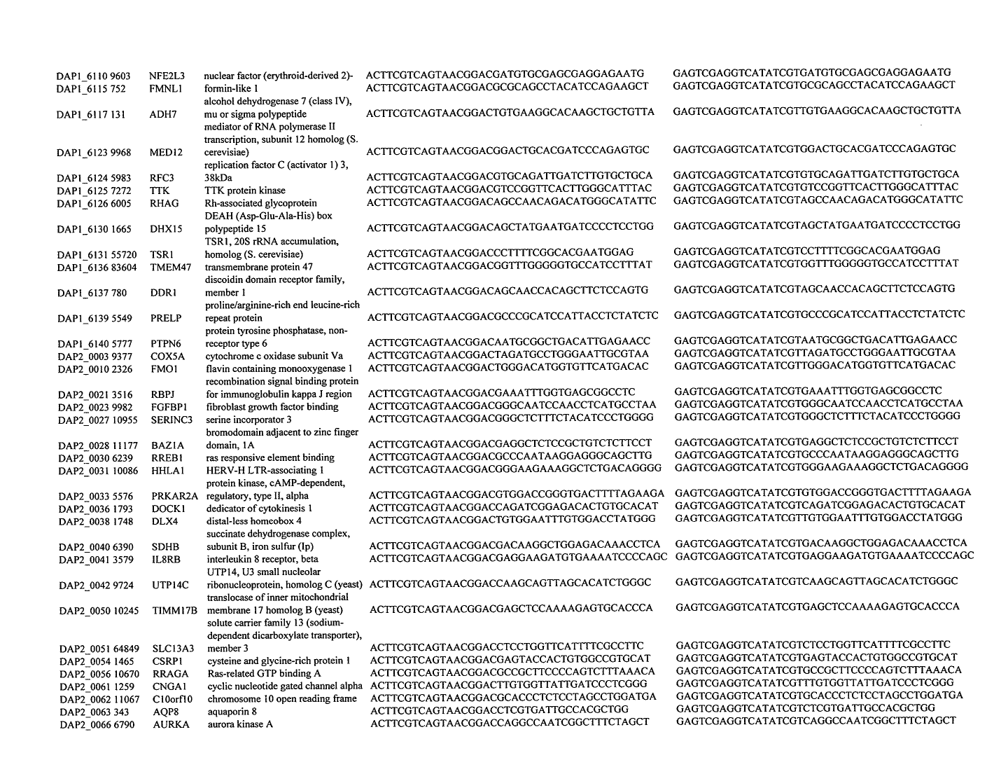

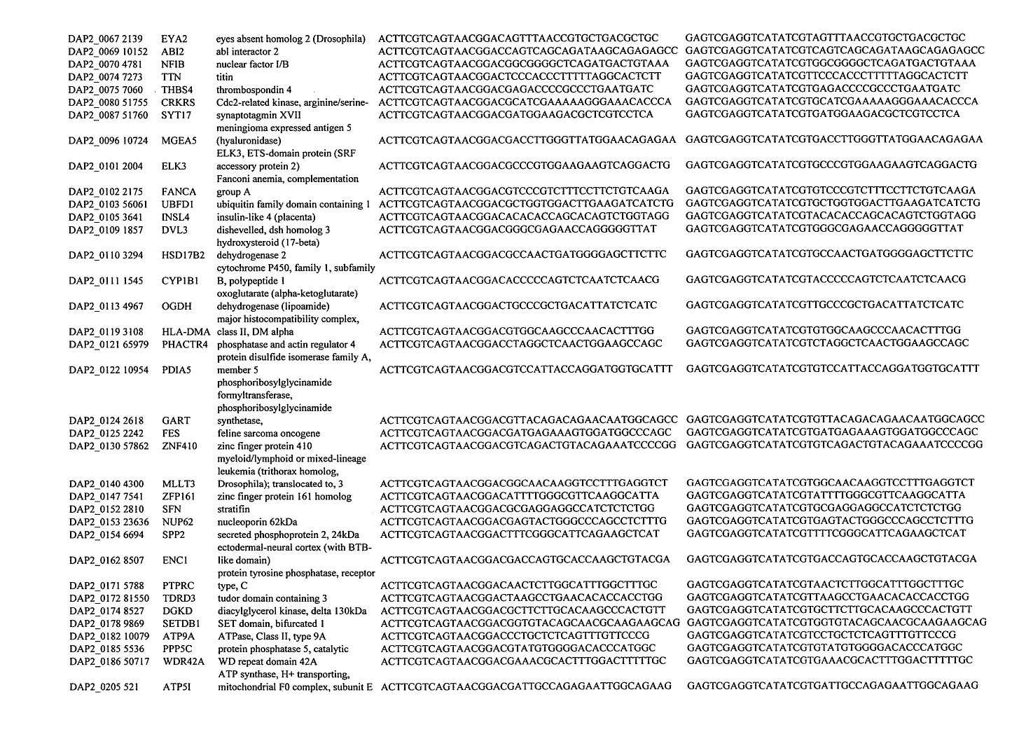

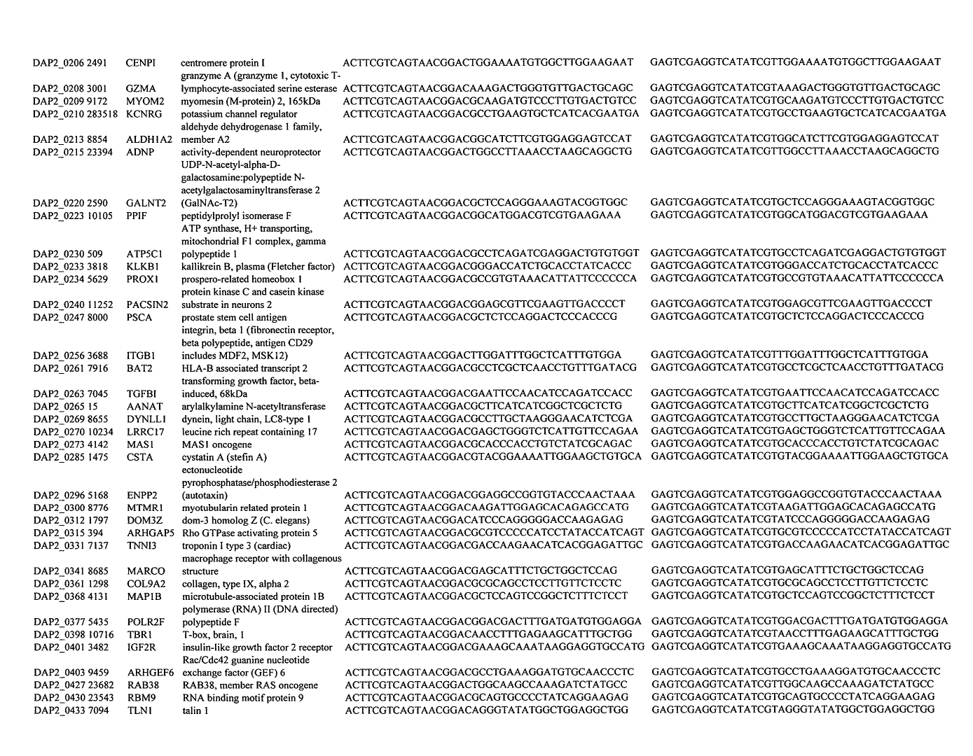

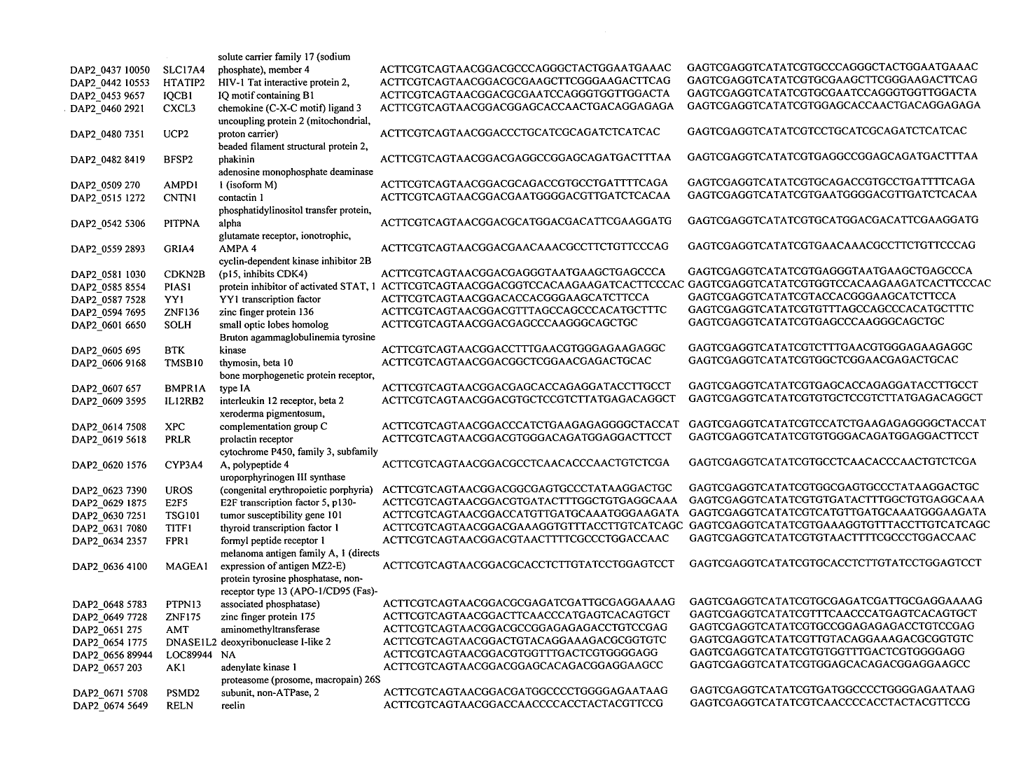

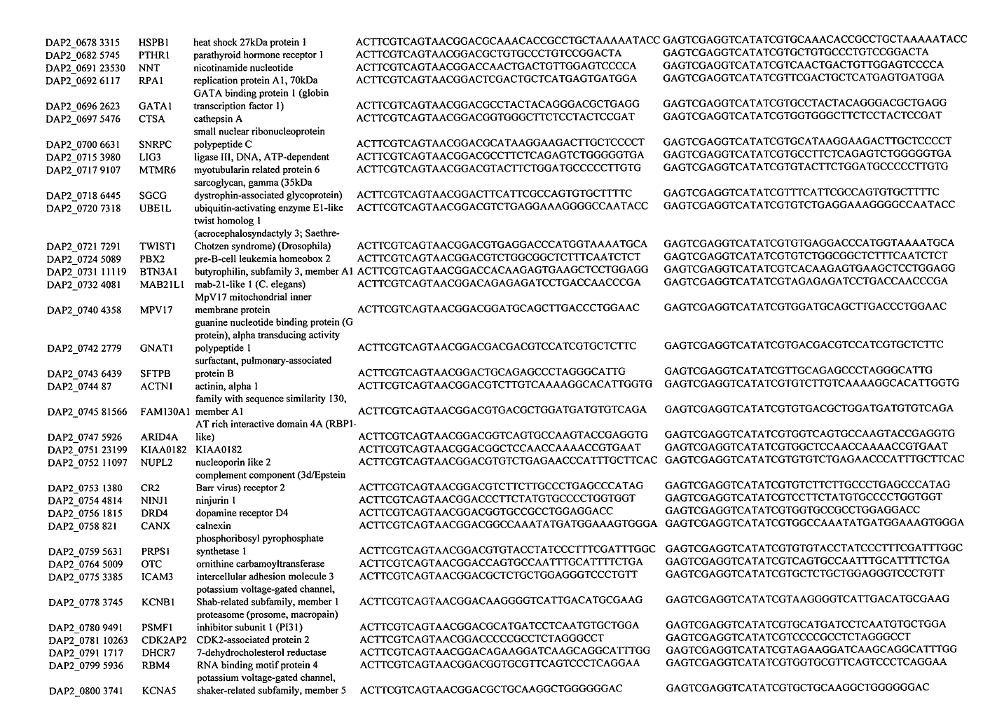

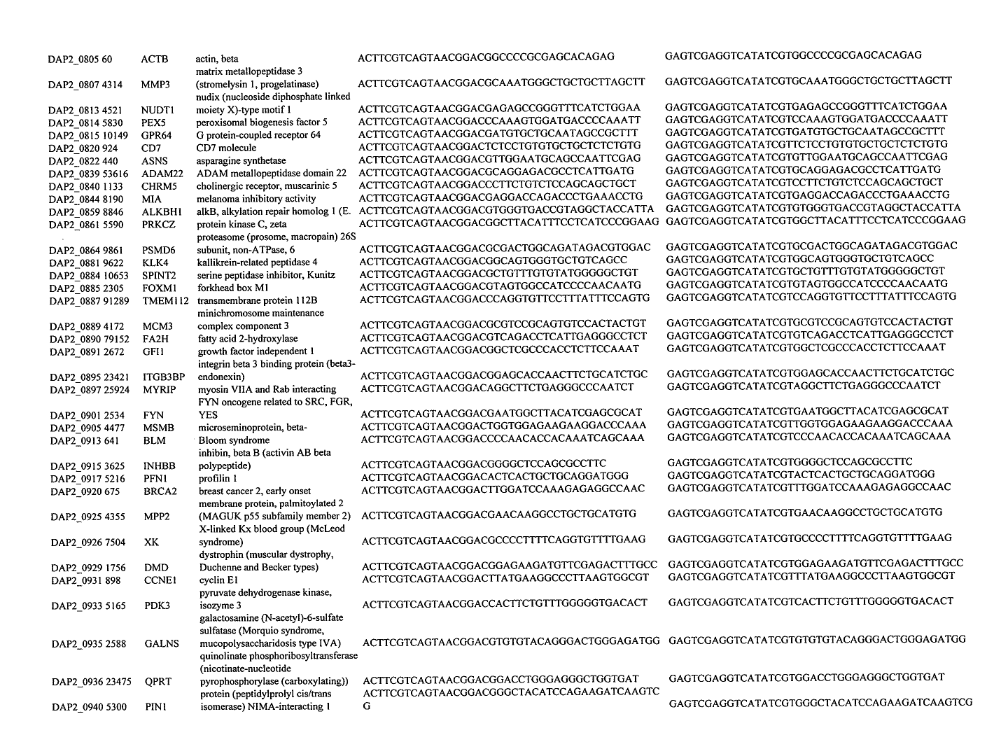

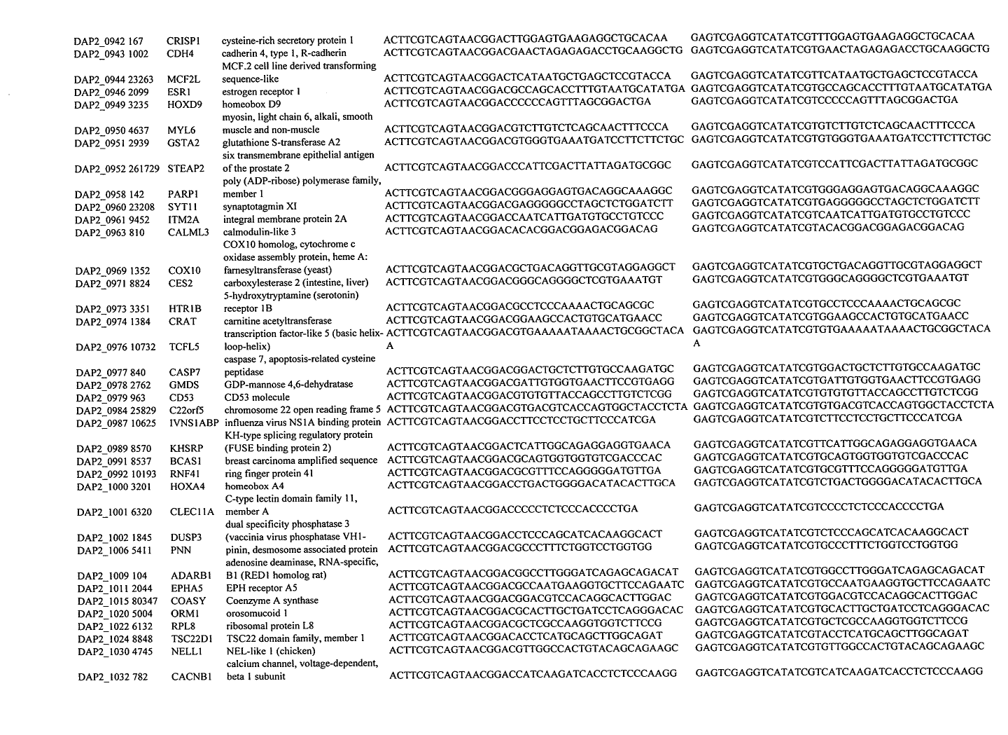

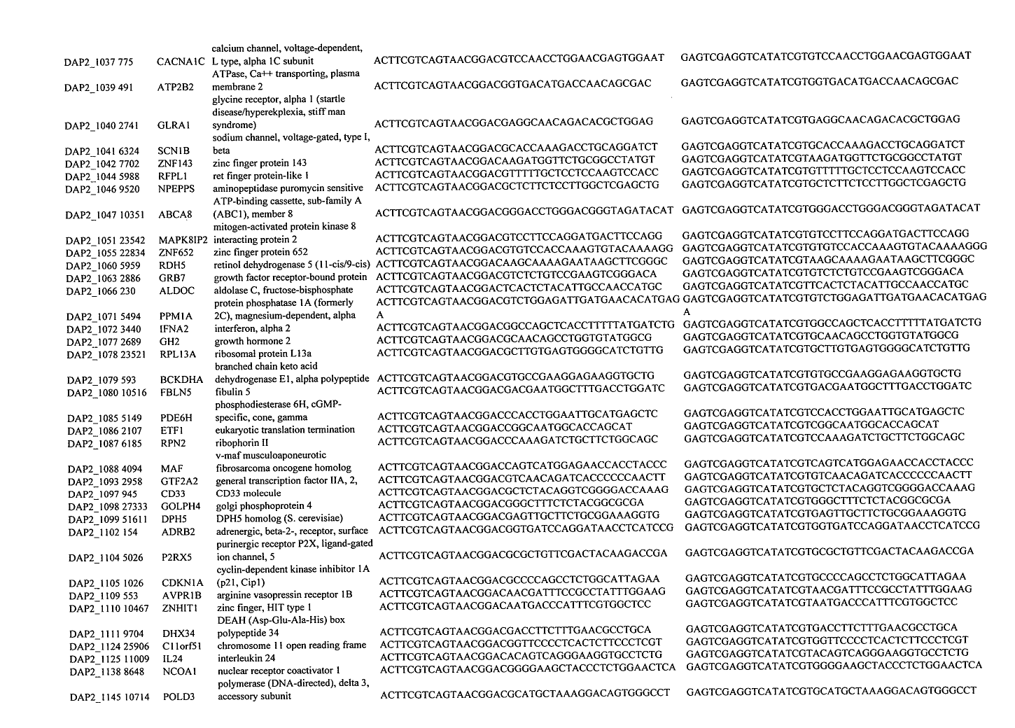

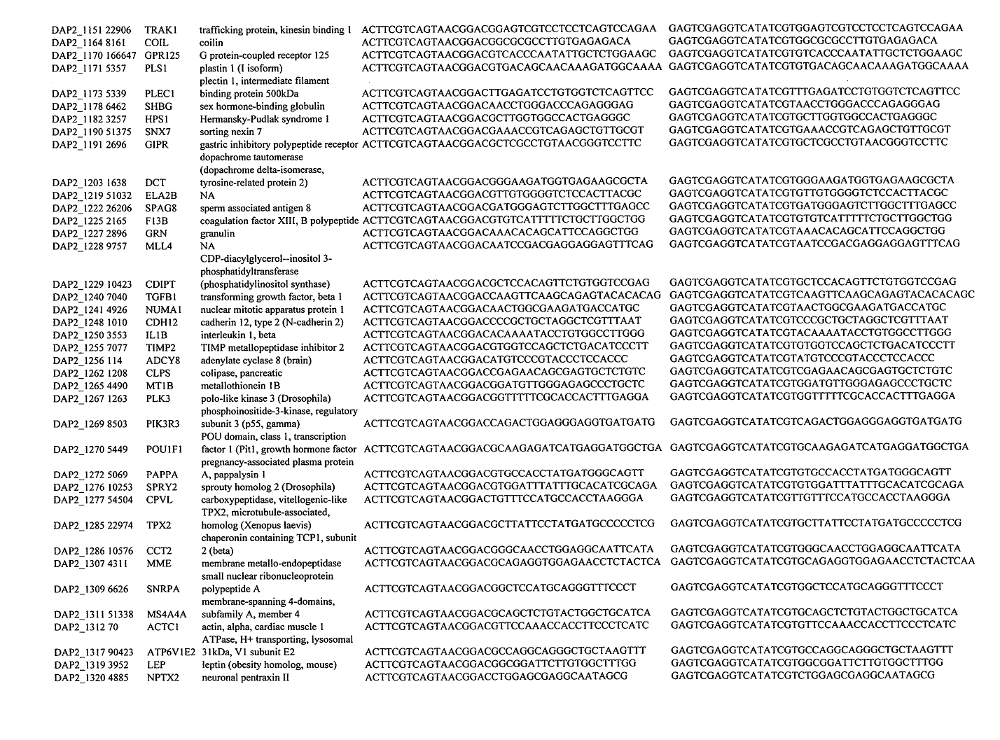

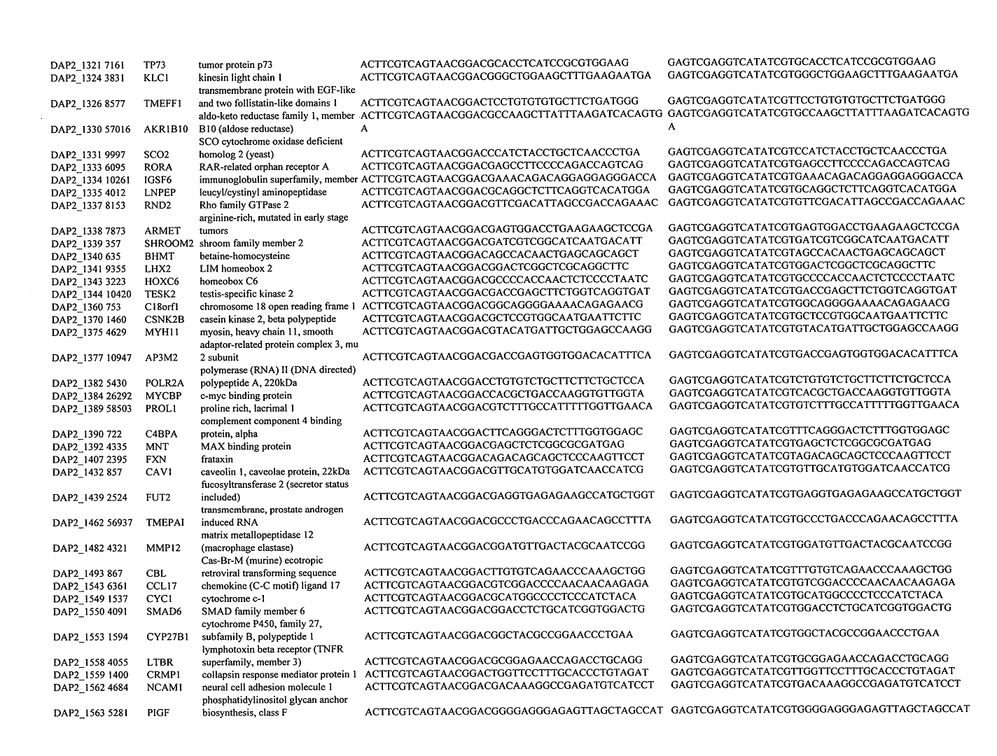

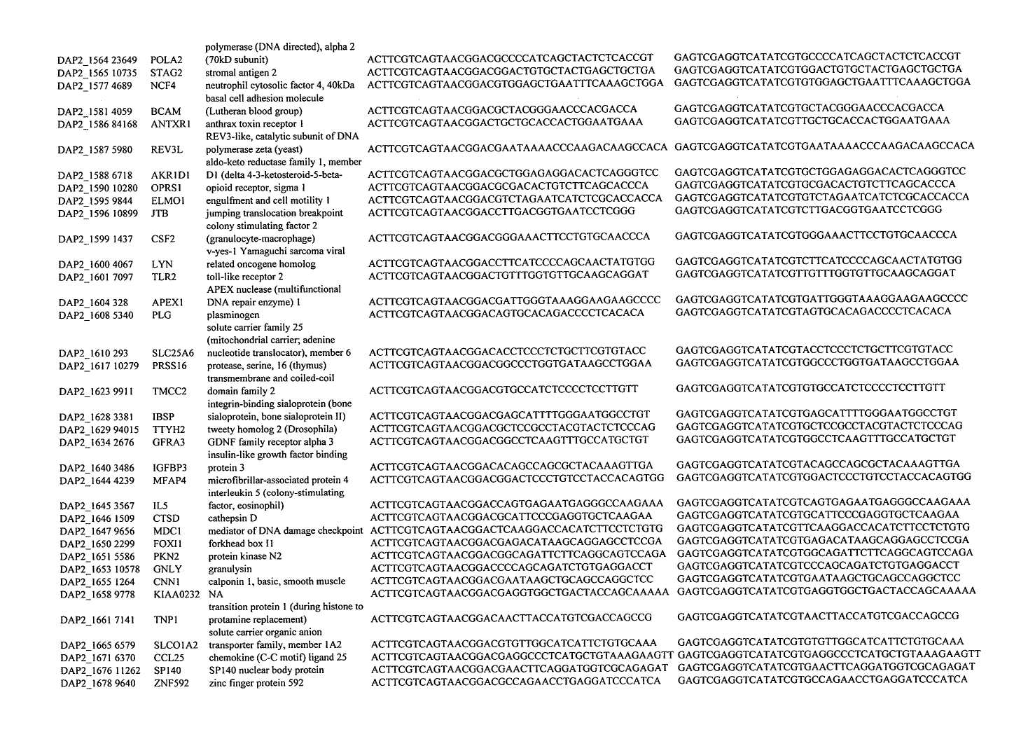

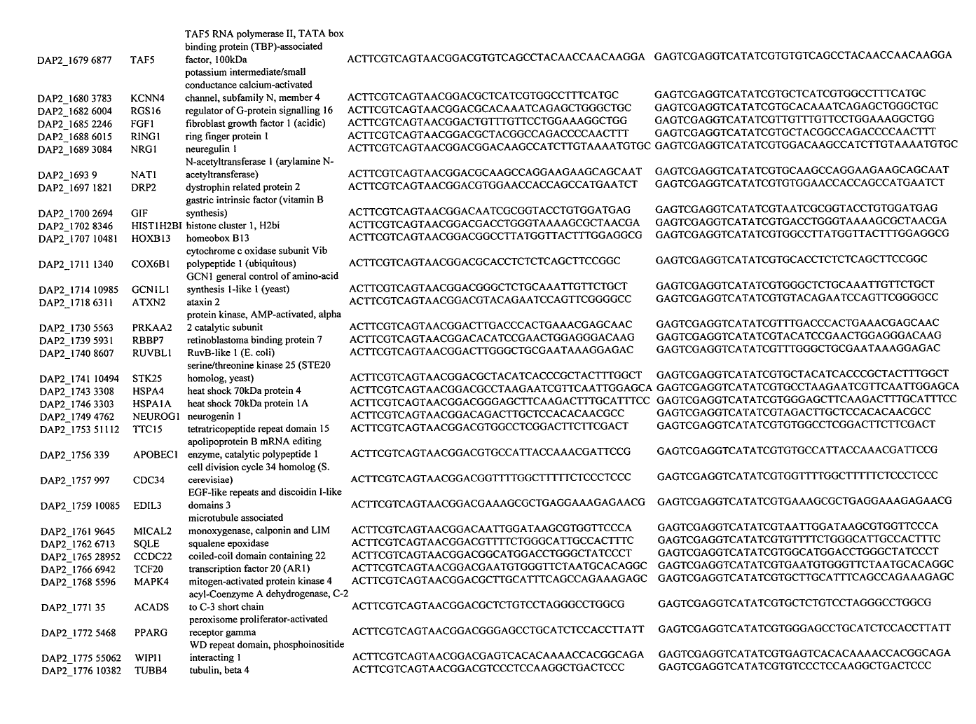

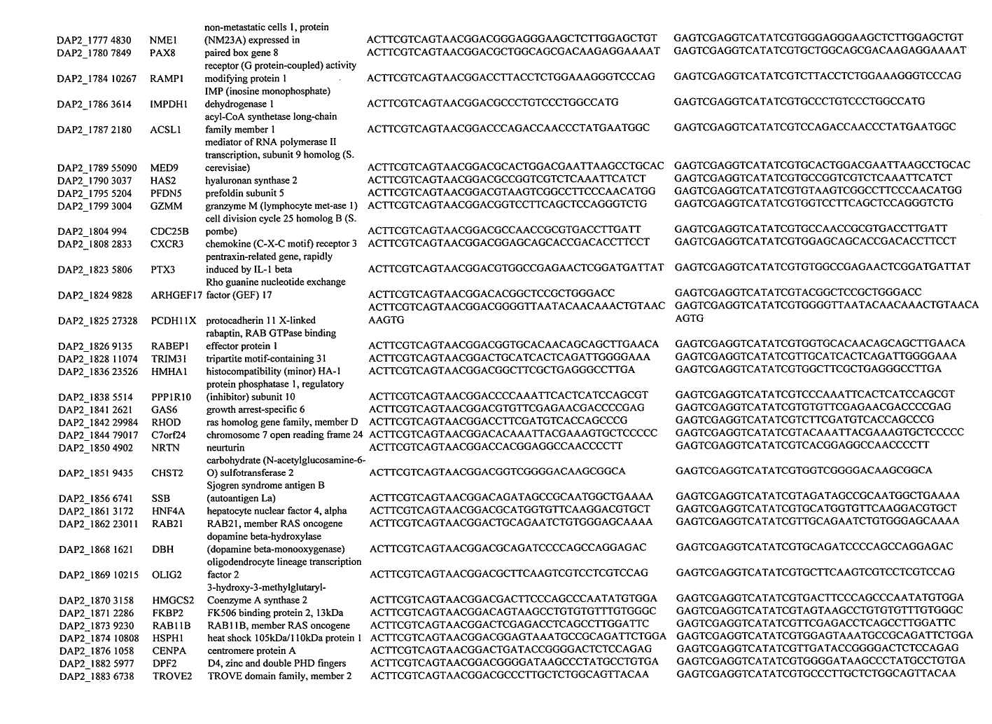

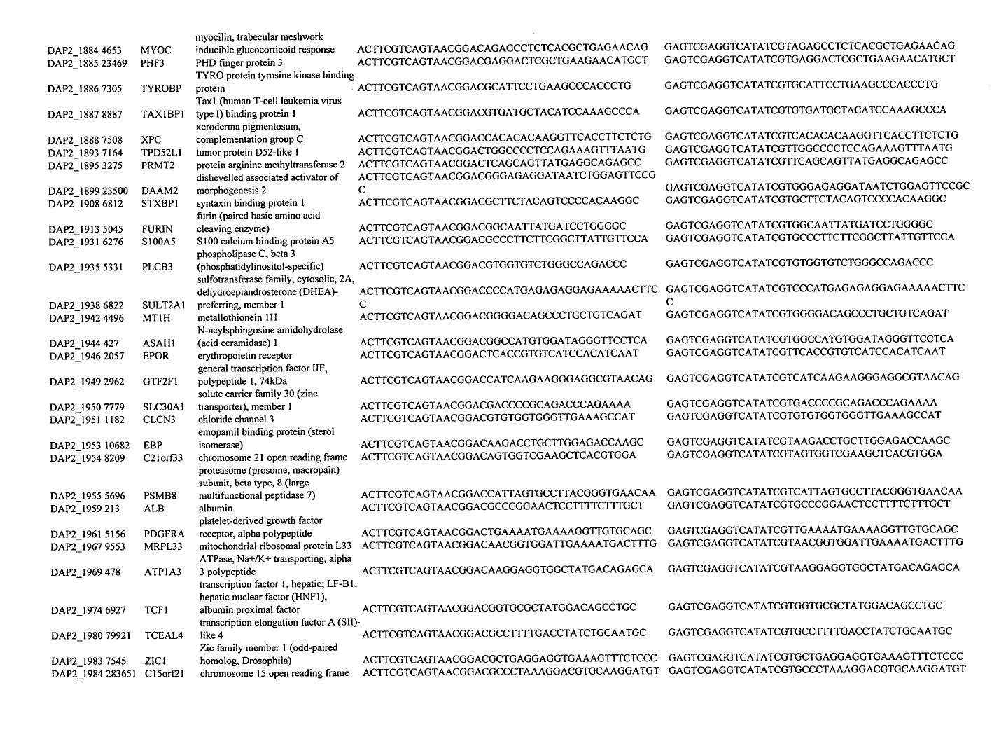

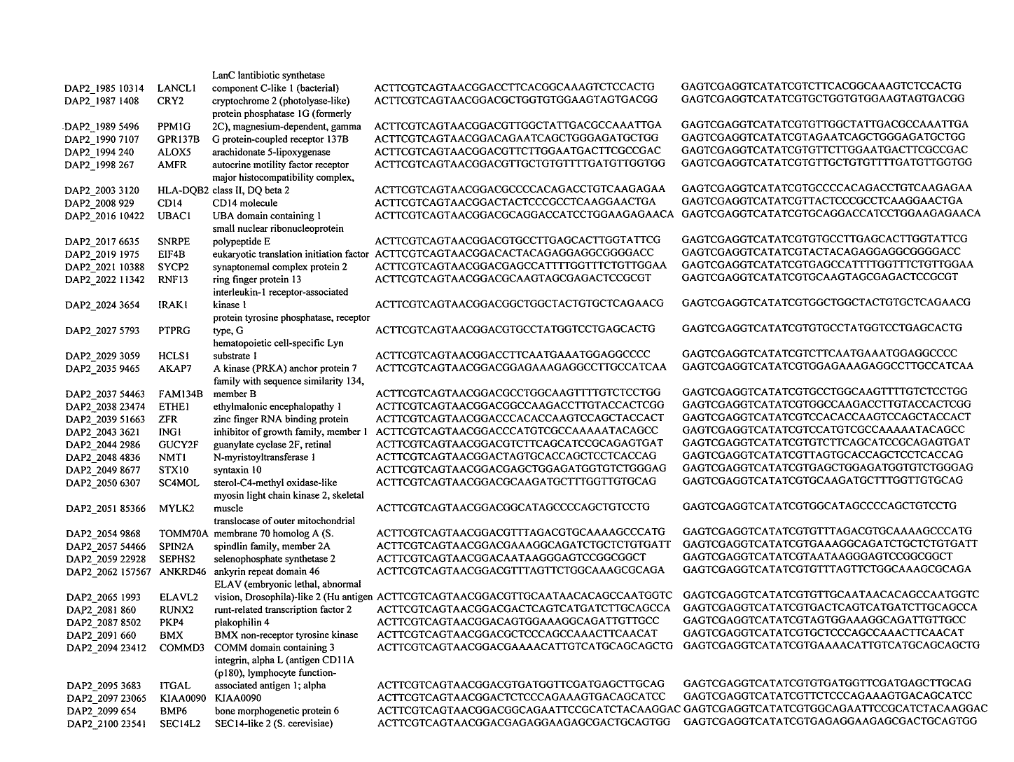

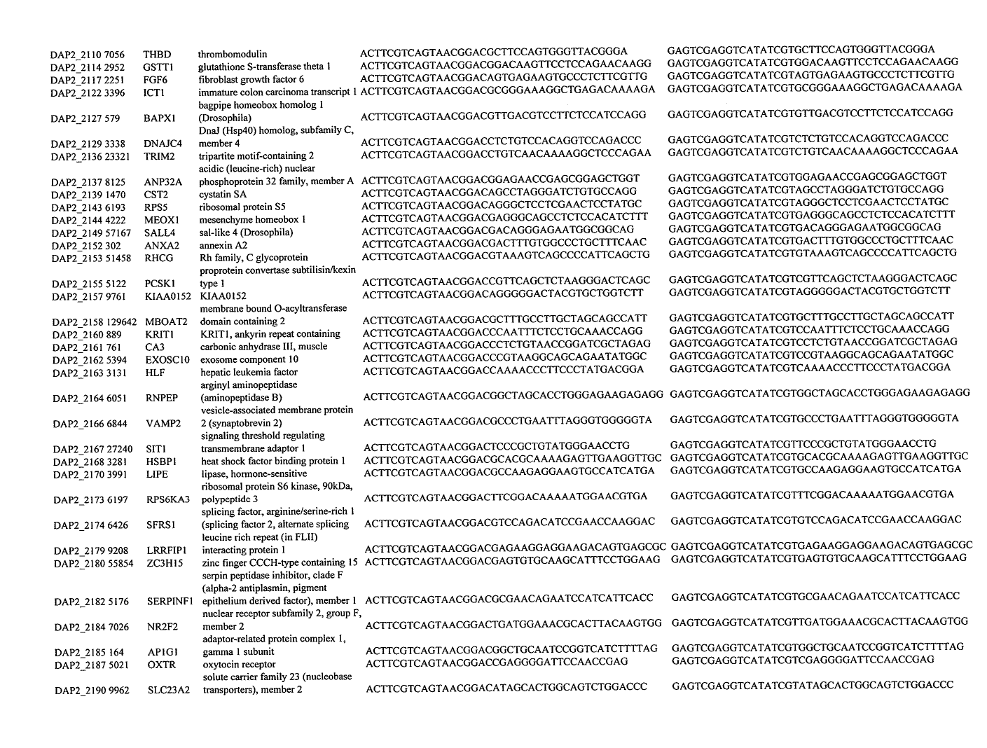

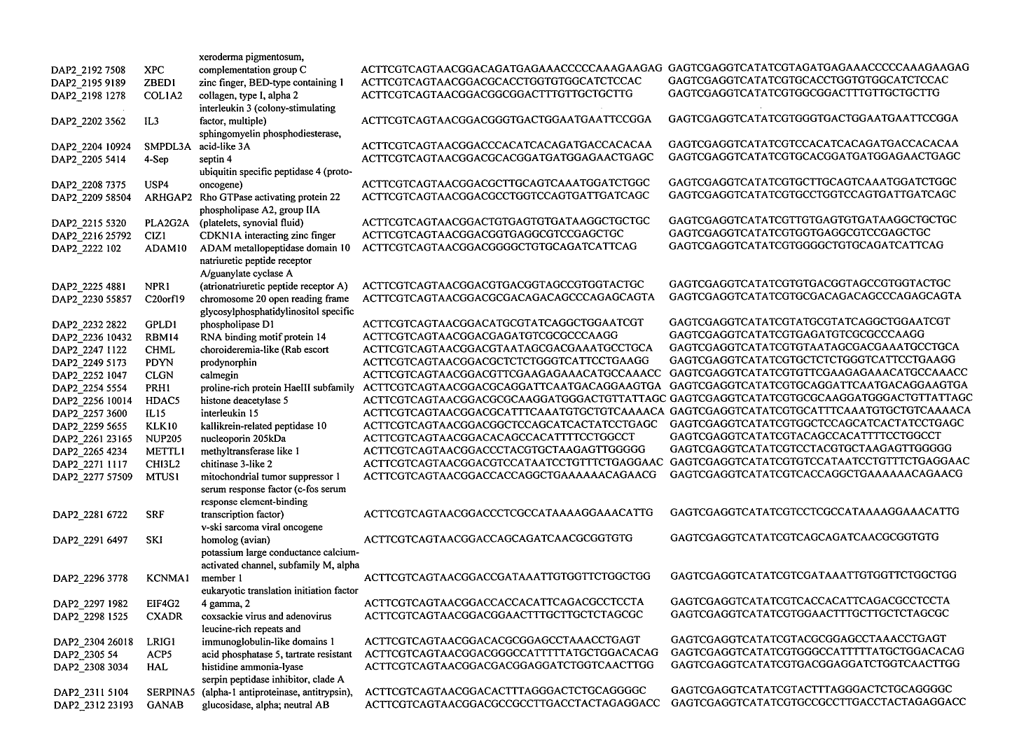

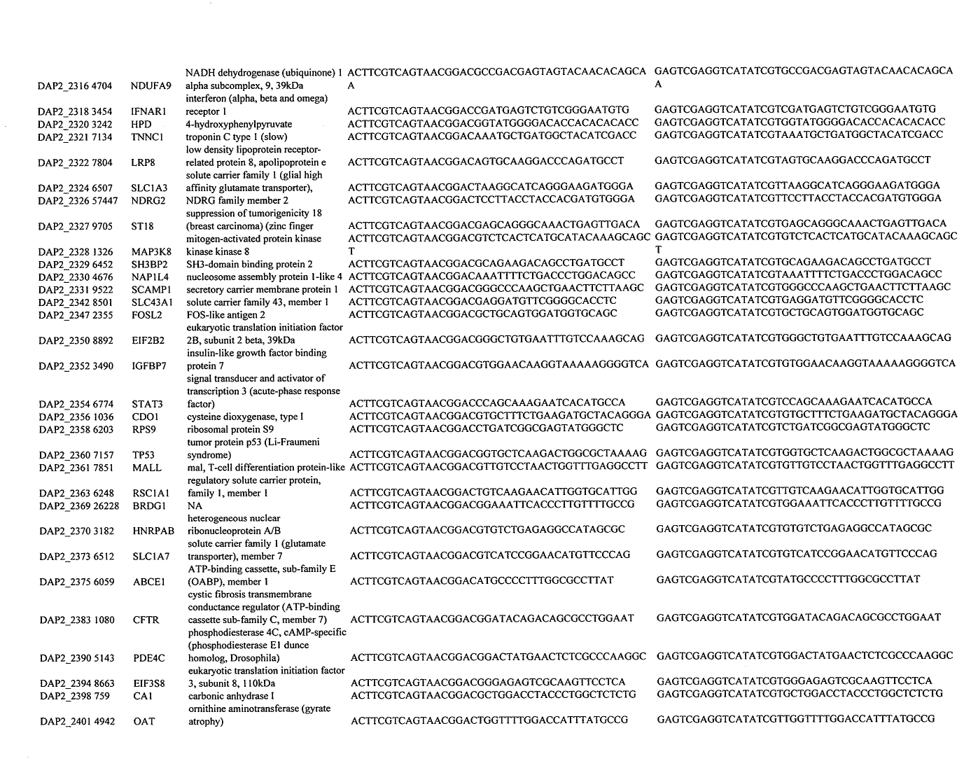

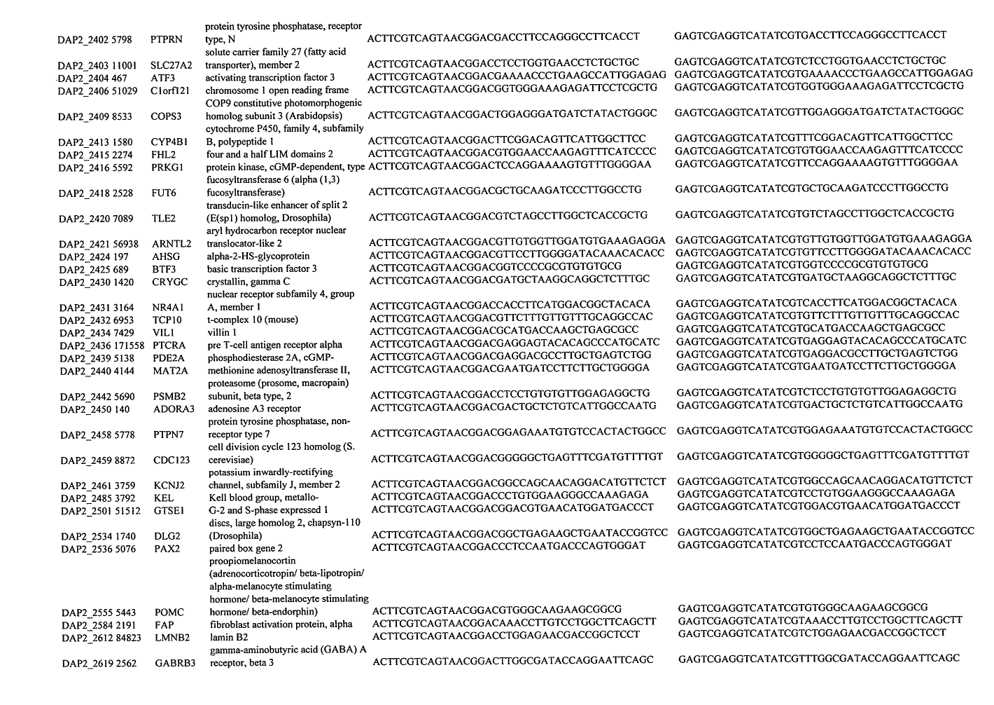

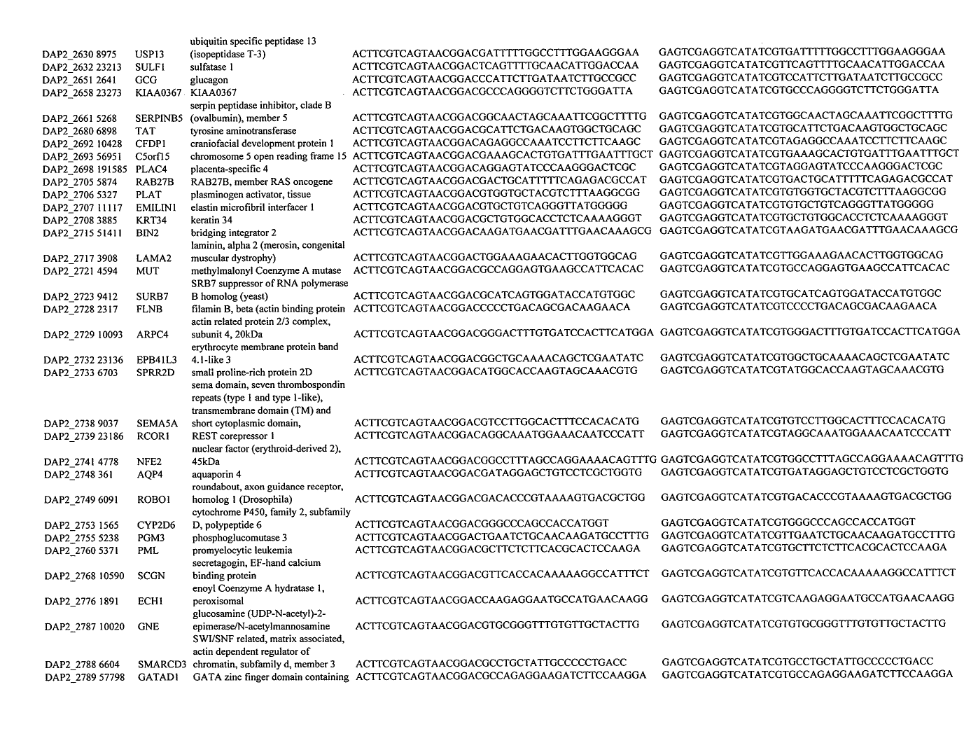

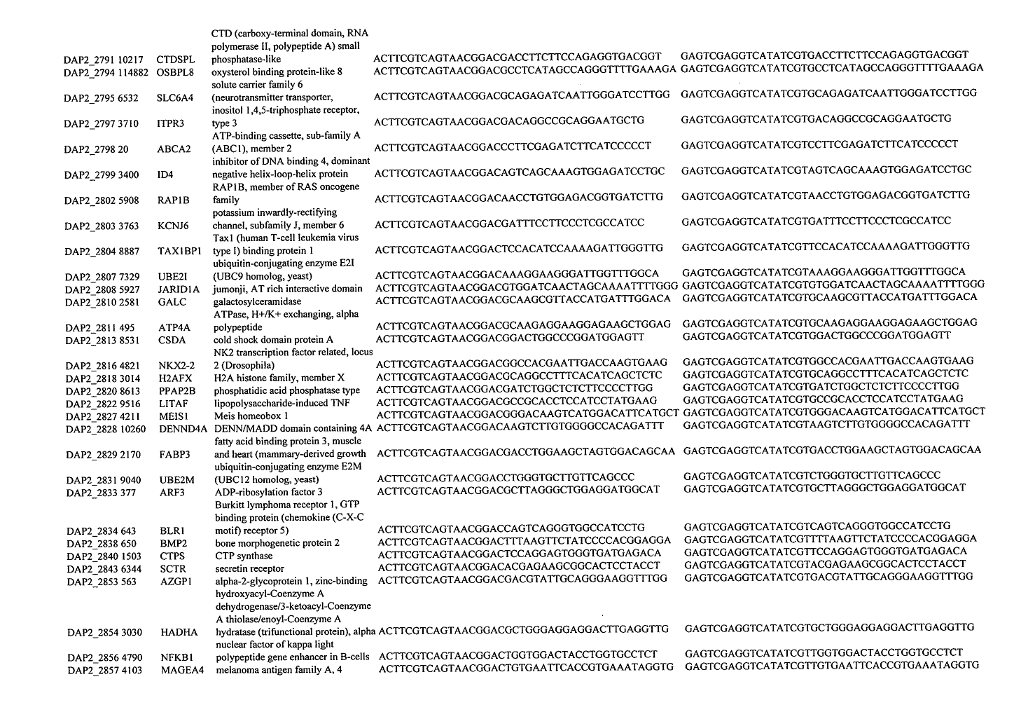

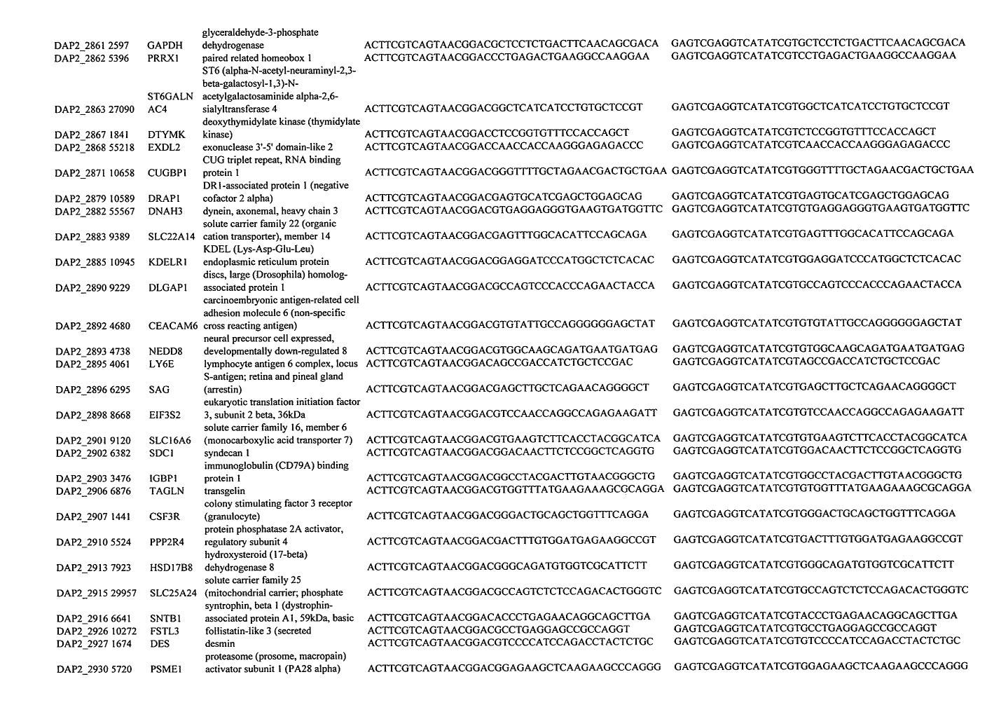

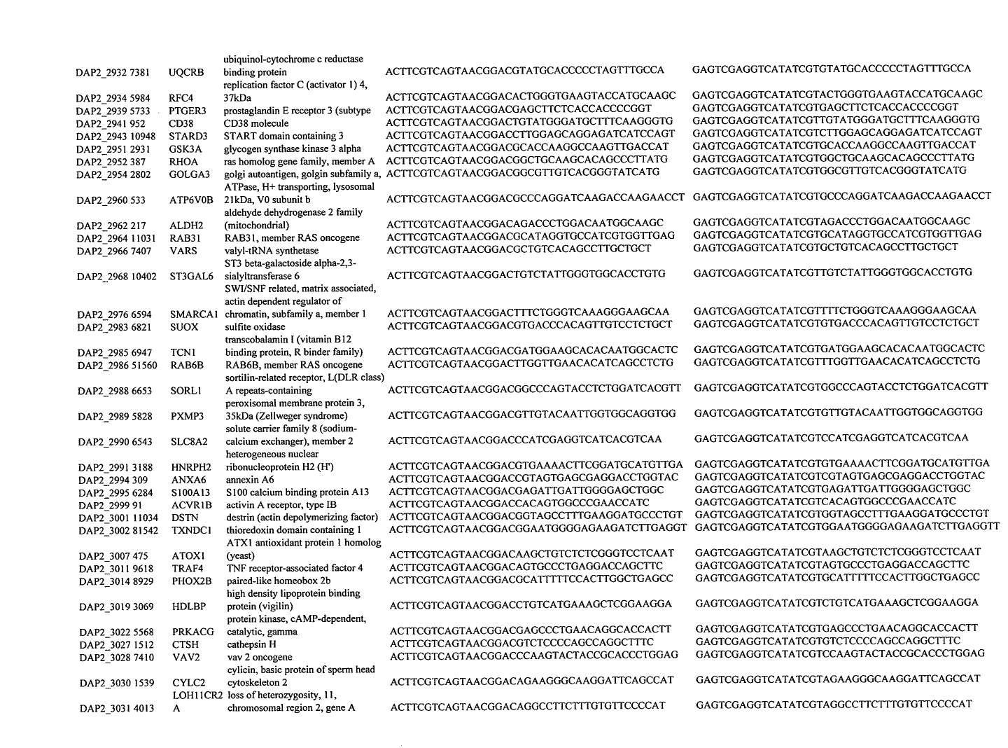

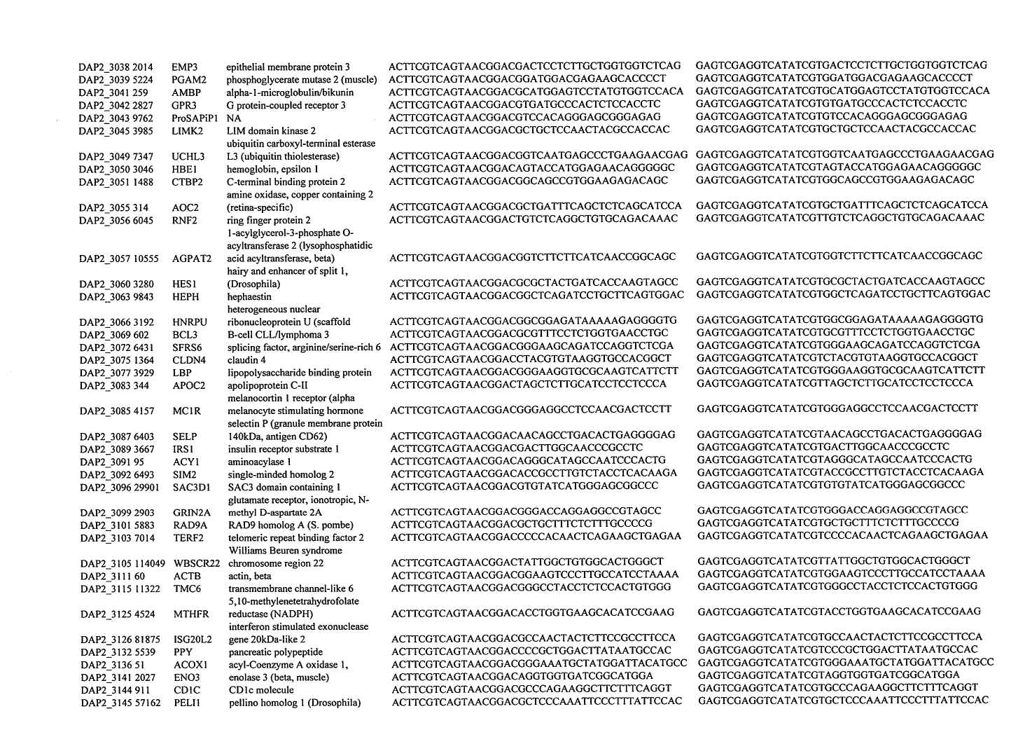

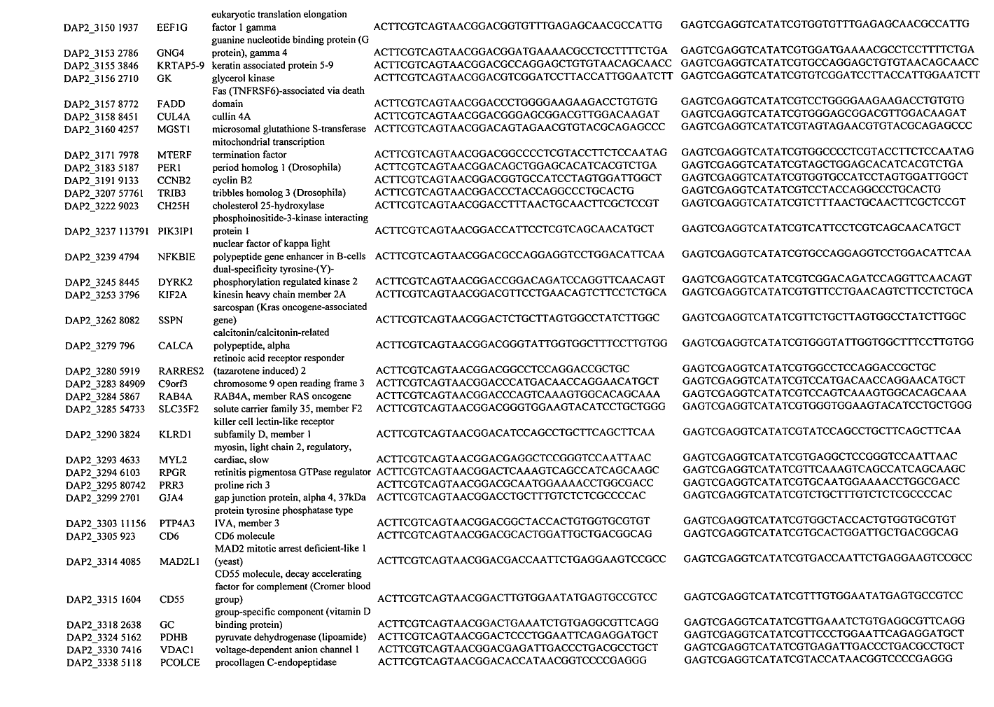

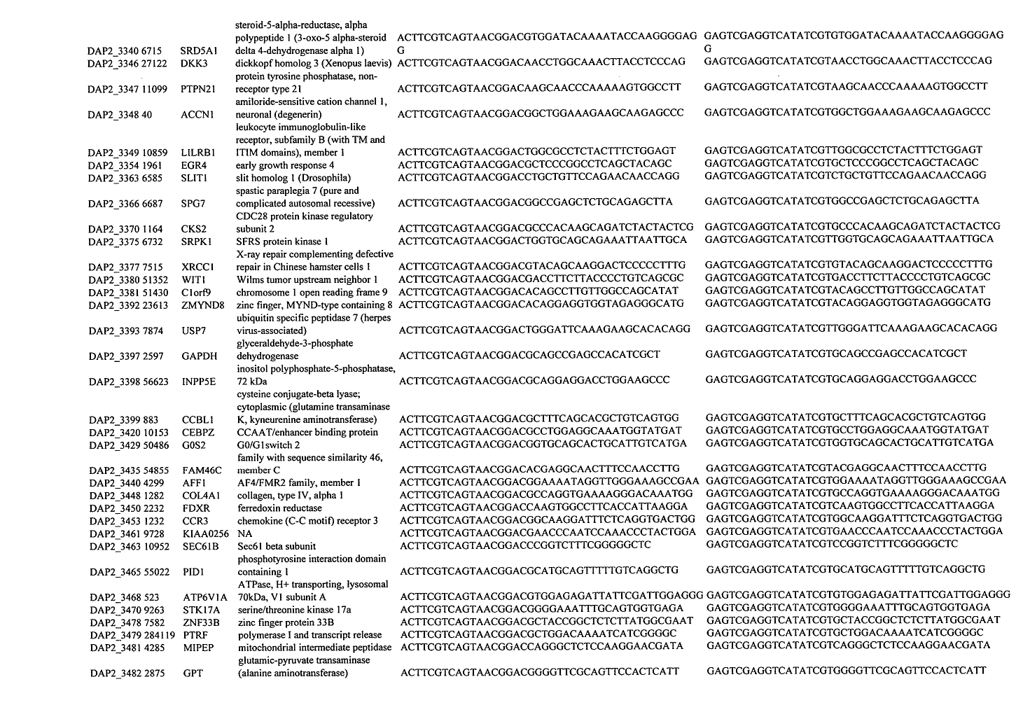

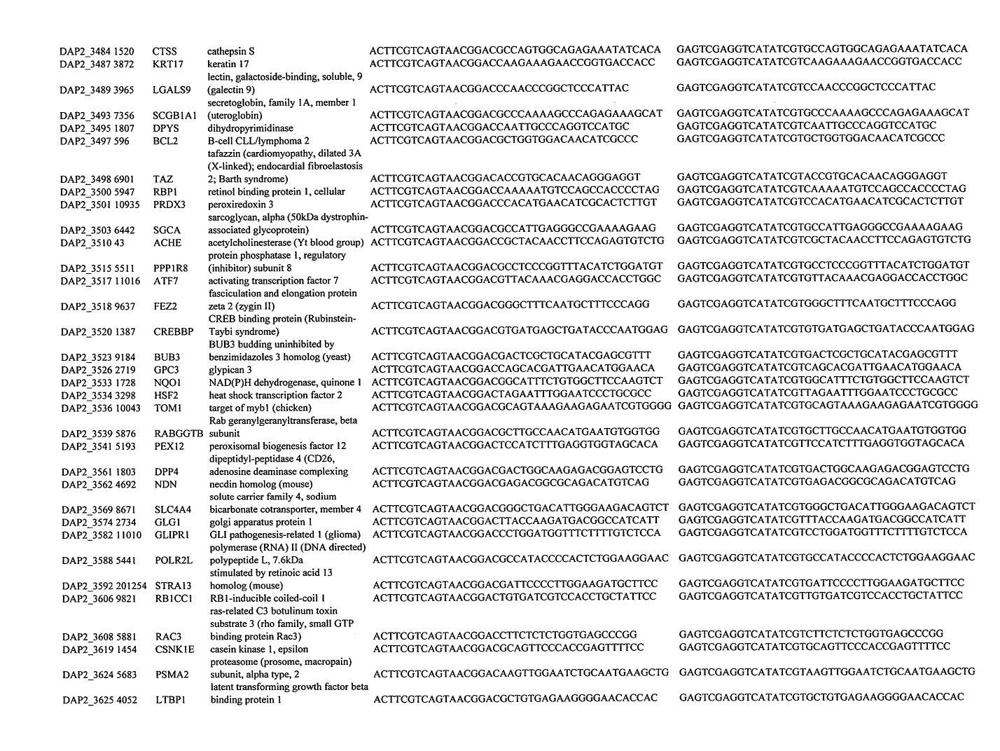

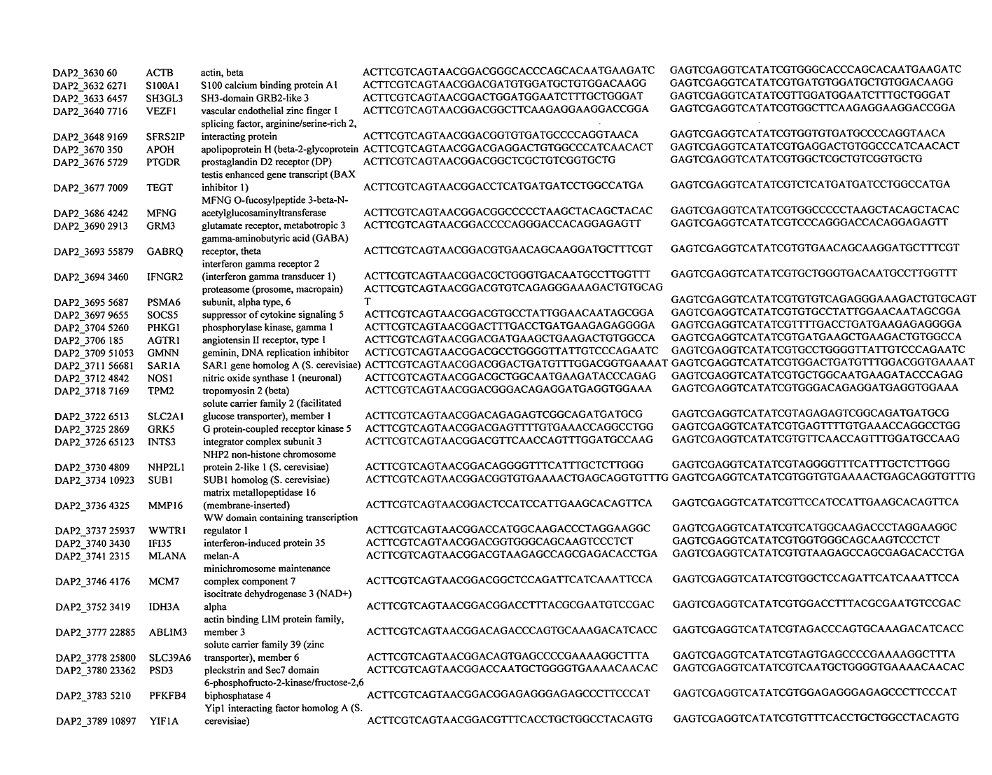

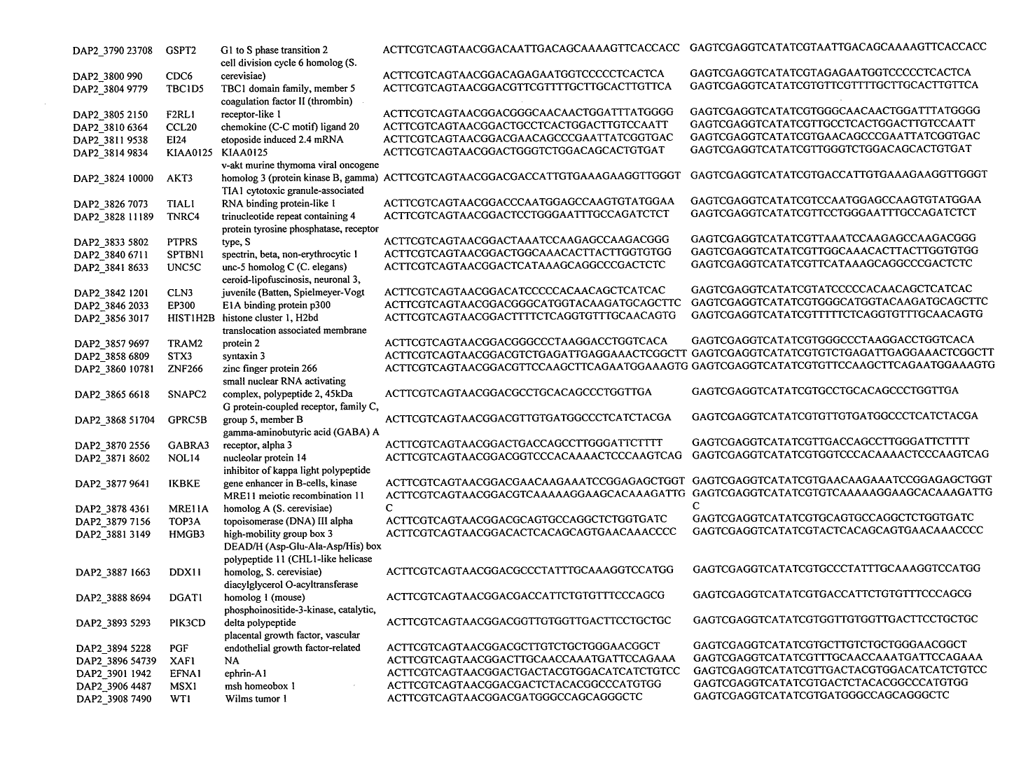

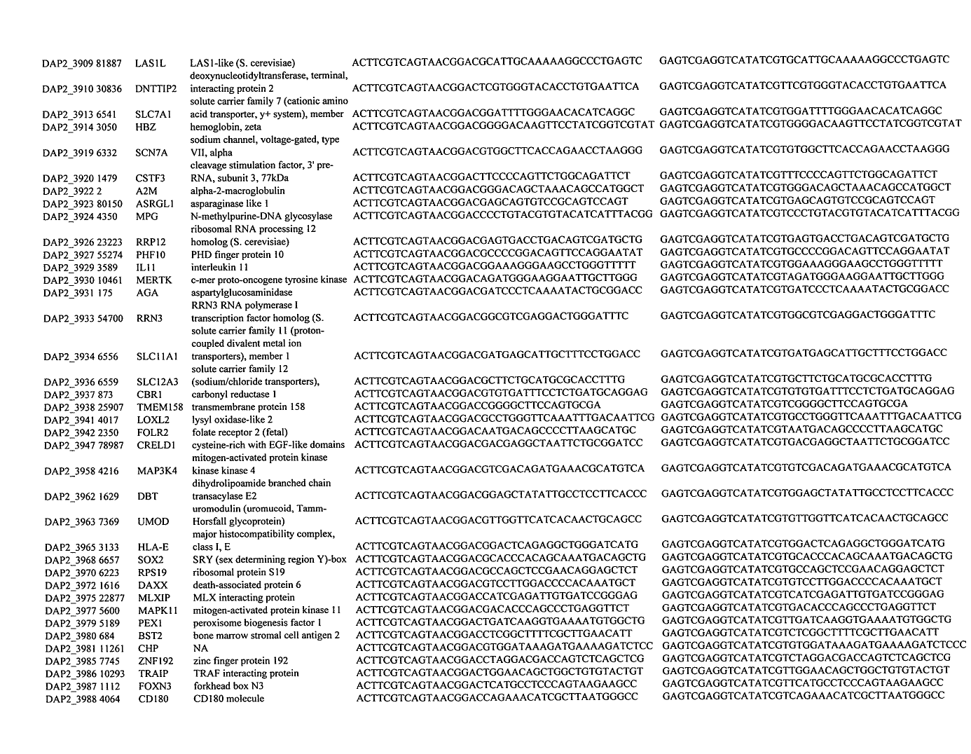

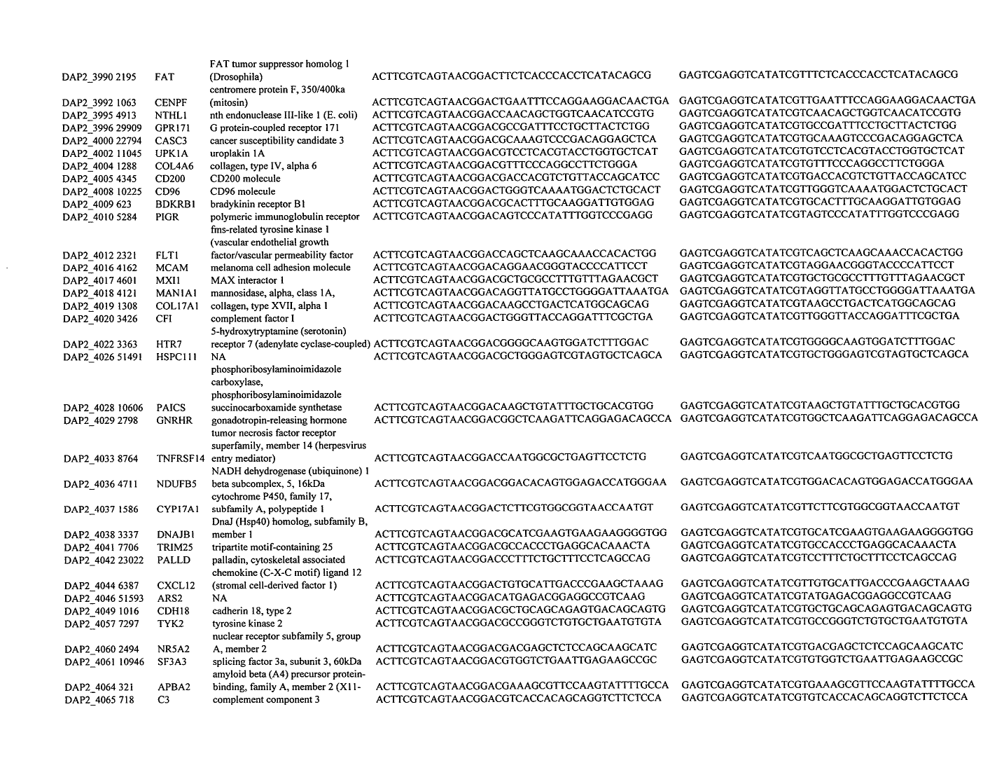

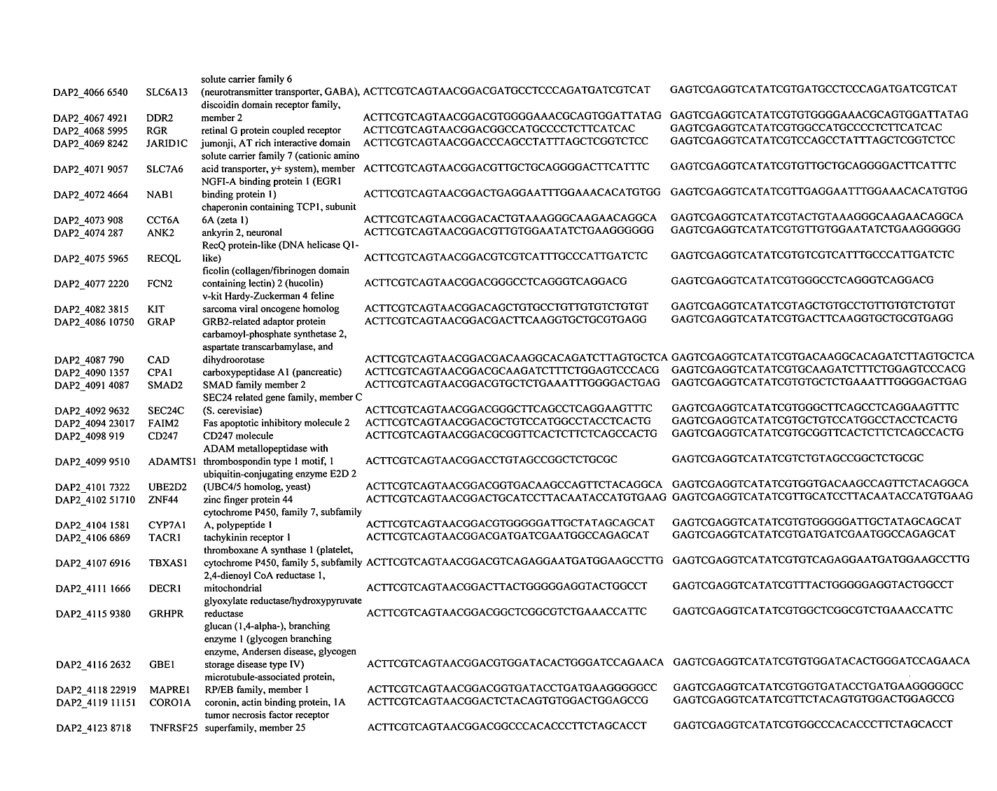

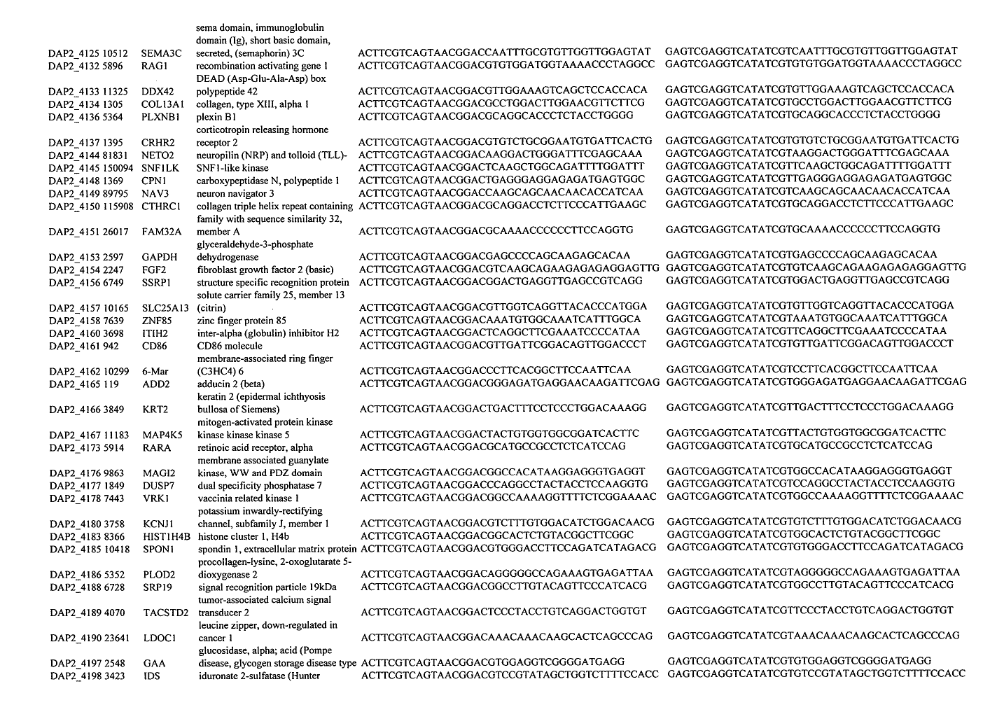

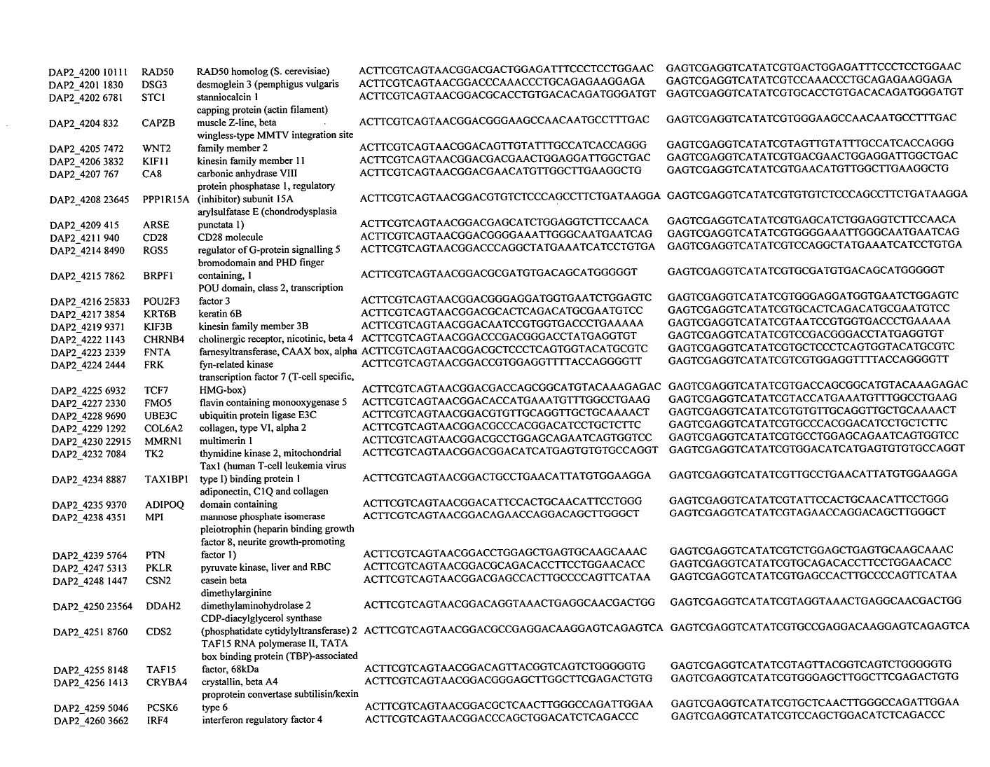

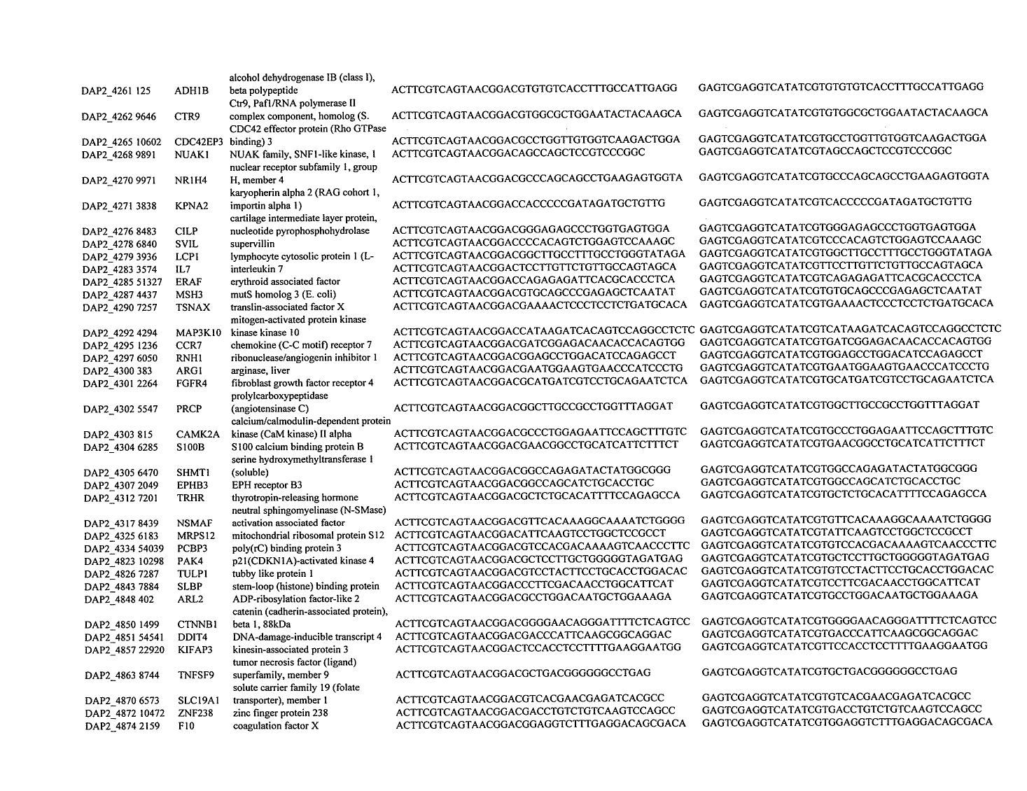

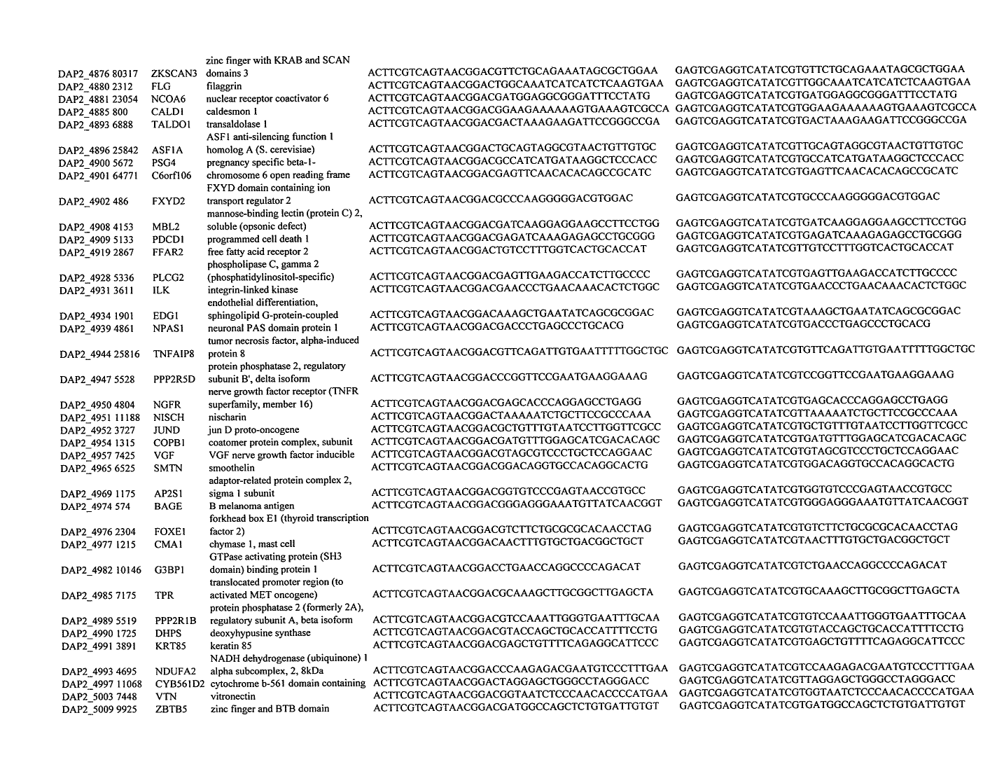

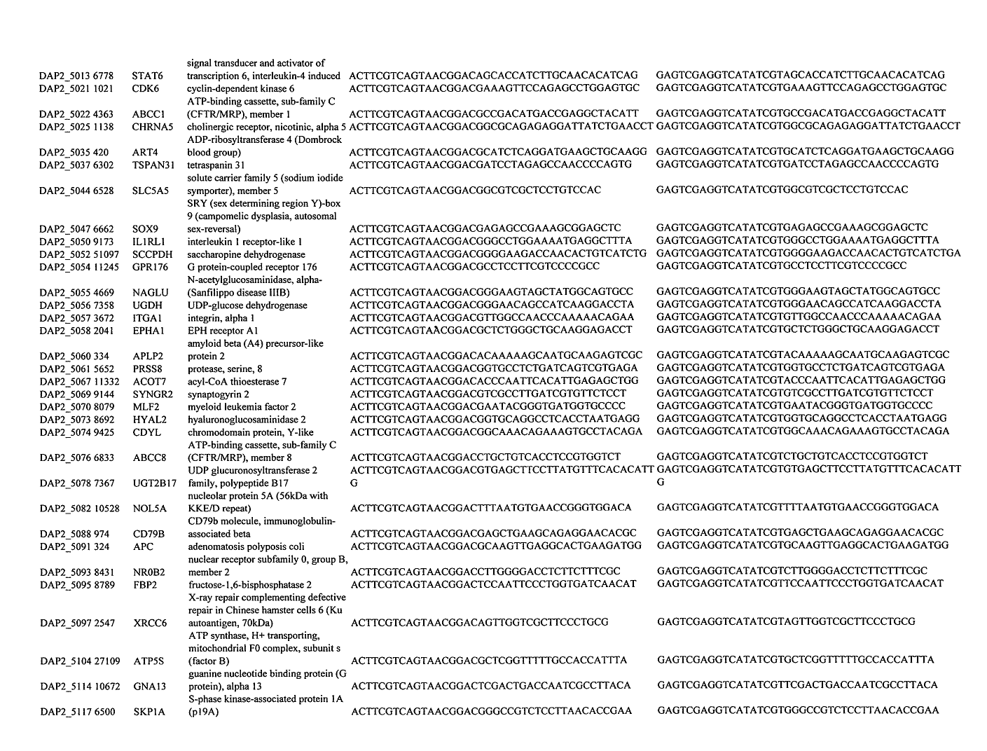

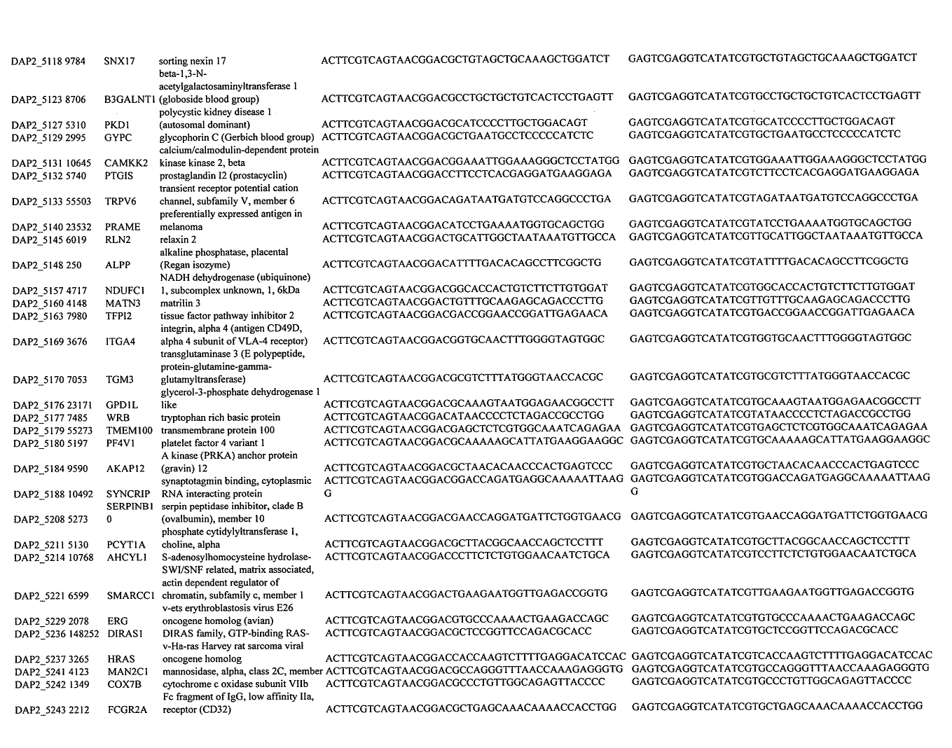

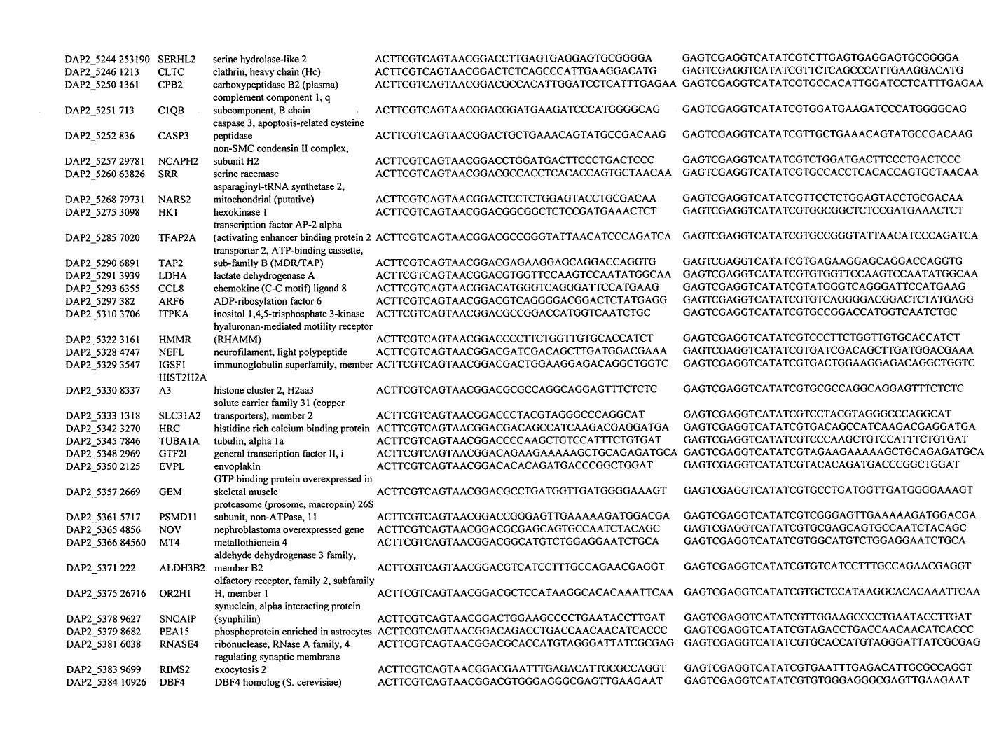

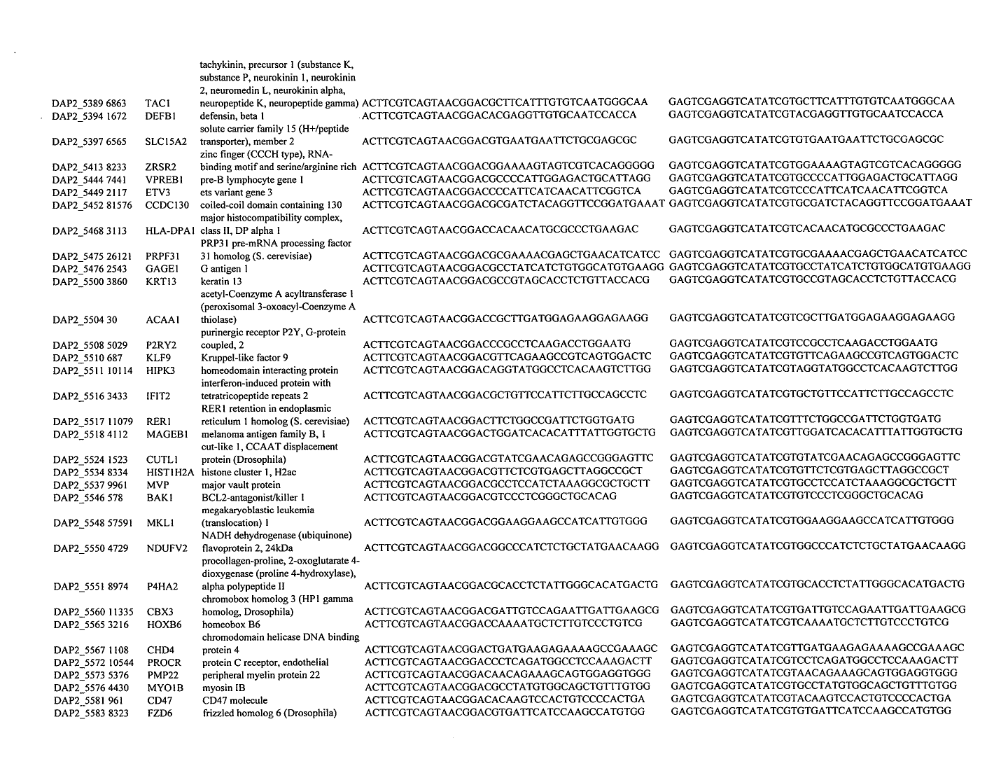

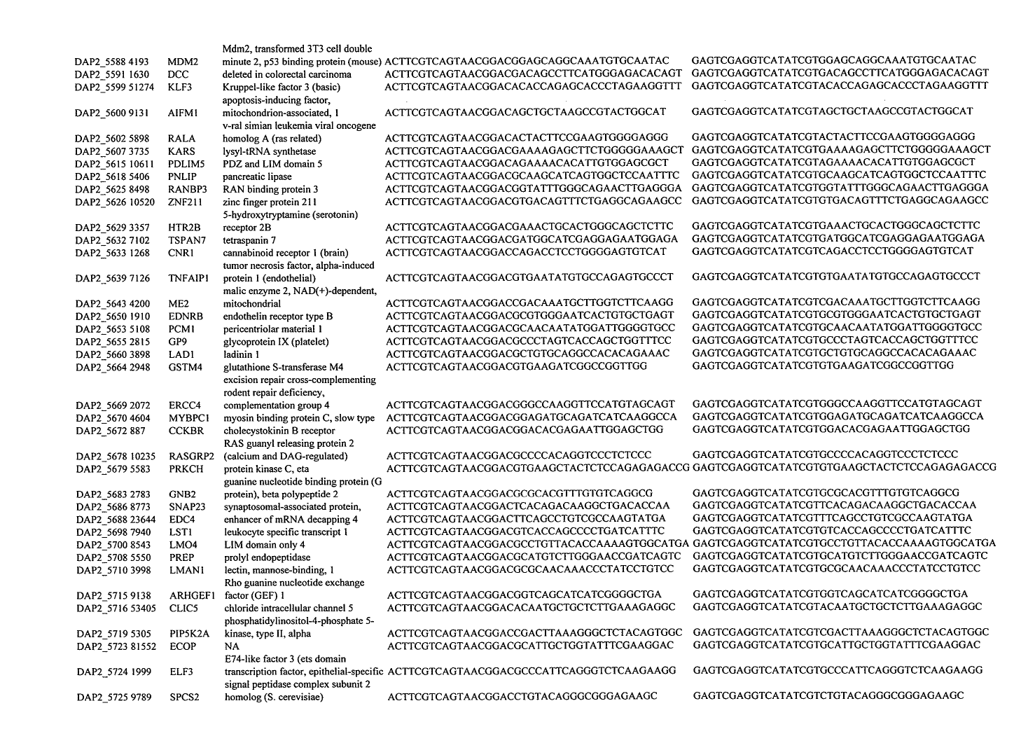

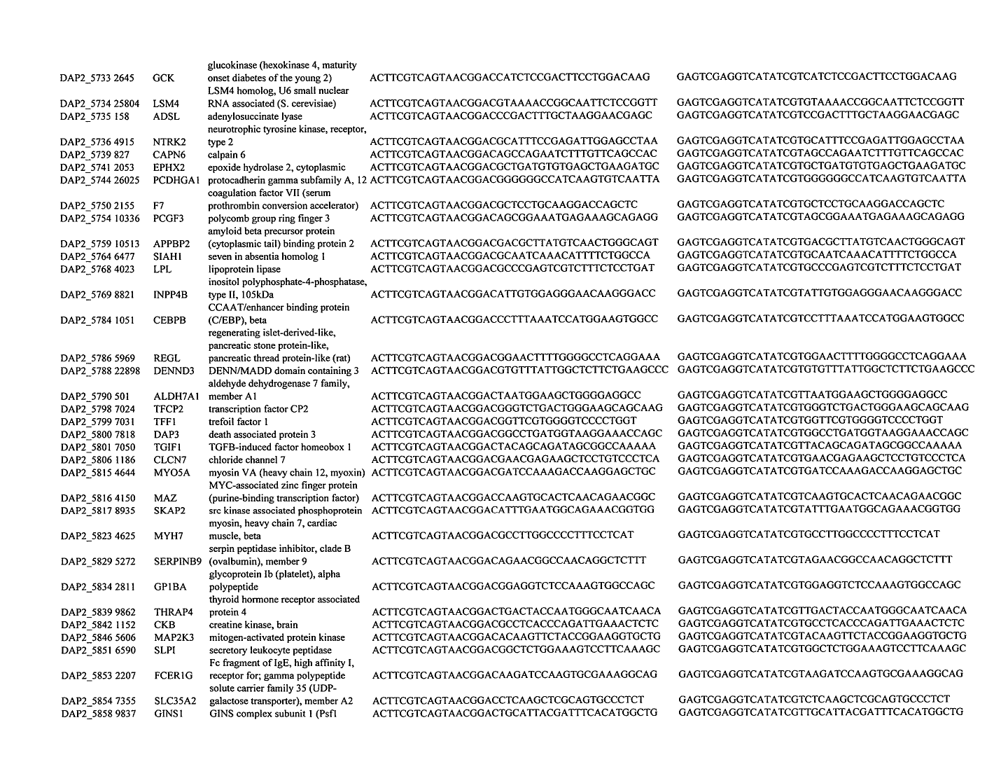

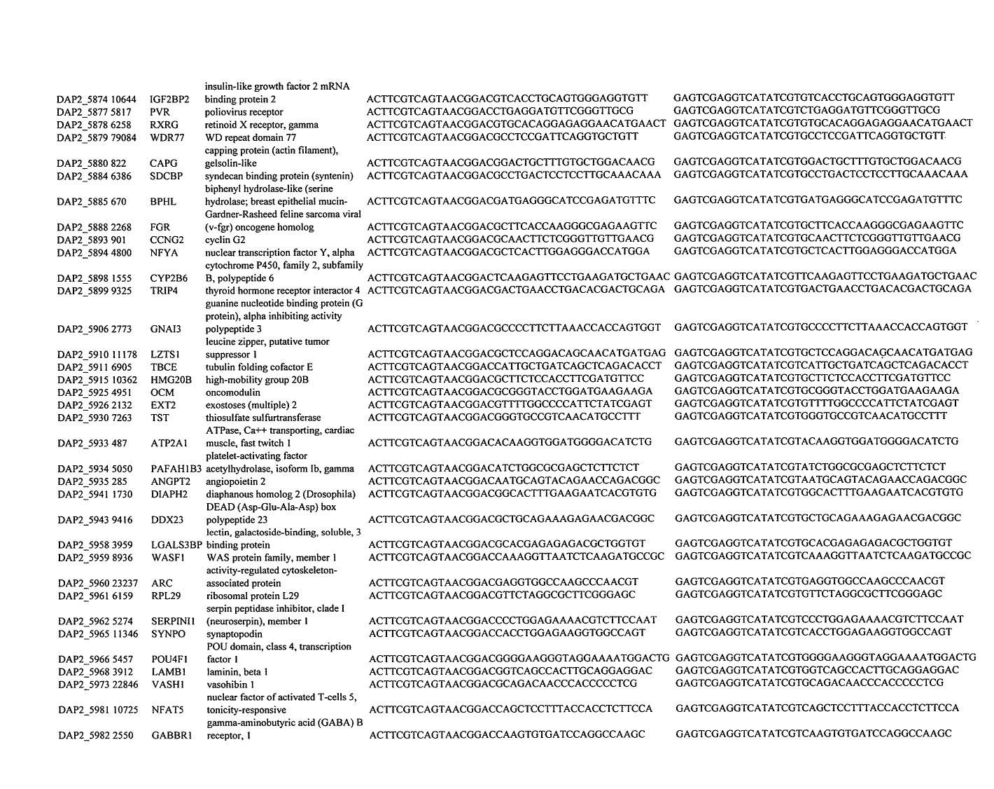

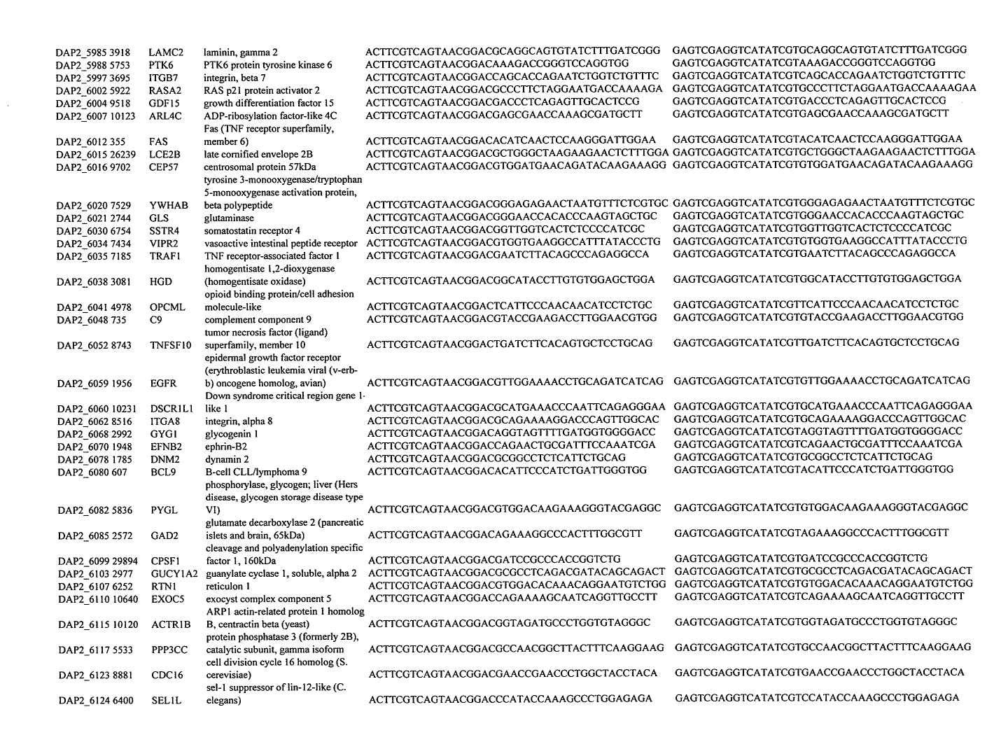

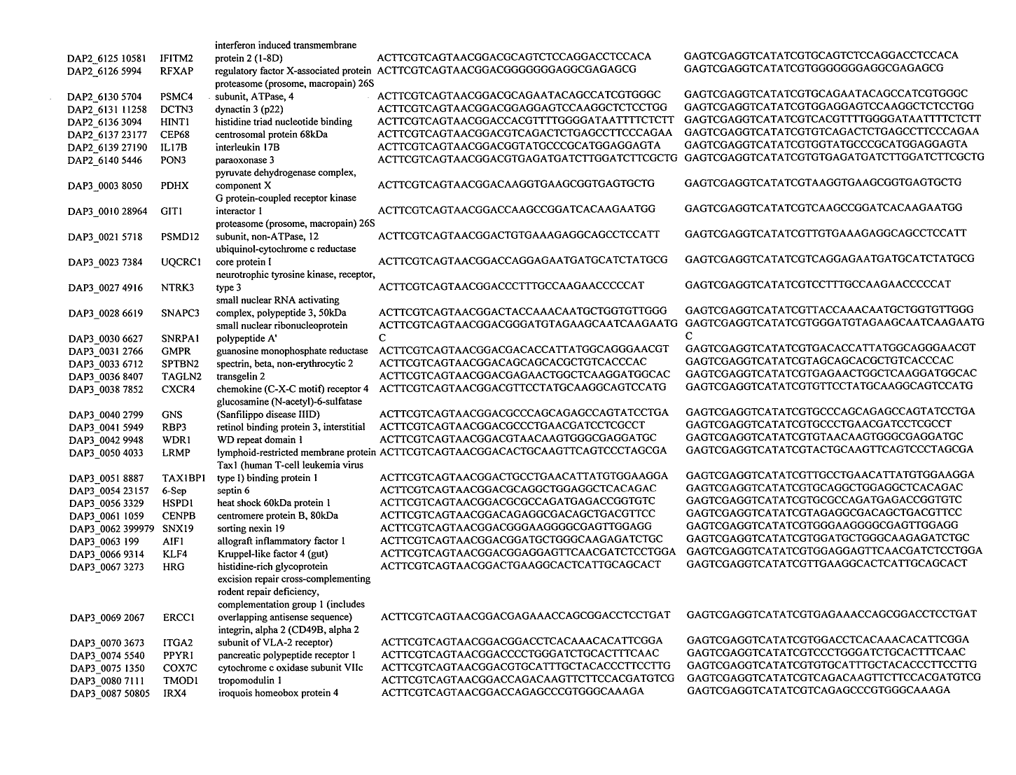

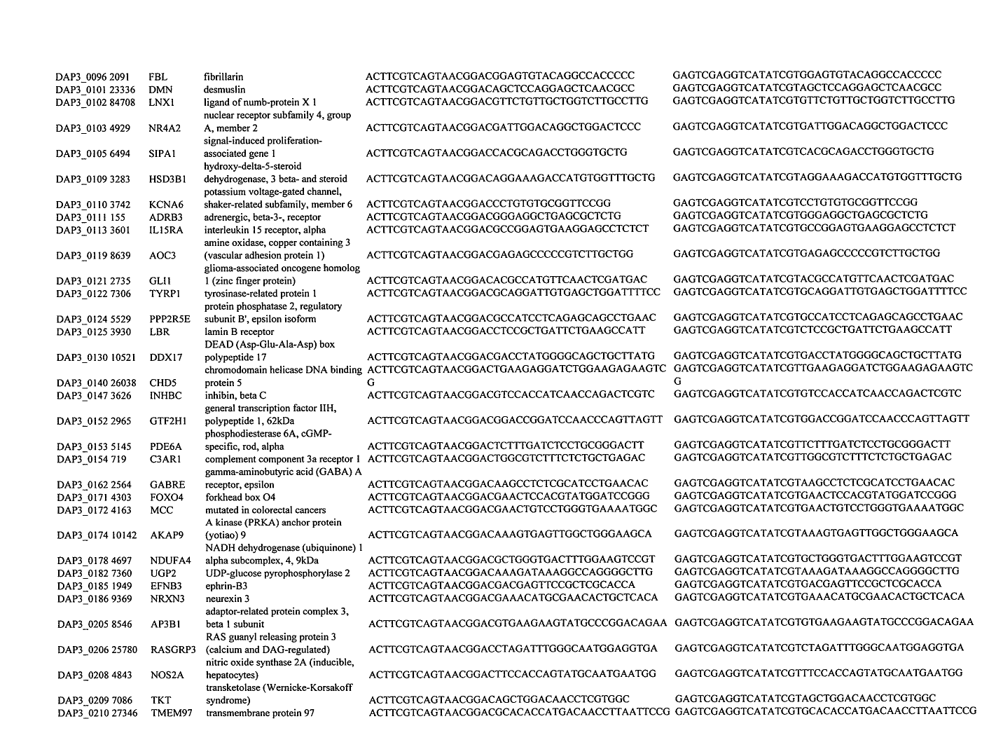

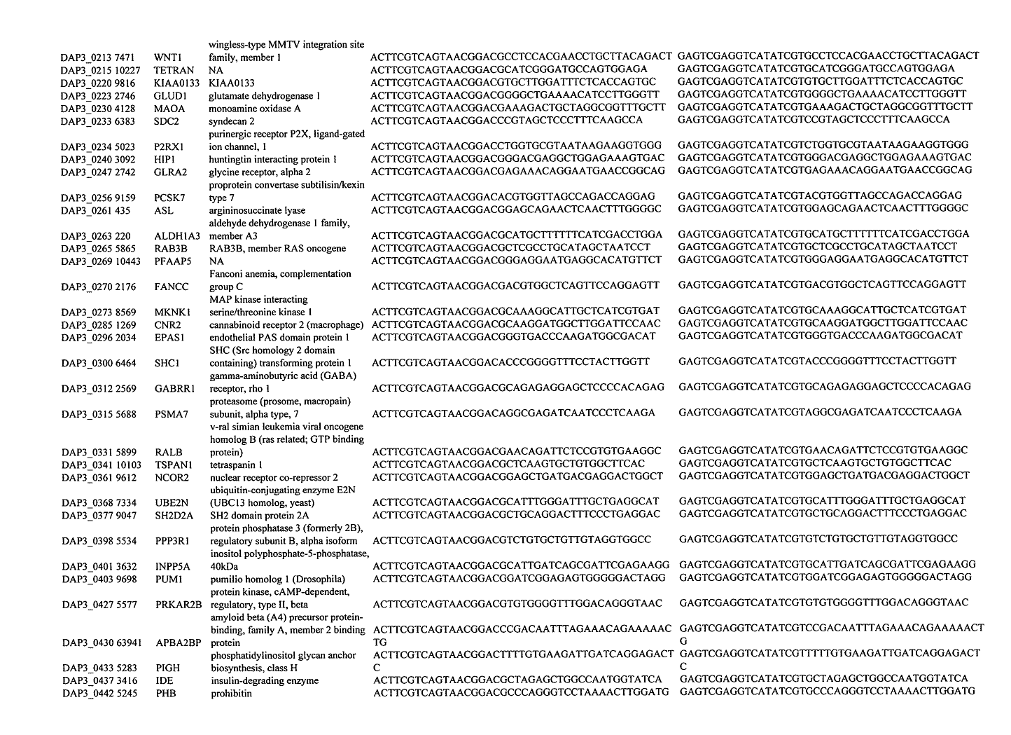

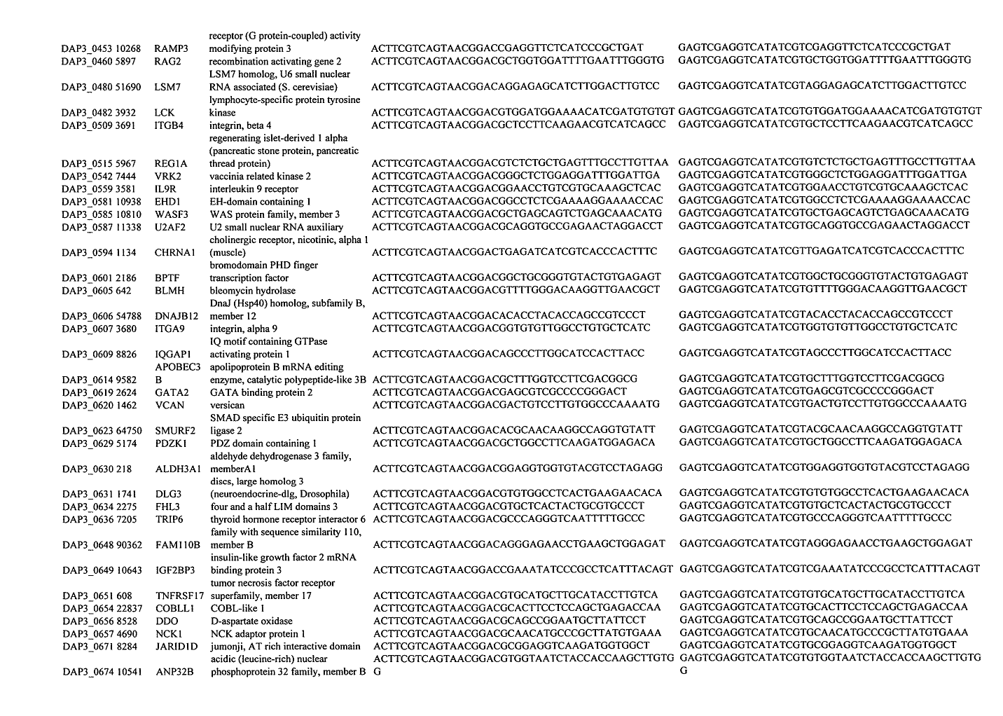

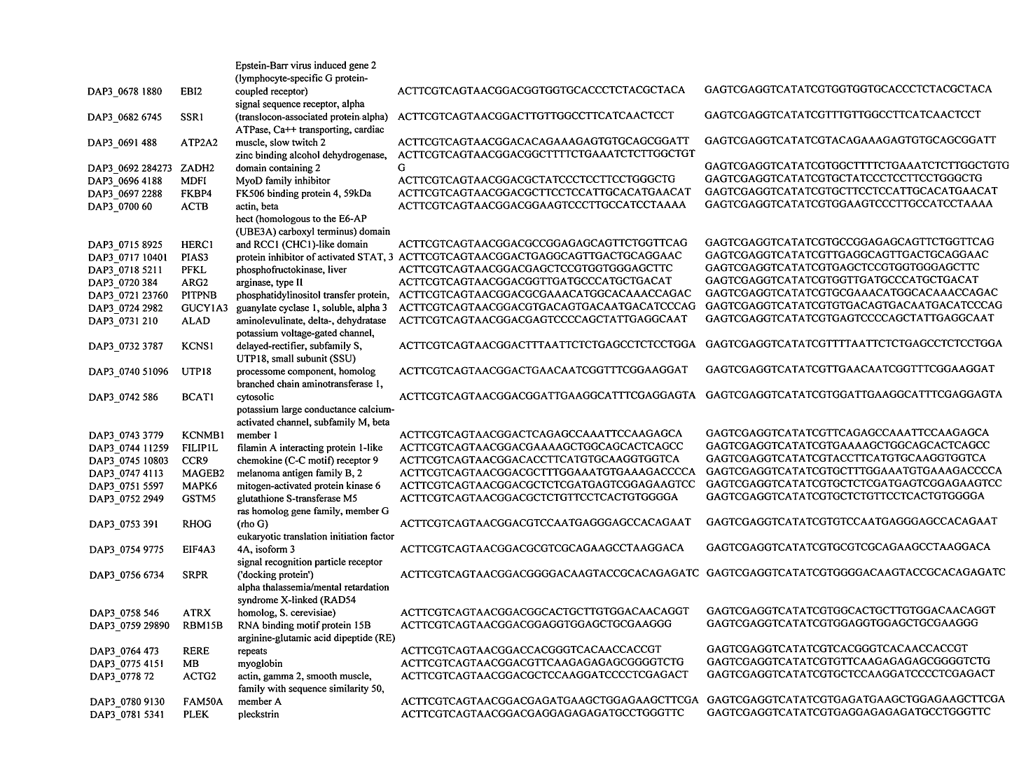

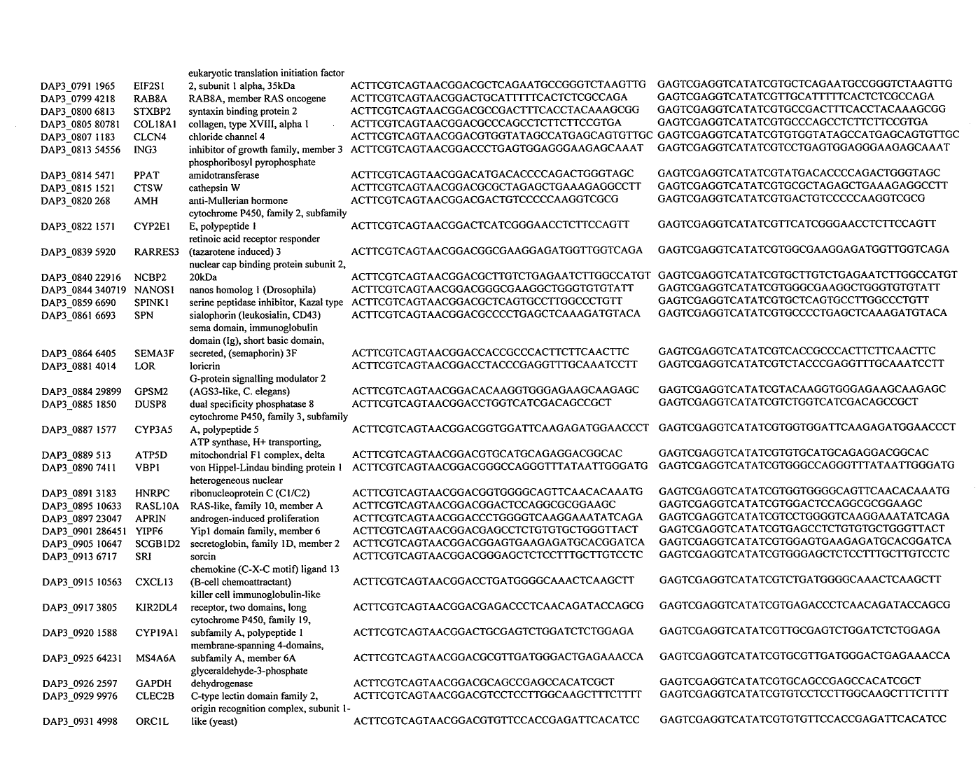

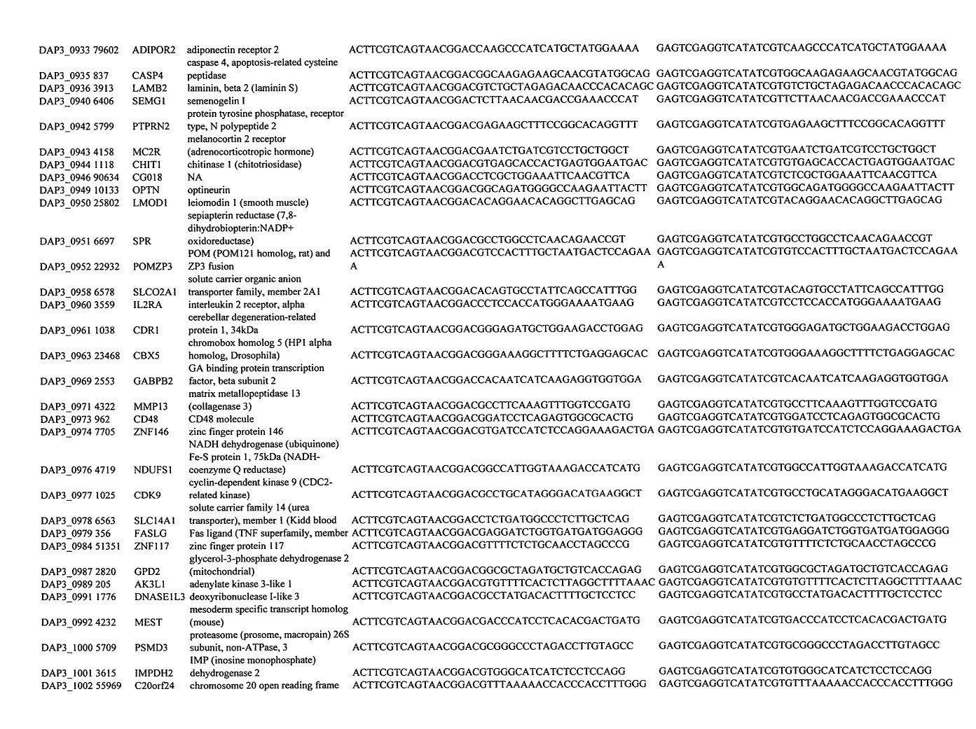

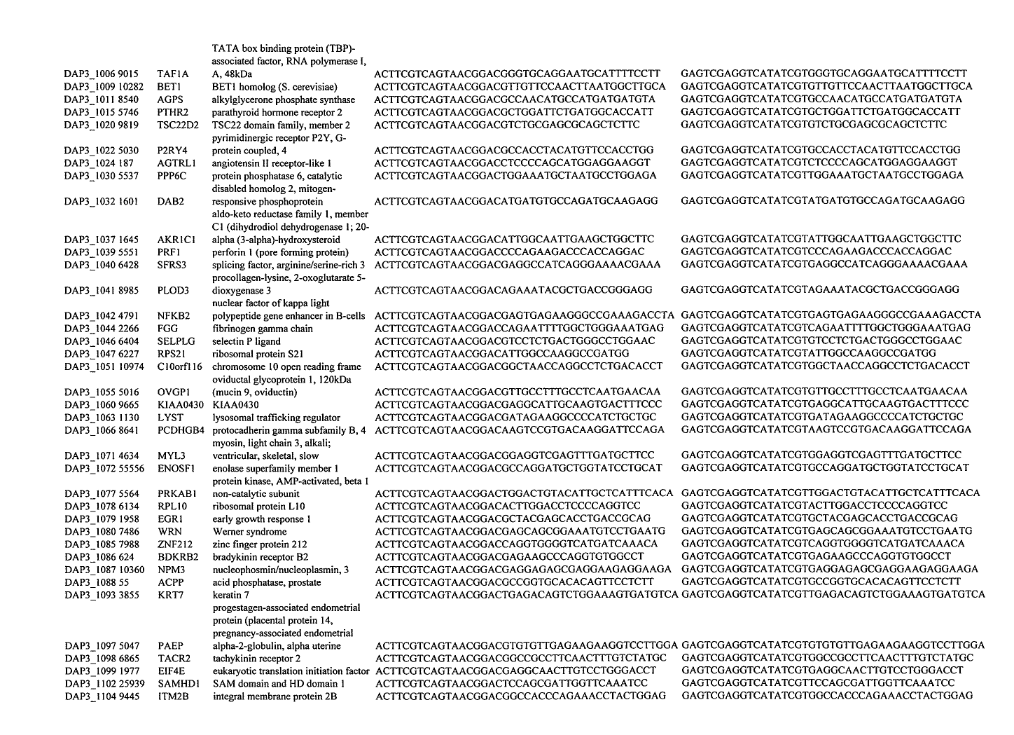

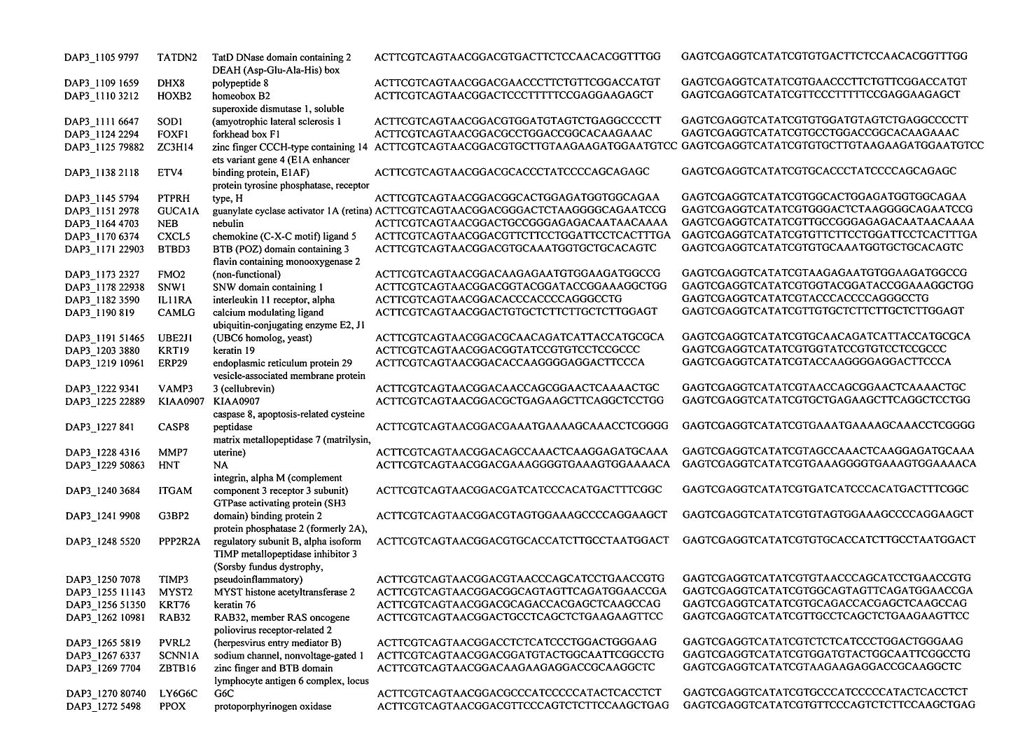

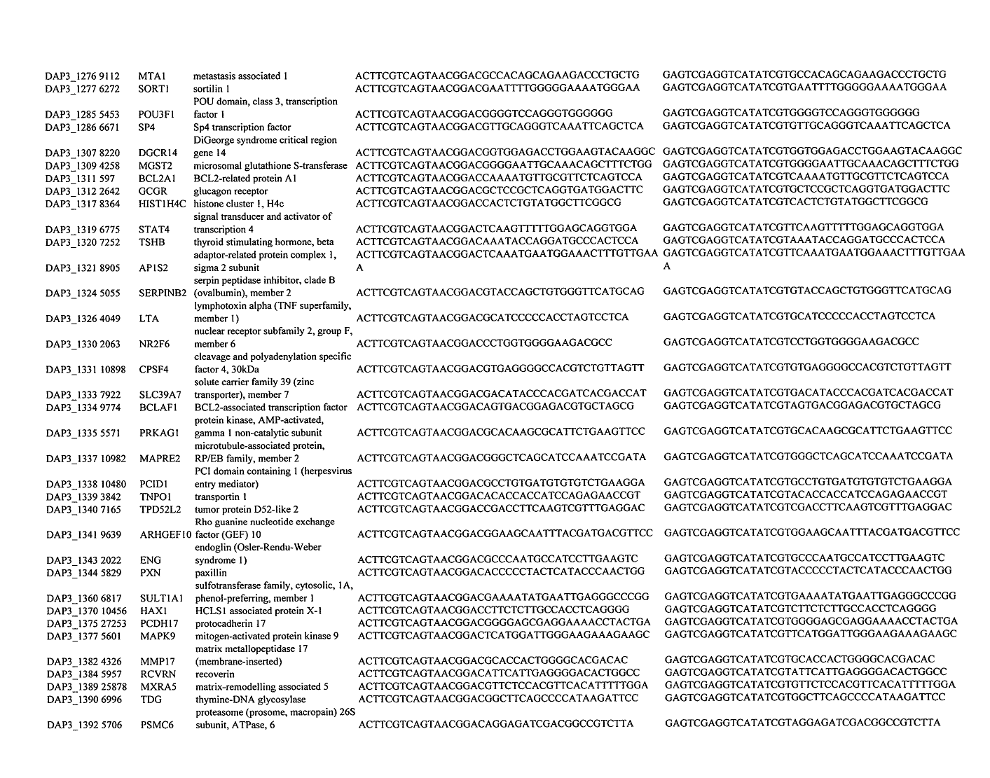

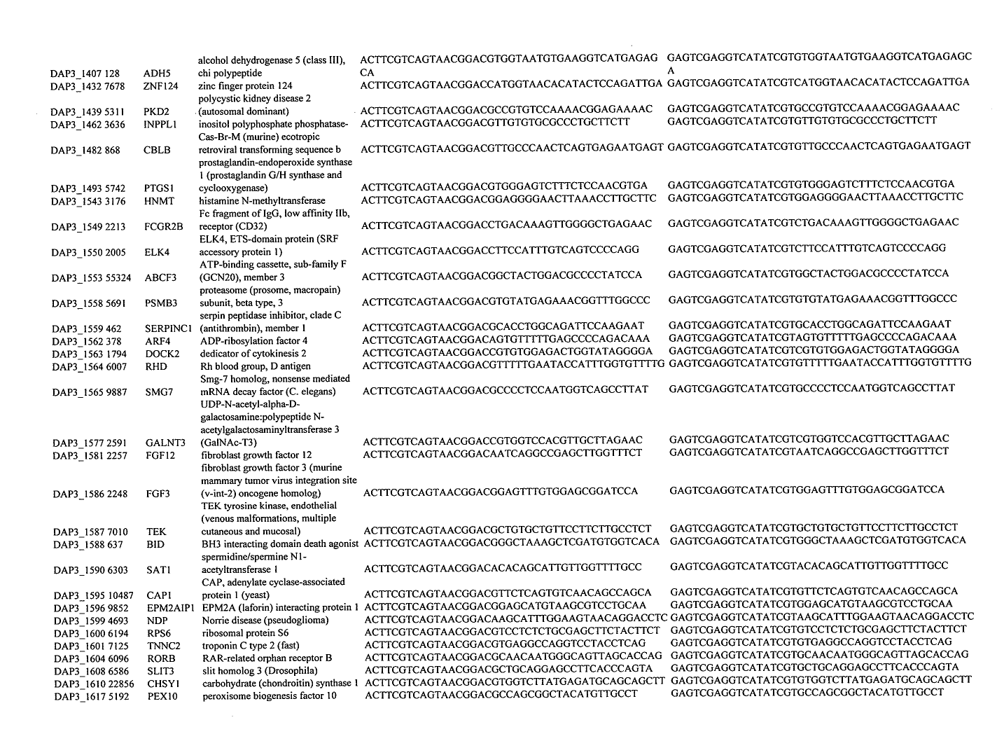

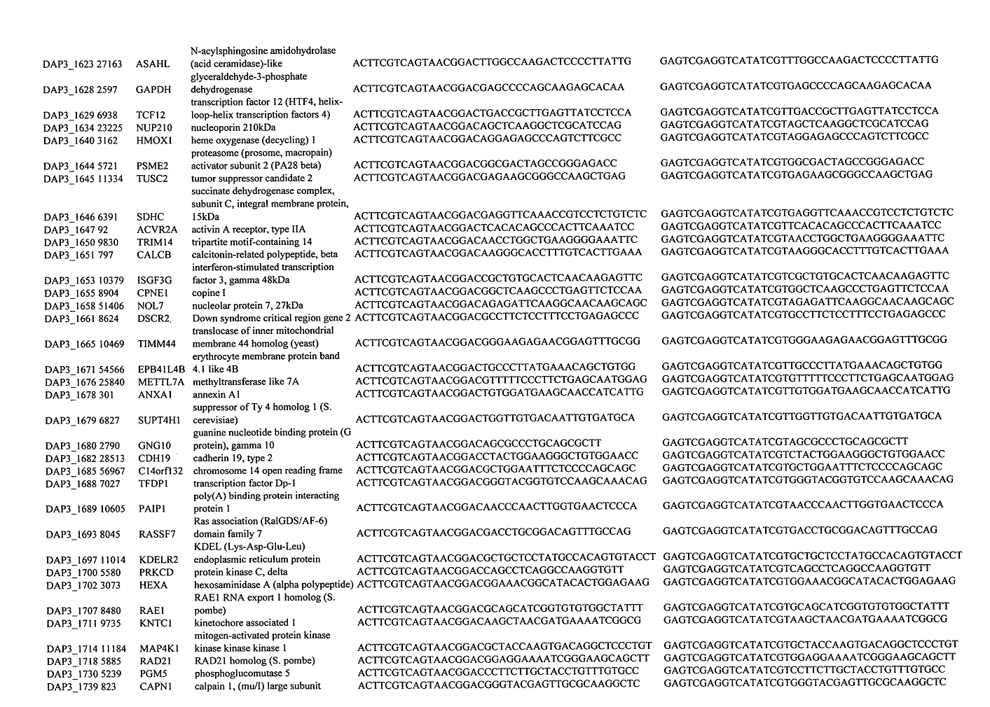

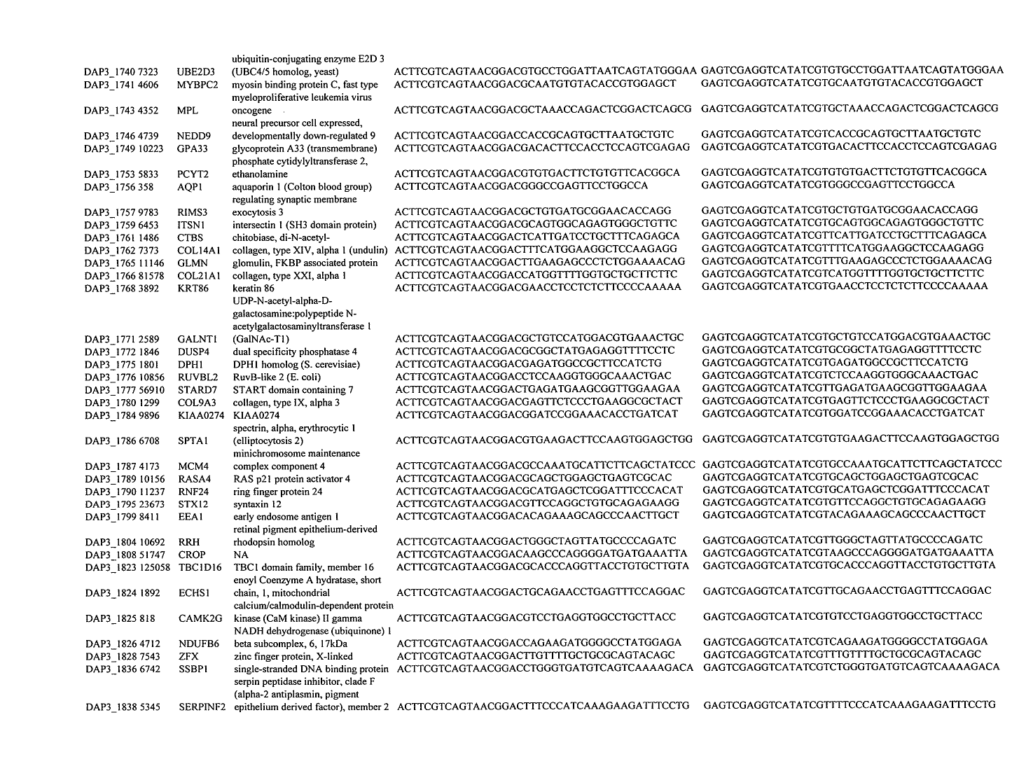

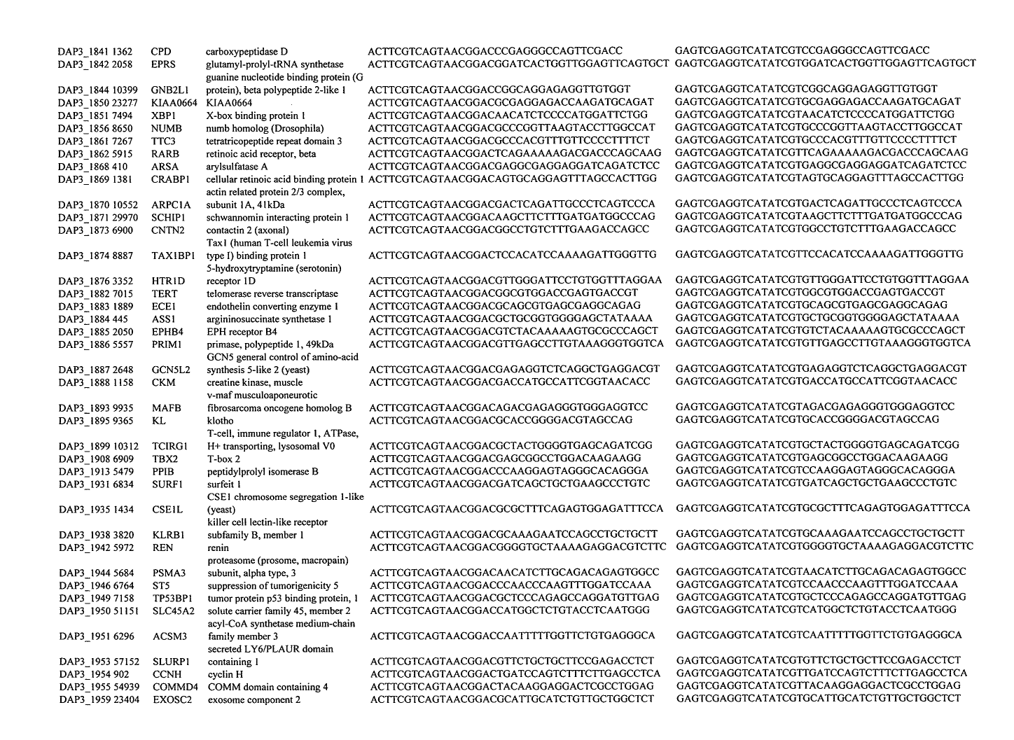

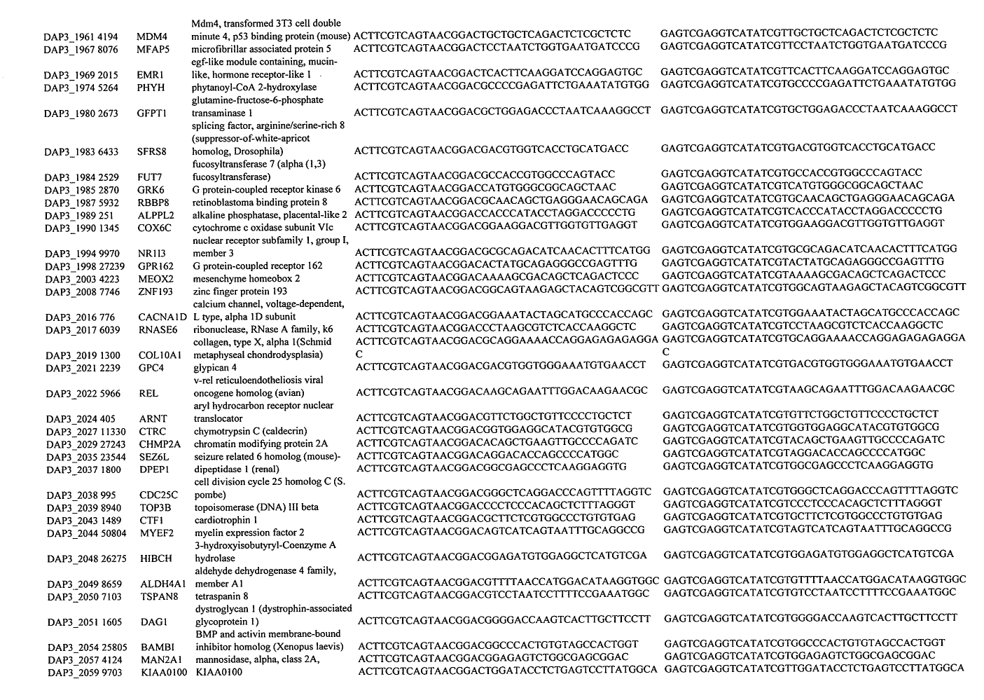

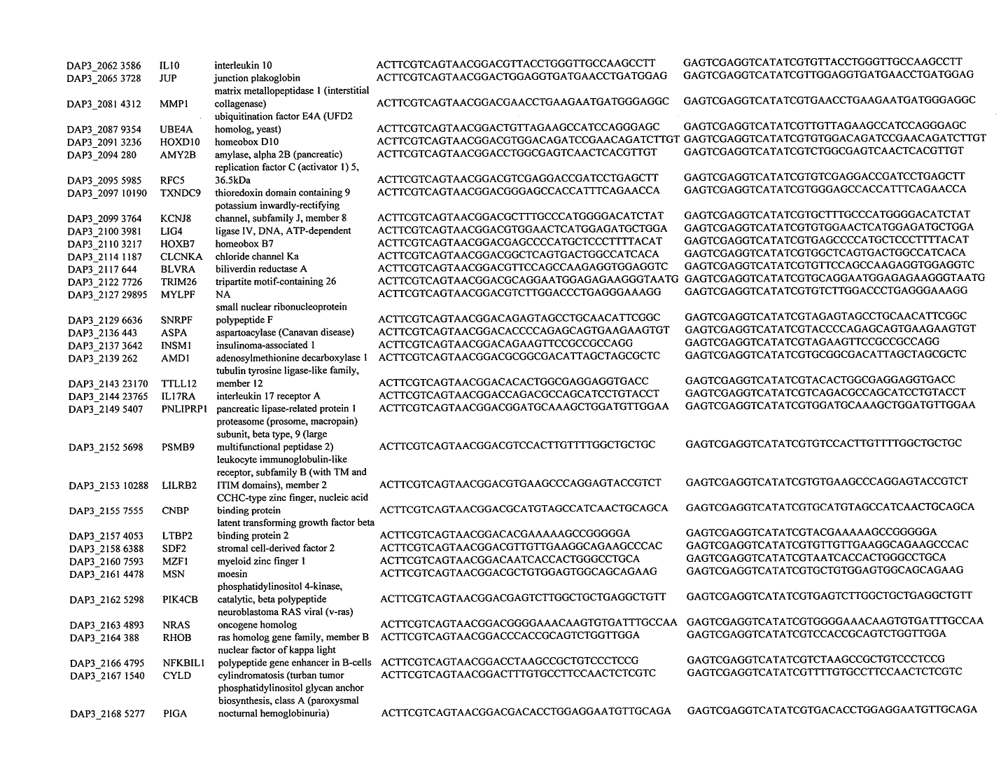

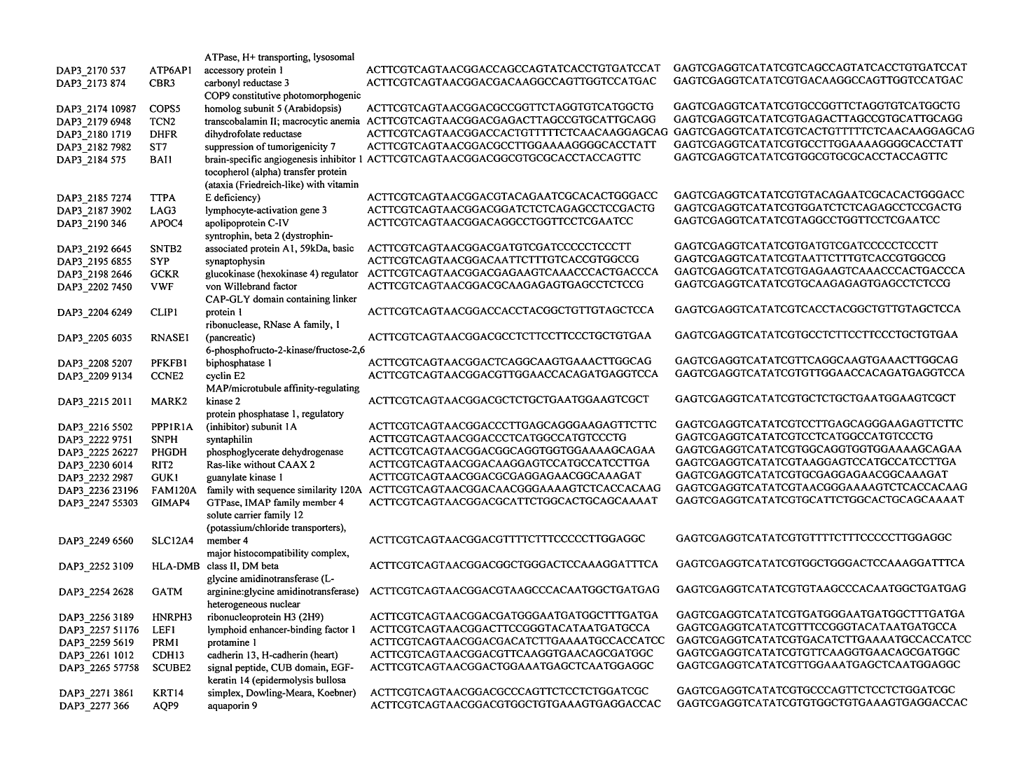

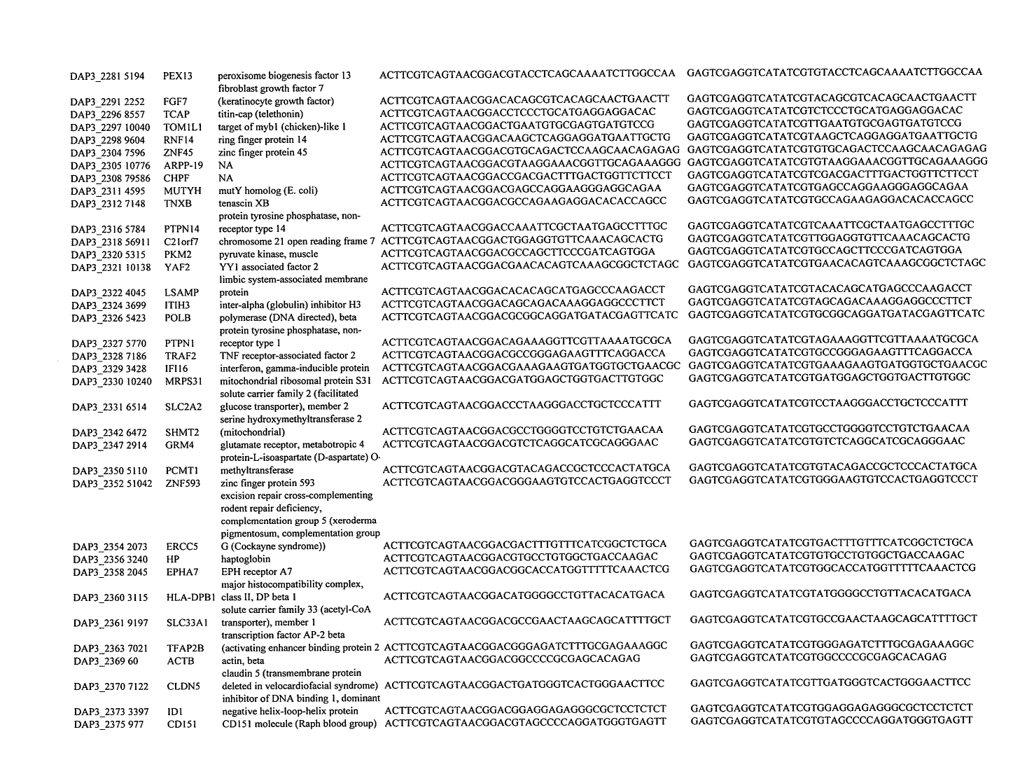

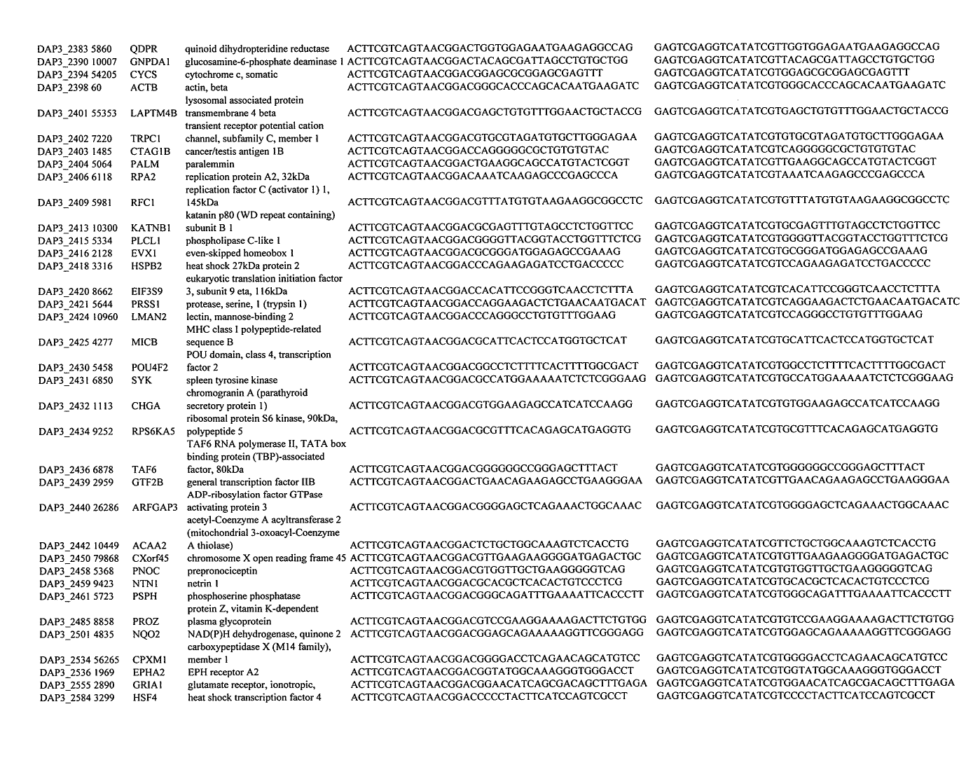

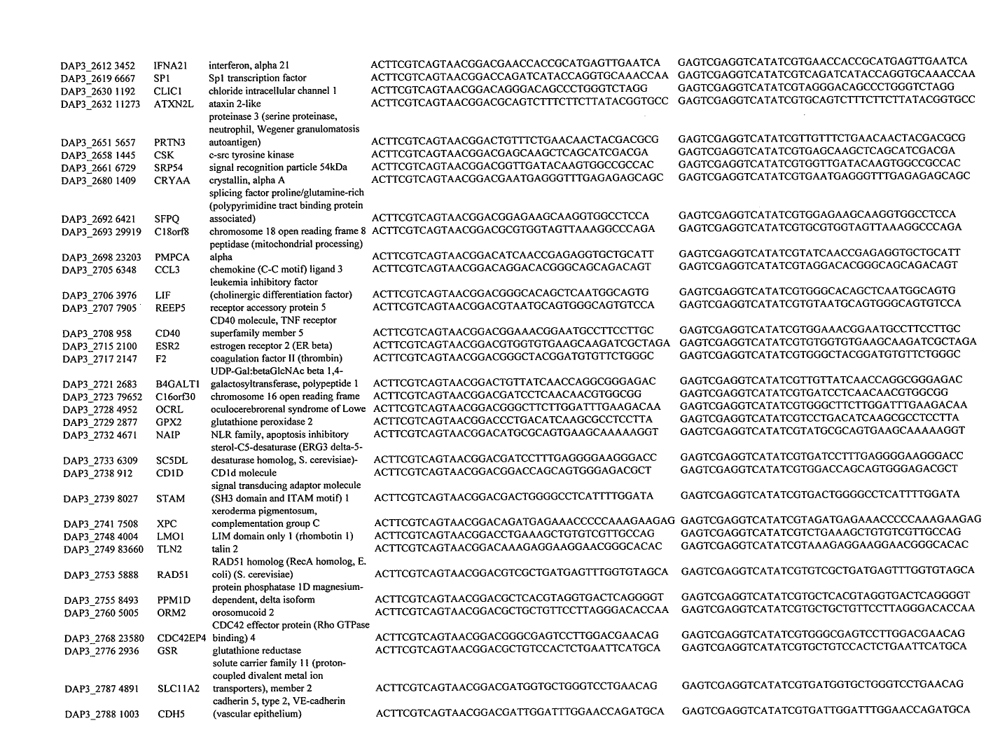

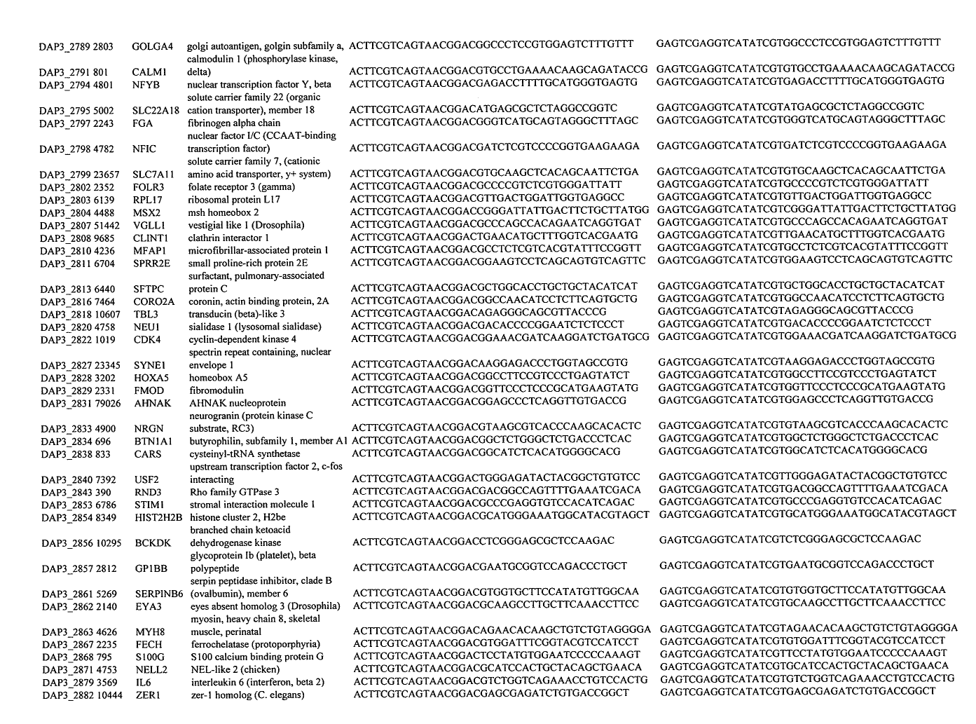

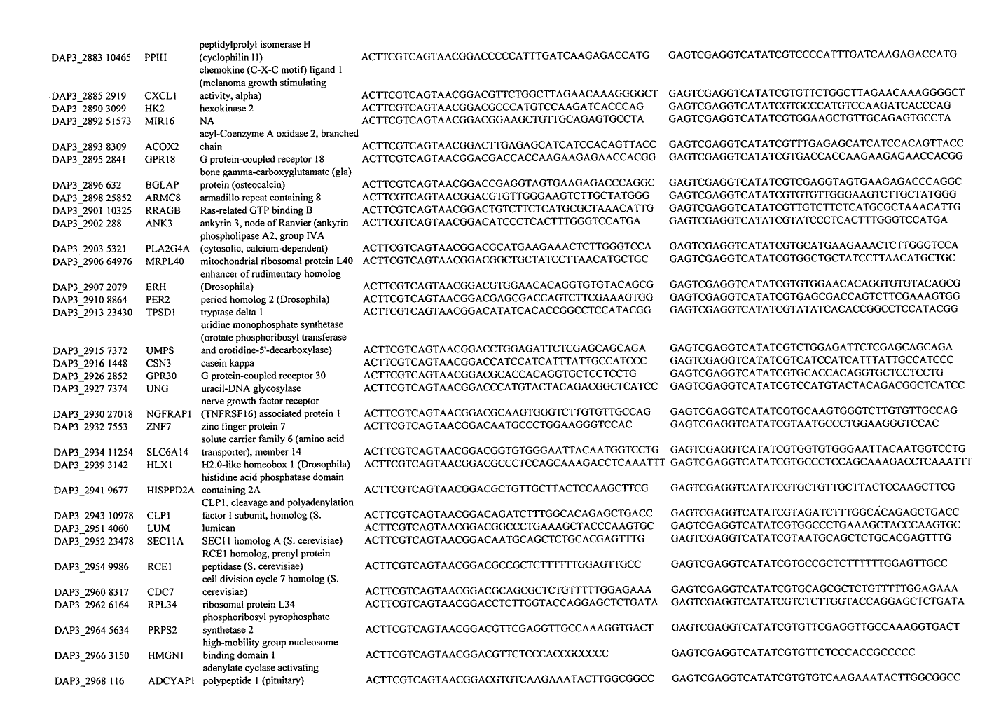

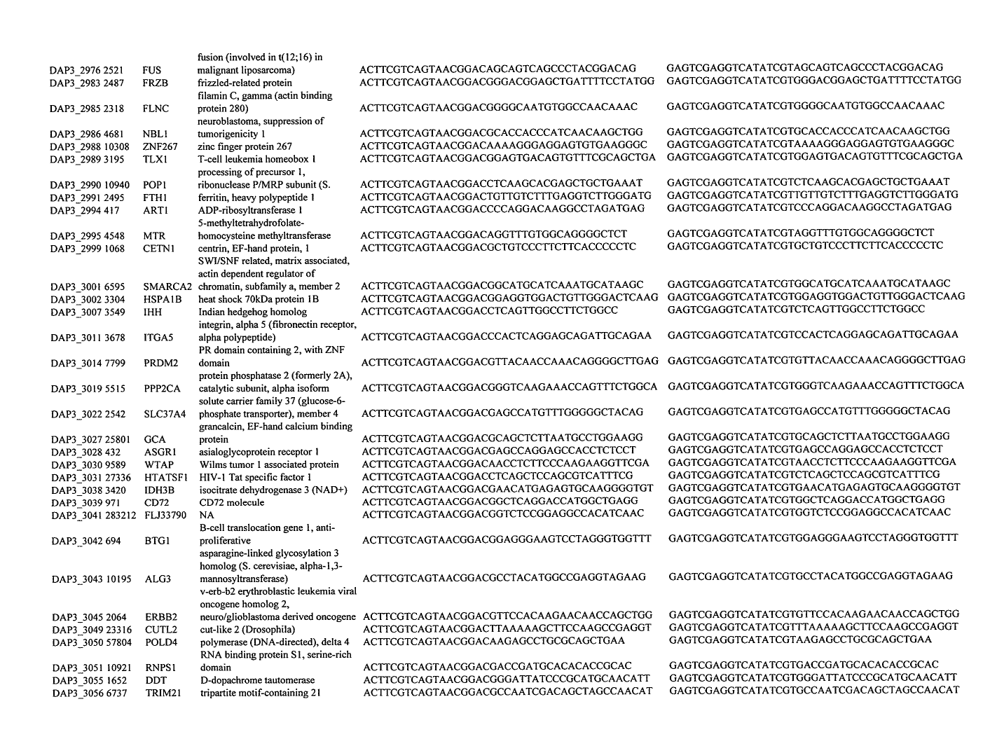

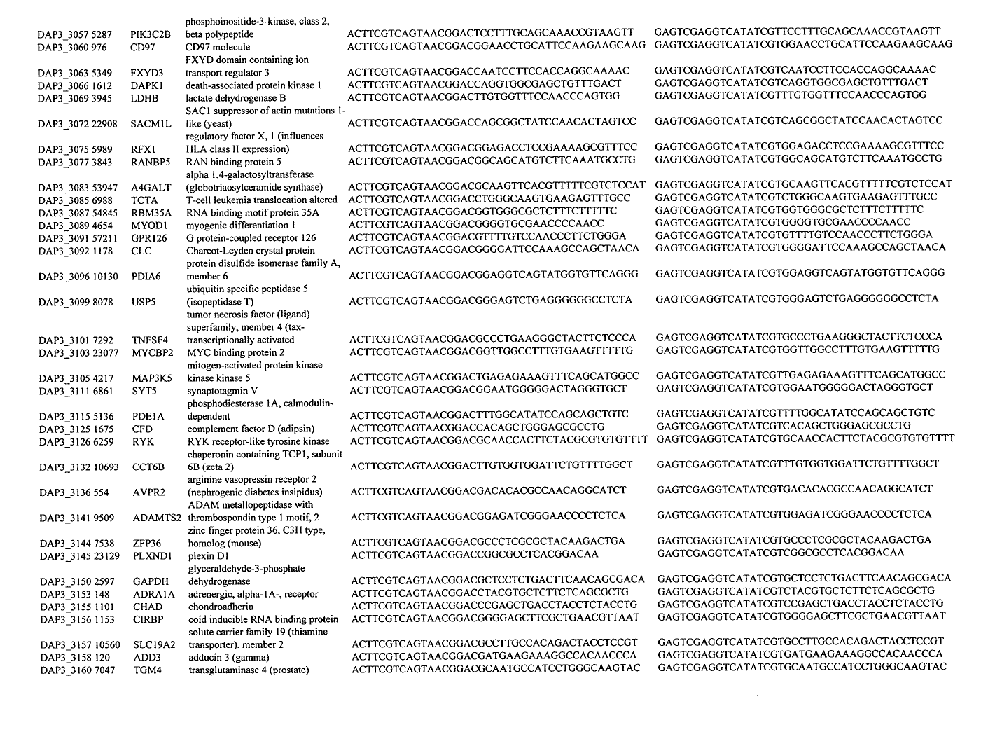

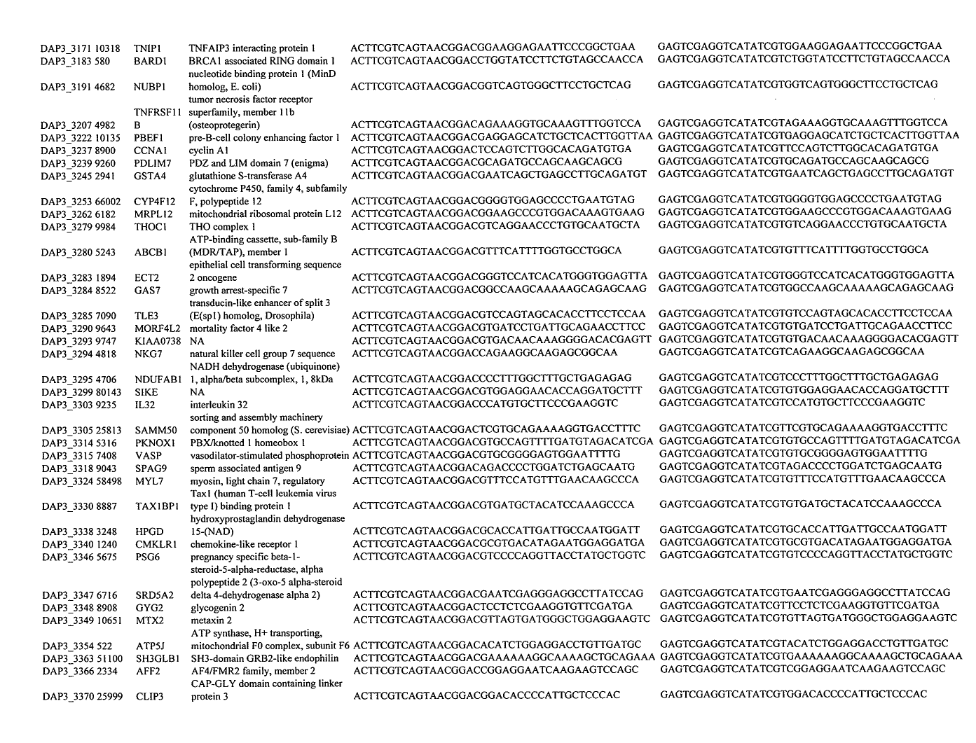

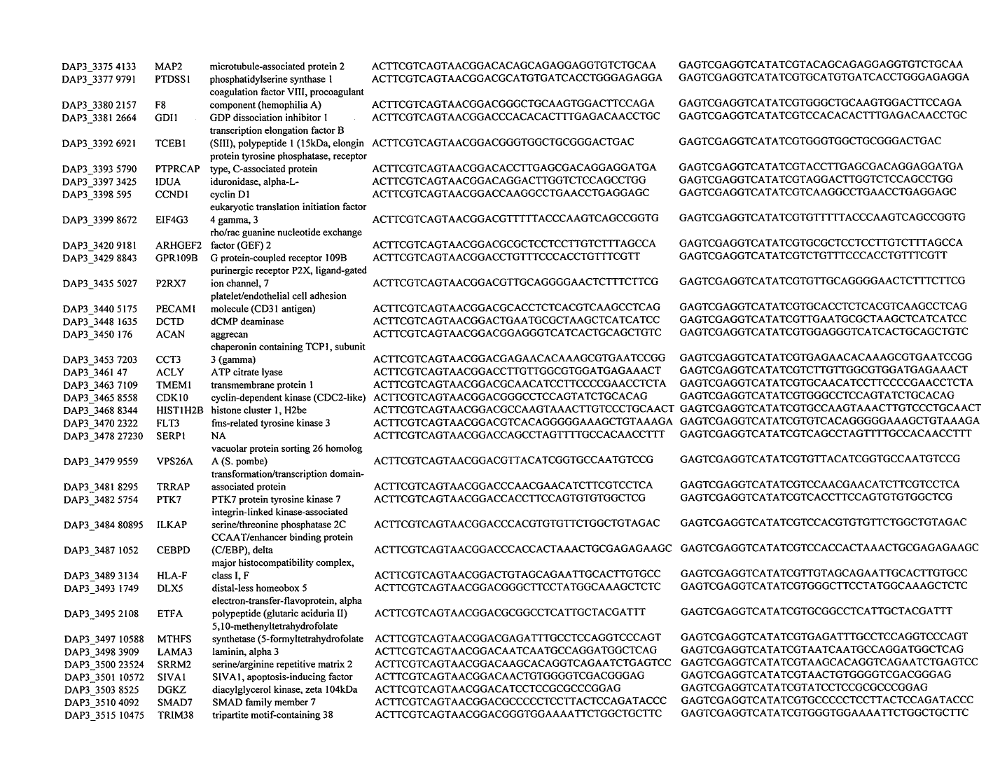

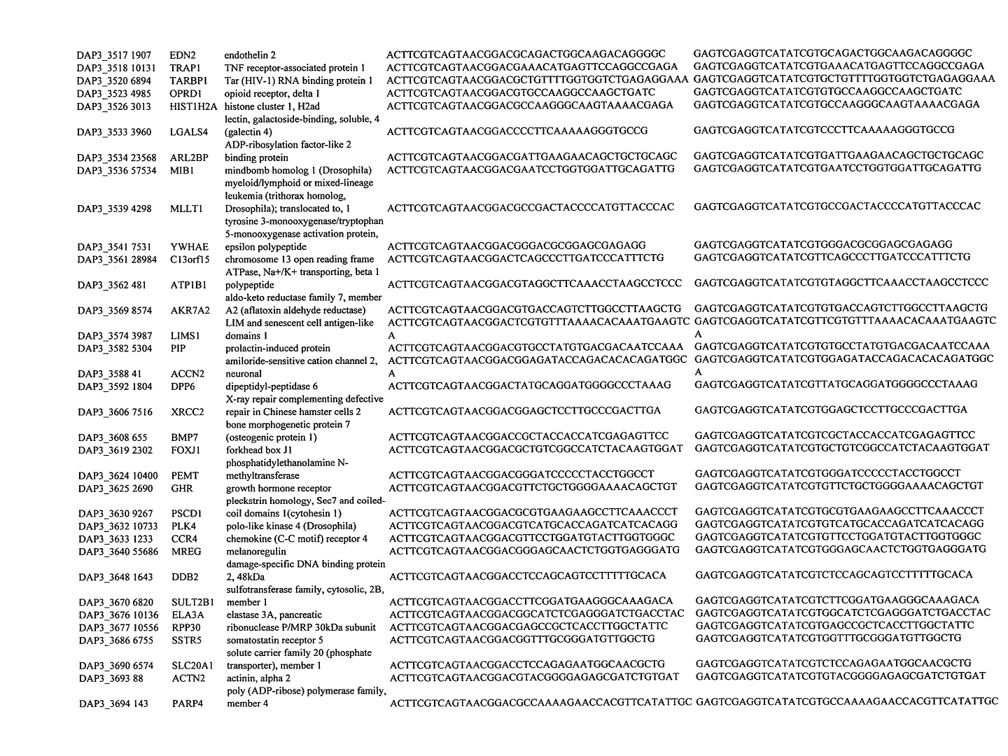

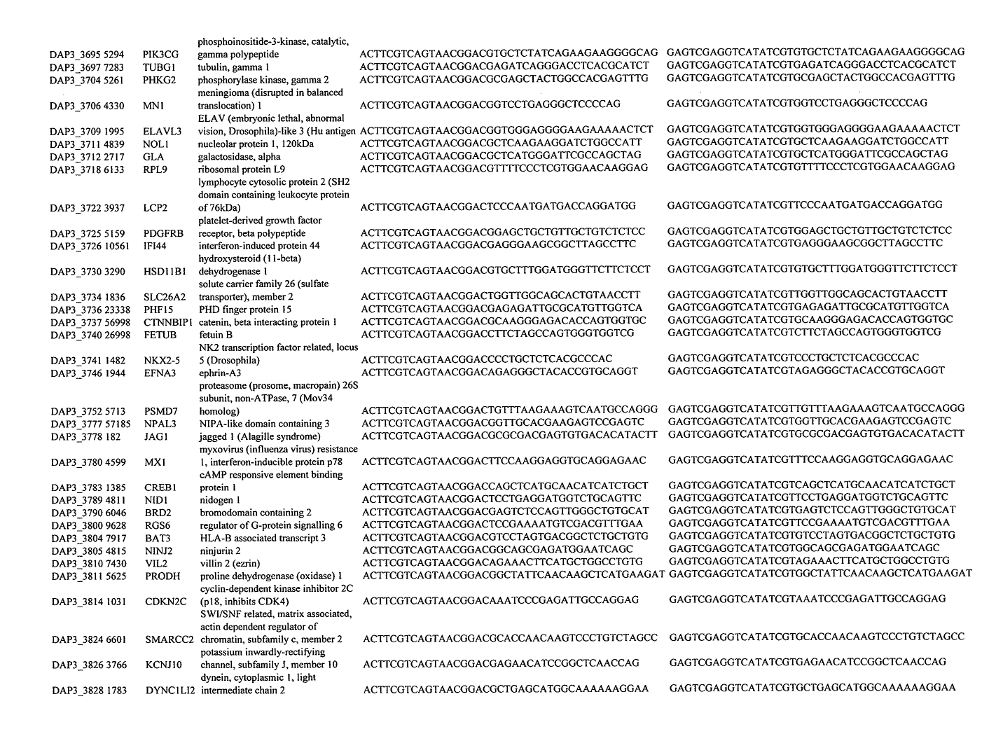

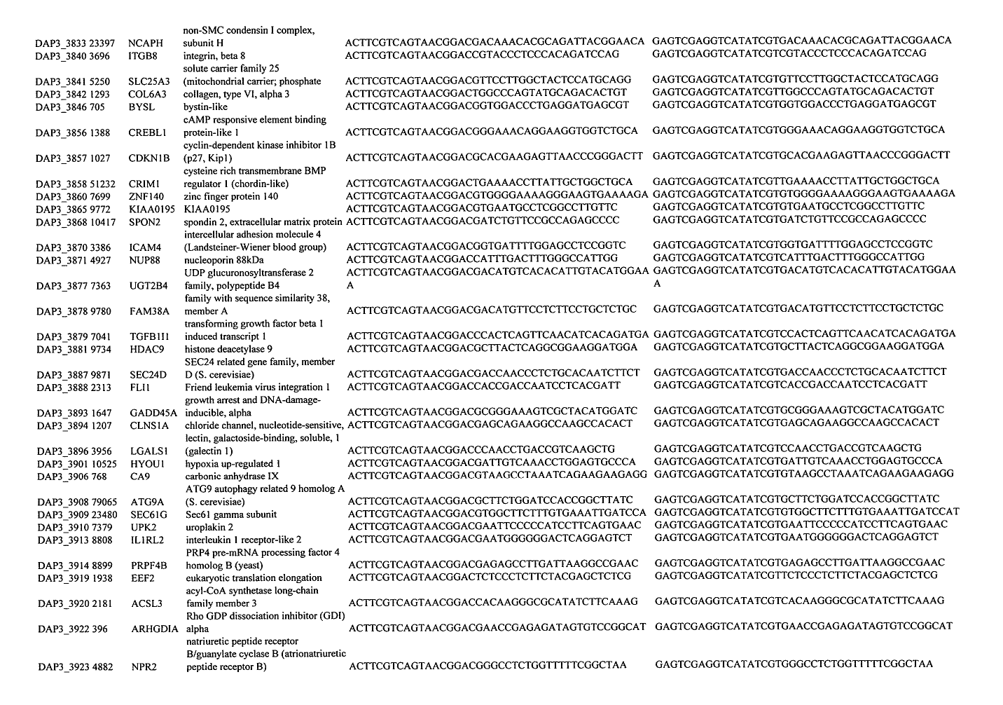

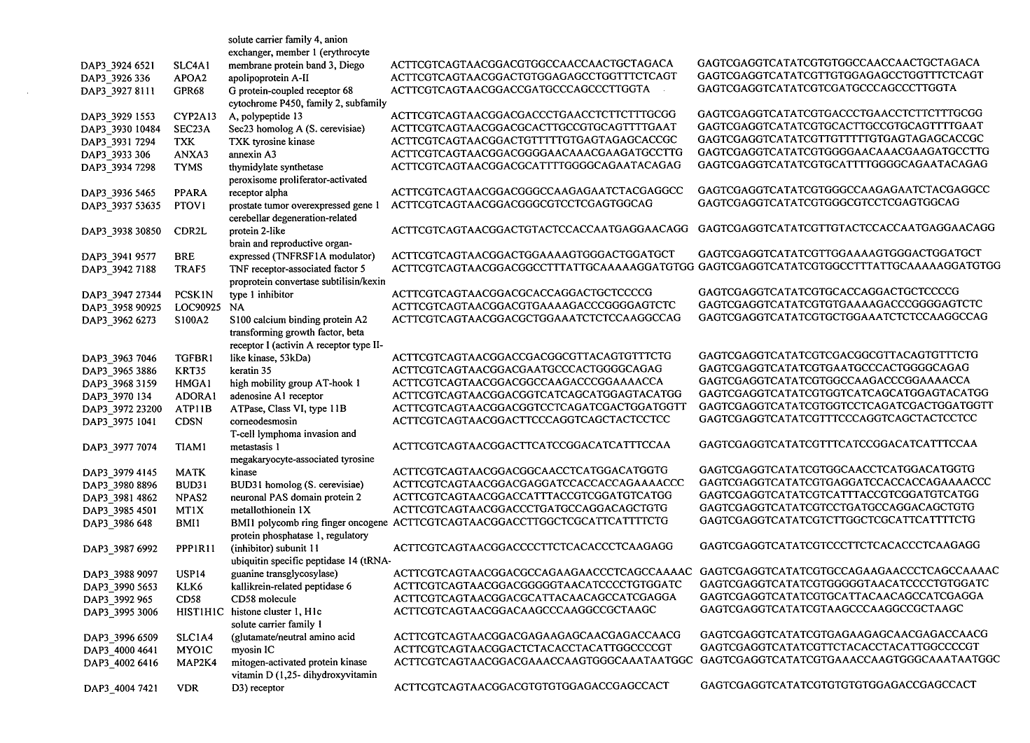

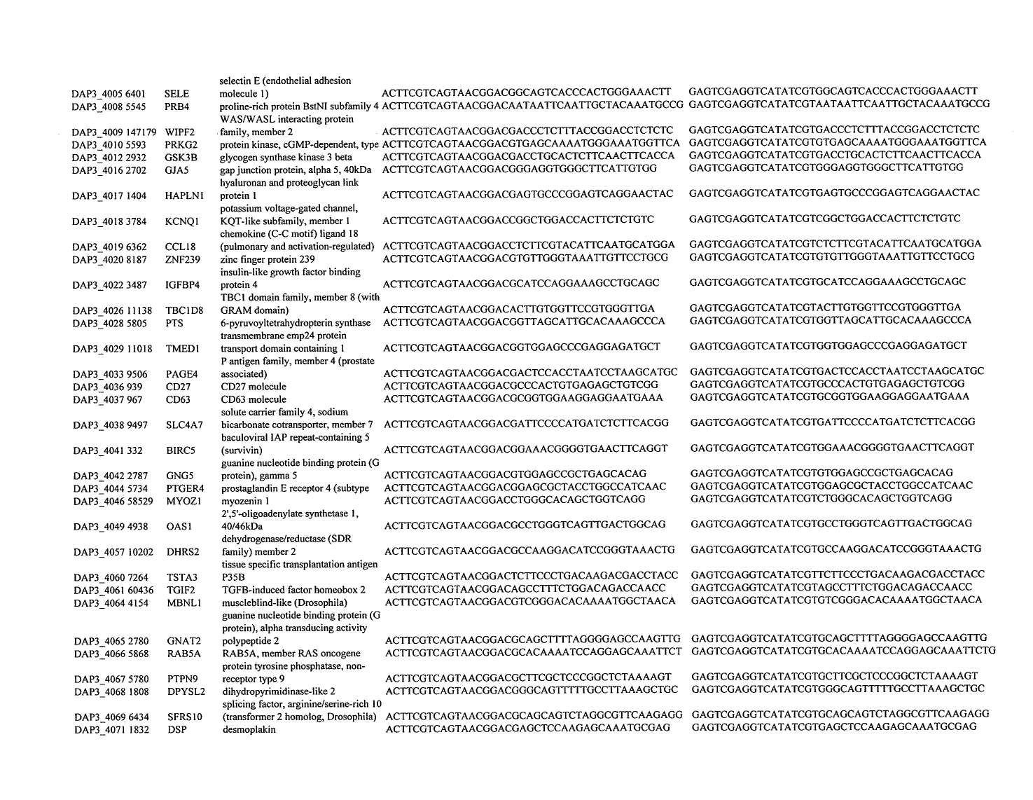

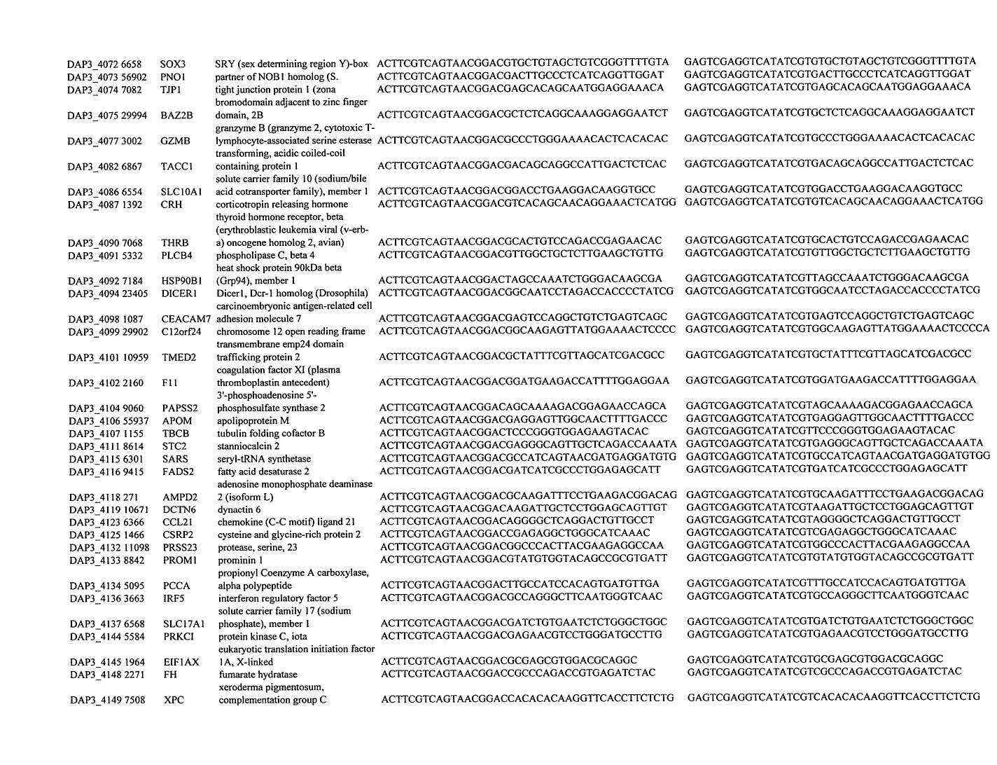

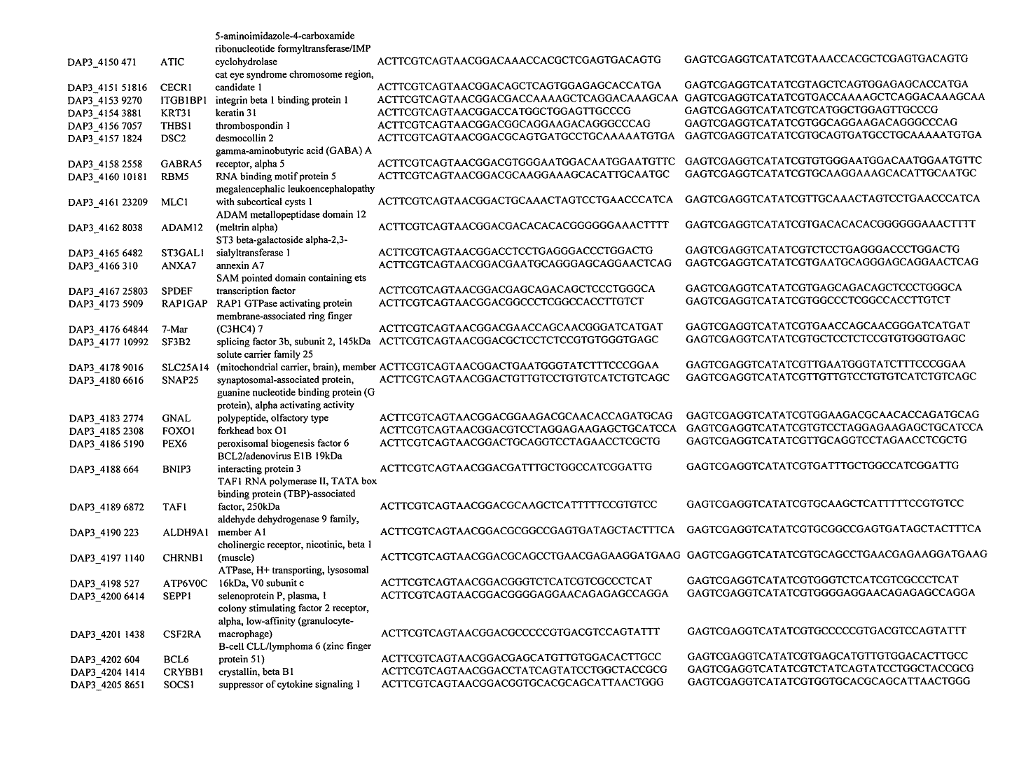

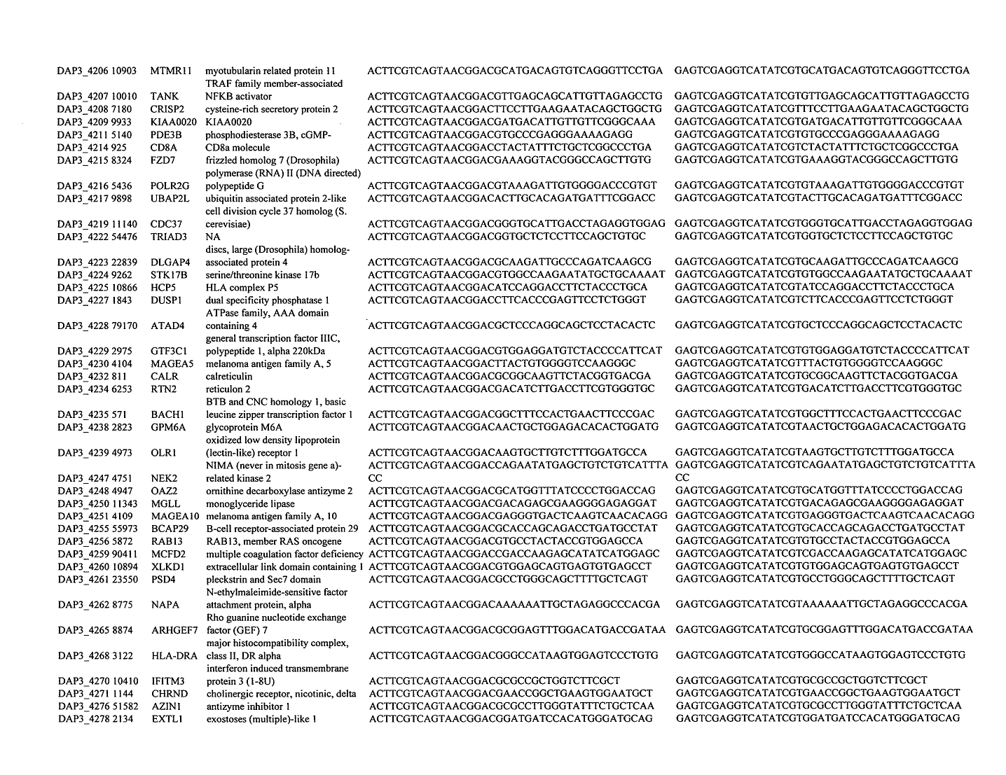

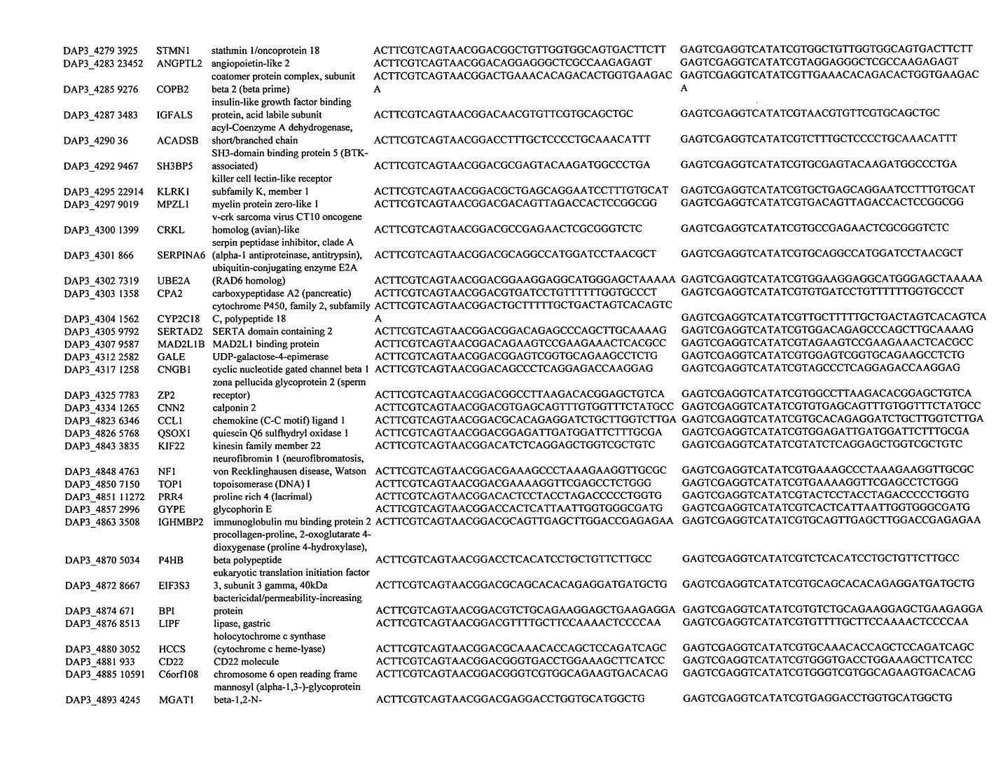

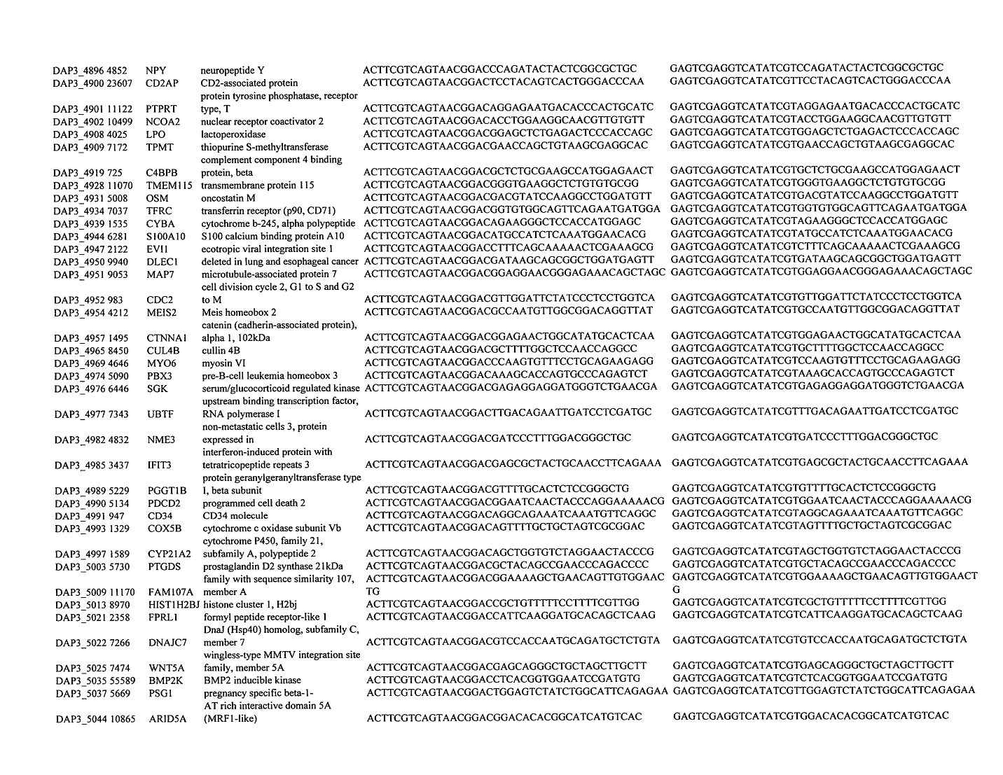

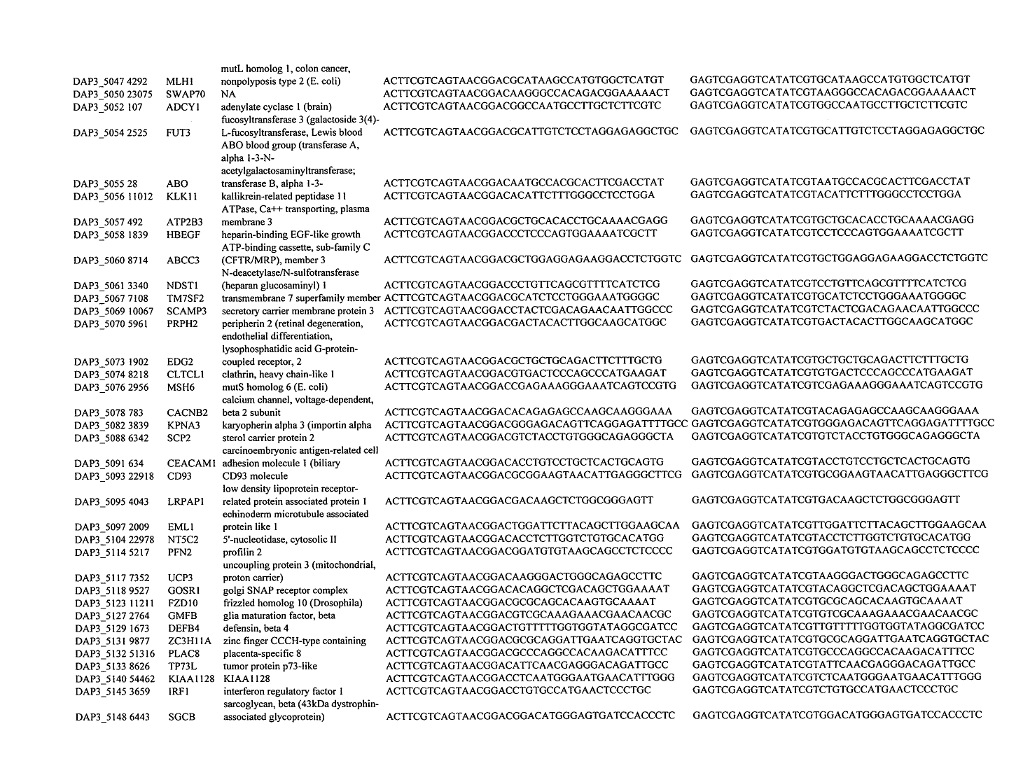

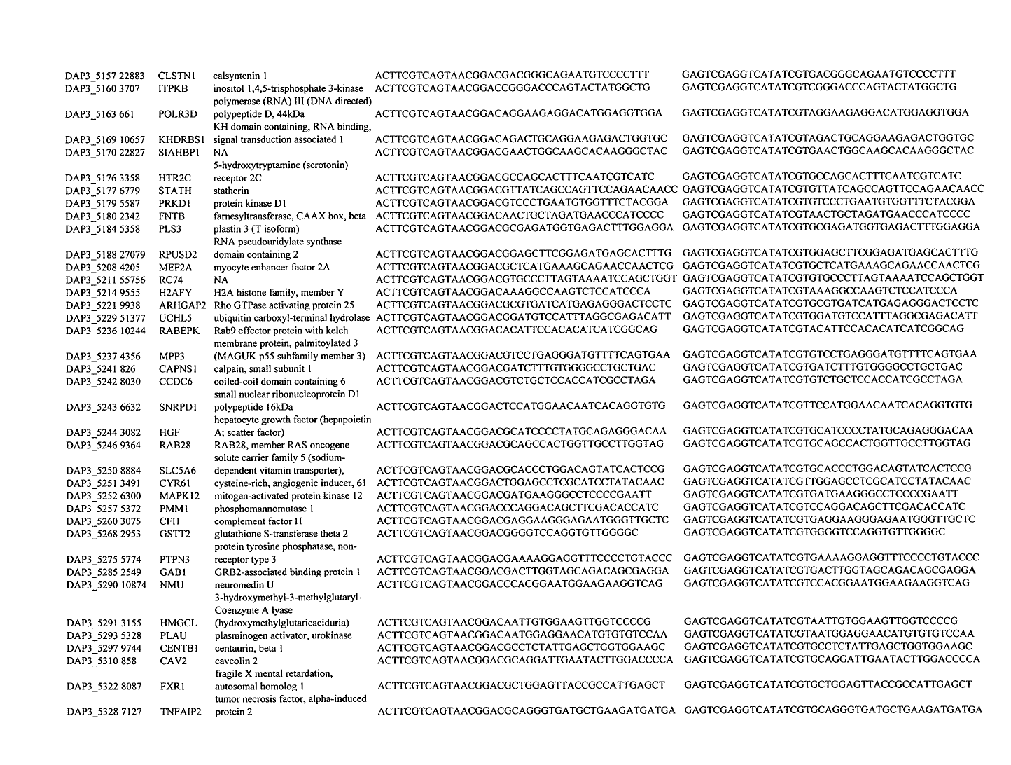

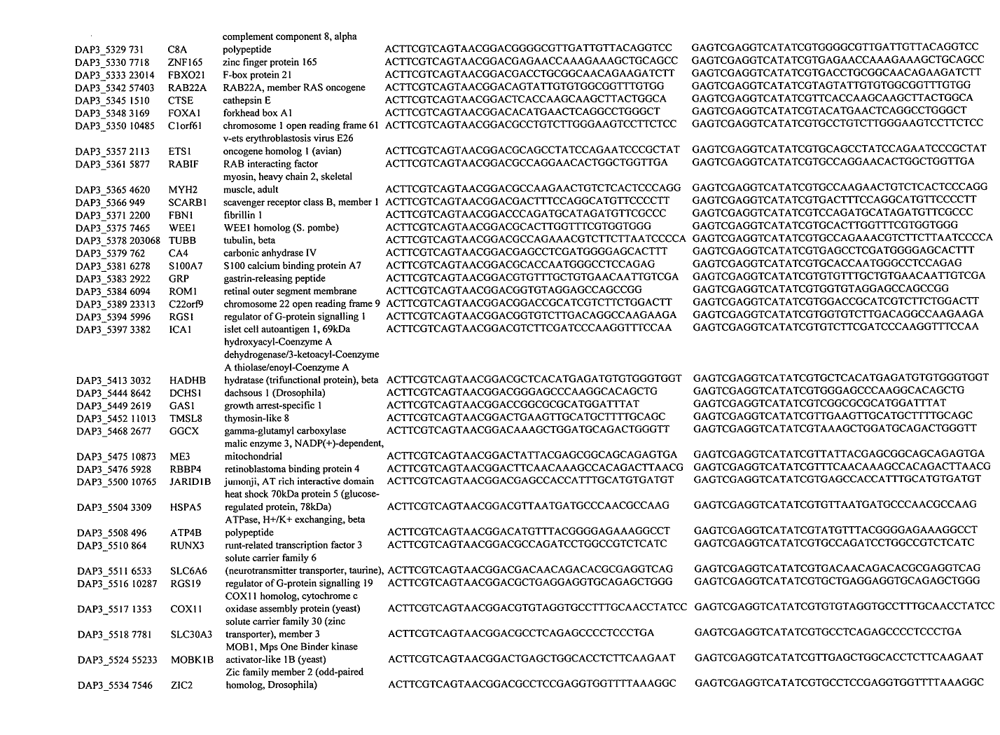

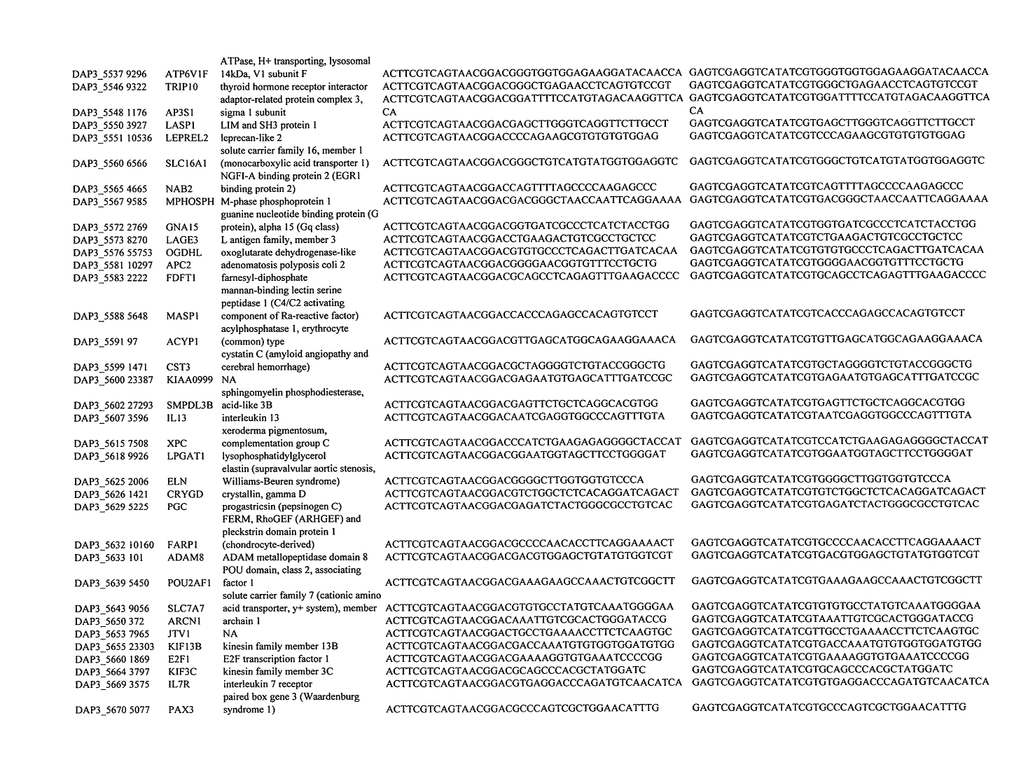

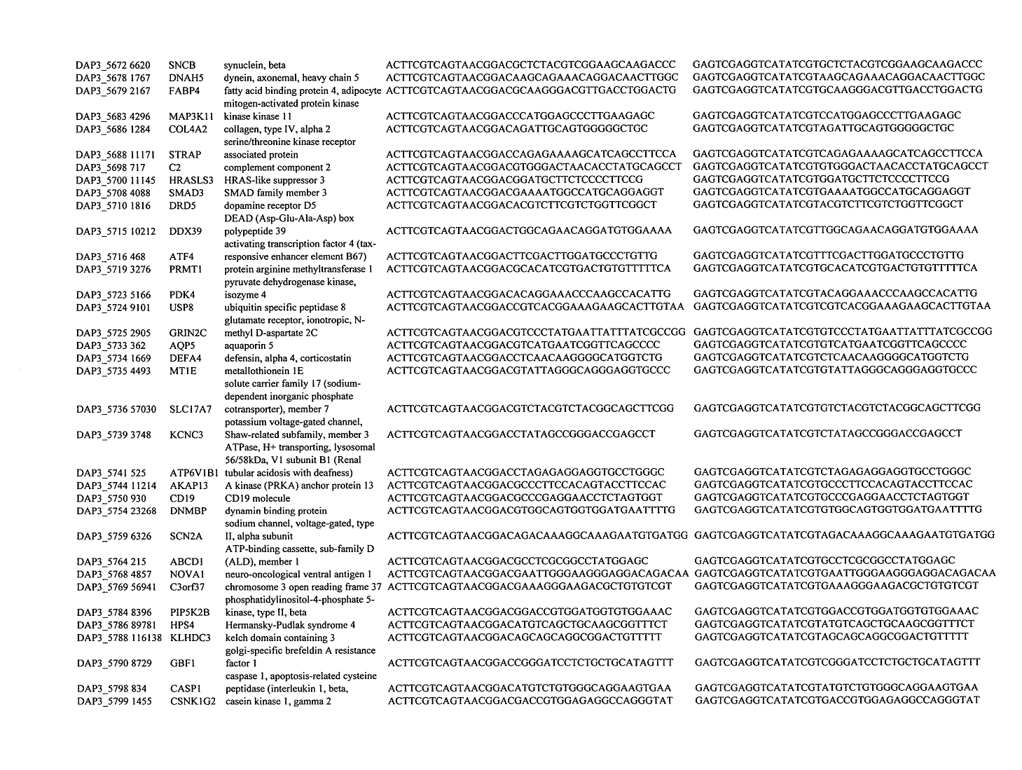

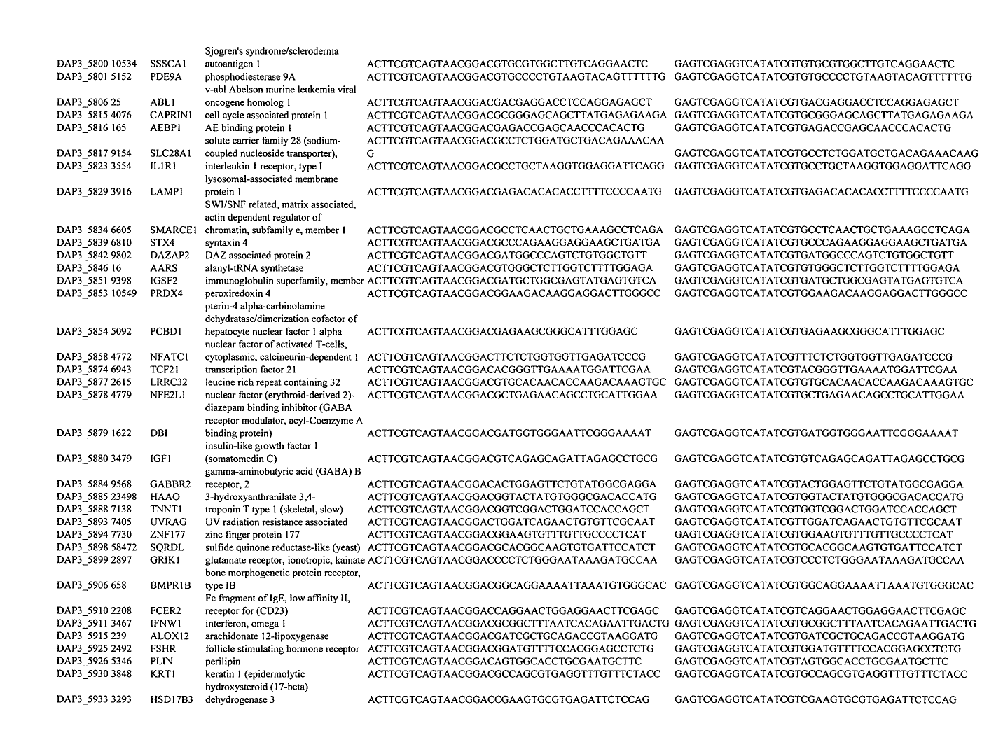

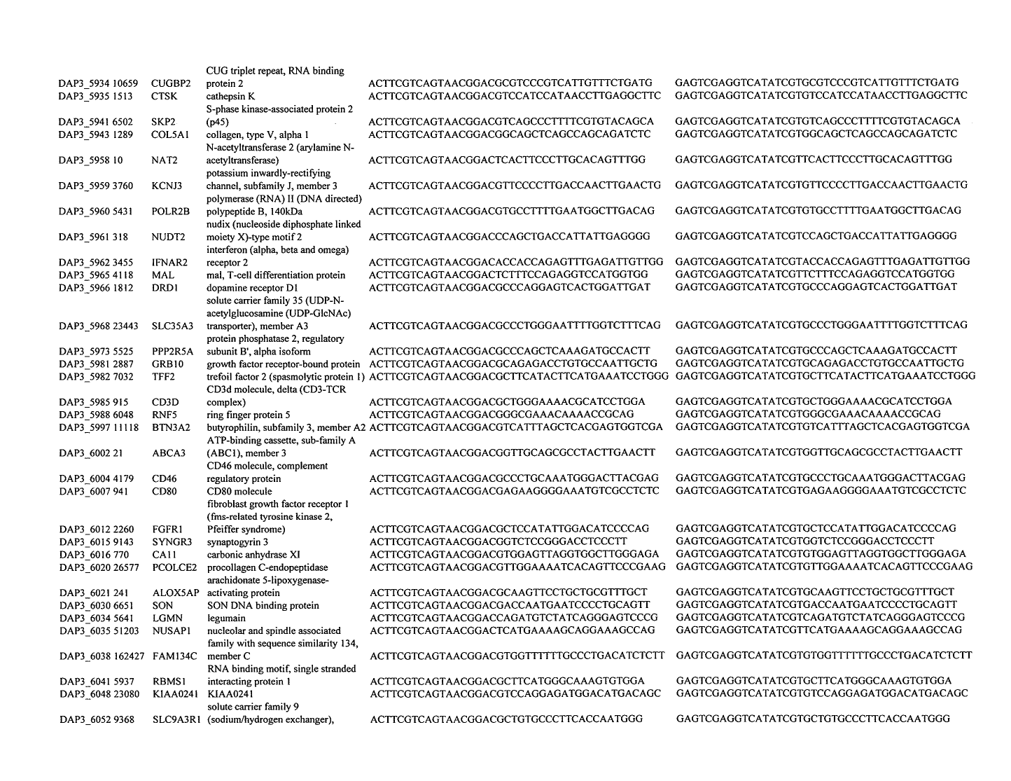

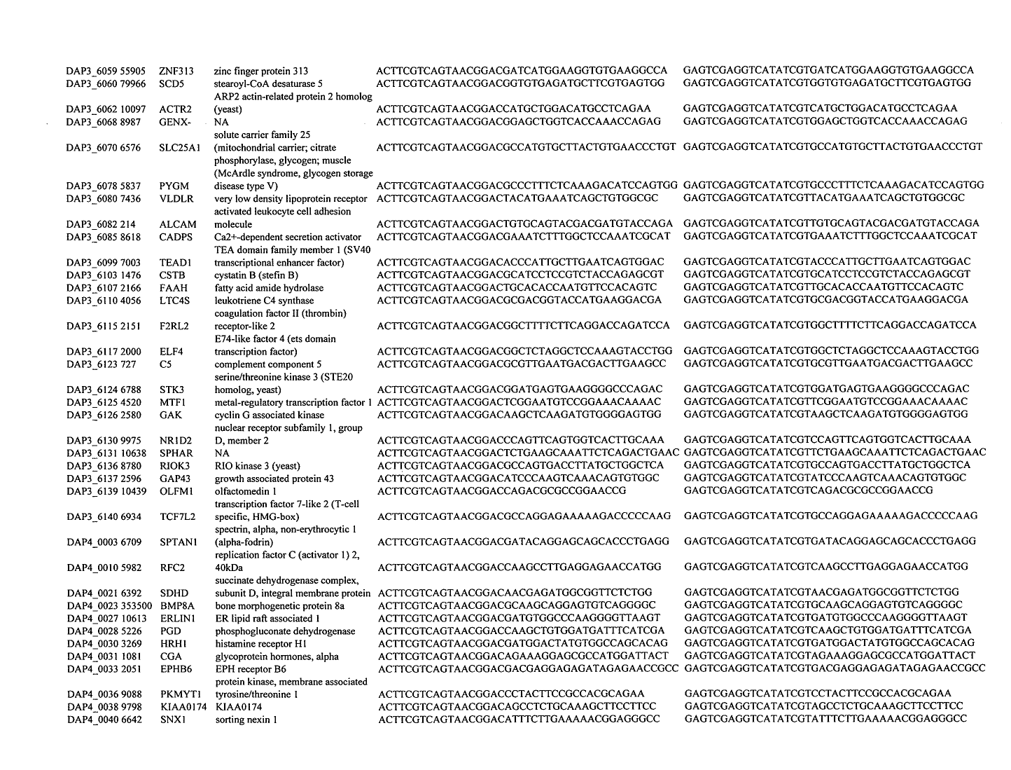

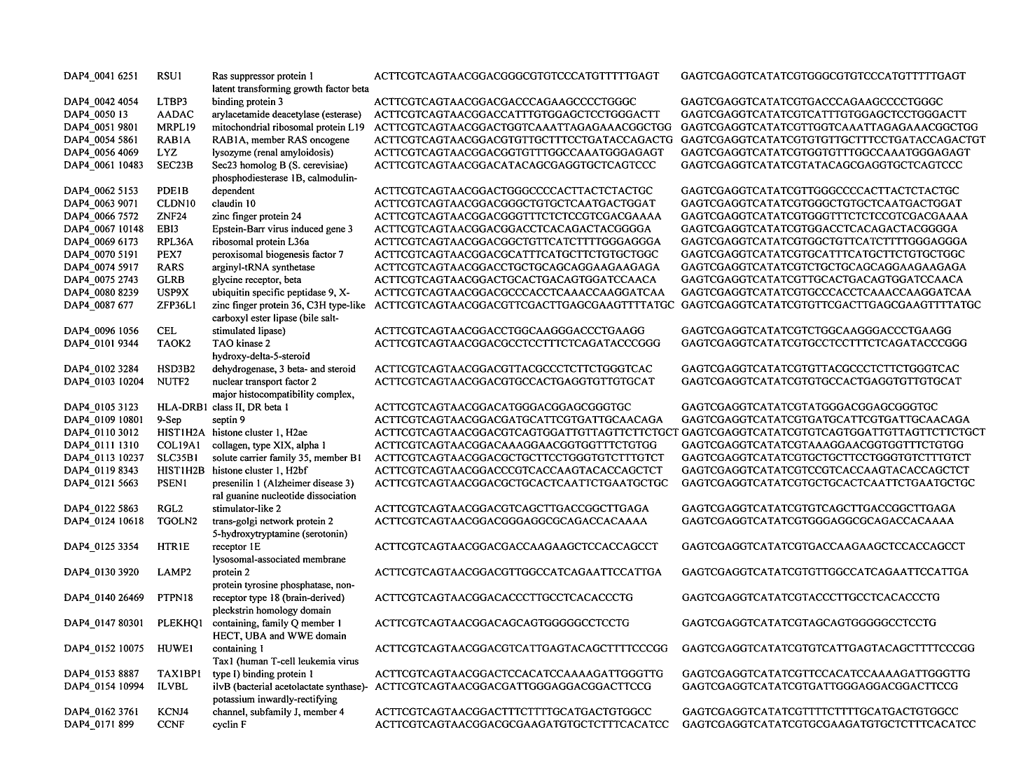

“Gene expression profile” as used herein is defined as the level or amount of gene expression of particular genes as assessed by methods described herein. The gene expression profile can comprise data for one or more genes and can be measured at a single time point or over a period of time. Phenotype classification (e.g., treatment outcome, presence or absence of hepatic disorders such as hepatocellular carcinoma and/or cirrhosis) can be made by comparing the gene expression profile of the sample with respect to one or more informative genes with one or more gene expression profiles (e.g., in a database). “Informative” genes and/or markers include those presented in the Figures, Tables, and Sequence Listing (e.g., Table 2A, Table 2B, Table 13A, Table 13B, and FIG. 19). Using the methods described herein, expression of numerous genes can be measured simultaneously. The assessment of numerous genes provides for a more accurate evaluation of the sample because there are more genes that can assist in classifying the sample.

A “marker nucleic acid” is a nucleic acid (e.g., DNA, mRNA, cDNA, microRNA) encoded by or corresponding to a marker of the invention. For example, such marker nucleic acid molecules include DNA (e.g., cDNA) comprising the entire or a partial sequence of any of the nucleic acid sequences encoding markers set forth in the Tables, Figures, or Sequence Listing described herein or the complement or hybridizing fragment of such a sequence. The marker nucleic acid molecules also include RNA comprising the entire or a partial sequence of any of the nucleic acid sequences encoding markers set forth in the Tables, Figures, or Sequence Listing or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth in the Tables, Figures, or Sequence Listing or a fragment thereof. The terms “protein” and “polypeptide” are used interchangeably herein.

A “marker” or “biomarker” is a gene or protein which may be altered, wherein said alteration is associated with a hepatic disorder. The alteration may be in amount, structure, and/or activity in a tissue or cell having a hepatic disorder, as compared to its amount, structure, and/or activity, in a normal or healthy tissue or cell (e.g., a control), and is associated with a disease state, such as cancer and/or cirrhosis. For example, a marker of the invention which is associated with cancer may have altered copy number, expression level, protein level, protein activity, or methylation status, in a cancer tissue or cancer cell as compared to a normal, healthy tissue or cell. Furthermore, a “marker” includes a molecule whose structure is altered, e.g., mutated (contains an allelic variant), e.g., differs from the wild type sequence at the nucleotide or amino acid level, e.g., by substitution, deletion, or addition, when present in a tissue or cell associated with a disease state, such as cancer.

Markers identified herein include diagnostic and therapeutic markers. A single marker may be a diagnostic marker, a therapeutic marker, or both a diagnostic and therapeutic marker.

As used herein, the term “therapeutic marker” includes markers, e.g., markers set forth in the Tables, Figures, or Sequence Listing described herein, which are believed to be involved in the development (including maintenance, progression, angiogenesis, and/or metastasis) of hepatic disorders. The hepatic disorder-related functions of a therapeutic marker may be confirmed by, e.g., increased or decreased copy number (by, e.g., fluorescence in situ hybridization (FISH), and FISH plus spectral karotype (SKY), or quantitative PCR (qPCR)) or mutation (e.g., by sequencing), overexpression or underexpression (e.g., by in situ hybridization (ISH), Northern Blot, RT-PCR, microarray analysis, qPCR, DASL, etc.), increased or decreased protein levels (e.g., by immunohistochemistry (IHC)), or increased or decreased protein activity (determined by, for example, modulation of a pathway in which the marker is involved). In one embodiment, a therapeutic marker may be used as a diagnostic marker.

As used herein, the term “diagnostic marker” or “prognostic marker” includes markers, e.g., markers set forth in the Tables, Figures, or Sequence Listing described herein, which are useful in the diagnosis and/or prognosis, respectively, of hepatic disorders, e.g., over- or under-activity emergence, expression, growth, remission, recurrence or resistance of cancer tumors before, during or after therapy. The predictive functions of the marker may be confirmed by, e.g., (1) increased or decreased copy number (e.g., by FISH, FISH plus SKY, or qPCR), overexpression or underexpression (e.g., by ISH, Northern Blot, RT-PCR, microarray analysis, qPCR, DASL, etc.), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with a hepatic disorder; (3) its presence or absence in clinical subset of patients with a hepatic disorder (e.g., those responding to a particular therapy or those developing resistance).

Diagnostic and prognostic markers also include “surrogate markers,” e.g., markers which are indirect markers of hepatic disorder progression.

“Neoadjuvant therapy” is adjunctive or adjuvant therapy given prior to the primary (main) therapy. Neoadjuvant therapy includes, for example, chemotherapy, radiation therapy, and hormone therapy. Thus, chemotherapy may be administered prior to surgery to shrink the tumor, so that surgery can be more effective, or, in the case of previously inoperable tumors, possible.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated be reference herein).

RNA interfering agents, e.g., siRNA molecules, may be administered to a patient having or at risk for having a hepatic disorder, to inhibit expression of a marker gene of the invention, e.g., a marker gene which is overexpressed in cancer and/or cirrhosis (such as the markers listed in, for example, Table 2A, Table 2B, Table 13A, Table 13B, and FIG. 19) and thereby treat, prevent, or inhibit the hepatic disorder in the subject.

A “transcribed polynucleotide” is a polynucleotide (e.g. an RNA, a cDNA, or an analog of one of an RNA or cDNA) which is complementary to or homologous with all or a portion of a mature RNA made by transcription of a marker of the invention and normal post-transcriptional processing (e.g. splicing), if any, of the transcript, and reverse transcription of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “homology” or “identity,” refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, more preferably, 60%, 65%, 70%, 75%, 80%, 83%, 85%, 87.5%, 90%, 91%, 92%, 93%, 94%, 95%, 96%, 97%, 98%, 99% or more.

A marker is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature (e.g. encodes a natural protein).

As used herein, the term “inhibiting a hepatic disorder” such as “inhibiting cancer” is intended to include the inhibition of undesirable or inappropriate effects of the hepatic disorder (such as cell growth). For example, the inhibition is intended to include inhibition of proliferation including rapid proliferation. The term “inhibiting cancer cell growth” is also intended to encompass inhibiting tumor growth which includes the prevention of the growth of a tumor in a subject or a reduction in the growth of a pre-existing tumor in a subject. The inhibition also can be the inhibition of the metastasis of a tumor from one site to another. A hepatic disorder is “inhibited” if at least one symptom of the hepatic disorder is alleviated, terminated, slowed, or prevented. As used herein, a hepatic disorder is also “inhibited” if recurrence or metastasis of the hepatic disorder is reduced, slowed, delayed, or prevented.

As used herein, the term “therapeutic agent” is defined broadly as anything that cancer cells, including tumor cells, may be exposed to in a therapeutic protocol. In the context of the present invention, such agents include, but are not limited to, chemotherapeutic agents, such as anti-metabolic agents, e.g., Ara AC, 5-FU and methotrexate, antimitotic agents, e.g., TAXOL, inblastine and vincristine, alkylating agents, e.g., melphalan, BCNU and nitrogen mustard, Topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light.

As used herein, the term “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases.

A kit is any manufacture (e.g. a package or container) comprising at least one reagent, e.g. a probe, for specifically detecting a marker of the invention, the manufacture being promoted, distributed, or sold as a unit for performing the methods of the present invention.

### II. USES OF THE INVENTION

In general, the present invention relates to methods for prognosis, diagnosis, treatment, and classification according to the gene expression profile of a sample (e.g., likelihood of survival or multi-centric recurrence in a subject based on a gene expression profile of a non-tumor liver sample from the subject). For example, a sample can be classified as belonging to a high risk class (e.g., a class wherein the subject from which the sample was obtained has a high likelihood of recurrence, or a class wherein the subject from which the sample was obtained has a poor prognosis for survival after treatment) or a low risk class (e.g., a class wherein the subject from which the sample was obtained has a prognosis for a low likelihood of recurrence or a class wherein the subject from which the sample was obtained has a good prognosis for survival after treatment). Duration of illness, severity of symptoms and eradication of disease can also be used as the basis for differentiating, i.e., classifying, samples.

Any marker or combination of markers listed in the Figures, Tables, or Sequence Listing described herein, may be used in the compositions, kits, and methods of the present invention. In addition, the present invention can be effectively used to analyze proteins, peptides, or nucleic acid molecules that are involved in transcription or translation. The nucleic acid molecule levels measured can be derived directly from the gene or, alternatively, from a corresponding regulatory gene. All forms of gene expression products can be measured, including, for example, spliced variants. Similarly, gene expression can be measured by assessing the level of protein or derivative thereof translated from mRNA. The sample to be assessed can be any sample that contains a gene expression product. Suitable sources of gene expression products, i.e., samples, can include cells, lysed cells, cellular material for determining gene expression, or material containing gene expression products. Examples of such samples are blood, plasma, lymph, urine, tissue, mucus, sputum, saliva or other cell samples. Methods of obtaining such samples are known in the art. In one embodiment, the sample is derived from an individual who has been clinically diagnosed as having or at risk of developing a hepatic disorder (e.g., hepatocellular carcinoma and/or cirrhosis). As used herein “obtaining” means acquiring a sample, either by directly procuring a sample from a patient or a sample (tissue biopsy, primary cell, cultured cells), or by receiving the sample from one or more people who procured the sample from the patient or sample.

In general, it is preferable to use markers for which the difference between the amount, e.g., level of expression or copy number, and/or activity of the marker in an experimental sample and the amount, e.g., level of expression or copy number, and/or activity of the same marker in a control sample, is as great as possible. Although this difference can be as small as the limit of detection of the method for assessing amount and/or activity of the marker, it is preferred that the difference be at least greater than the standard error of the assessment method, and preferably a difference of at least 1.5- 2-, 3-, 4-, 5-, 6-, 7-, 8-, 9-, 10-, 15-, 20-, 25-, 100-, 500-, 1000-fold or greater than the amount, e.g., level of expression or copy number, and/or activity of the same biomarker in a control sample.

When the compositions, kits, and methods of the invention are used for characterizing a relationship of markers of the invention to hepatic disorders (e.g., hepatocellular carcinoma and/or cirrhosis), assessing, for example, the likelihood of survival or likelihood of hepatic disorder recurrence, the marker or panel of markers of the invention may be selected such that a positive correlation is obtained in at least about 20%, and preferably at least about 40%, 60%, 70%, 75%, 80%, 85%, 90%, 91%, 92%, 93%, 94%, 95%, 96%, 97%, 98%, 99%, and more preferably, in substantially all, subjects afflicted with cancer, of the corresponding condition.

When a plurality of markers of the invention are used in the compositions, kits, and methods of the invention, the amount, structure, and/or activity of each marker or level of expression or copy number can be compared with the normal amount, structure, and/or activity of each of the plurality of markers or level of expression or copy number, in control samples of the same type, either in a single reaction mixture (i.e., using reagents, such as different fluorescent probes, for each marker) or in individual reaction mixtures corresponding to one or more of the markers.

When a plurality of markers are used, it is preferred that 2, 3, 4, 5, 8, 10, 12, 15, 20, 30, 50, 60, 70, 80, 90, 100, 110, 120, 130, 140, 150, 160, 170, 180 or more individual markers be used or identified, wherein fewer markers are preferred. For example, one, more than one, or all of the markers in the Tables included herewith, e.g., Table 2A, Table 2B, Table 13A, Table 13B, and FIG. 19 can be used.

It is recognized that the compositions, kits, and methods of the invention will be of particular utility to subjects having an enhanced risk of developing a hepatic disorder, and their medical advisors. Subjects recognized as having an enhanced risk of developing a hepatic disorder, include, for example, subjects having a familial history of a hepatic disorder, subjects identified as having a mutant oncogene (i.e. at least one allele), and subjects of advancing age.

The compositions, kits, and methods may have to be adapted for use with certain types of samples. For example, when the sample is a parafinized, archived human tissue sample, it may be necessary to adjust the ratio of compounds in the compositions of the invention, in the kits of the invention, or the methods used. For example, the present invention required novel adaptation to analyze formalin-fixed paraffin-embedded tissues.

Various aspects of the invention are described in further detail in the following subsections.

### III. ISOLATED NUCLEIC ACID MOLECULES

One aspect of the invention pertains to isolated nucleic acid molecules that correspond to a marker of the invention (e.g., markers listed in the Tables, Figures, and Sequence Listing described herein), including nucleic acids which encode a polypeptide corresponding to a marker of the invention or a portion of such a polypeptide. Isolated nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule of the present invention, e.g., a nucleic acid molecules encoding a protein corresponding to a marker listed in the Tables, Figures, and Sequence Listing described herein, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a protein which corresponds to a marker of the invention, and thus encode the same protein.

In addition to the nucleotide sequences described in the Tables, Figures, and Sequence Listing described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including, but not limited to, the genes listed in Table 2A, Table 2B, Table 13A, Table 13B, and FIG. 19, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### IV. ISOLATED PROTEINS AND ANTIBODIES

One aspect of the invention pertains to isolated proteins which correspond to individual markers of the invention, and biologically active portions thereof, as well as polypeptide fragments suitable for use as immunogens to raise antibodies directed against a polypeptide corresponding to a marker of the invention. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the marker (e.g., the protein encoded by the nucleic acid molecules listed in Table 2A, Table 2B, Table 13A, Table 13B, and FIG. 19), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have an amino acid sequence of a protein encoded by a nucleic acid molecule listed in Table 2A, Table 2B, Table 13A, Table 13B, and FIG. 19. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual markers of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody, antibody derivative, or fragment thereof, which specifically binds a marker of the invention (e.g., a marker set forth in markers listed in the Tables, Figures, and Sequence Listing described herein), may be used to inhibit activity of a marker and therefore may be administered to a subject to treat, inhibit, or prevent a hepatic disorder in the subject. Furthermore, conjugated antibodies may also be used to treat, inhibit, or prevent a hepatic disorder in a subject. Conjugated antibodies, preferably monoclonal antibodies, or fragments thereof, are antibodies which are joined to drugs, toxins, or radioactive atoms, and used as delivery vehicles to deliver those substances directly to cells having a hepatic disorder. The antibody, e.g., an antibody which specifically binds a marker of the invention (e.g., a marker listed in the Tables, Figures, and Sequence Listing described herein), is administered to a subject and binds the marker, thereby delivering the toxic substance to the cell having a hepatic disorder, minimizing damage to normal cells in other parts of the body.

Conjugated antibodies are also referred to as “tagged,” “labeled,” or “loaded.” Antibodies with chemotherapeutic agents attached are generally referred to as chemolabeled. Antibodies with radioactive particles attached are referred to as radiolabeled, and this type of therapy is known as radioimmunotherapy (RIT). Aside from being used to treat hepatic disorders, radiolabeled antibodies can also be used to detect areas of hepatic disorder spread in the body. Antibodies attached to toxins are called immunotoxins.

Immunotoxins are made by attaching toxins (e.g., poisonous substances from plants or bacteria) to monoclonal antibodies. Immunotoxins may be produced by attaching monoclonal antibodies to bacterial toxins such as diphtherial toxin (DT) or pseudomonal exotoxin (PE40), or to plant toxins such as ricin A or saporin.

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g. in a blood- or bone marrow-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹³¹I, ¹²⁵I, ³⁵S or ³H.

### V. RECOMBINANT EXPRESSION VECTORS AND HOST CELLS

Another aspect of the invention pertains to vectors, preferably expression vectors, containing a nucleic acid encoding a polypeptide corresponding to a marker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell. Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

The host cells of the invention can also be used to produce nonhuman transgenic animals. For example, in one embodiment, a host cell of the invention is a fertilized oocyte or an embryonic stem cell into which sequences encoding a polypeptide corresponding to a marker of the invention have been introduced. Such host cells can then be used to create non-human transgenic animals in which exogenous sequences encoding a marker protein of the invention have been introduced into their genome or homologous recombinant animals in which endogenous gene(s) encoding a polypeptide corresponding to a marker of the invention sequences have been altered. Such animals are useful for studying the function and/or activity of the polypeptide corresponding to the marker, for identifying and/or evaluating modulators of polypeptide activity, as well as in pre-clinical testing of therapeutics or diagnostic molecules, for marker discovery or evaluation, e.g., therapeutic and diagnostic marker discovery or evaluation, or as surrogates of drug efficacy and specificity.

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal. Transgenic animals also include inducible transgenic animals, such as those described in, for example, Chan I. T., et al. (2004) J Clin Invest. 113(4):528-38 and Chin L. et at (1999) Nature 400(6743):468-72.

A transgenic animal of the invention can be created by introducing a nucleic acid encoding a polypeptide corresponding to a marker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a marker of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells: A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

### VI. METHODS OF TREATMENT

The present invention provides for both prophylactic and therapeutic methods of treating a subject, e.g., a human, who has or is at risk of (or susceptible to) a hepatic disorder (e.g., liver cancer and/or cirrhosis. In one embodiment, the subject has or is at risk for cancer, e.g., HCC. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, inhibiting, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” or “compound” includes, but is not limited to, small molecules, peptides, peptidomimetics, polypeptides, RNA interfering agents, e.g., siRNA molecules, antibodies, ribozymes, and antisense oligonucleotides.

Accordingly, another aspect of the invention pertains to methods for treating a subject suffering from a hepatic disorder. These methods involve administering to a subject a compound which modulates amount and/or activity of one or more markers of the invention. For example, methods of treatment or prevention of a hepatic disorder include administering to a subject a compound which modulates the amount and/or activity of one or more markers listed in the Tables, Figures, and Sequence Listing described herein. Agents which may be used to inhibit amount and/or activity of a marker listed in listed in the Tables, Figures, and Sequence Listing described herein, to thereby treat or prevent a hepatic disorder include antibodies (e.g., conjugated antibodies), small molecules, RNA interfering agents, e.g., siRNA molecules, ribozymes, and antisense oligonucleotides. In one embodiment, an antibody used for treatment is conjugated to a toxin, a chemotherapeutic agent, or radioactive particles.

Methods of treatment or prevention of a hepatic disorder also include administering to a subject a compound which increases the amount and/or activity of one or more markers listed in the Tables, Figures, and Sequence Listing described herein. Agents, e.g., agonists, which may be used include small molecules, peptides, peptoids, peptidomimetics, and polypeptides.

Small molecules used in the methods of the invention include those which inhibit a protein-protein interaction and thereby either increase or decrease marker amount and/or activity. Furthermore, modulators, e.g., small molecules, which cause re-expression of silenced genes, e.g., tumor suppressors, are also included herein. For example, such molecules include compounds which interfere with DNA binding or methyltransferas activity.

An aptamer may also be used to modulate, e.g., increase or inhibit expression or activity of a marker of the invention to thereby treat, prevent or inhibit a hepatic disorder. Aptamers are DNA or RNA molecules that have been selected from random pools based on their ability to bind other molecules. Aptamers may be selected which bind nucleic acids or proteins.

In addition, hepatic disorders are often effectively treated by a combination of reagents or methodologies. The growth or viability of HCC cells may also be affected by treatment with a combination of agents or methodologies. Examples include: 1) chemotherapy and radiation therapy in the treatment of cervical cancer (Aoki and Tanaka 2002) or head and neck cancer (Busto et al. 2001) or pancreatic cancer (McGinn et al. 2002); 2) chemotherapy and surgery in the treatment of cervical cancer (Aoki and Tanaka 2002); 3) antibody therapy and cytokine therapy in the treatment of breast cancer (Hortobagyi 2002); 4) combination chemotherapy treatment of melanoma (McClay 2002) or colorectal carcinoma (Kim et al. 2002); 5) the suggestion of multiple therapies including gene therapy, angiogenesis inhibitors and antibody therapy in the treatment of non-small cell lung cancer (Felip and Rossell 2001); and 6) the suggested treatment of metastatic breast cancer by a combination of chemotherapy and antibody or kinase inhibitor, or angiogenic inhibitor therapy. In addition, standard of care treatments for hepatic disorders are well known and described in the art (see, e.g., Primary Care Medicine, 6^{th} edition and references cited tehrein, edited by Goroll et al. especially at chapter 71 entitled “Management of cirrhosis and chronic liver failure”). For example, cirrhosis may be treated with anti-fibrotic therapies (low-dose interferon and/or kinase inhibitors such as erlotinib) and/or therapies targeting hepatitis viruses including full-dose interferon, nucleoside analogues, viral protease inhibitors, etc.

Conventional nonspecific immunosuppressive agents, that may be administered in combination with the compositions of the invention include, but are not limited to, steroids, cyclosporine, cyclosporine analogs, cyclophosphamide methylprednisone, prednisone, azathioprine, FK-506, 15-deoxyspergualin, and other immunosuppressive agents.

In a further embodiment, the compositions of the invention are administered in combination with an antibiotic agent. Antibiotic agents that may be administered with the compositions of the invention include, but are not limited to, tetracycline, metronidazole, amoxicillin, beta-lactamases, aminoglycosides, macrolides, quinolones, fluoroquinolones, cephalosporins, erythromycin, ciprofloxacin, and streptomycin. In an additional embodiment, the compositions of the invention are administered alone or in combination with an anti-inflammatory agent. Anti-inflammatory agents that can be administered with the compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the compositions of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the invention are administered in combination with cytokines Cytokines that may be administered with the compositions of the invention include, but are not limited to, IL2, IL3, IL4, IL5, IL6, IL7, IL10, IL12, IL13, IL15, anti-CD40, CD40L, IFN-gamma and TNF-alpha.

In additional embodiments, the compositions of the invention are administered in combination with other therapeutic or prophylactic regimens, such as, for example, radiation therapy.

Thus, the therapeutic agents and constructs of the present invention are contemplated for use in combination with one or more standard hepatic disorder treatments. For example, particular inventive methods may be used in combination with one or more of the following: a) a chemotherapeutic agent; b) radiation therapy; c) surgical resection or liver transplantation; or d) radio frequency ablation, cryosurgery, ethanol ablation and embolization. In addition, standard of care treatments for hepatic disorders are well known and described in the art (see, e.g., Primary Care Medicine, 6^{th} edition and references cited tehrein, edited by Goroll et al. especially at chapter 71 entitled “Management of cirrhosis and chronic liver failure”). For example, cirrhosis may be treated with anti-fibrotic therapies (low-dose interferon and/or kinase inhibitors such as erlotinib) and/or therapies targeting hepatitis viruses including full-dose interferon, nucleoside analogues, viral protease inhibitors, etc.

### VII. SCREENING ASSAYS

The invention also provides methods (also referred to herein as “screening assays”) for identifying modulators, i.e., candidate or test compounds or agents (e.g., proteins, peptides, peptidomimetics, peptoids, small molecules or other drugs) which (a) bind to a marker of the invention, or (b) have a modulatory (e.g., stimulatory or inhibitory) effect on the activity of a marker of the invention or, more specifically, (c) have a modulatory effect on the interactions of a marker of the invention with one or more of its natural substrates (e.g., peptide, protein, hormone, co-factor, or nucleic acid), or (d) have a modulatory effect on the expression of a marker of the invention. Such assays typically comprise a reaction between the marker and one or more assay components. The other components may be either the test compound itself, or a combination of test compound and a natural binding partner of the marker. Compounds identified via assays such as those described herein may be useful, for example, for modulating, e.g., inhibiting, ameliorating, treating, or preventing cancer.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker of the invention or biologically active portion thereof. In another embodiment, the invention provides assays for screening candidate or test compounds which bind to a marker of the invention or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker of the invention or a biologically active portion thereof. In all likelihood, the marker can, in vivo, interact with one or more molecules, such as, but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of the marker to identify its natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker or downstream elements of a marker-mediated signaling pathway. Alternatively, such marker binding partners may also be found to be inhibitors of the marker.

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is a cancer marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner. Conversely, the formation of more complex in the presence of compound than in the control reaction indicates that the compound may enhance interaction of the marker and its binding partner.

The assay for compounds that interfere with the interaction of the marker with its binding partner may be conducted in a heterogeneous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et at (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternatively, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays. In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cancer, cellular transformation and/or tumorigenesis. Animal models for colorectal cancer are described in, for example, Zhu et al. (1998) Cell 94, 703-714 and Moser et al. (1990) Science 247, 322-324, the contents of which are expressly incorporated herein by reference. Additional animal based models of cancer are well known in the art (reviewed in Animal Models of Cancer Predisposition Syndromes, Hiai, H and Hino, O (eds.) 1999, Progress in Experimental Tumor Research, Vol. 35; Clarke A R Carcinogenesis (2000) 21:435-41) and include, for example, carcinogen-induced tumors (Rithidech, K et al. Mutat Res (1999) 428:33-39; Miller, M L et al. Environ Mol Mutagen (2000) 35:319-327), injection and/or transplantation of tumor cells into an animal, as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294). Animal models described in, for example, Chin L. et at (1999) Nature 400(6743):468-72, may also be used in the methods of the invention.

This invention further pertains to novel agents identified by the above-described screening assays. Accordingly, it is within the scope of this invention to further use an agent identified as described herein in an appropriate animal model. For example, an agent identified as described herein (e.g., a marker modulating agent, a small molecule, an antisense marker nucleic acid molecule, a ribozyme, a marker-specific antibody, or fragment thereof, a marker protein, a marker nucleic acid molecule, an RNA interfering agent, e.g., an siRNA molecule targeting a marker of the invention, or a marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### VIII. PHARMACEUTICAL COMPOSITIONS

The small molecules, peptides, peptoids, peptidomimetics, polypeptides, RNA interfering agents, e.g., siRNA molecules, antibodies, ribozymes, and antisense oligonucleotides (also referred to herein as “active compounds” or “compounds”) corresponding to a marker of the invention can be incorporated into pharmaceutical compositions suitable for administration. Such compositions typically comprise the small molecules, peptides, peptoids, peptidomimetics, polypeptides, RNA interfering agents, e.g., siRNA molecules, antibodies, ribozymes, or antisense oligonucleotides and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid molecule or polypeptide of the invention. Small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram).

As defined herein, a therapeutically effective amount of an RNA interfering agent, e.g., siRNA, (i.e., an effective dosage) ranges from about 0.001 to 3,000 mg/kg body weight, preferably about 0.01 to 2500 mg/kg body weight, more preferably about 0.1 to 2000, about 0.1 to 1000 mg/kg body weight, 0.1 to 500 mg/kg body weight, 0.1 to 100 mg/kg body weight, 0.1 to 50 mg/kg body weight, 0.1 to 25 mg/kg body weight, and even more preferably about 1 to 10 mg/kg, 2 to 9 mg/kg, 3 to 8 mg/kg, 4 to 7 mg/kg, or 5 to 6 mg/kg body weight. Treatment of a subject with a therapeutically effective amount of an RNA interfering agent can include a single treatment or, preferably, can include a series of treatments. In a preferred example, a subject is treated with an RNA interfering agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks.

Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the epithelium). A method for lipidation of antibodies is described by Cruikshank et al. (1997) J. Acquired Immune Deficiency Syndromes and Human Retrovirology 14:193.

The nucleic acid molecules corresponding to a marker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The RNA interfering agents, e.g., siRNAs used in the methods of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the RNA interfering agent, e.g., the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### IX. PREDICTIVE MEDICINE

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the amount, structure, and/or activity of polypeptides or nucleic acids corresponding to one or more markers of the invention, in order to determine whether an individual is at risk of developing a hepatic disorder, e.g., liver cancer and/or cirrhosis. Such assays can be used for prognostic or predictive purposes to thereby prophylactically treat an individual prior to the onset of a hepatic disorder, including cancer.

Yet another aspect of the invention pertains to monitoring the influence of agents (e.g., drugs or other compounds administered either to inhibit a hepatic disorder or to treat or prevent any other disorder {i.e. in order to understand any carcinogenic effects that such treatment may have}) on the amount, structure, and/or activity of a marker of the invention in clinical trials. These and other agents are described in further detail in the following sections.

A. Diagnostic Assays

1. Methods for Detection of Copy Number

Methods of evaluating the copy number of a particular marker or chromosomal region are well known to those of skill in the art. The presence or absence of chromosomal gain or loss can be evaluated simply by a determination of copy number of the regions or markers identified herein.

Methods for evaluating copy number of encoding nucleic acid in a sample include, but are not limited to, hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the relative copy number of the target nucleic acid.

An alternative means for determining the copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases.

In some applications it is necessary to block the hybridization capacity of repetitive sequences. Thus, in some embodiments, tRNA, human genomic DNA, or Cot-I DNA is used to block non-specific hybridization.

In CGH methods, a first collection of nucleic acids (e.g., from a sample, e.g., a possible tumor) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl. Acad Sci USA 89:5321-5325 (1992) is used.

The methods of the invention are particularly well suited to array-based hybridization formats. Array-based CGH is described in U.S. Pat. No. 6,455,258, the contents of which are incorporated herein by reference.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) may also be used to identify regions of amplification or deletion.

2. Methods for Detection of Gene Expression

Marker expression level can also be assayed as a method for diagnosis of a hepatic disorder, including cancer, or risk for developing a hepatic disorder, including cancer. Expression of a marker of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA or microRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (e.g., mRNA, cDNA made therefrom, or microRNA) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Preferably nucleic acid probes are 20 bases or longer in length. (See, e.g., Sambrook et al. for methods of selecting nucleic acid probe sequences for use in nucleic acid hybridization.) Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR may also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

Detection of differentially expressed genes also may use other methods of evaluating differential gene expression. Examples include indexing differential display reverse transcription polymorase chain reaction (DDRT-PCR; Mahadeva et al, 1998, J. Mol. Biol. 284:1391-1318; WO 94/01582; subtractive mRNA hybridization (See Advanced Mol. Biol.; R. M. Twyman (1999) Bios Scientific Publishers, Oxford, p. 334, the use of nucleic acid arrays or microarrays (see Nature Genetics, 1999, vol. 21, Suppl. 1061) and the serial analysis of gene expression (SAGE Valculesev et al, Science (1995) 270:484-487; U.S. Pat. Nos. 6,308,170; 6,183,698; 6,306,643; 6,297,018; 6,287,850; 6,291,183), real time PCR(RT-PCR), and DNA-mediated annealing, selection, extension and ligation (DASL); Fan et al (2004), Genome Res. 14:878-885; Bibikova et al (2004) 165:1799-1807. Combinations of these methods can be used.

In another preferred embodiment, expression of a marker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the marker, and fragments thereof cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g. single nucleotide polymorphisms, deletions, etc.) of a marker of the invention may be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker of the invention. If polynucleotides complementary to or homologous with are differentially detectable on the substrate (e.g. detectable using different chromophores or fluorophores, or fixed to different selected positions), then the levels of expression of a plurality of markers can be assessed simultaneously using a single substrate (e.g. a “gene chip” microarray of polynucleotides fixed at selected positions). When a method of assessing marker expression is used which involves hybridization of one nucleic acid with another, it is preferred that the hybridization be performed under stringent hybridization conditions. In one embodiment, the fragment is at least 9 nucleotides; preferably, it is at least 15 to 20 nucleotides. Such a composition can be employed for the diagnosis and treatment of HCC from any etiology or disease in which the dysfunction or non-function of liver cells is implicated or suspected. The composition is particularly useful as hybridizable array elements in a microarray for monitoring the expression of a plurality of target polynucleotides. The microarray comprises a substrate and the hybridizable nucleic acid array elements. The microarray can be used, for example, in the diagnosis and treatment monitoring of a hepatic disorder (e.g., hepatocellular carcinoma and/or cirrhosis). When the composition is employed as hybridizable array elements in a microarray, the array elements are preferably organized in an ordered fashion so that each element is present at a distinguishable, and preferably specified, location on the substrate. In preferred embodiments, because the array elements are at specified locations on the substrate, the hybridization patterns and intensities (which together create a unique expression profile) can be interpreted in terms of expression levels of particular genes and can be correlated with a particular disease or condition or treatment.

Once the gene expression levels of the sample are obtained, the levels are compared or evaluated against the model, and then the sample is classified. The evaluation of the sample determines whether or not the sample should be assigned to the particular phenotypic class being studied. The correlation between gene expression and class distinction can be determined using a variety of methods well known in the field (e.g., U.S. Ser. No. 09/544,627 and the Examples). The information provided by the present invention, alone or in conjunction with other test results, aids in sample classification.

In another embodiment, a combination of methods to assess the expression of a marker is utilized.

3. Methods for Detection of Expressed Protein

The activity or level of a marker protein can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express a marker of the present invention.

A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a preferred embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies may be directly labeled or alternatively may be subsequently detected using labeled antibodies (e.g., labeled sheep anti-human antibodies) that specifically bind to the anti-polypeptide.

In a more preferred embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g. as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one particularly preferred embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

The assays of this invention are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described herein.

In another embodiment, level (activity) is assayed by measuring the enzymatic activity of the gene product. Methods of assaying the activity of an enzyme are well known to those of skill in the art.

In vivo techniques for detection of a marker protein include introducing into a subject a labeled antibody directed against the protein. For example, the antibody can be labeled with a radioactive marker whose presence and location in a subject can be detected by standard imaging techniques.

Certain markers identified by the methods of the invention may be secreted proteins. It is a simple matter for the skilled artisan to determine whether any particular marker protein is a secreted protein. In order to make this determination, the marker protein is expressed in, for example, a mammalian cell, preferably a human cell line, extracellular fluid is collected, and the presence or absence of the protein in the extracellular fluid is assessed (e.g. using a labeled antibody which binds specifically with the protein).

The following is an example of a method which can be used to detect secretion of a protein. About 8×10⁵ 293T cells are incubated at 37° C. in wells containing growth medium (Dulbecco's modified Eagle's medium {DMEM} supplemented with 10% fetal bovine serum) under a 5% (v/v) CO2, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

It will be appreciated that subject samples, e.g., a sample containing tissue, whole blood, serum, plasma, buccal scrape, saliva, cerebrospinal fluid, urine, stool, liver tissue, cirrhotic tissue, and bone marrow, may contain cells therein, particularly when the cells are cancerous, and, more particularly, when the cancer is metastasizing, and thus may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the marker in the sample. Thus, the compositions, kits, and methods of the invention can be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a marker corresponding to a protein having at least one portion which is displayed on the surface of a cell which expresses it may be detected without necessarily lysing the cell (e.g. using a labeled antibody which binds specifically with a cell-surface domain of the protein).

The invention also encompasses kits for detecting the presence of a polypeptide or nucleic acid corresponding to a marker of the invention in a biological sample, e.g., a sample containing tissue, whole blood, serum, plasma, buccal scrape, saliva, cerebrospinal fluid, urine, stool, liver tissue, cirrhotic tissue, and bone marrow. Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit. Methods and techniques applicable to array (including protein array) synthesis have been described in PCT Application Nos. WO 00/58516, and WO 99/36760, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, which are all incorporated herein by reference in their entirety for all purposes. Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

4. Method for Detecting Structural Alterations

The invention also provides a method for assessing whether a subject is afflicted with a hepatic disorder or is at risk for developing a hepatic disorder by comparing the structural alterations, e.g., mutations or allelic variants, of a marker in a hepatic disorder sample with the structural alterations, e.g., mutations of a marker in a normal, e.g., control sample. The presence of a structural alteration, e.g., mutation or allelic variant in the marker in the sample is an indication that the subject is afflicted with a hepatic disorder.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a marker prior to identifying the allelic variant. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a marker and detect allelic variants, e.g., mutations, by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled DNA Sequencing by Mass Spectrometry Via Exonuclease Degradation by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

In some cases, the presence of a specific allele of a marker in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a marker allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl. Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oelher and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility are used to identify the type of marker allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et at (1989) Proc. Natl. Acad. Sci. USA 86:6230; and Wallace et al. (1979) Nucl. Acids Res. 6:3543). Such allele specific oligonucleotide hybridization techniques may be used for the simultaneous detection of several nucleotide changes in different polylmorphic regions of marker. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et at (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et at (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

The invention further provides methods for detecting single nucleotide polymorphisms in a marker. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., (1989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mutat. 1:159-164; Ugozzoli, L. et al., (1992) GATA 9:107-112; Nyren, P. (1993) et al., Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the allelic variant of a polymorphic region located in the coding region of a marker, yet other methods than those described above can be used. For example, identification of an allelic variant which encodes a mutated marker can be performed by using an antibody specifically recognizing the mutant protein in, e.g., immunohistochemistry or immunoprecipitation. Antibodies to wild-type marker or mutated forms of markers can be prepared according to methods known in the art.

Alternatively, one can also measure an activity of a marker, such as binding to a marker ligand. Binding assays are known in the art and involve, e.g., obtaining cells from a subject, and performing binding experiments with a labeled ligand, to determine whether binding to the mutated form of the protein differs from binding to the wild-type of the protein.

B. Pharmacogenomics

Agents or modulators which have a stimulatory or inhibitory effect on amount and/or activity of a marker of the invention can be administered to individuals to treat (prophylactically or therapeutically) a hepatic disorder, including cancer, in the subject. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the amount, structure, and/or activity of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the amount, structure, and/or activity of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of amount, structure, and/or activity of a marker of the invention.

C. Monitoring Clinical Trials

Monitoring the influence of agents (e.g., drug compounds) on amount, structure, and/or activity of a marker of the invention can be applied not only in basic drug screening, but also in clinical trials. For example, the effectiveness of an agent to affect marker amount, structure, and/or activity can be monitored in clinical trials of subjects receiving treatment for a hepatic disorder, including cancer. In a preferred embodiment, the present invention provides a method for monitoring the effectiveness of treatment of a subject with an agent (e.g., an agonist, antagonist, peptidomimetic, protein, peptide, antibody, nucleic acid, antisense nucleic acid, ribozyme, small molecule, RNA interfering agent, or other drug candidate) comprising the steps of (i) obtaining a pre-administration sample from a subject prior to administration of the agent; (ii) detecting the amount, structure, and/or activity of one or more selected markers of the invention in the pre-administration sample; (iii) obtaining one or more post-administration samples from the subject; (iv) detecting the amount, structure, and/or activity of the marker(s) in the post-administration samples; (v) comparing the amount, structure, and/or activity of the marker(s) in the pre-administration sample with the amount, structure, and/or activity of the marker(s) in the post-administration sample or samples; and (vi) altering the administration of the agent to the subject accordingly. For example, increased administration of the agent can be desirable to increase amount and/or activity of the marker(s) to higher levels than detected, i.e., to increase the effectiveness of the agent. Alternatively, decreased administration of the agent can be desirable to decrease amount and/or activity of the marker(s) to lower levels than detected, i.e., to decrease the effectiveness of the agent.

### EXEMPLIFICATION

This invention is further illustrated by the following examples which should not be construed as limiting. The contents of all references, figures, sequence listing, patents and published patent applications cited throughout this application are hereby incorporated by reference.

### Example 1

### Materials and Methods For Examples 1-6

### A. RNA Extraction

Tumor and adjacent liver tissues were macro-dissected from 10 μm FFPE tissue sections. Absence of microvascular tumor invasion in the adjacent liver tissue was confirmed using H&E staining of consecutive sections. Using 3-4 sections for each sample, total RNA was extracted using the High Pure RNA Paraffin™ kit (Roche) as directed by the manufacturer (training set) or TRIzol LS™ reagent (Invitrogen) in a semi-automated 96-well plate format based on the manufacturer's instructions (validation set).

### B. Gene Expression Arrays for FFPE Tissues

DASL Assay

To profile randomly fragmented mRNA extracted from FFPE tissue (FFPE-RNA), the DNA-mediated Annealing, Selection, extension and Ligation (DASL) assay (Illumina) was employed (Fan, J. B., et al. (2004) Genome Res. 14(5), 878-885; Bibikova, M., et al. (2004) Am. J. Pathol. 165(5), 1799-1807). Briefly, fragmented FFPE-RNA was converted into cDNA using random primers. For each target site on the cDNA, a pair of query oligos separated by a single nucleotide is annealed to the cDNA and the gap between the query oligos is extended and ligated to generate a PCR template. A pair of universal PCR primers was then used for amplification, and linearly amplified PCR products were hybridized to a bead microarray. The array was then scanned by a BeadArray Reader (Illumina).

Number of Microarray Probes Assigned to Each Gene

Missing signals due to RNA degradation was one of the major concerns in profiling FFPE tissues. For this reason, a commercially available panel of 502 cancer-related genes for DASL assay (Cancer Panel, Illumina) assigns 3 independent probes to each gene, with the expectation that this would maximize data quality. However, the use of multiple probes per gene diminishes the number of transcripts that can be assayed per array (given a fixed number of probes per array). Therefore, it was sought to be experimentally determined what effect reducing the number of probes per gene caused, so as to facilitate covering a larger number of genes with the same total number of probes. A single probe was randomly picked from among the 3 probes assigned to each gene, and performance of the single probe dataset performed in sample clustering and marker gene selection analyses was evaluated.

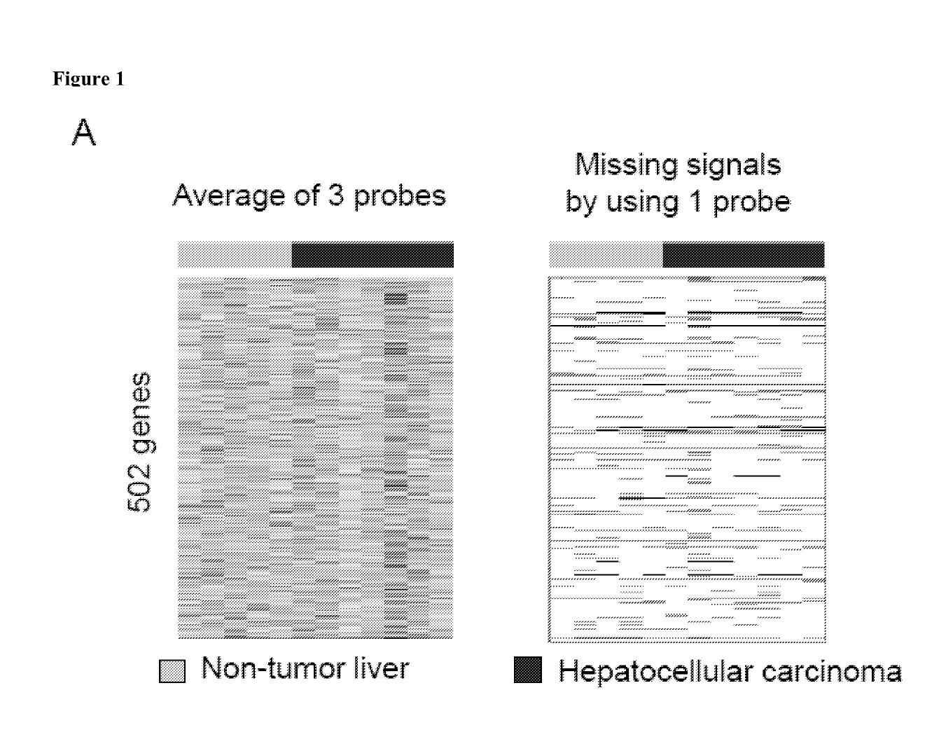

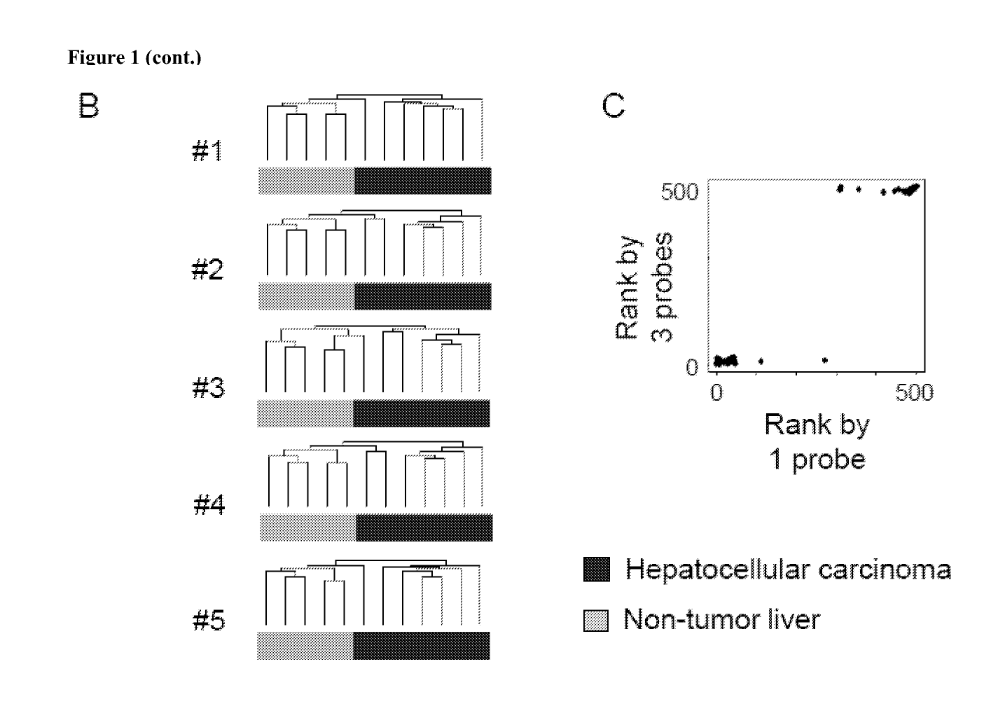

First, by picking a single probe for each gene, 5-7% of measurements fell below the level of negative control probes, suggesting either missing signals due to RNA degradation or suboptimal probe sequence (FIG. 1A). However, it was noted that such probe drop-out had little effect on overall performance of the arrays. For example, a prostate cancer vs. normal distinction was not affected by the single probe picking (FIG. 1B). This suggests that profiling 100's˜1000's genes can compensate for the slight increase in noise caused by RNA degradation. In marker gene analysis, only a small number of genes were dropped from the top marker gene list (indicating a small number of false negatives), and no genes came to the top of the marker list in the single probe data but were absent in the dataset using 3 probes per gene (indicating no false positives) (FIG. 1C).

Designing a 6,000-Gene DASL Assay

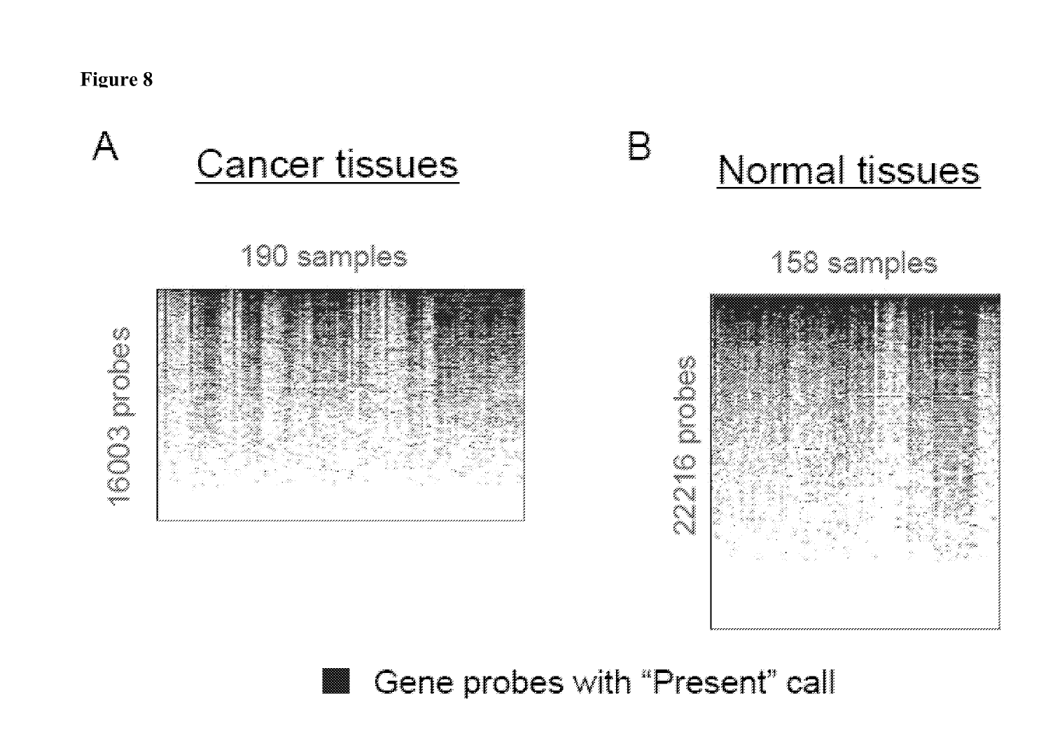

It was desired to identify ˜6,000 maximally informative transcripts that could be used for genome-wide discovery on the DASL platform (configured as 4×1536 assays utilizing one probe per gene). To address this, a large collection of Affymetrix transcriptome datasets profiling cancer and normal tissues was analyzed (Ramaswamy, S., et al. (2001) Proc. Natl. Acad. Sci. USA. 98(26), 15149-15154; Su, A. I., et al. (2004) Proc. Natl. Acad. Sci. USA. 101(16), 6062-6067). This analysis revealed that the expression signals from one third of the genes on most genome-wide arrays were “absent” (FIG. 8). This suggested that a substantial proportion of the genome is infrequently expressed, and therefore might be omitted without great consequence. By excluding such genes, defining a generic minimum subset of genome representing the global structure of the entire transcriptome was sought.

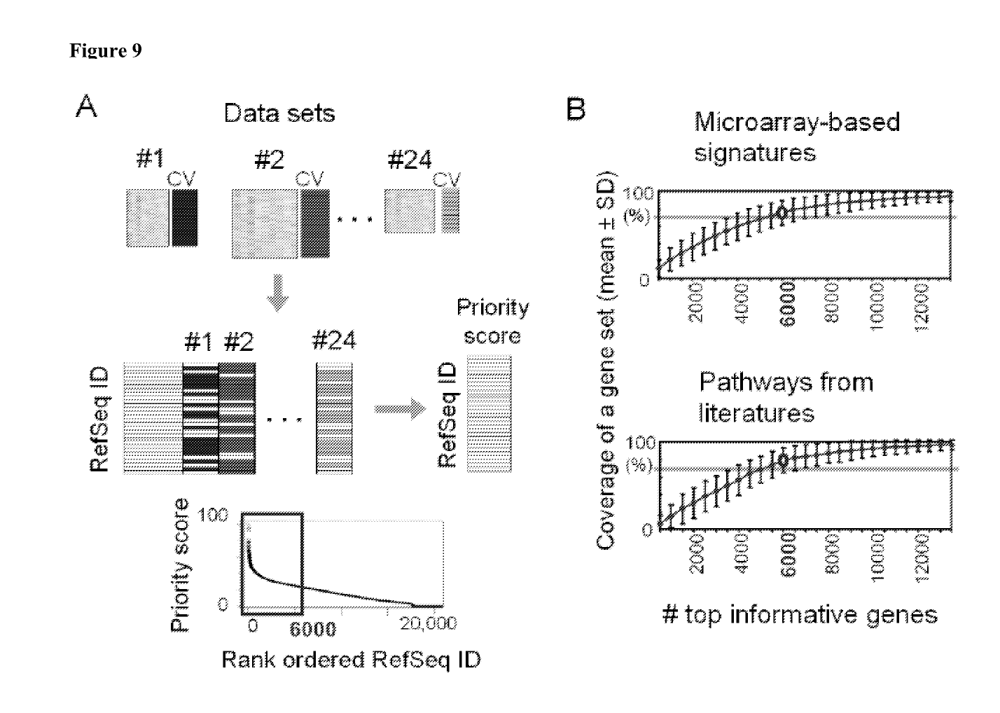

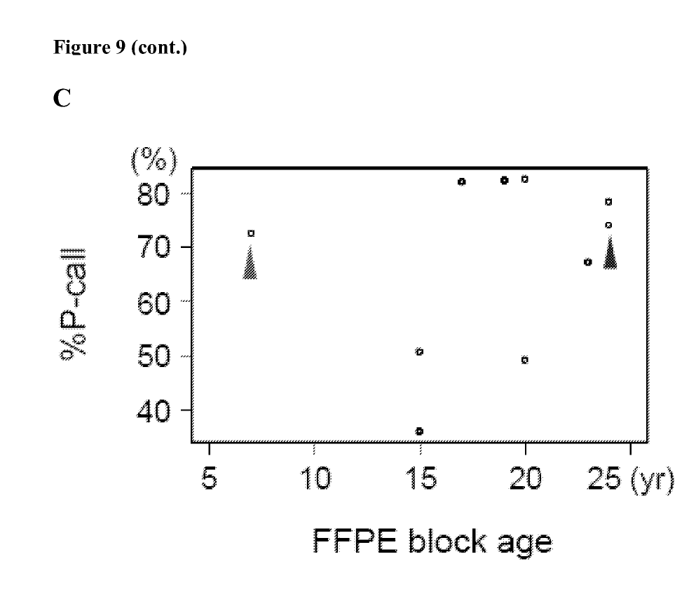

A set of query oligos (i.e., probes) was designed to profile transcriptionally informative genes that might be useful for signature discovery and validation. To this end, genes were selected with the largest variation across samples in a large collection of previously generated Affymetrix microarray datasets spanning 24 studies, 2,149 samples, and 15 tissue types (Table 8). After filtering out genes with less than a 3-fold difference and less than 100 units between the maximum and minimum signals across the dataset, the coefficient of variation (CV) was calculated and summarized onto the NCBI's RefSeq gene IDs to compute a priority score for each gene, and genes were rank-ordered according to this score (FIG. 9A). An examination of published marker genes from recent studies indicated that the generated list of 6,000 genes represented 70-90% of these genes, indicating that the 6,000 gene array was more informative than a random collection of 6,000 genes (which might be expected to capture only ˜25% of reported markers) (FIG. 9B). Query oligos were then designed for the top informative 6,100 genes (NCBI's Gene Expression Omnibus, see the NCBI website, platform ID GPL5474; FIG. 19).

Quality Assessment of DASL Profile

As a quality measure of the DASL gene expression profile, the proportion of gene probes with a “present” signal (% P-call) was calculated, which is expected to be similar across samples of a given tissue type (e.g., HCC). The “present” call rate drops precipitously when degraded RNA typical of FFPE tissues is analyzed on conventional microarrays such as Affymetrix arrays. The “present” call was computed based on built-in negative control probes (GenePattern, IlluminaDASL pipeline). In a pilot experiment performed on 10 prostate cancer tissues, a % P-call of ˜75% was observed in 2 samples fixed 24 years before RNA extraction, which was comparable to a sample fixed 7 years ago (FIG. 9C), indicating that data quality is not directly correlated with age of the sample.

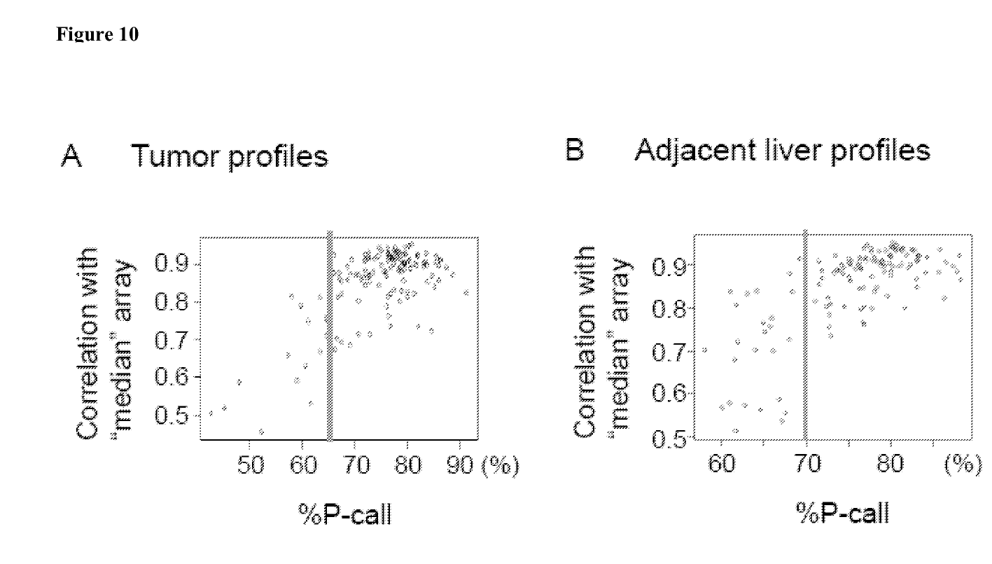

Poor quality profiles were detected and removed as follows. A “median” array was set as a representative sample in a dataset by calculating the median for each gene. The poor quality, outlier profiles were defined based on dissimilarity to the “median” array measured by Pearson correlation coefficient. In the plot of the correlation to % P-call, it was observed that the correlation sharply started to drop as % P-call became smaller than a certain value. This likely indicates that the samples with % P-call smaller than this value have severe RNA degradation affecting sensitivity of gene expression signal detection. Based on this plot, a quality threshold of % P-call was set for each tissue type to assure a minimum correlation coefficient of 0.7 for the majority of the samples (the % P-call quality thresholds of 65% and 70% was set for tumor and adjacent liver tissues, respectively, FIG. 10). Failure of the profiling, i.e., % P-call less than 70% in adjacent liver set, was not associated with clinical variables including age (p=0.49), sex (p=0.78), existence of cirrhosis (p=1.00), Child-Pugh stage (p=0.11), HCC etiology (p>0.70), or age of the FFPE block (>10 years, p=0.30).

The same % P-call threshold was applied for the validation set. After eliminating samples with poor quality data, the raw data were normalized using the cubic spline algorithm (Workman, C., et al. (2002) Genome Biol. 3(9), research 0048) using the IlluminaDASL pipeline within GenePattern. Only gene probes with a minimal 3-fold differential expression and absolute difference >500 units across the samples were included after applying floor and ceiling values of 200 and 80,000 units, respectively.

Comparison of Gene Expression Profiles Between Intact and FFPE-RNA

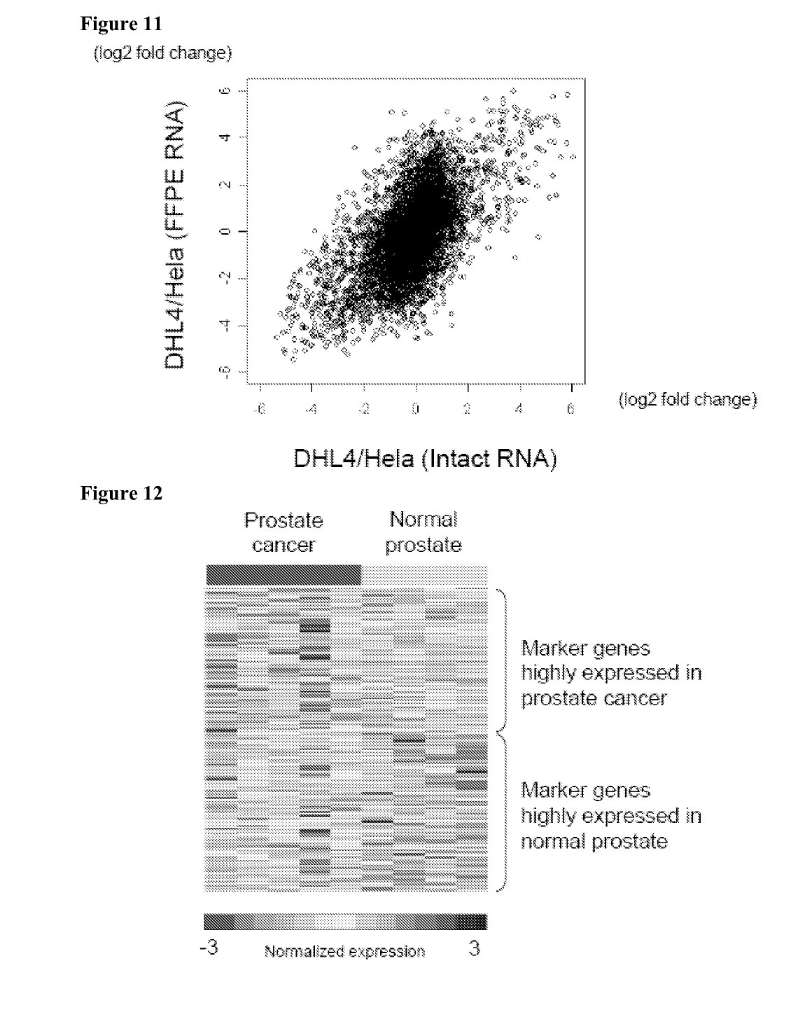

First, the extent of correlation of gene expression profile of FFPE tissue with that of fresh tissue at the level of individual genes was evaluated. To ensure a uniform population of cells being subjected to the fresh and fixed analysis, cell lines were used (as opposed to tissues, which have greater intra-tissue variability which would become a confounding factor in these analyses). DHL4 and Hela cell lines were cultured, harvested, and split into two halves. Total RNA was immediately extracted from one half, and the other half was fixed with formalin and embedded in a paraffin block. Total RNA was also extracted from the FFPE block using the protocol described below. All RNA samples were profiled using the DASL assay, and fold changes were calculated for each gene in a comparison between DHL4 and Hela cell lines. The plot of the fold changes for the intact and FFPE cell lines showed moderate correlation (Pearson correlation coefficient 0.61, p<0.001, FIG. 11). At the higher fold changes in the fresh RNA profiles, the vast majority of the genes showed concordant gene expression changes in the FFPE profiles (Table 9).

Next, it was determined whether the DASL profile of FFPE tissue recapitulates the biologically relevant information observed in the profile of fresh frozen tissue. For this analysis, prostate cancer data was used, for which there exists an abundance of published microarray data derived from frozen tumor and normal tissues. 200 marker genes were identified that reflect the tumor vs. normal prostate distinction based on a meta-analysis of 7 published frozen sample-based microarray datasets collected in a cancer transcriptome database (see the Oncomine company website on the world wide web). Among those genes, 180 genes (90%) are included in the 6,100 informative gene panel. Based on the expression pattern of those marker genes, a collection of FFPE tumor and normal prostate samples was classified using a nearest template prediction method (see Section D. “Data analysis” section below) was classified. Prediction with statistical significance with 100% accuracy was observed (false discovery rate<0.05, FIG. 12), indicating that the 6,000-gene DASL assay robustly identifies biologically meaningful patterns in FFPE tissues. A meta-analysis of 3 independent frozen sample-based HCC datasets was also performed including 232 samples to define common subclasses of HCC, and it was found that the molecular subclasses identified in the frozen tissues were also seen in the profiles of 118 FFPE HCC tissues profiled by DASL. It is therefore concluded that the 6,000-gene DASL assay accurately recapitulates the gene expression profile of fresh frozen tissues in archived, FFPE material.

### C. Data Availability

Microarray datasets are available through Gene Expression Omnibus (GSE10143) on the world wide web at the Broad Institute website of the Massachusetts Institute of Technology.

### D. Data Analysis

Definition of Clinical Outcome

While HCC is the cause of death in most patients with the disease, some patients die of liver failure or other causes attributable to cirrhosis in the absence of progressive HCC (7 of the 39 deaths in the present study died of non-HCC causes). Accordingly, HCC-related mortality was chosen (disease-specific death) as the principal clinical endpoint for the survival-predictive signature discovery, defined as follows: (1) tumor occupying more than 80% of the liver, (2) portal venous tumor thrombus (PVTT) proximal to the second bifurcation, (3) obstructive jaundice due to tumor, (4) distant metastasis, or (5) variceal hemorrhage with PVTT proximal to the first bifurcation. The commonly used definition of “late recurrence” was tumor recurrence appearing more than 2 years after surgery (Bruix, J. and Sherman, M. (2005) Hepatology 42(5), 1208-1236; Imamura, H., et al. (2003) J. Hepatol. 38(2), 200-207). For late recurrence prediction, early recurrences were treated as censored observations.

Prognostic Prediction

Most outcome prediction studies discretize outcome in a binary fashion, creating two classes of patients: those with good outcome, and those with bad outcome. Unfortunately, this approach requires creating a boundary between the two groups that is often not obvious, and the approach works poorly with patients of intermediate outcome. In this study, non-discretized, censored survival time was used to select signature genes in order to not sacrifice sample size and to avoid the problem of setting an arbitrary cut-off of survival time. In addition, it was sought to determine whether the expression of poor- and good-prognosis signature genes were coordinately regulated in a given sample. That is, it was expected that the poor signature genes would be ON (or up) and the good signature genes would be OFF (or down) in a “poor” survival sample. To evaluate this, a simple nearest neighbor-based method was designed assessing a sample's proximity to a hypothetical representative sample (template) of poor or good survival. This approach allowed performance of a single sample-based outcome prediction. The details of the method are described below.

Genes positively or negatively correlated with HCC-related survival or time-to-recurrence were selected using the Cox score (Bair, E. and Tibshirani, R. (2004) PLoS Biol. 2(4), E108; see the Significance Analysis of Microarrays User Manual at the Stanford University Statistics Department website on the world wide web) using the following formula.

$$d = \frac{[ {\sum }_{k = 1}^{K} ( {x}_{k}^{*} - {d}_{k} {\overline{x}}_{k} ) ]}{{[ {\sum }_{k = 1}^{K} ( {d}_{k} / {m}_{k} ) {\sum }_{t ∈ {R}_{k}} {( {x}_{t} - {x}_{k} )}^{2} ]}^{1 / 2}}$$

where i is indices of samples, xᵢ is gene expression level for sample i, tᵢ is time for sample i, kε{1, . . . , K} is indices of unique death times z₁, z₂, . . . zₖ, dₖ is number of deaths at time zₖ, mₖ is number of samples in Rₖ={i:tᵢ≧zₖ}, x*ₖ=Σ_{t(sub i)=z(sub k)}xᵢ, and xₖ=Σ_{iεR(sub k)}xᵢ/mₖ. Prediction analysis was performed by evaluating the expression status of the signature using the nearest template prediction (NTP) method as implemented in the NearestTemplatePrediction module of the GenePattern analysis toolkit. Briefly, a hypothetical sample serving as the template of “poor” outcome was defined as a vector having the same length as the predictive signature. In this template, a value of 1 was assigned to “poor” outcome-correlated genes and a value of −1 was assigned to “good” outcome-correlated genes, and then each gene was weighted by the absolute value of the corresponding Cox score. The template of “good” outcome was similarly defined. For each sample, a prediction was made based on the proximity measured by the cosine distance to either of the two templates. Significance for the proximity was estimated by comparison to a null distribution generated by randomly picking (1,000 times) the same number of marker genes from the microarray data for each sample, and correcting for multiple hypothesis testing using the false discovery rate (FDR) (Reiner, A., et al. (2003) Bioinformatics. 9(3), 368-375). A sample closer to the template of “poor” outcome with an FDR <0.05 was predicted as having poor outcome.

Study Design to Define Outcome Predictive Signature

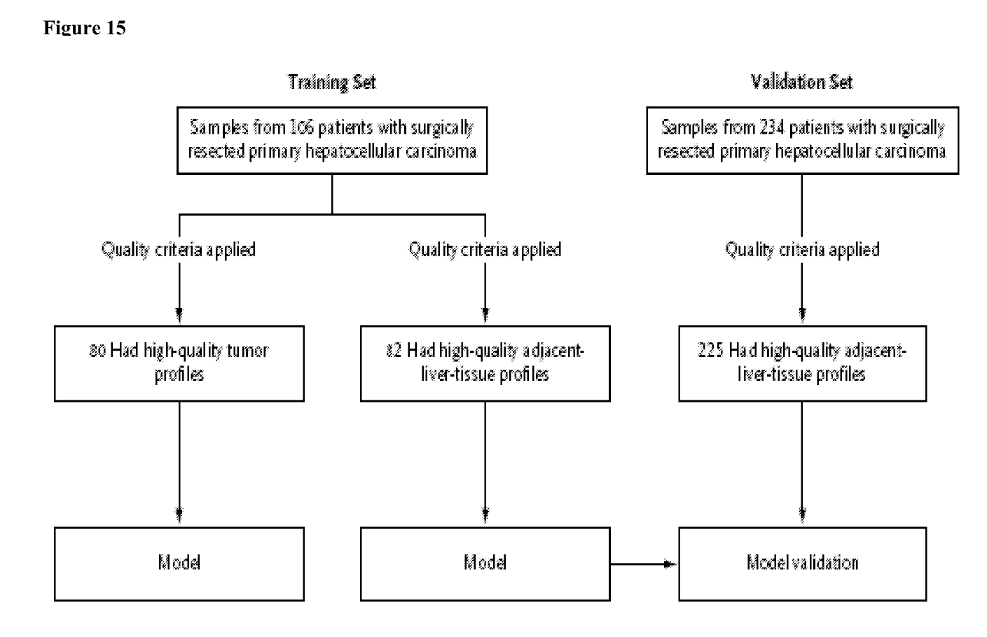

Tumor and adjacent non-tumor liver tissues from the training set were profiled separately to define an outcome-predictive signature (FIG. 15). The signature was first internally validated in the training set using a leave-one-out cross-validation prediction procedure. A single sample was left out one-by-one and an outcome-correlated signature was selected from the remaining samples (selecting marker genes based on permutation test p-value less than 0.05). A predicted label was assigned to the left-out sample based on the closest “template” using NTP algorithm. Only genes selected in each of the leave-one-out trials were included in the outcome-predictive signatures tested on the validation set.

Gene Set Enrichment Analysis

Functional annotation of the survival signature was performed by Gene Set Enrichment Analysis (GSEA) (Subramanian, A., et al., (2005) Proc. Natl. Acad. Sci. USA. 102(43), 15545-15550). Two categories of annotated gene sets were evaluated: target genes of experimental perturbation (473 sets) and literature-based curated pathway gene sets (150 sets) collected in the molecular signature database (MSigDB, see the Broad Institute website on the world wide web).

Survival Data Analysis

Survival difference was evaluated by the log-rank test, and survival association of clinical variables and the signatures was assessed by Cox regression analysis (Survival Analysis modules, GenePattern). First, well-accepted clinical predictors of HCC outcome were evaluated (Bruix, J. and Sherman, M. (2005) Hepatology 42(5), 1208-1236; Llovet, J. M., and Burroughs, A. (2003) Lancet 362(9399), 1907-1917): AFP, multinodularity, and vascular invasion, by univariate analysis. Only variables with statistical significance (p<0.05) were further evaluated by multivariate analysis. The hazard rate for tumor recurrence was calculated as previously described (Imamura, H., et al. (2003) J Hepatol. 38(2), 200-207; Mazzaferro, V., et al. (2006) Hepatology. 44(6), 1543-1554) to estimate the pattern of HCC recurrence over time after surgery. GenePattern modules and pipeline used in this study are available from the Broad Institute website on the world wide web. All other clinical data analyses were performed using the R statistical package (see the R-project website on the world wide web).

### E. Clonality Analysis

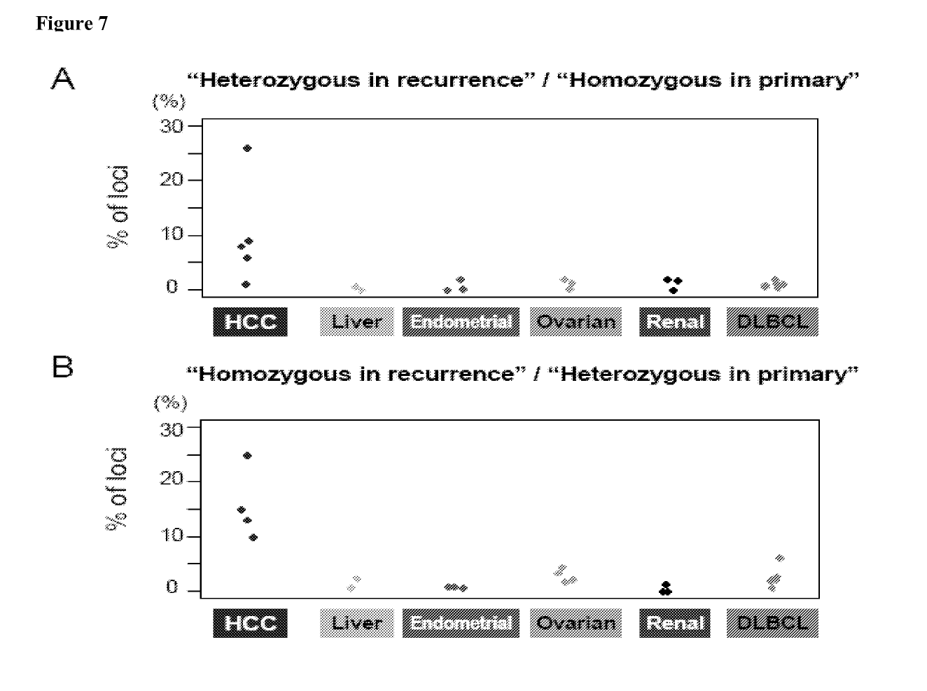

Five pairs of primary and recurrent HCC tumors, 2 pairs of adjacent non-tumor liver tissues, and Hela cells were profiled for SNPs using the LinkagePanel™ beadarray (Illumina) according to the manufacturer's instructions (Lips, E. H., et al. (2005) Cancer Res. 65(22), 10188-10191). Genotype cells were generated using BeadStudio™ software (Illumina). In order to address whether primary tumors and recurrences likely derived from the same clone, the pattern of heterozygosity in each of the samples was analyzed. In particular, the number of loci that appeared homozygous in the primary tumor, but were called as heterozygous at recurrence, were counted. Such cases would suggest that primaries and recurrences derived from different clones, given that regions of LOH in a primary tumor (appearing homozygous on SNP arrays) would likely appear the same in recurrences if the recurrences derived from the same clone (Table 7A). Pairs of primary and recurrence/metastasis tumor tissues in endometrial (n=3), ovarian (n=4), lymphoma (n=6) and renal (n=3) cancers were similarly analyzed to estimate the same measure of clonality in other, non-HCC tumor types (Table 7B). The HCC pairs showed a significantly higher proportion of loci that appeared homozygous in the primary tumor, yet appeared heterozygous at recurrence (p=0.008, Wilcoxon rank sum test). Similarly, there were more loci that were heterozygous in the HCC primary and homozygous at recurrence, compared to other tumor types (p=0.001) (FIG. 7).

### F. Outcome Prediction Using HCC Tissue Data

It was determined whether other machine-learning classifiers based on the binary classes (i.e., “good” and “poor” prognosis) predict outcome in the profiles of HCC tissues. Multiple classification methods were tested including Classification of Regression Tree (CART), k-nearest neighbor (k-NN), weighted voting (WV), and support vector machine (SVM), but as shown in Table 10, these methods also failed to yield statistically significant predictions (p=0.34 for survival and p=0.92 for recurrence. Log-rank test). This result indicates that the failed HCC tissue-based outcome prediction by the present method is not due to selection of classification algorithm.

### G. Survival Signature in Fresh Frozen Non-Tumor Liver

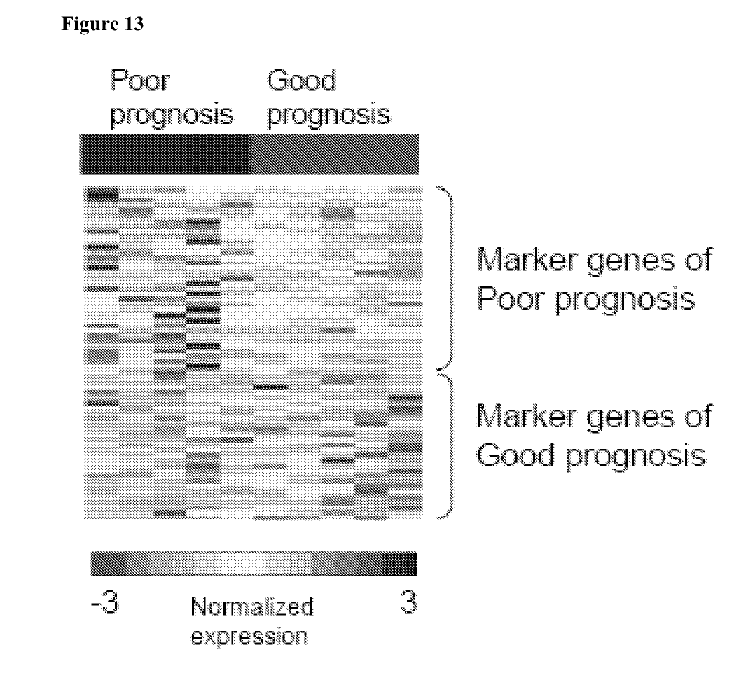

It was confirmed that the survival signature was readily detectable in a publicly available, independent dataset of fresh frozen non-tumor liver tissues (GSE6764) (FIG. 13). Prediction was performed using the nearest template prediction method.

### H. Patient Survival in Validation Set According to Geographic Site

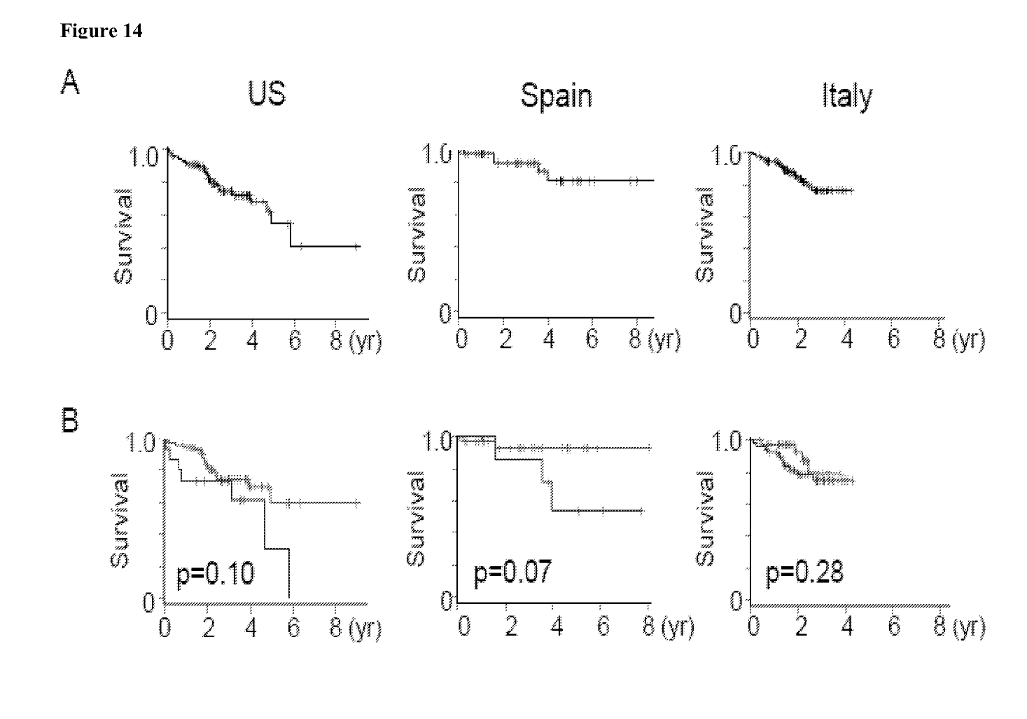

A trend toward survival separation was also seen within each geographic site in the validation set (i.e., U.S., Spain and Italy), although this did not reach statistical significance due to the small sample size and/or insufficient follow-up time in each site (FIG. 14).

### I. Patients and Samples

The training set consisted of tissue samples from 106 patients who were consecutively treated with surgery for primary hepatocellular carcinoma between 1990 and 2001 at Toranomon Hospital in Tokyo and for whom data on clinical outcomes (over a median follow-up period of 7.8 years) and formalin-fixed, paraffin-embedded blocks of tumor and adjacent tissue were available (FIG. 15). The validation set included tissue samples from 234 patients with hepatocellular carcinoma who consecutively underwent surgery between 1994 and 2005: 92 patients at the Mount Sinai School of Medicine in New York, 46 at Hospital Clinic Barcelona, and 96 at the National Cancer Institute of Milan (members of the HCC Genomic Consortium). Archived formalin-fixed, paraffin-embedded tissues obtained as part of routine clinical care were analyzed, with approval by the local institutional review boards granted on the condition that all samples be made anonymous. Formalin-fixed, paraffin-embedded blocks obtained at the time of resection were cut into three or four sections (each 10 μm thick), macrodissected to isolate tumor and adjacent liver tissue, and subjected to RNA extraction as described above.

### J. Analysis of Gene Expression and Clonality

Gene-expression profiling was performed according to the complementary DNA-mediated annealing, selection, extension, and ligation (DASL) assay (Illumina; Fan, J. B., et al. (2004) Genome Res. 14, 878-885; Bibikova, M., et al. (2004) Am. J. Pathol. 165, 1799-18077), and 6,100 transcriptionally informative genes were selected for analysis (see Section B, above). Microarray data are at the NCBI website on the world wide web at accession numbers GSE10143 and GPL5474. Genes whose expression was associated with disease-specific survival and time to recurrence were selected with the use of the Cox score (see Section D. above). The value of the signature was assessed on the basis of overall survival. Late recurrence was defined as tumor recurrence 2 or more years after surgery (Llovet, J. M., et al. (2005) Semin. Liver Dis. 2, 181-200; Imamura, H., et al. (2003) J. Hepatol. 38, 200-207). Outcome association analysis was performed with the use of a nearest neighbor-based method (see Section D, above).

### K. Statistical Analysis

Functional annotation was performed by means of gene set enrichment analysis (GSEA; see the Broad Institute website on the world wide web; Subramanian, A., et al. (2005) Proc. Natl. Acad. Sci. USA 102, 15545-15550). Survival analyses were performed with the use of the log-rank test and Cox regression modeling. Subgroup analysis was per-formed on data from patients with a longer duration of follow-up (treated no later than 2004) and those with carcinoma classified as stage 0 or stage A according to the Barcelona Clinic Liver Cancer staging system (BCLC), which ranks hepatocellular carcinoma in five stages, ranging from 0 (very early stage) to D (terminal stage) (Llovet, J. M., et al. (2003) Lancet 362, 1907-1917; Bruix, J., et al. (2005), Hepatology 42, 1208-1236). The hazard function for tumor recurrence was calculated as previously described (Imamura, H., et al. (2003) J. Hepatol. 38, 200-207; Mazzaferro, V., et al. (2006) Hepatology 44, 1543-1554). All analyses were performed with the use of GenePattern (Reich, M., et al. (2006), Nat. Genet. 38, 500-501; see the Broad Institute website on the world wide web) or the R statistical package (see the R-project website on the world wide web).

### Example 2

### Validation of the Profiling Method

A method that was suitable for gene-expression profiling of formalin-fixed, paraffin-embedded material was sought. An approach has been reported for the analysis of several hundred transcripts based on DASL, a multiplex, locus-specific polymerase-chain-reaction (PCR) assay (Fan, J. B., et al. (2004) Genome Res. 14, 878-885; Bibikova, M., et al. (2004) Am. J. Pathol. 165, 1799-1807). However, an unbiased discovery of diagnostic signatures requires a genomewide profiling method. Accordingly, the DASL method was modified for probe selection and analysis and performed a bioinformatic meta-analysis to identify 6,000 transcripts that captured the majority of variance in gene expression across the human transcriptome. This 6,000-gene DASL assay served as a potential tool for genomewide analysis of formalin-fixed, paraffin-embedded tissues. The assay was found to be highly reproducible (R2>0.96 in replicate experiments), with an overall success rate of 90% among all the specimens, including formalin-fixed, paraffin-embedded tissue blocks collected up to 24 years ago. It was found that representing each transcript with one probe only (as opposed to three, as previously reported ((Fan, J. B., et al. (2004) Genome Res. 14, 878-885; Bibikova, M., et al. (2004) Am. J. Pathol. 165, 1799-1807) resulted in little loss of assay performance (FIG. 1).

### Example 3

### Profiles of Hepatocellular Carcinoma Tumors

Table 11 summarizes the clinical characteristics of the patients in the training and validation sets. All patients were treated with curative surgical resection, which was, in some cases, followed by second-line treatments at the time of recurrence.

By design, the training set included tissue samples from a large proportion of patients with very-early-stage hepatocellular carcinoma (BCLC stage 0), because these patients represent the greatest clinical challenge with respect to outcome prediction. Indeed, no clinical variables, either alone or in combination, were associated with survival among these patients. Although there were no significant differences between the training set and validation set with respect to the number of patients with advanced-stage carcinoma (BCLC stage B) or the status of liver function, there was heterogeneity between the two sets with respect to certain tumor characteristics, such as diameter and type of viral infection (Table 1). Such heterogeneity may help to ensure that molecular predictors have real-world applicability across heterogeneous populations of patients.

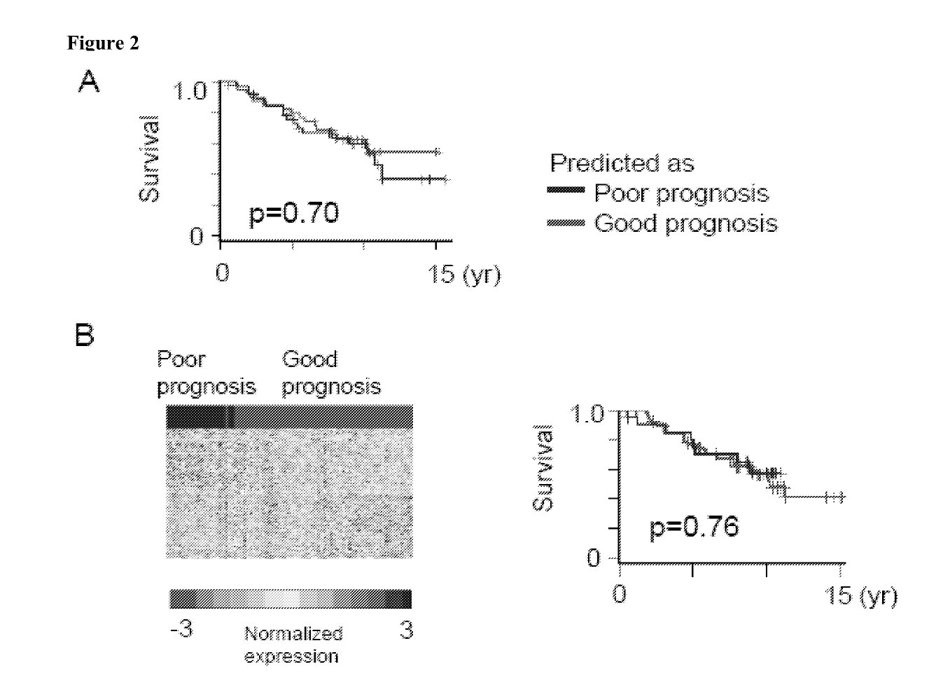

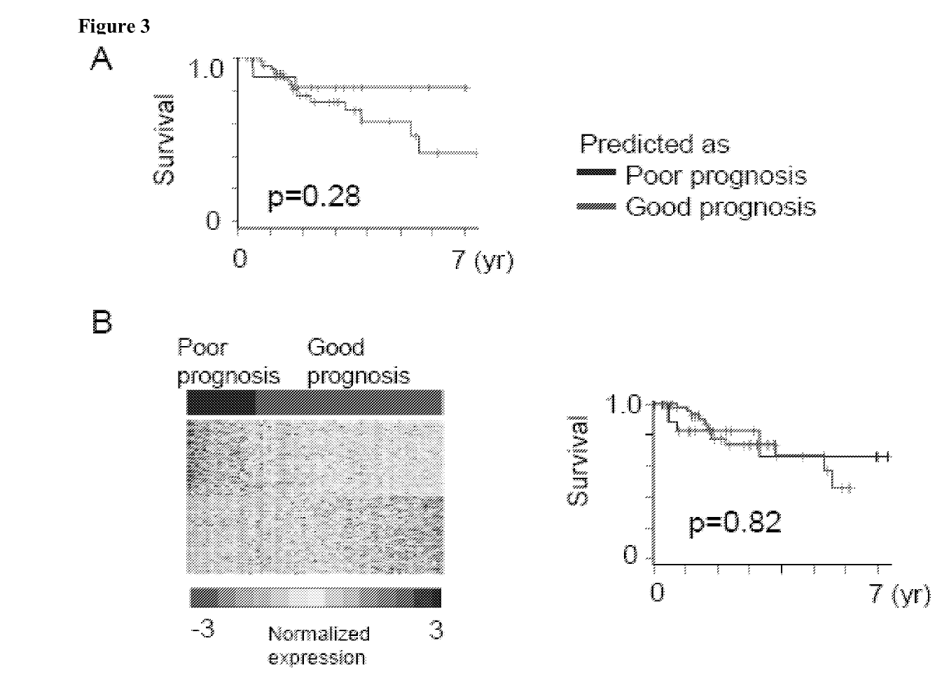

It was first investigated whether gene-expression profiles of hepatocellular carcinoma tumors were associated with the clinical outcome. For each of the 106 patients in the training set, tumor-containing portions of the formalin-fixed paraffin embedded blocks were macrodissected away from surrounding liver tissue. Eighty tumors (75%) yielded high-quality gene-expression profiles. Using a leave-one-out cross-validation procedure and a nearest-neighbor-based algorithm, a significant gene-expression correlate of either tumor recurrence (P=0.22) or survival (P=0.70) failed to be detected (FIG. 2A). Furthermore, a previously reported signature associated with survival among patients with hepatocellular carcinoma (Lee, J. S., et al. (2004) Hepatology 40, 667-676) was not associated with survival in the studied series of patients (P=0.76) (FIG. 2B). This failure to identify an outcome-associated signature is unlikely to be due to a technical flaw of the formalin-fixed, paraffin-embedded DASL method, because the same molecular-subclass structure in the formalin-fixed, paraffin-embedded samples was observed as that in collections of frozen samples of hepatocellular carcinoma (FIGS. 2B and 3B). Although this result does not exclude the possibility of tumor-derived expression profiles as predictors of the outcome of hepatocellular carcinoma, the data suggest that at least in this training set, the outcome was largely related to other factors.

### Example 4

### Survival Signature in Adjacent Liver Tissue

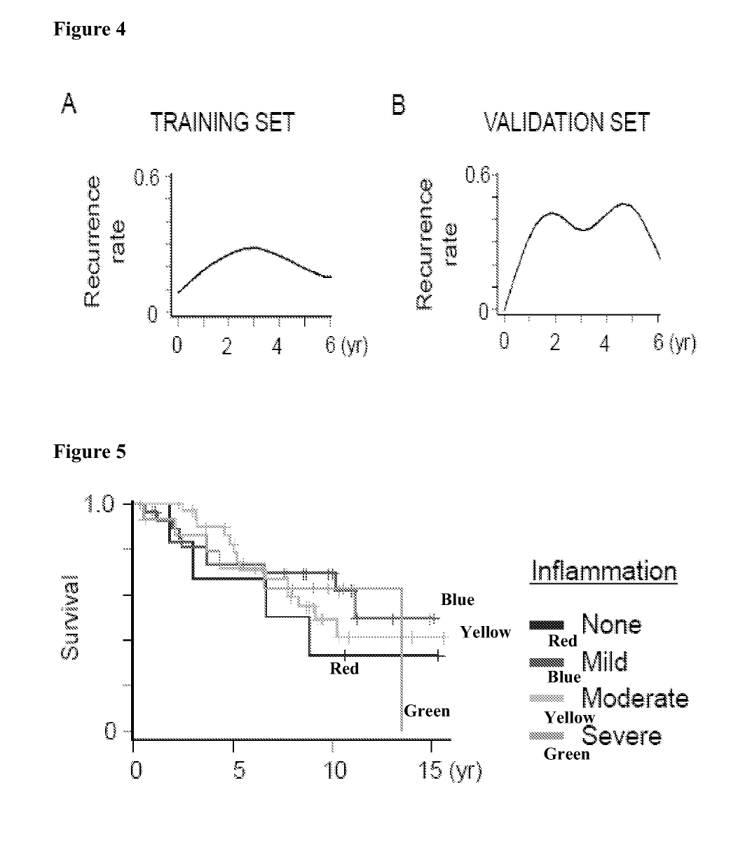

The lack of association between tumor-derived gene-expression profiles and survival provided the lead to consider the pattern of recurrence of early-stage hepatocellular carcinoma. In contrast to advanced tumors, which tend to recur rapidly after resection, early-stage tumors, which are increasingly diagnosed in modern clinical practice, recur much later, generally more than 2 years after resection (Llovet, J. M., et al. (2005) Semin. Liver Dis. 2, 181-200; Imamura, H., et al. (2003) J. Hepatol. 38, 200-207; FIG. 4). This emerging pattern of late recurrence of hepatocellular carcinoma (due at least in part to the diagnosis of hepatocellular carcinoma at an early stage) has led to the notion that a late recurrence may not be an actual recurrence but rather a second primary tumor in an at-risk liver, presumably due to the carcinogenic effects of cirrhosis (Llovet, J. M., et al. (2003) Lancet 362, 1907-1917; Llovet, J. M., et al. (2008) J. Hepatol. 48, S20-S37; Llovet, J. M., et al. (2005) Semin. Liver Dis. 2, 181-200). It was therefore hypothesized that the surrounding liver tissue—not the tumor itself—might harbor a gene-expression signature associated with subsequent recurrence.

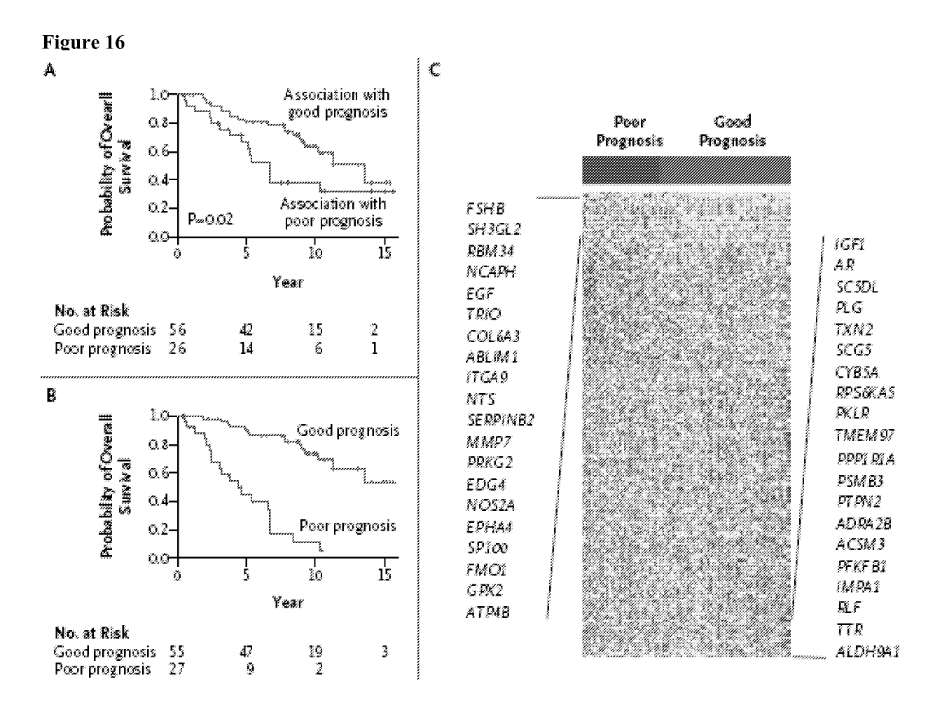

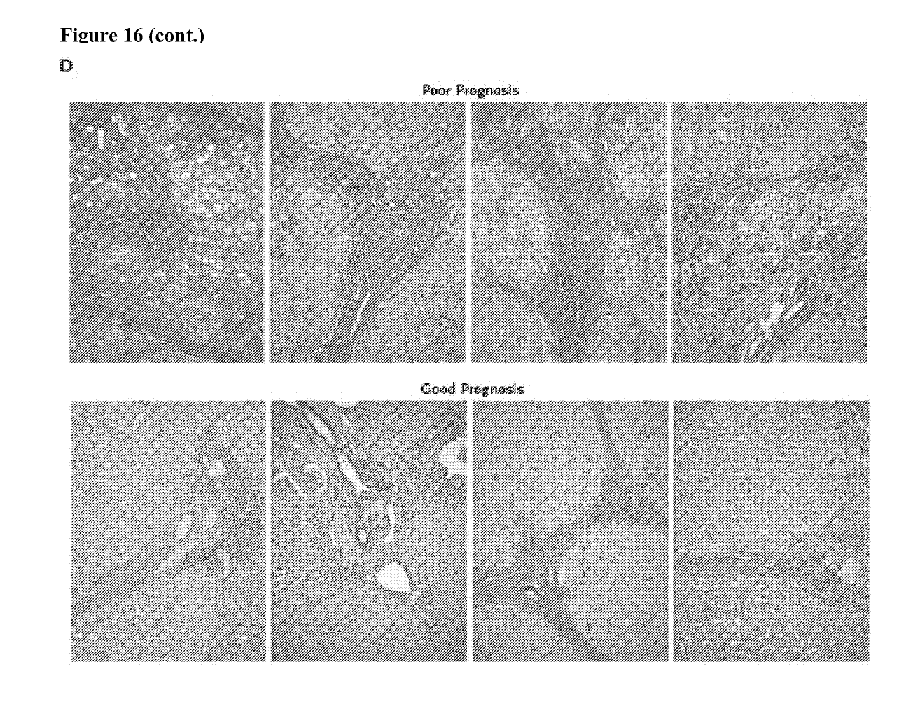

To test this hypothesis, the gene expression profiles of the liver tissue surrounding the resected tumor in the 106 formalin-fixed, paraffin-embedded blocks that constituted the training set were assessed. Eighty-two samples (77%) yielded high-quality gene-expression profiles. Using a standard leave-one-out cross-validation procedure, the liver signature was found to be significantly correlated with survival (P=0.02) (FIG. 2A). The aggregate survival-correlated signature contained 186 genes (FIGS. 16B and 16C and Table 2) and was tested in the validation set. Using GSEA, which shows whether a defined set of genes has a significant association with a phenotype of interest, the good-prognosis signature was found to contain genes associated with normal liver function (Tables 2 and 3), including the plasma proteins C4, C5, C8, C9, and F9 and several drug-metabolizing enzymes: the alcohol dehydrogenases ADH5 and ADH6, the aldo-keto-reductases AKR1A1 and AKR1D1, the aldehyde dehydrogenase ALDH9A1, the cytochrome P450 CYP2B6, and hepatic lipase (LIPC). These findings are consistent with the association between impaired liver function and a poor outcome (Llovet, J. M., et al. (2003) Lancet 362, 1907-1917). In addition, the poor-prognosis signature contained gene sets associated with inflammation, including those related to interferon signaling, activation of nuclear factor-κB, and signaling by tumor necrosis factor α. Histologic features of liver inflammation were not found to be associated with the outcome (FIG. 16D, Table 4, and FIG. 5). Of particular interest, GSEA showed that the downstream targets of interleukin-6 were strongly associated with the poor-prognosis signature, which is consistent with the finding that disruption of interleukin-6 signaling protects mice from chemically induced hepatocellular carcinoma (Naugler, W. E., et al. (2007) Science 317, 121-124).

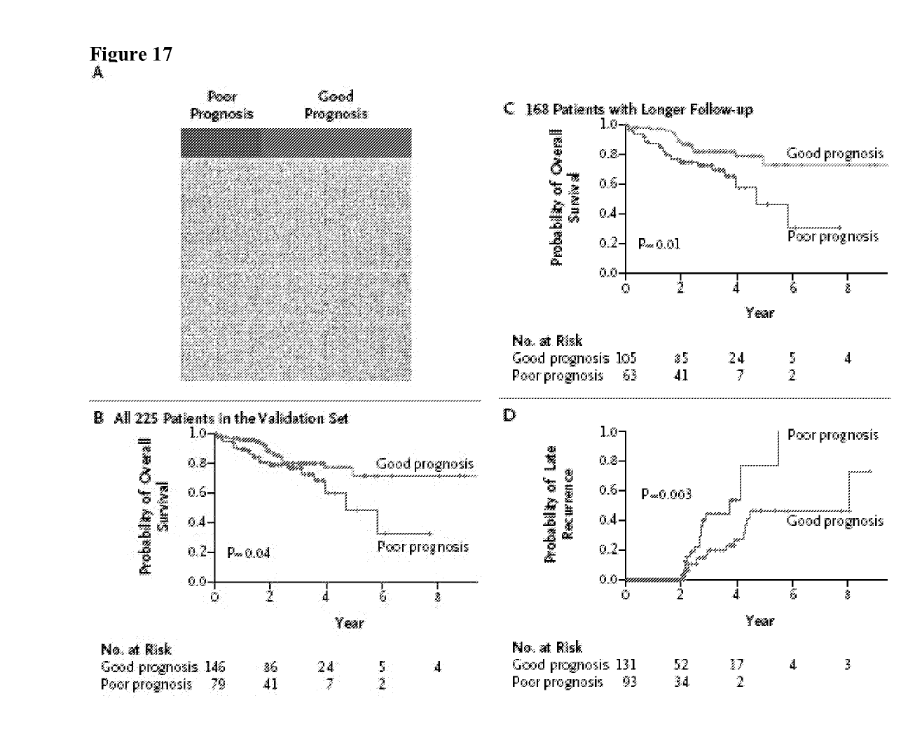

The 186-gene survival signature was next tested in an independent set of tissue samples from eligible patients at three treatment centers in the United States and Europe. Of the 234 samples in this validation set, 225 (96%) yielded gene-expression profiles of high quality. The survival signature (FIG. 17A) was associated with significant differences in survival among patients (P=0.04) (FIG. 17B), despite the modest duration of follow-up (median, 2.3 years). The separation of the survival curves was even more pronounced when the analysis was limited to the 168 patients with a longer duration of follow up (median, 2.8 years; P=0.01) (FIG. 17C). These results support the validity of the survival signature and highlight the potential role of nontumoral liver tissue in predicting the outcome for patients with early hepatocellular carcinoma.

### Example 5

### Recurrence-Associated Signature

A similar analysis using tumor recurrence as the clinical end point was performed.

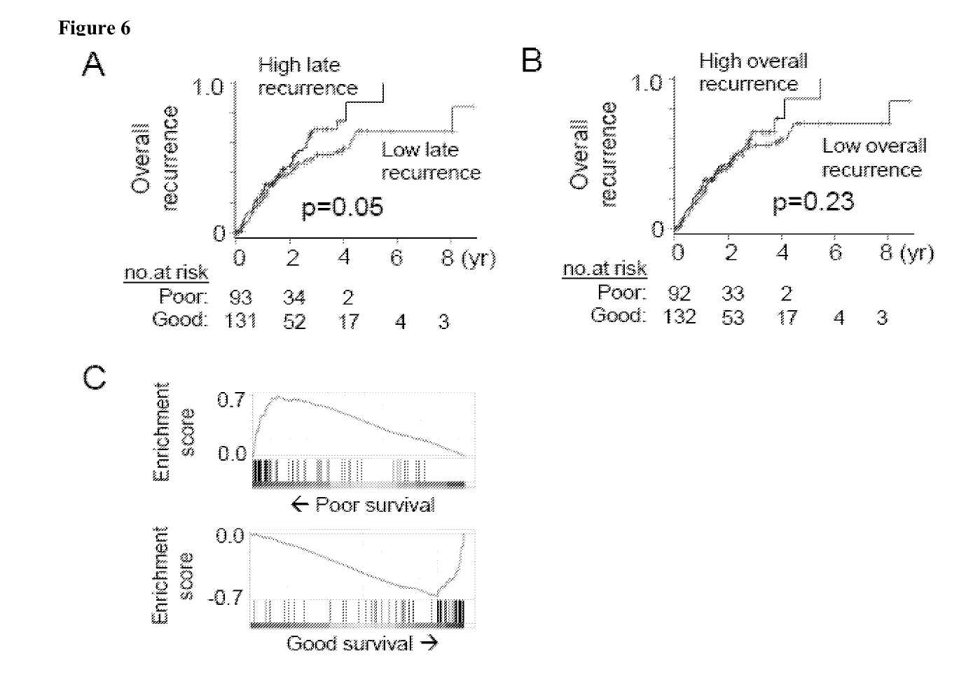

A 132-gene late-recurrence signature (FIG. 13) defined in the training set was tested in the validation set. Whereas the recurrence signature did not show an association with recurrence within the first 2 years after surgery (a finding that was consistent with its development in association with late recurrence) (FIGS. 6A and 6B), it was significantly associated with late recurrence (P=0.003) (FIG. 17D). Not surprisingly, a nonparametric enrichment test indicated that the survival and late-recurrence signatures were closely associated (P<0.001) (FIG. 6C Appendix).

### Example 6

### Multivariate Analysis

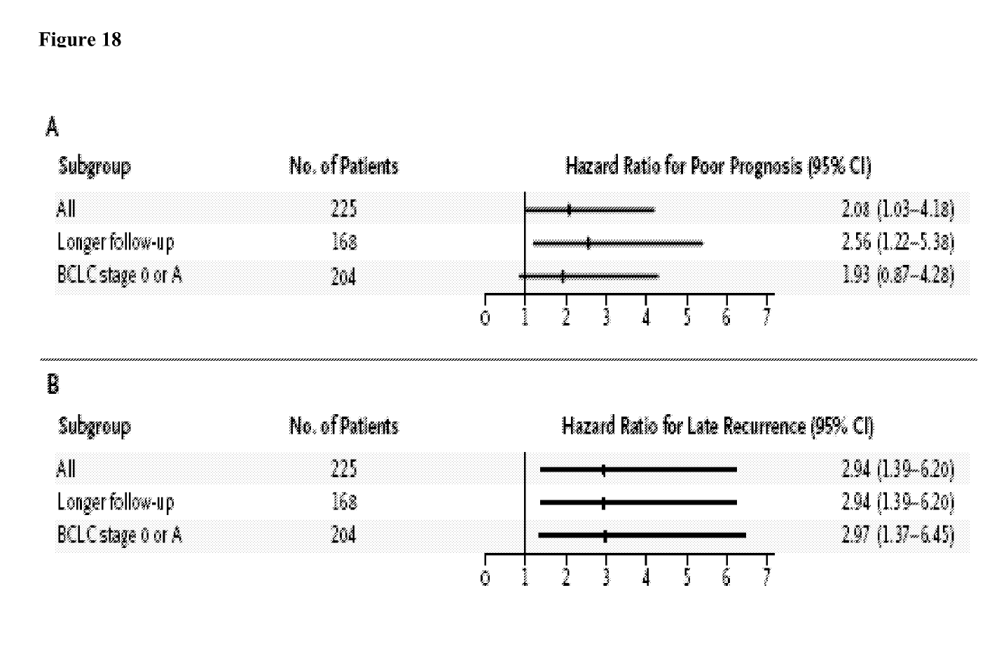

The signature in the context of the factors that are generally accepted as indicating a poor prognosis for patients with hepatocellular carcinoma (tumor multinodularity, the presence of microvascular invasion, and a high serum alpha-fetoprotein level) (Llovet, J. M., et al. (2003) Lancet 362, 1907-1917; Llovet, J. M., et al. (2005) Semin. Liver Dis. 2, 181-200) in the validation set was examined next. These factors were associated with early recurrence (<2 years after treatment) (Table 5). In contrast, multivariate analysis showed that the late-recurrence signature was the only independent prognostic variable for late recurrence (Table 12). Prespecified subgroup analyses showed that this association remained significant in both the subgroup of 168 patients with a longer period of follow-up and the subgroup of 204 patients with early-stage hepatocellular carcinomas (BCLC stage 0 or A) (FIG. 18, and Table 6). Similarly, the survival signature was independently associated with survival in multivariate analysis (Table 12), and this association persisted in the subgroup of patients with longer follow-up (Table 12).

These results indicate that clinical and histopathological factors are associated with early recurrence of hepatocellular carcinoma and that late recurrence is associated with the gene-expression signature of nontumoral liver tissue adjacent to the primary tumor. The latter finding is consistent with the notion that late recurrences are not actually recurrences but rather new primary tumors. In support of this view, highly discordant patterns of gains and losses in gene-copy number (including in regions exhibiting loss of heterozygosity) between the primary and recurrent hepatocellular carcinoma tumors were detected, but such patterns were not detected in endometrial, ovarian, renal, or lymphoma tumors (Table 7 and FIG. 7). These results strongly suggest that the primary and recurrent hepatocellular carcinoma tumors arise from distinct clones.

The full potential of gene-expression profiling of cancer has been hindered in part by technical limitations—in particular, the requirement of frozen material for analysis. Although frozen tissues are increasingly being banked at tertiary care centers, the duration of clinical follow-up of these collections is usually short, and the vast majority of tumor-biopsy specimens and resections are performed outside of major research hospitals. There is therefore a need for methods that allow for the genomewide expression profiling of formalin-fixed tissue samples, which are routinely collected in the clinical setting. Such approaches have been described (Coudry, R. A., et al. (2007) J. Mol. Diagn. 9, 70-79), but their extensive validation has yet to be reported.

A DASL-based method capable of profiling approximately 6,000 human transcripts is described herein, and the method was tested on 2,000 formalin-fixed, paraffin-embedded blocks collected as long as 24 years ago. Through the assay of 6,000 genes across the genome that show maximal variation in expression, this approach is expected to capture the bulk of transcriptional differences in any collection of samples. However, recent increases in array density support the analysis of all human genes on a single array (whole-genome DASL assay, Illumina).

The DASL-based discovery method described herein is distinguishable from candidate-gene profiling methods based on the reverse transcriptase (RT)-PCR assay, such as those used in the commercially available OncotypeDx™ test for determining the prognosis in patients with breast cancer (Habel, L. A., et al. (2006) Breast Cancer Res. 8, R25). Whereas standard RT-PCR methods can measure a small number of transcripts in formalin-fixed, paraffin-embedded samples, genomewide discovery studies are not feasible with the use of RT-PCR-based methods. In addition, it may be that the use of formalin-fixed, paraffin-embedded tissue specimens will aid the transition from exploratory research to clinical implementation.

The DASL profiling method was applied to an increasingly important challenge in the care of patients with hepatocellular carcinoma. Tumors are often small at the time of diagnosis (owing to increased surveillance and advanced imaging in patients at risk), and existing prognostic factors are less informative for patients with small tumors than for those with larger tumors. A significant association between the expression profiles of the tumors themselves and the outcome for patients with surgically resected early hepatocellular carcinoma was not observed. In contrast, others have described tumor-derived prognostic signatures for hepatocellular carcinoma (Lee, J. S., et al. (2004) Hepatology 40, 667-676; Ye, Q. H., et al. (2003) Nat. Med. 2003; 9: 416-23). The populations of patients in those studies, however, tended to have more advanced disease. The training set used herein primarily exhibited a pattern of late recurrence that is typical of small tumors (Llovet, J. M., et al. (2003) Lancet 362, 1907-1917; Llovet, J. M., et al. (2005) Semin. Liver Dis. 2, 181-200). Accordingly, it is likely that early recurrence (reflecting locally invasive and incompletely resected tumor) is associated with molecular features of the primary tumor, but such features are not associated with late recurrences, which seem to result from new primary tumors arising in a damaged organ (the “field effect”) rather than the proliferation of residual tumor cells derived from the original tumor.

Also supporting the concept that late recurrence of hepatocellular carcinoma represents new primary tumors in patients at risk, little correlation was found between the molecular characteristics of tumors resected at initial diagnosis and those from the same patients at the time of recurrence. In particular, the results of clonality analysis indicated that the late recurrences of hepatocellular carcinoma tended to derive from a different clone than the preceding primary tumors. In addition, the obvious measures of liver damage (e.g., the extent of cirrhosis and the Child-Pugh stage) (Pugh, R. N., et al. (1973) Br. J. Surg. 60, 646-649) were not associated with survival in the present study, given that the analysis was restricted to patients with preserved liver function.

These findings indicate a field effect, in which environmental exposure (e.g., viral infection) leads to an increased potential for future malignant transformation. This has in general been overlooked by genomic approaches to studying cancer that have focused only on tumor cells. These results suggest that a gene-expression signature can serve as a sensitive “readout” of the biologic state of the liver in at-risk patients. It is likely that the survival signature reflects the extent of liver damage and the presence or absence of a proinflammatory milieu, which is mediated in part by gene products involved in an inflammatory response. This test is envisioned to identify the patients at highest risk for recurrence of hepatocellular carcinoma and to target intensive clinical follow-up or chemopreventive strategies in such patients.

### Example 7

### Materials and Methods for Examples 8-15

### A. Patients and Samples

Patients diagnosed as having compensated liver cirrhosis and no history of gastrointestinal bleeding, jaundice, ascites or hepatocellular carcinoma were enrolled between 1985 and 1998, and prospectively followed for the development of hepatocellular carcinoma or progression of cirrhosis defined by either an episode of hepatic decompensation, or overall death (Colombo, M., et al. (1991) N. Engl. J. Med. 325, 675-680; Sangiovanni, A., et al. (2004) Gastroenterology 126, 1005-1014; Vigano, M., et al. (2005) Hepatology 42, 432A). Fine needle biopsy specimens of the liver were obtained within 2 years of enrollment, and archived as formalin-fixed, paraffin-embedded tissue blocks. Five tissue sections (10 micron thick) were obtained from each block, and subjected to RNA extraction as previously described (Hoshida. Y., et al. (2008) N. Engl. J. Med. 359, 1995-2004).

### B. Analysis of Gene Expression

Gene-expression profiling was performed using the whole-genome complementary DNA-mediated Annealing, Selection, extension, and Ligation (DASL) assay (Illumina) (Fan, J. B., et al. (2004) Genome Res. 14, 878-85; Bibikova, M., et al. (2004) Am. J. Pathol. 165, 1799-1807). Microarray data may be obtained on the world wide web from the Gene Expression Omnibus database of the NCBI website under accession number GSE15654.

### C. Statistical Analysis

The 186-gene survival signature genes were previously reported, and predictions were made using a nearest-template-based method as previously described (Hoshida. Y., et al. (2008) N. Engl. J. Med. 359, 1995-2004). The log-rank test and Cox regression modeling were used to evaluate association of the signature and clinical variables with overall survival, time to first episode of hepatic decompensation, and time to hepatocellular carcinoma development. Predefined subgroup analysis was performed on data from patients of Child-Pugh class A, and from patients with hepatitis C virus infection. Association with death unrelated to hepatocellular carcinoma was evaluated by treating cancer-related death as censored observations. Analyses were performed using either the GenePattern analytical toolkit (Reich, M., et al. (2006) Nat. Genet. 38, 500-501; available on the world wide web at the software database of the MIT Broad Center website) or the R statistical package (available on the world wide web at the R-Project website) as described further below.

### D. Gene-Expression Microarray

Using five 10-micron-thick formalin-fixed, paraffin-embedded (FFPE) liver biopsy tissue sections, total RNA was extracted using TRIzol LS reagent (Invitrogen) in a semi-automated 96-well plate format (CyBio) as previously described (Hoshida. Y., et al. (2008) N. Engl. J. Med. 359, 1995-2004). Whole-genome gene-expression profiling was performed using the whole-genome cDNA-mediated Annealing, Selection, extension and Ligation (DASL) assay (Illumina) (Fan, J. B., et al. (2004) Genome Res. 14, 878-85; Bibikova, M., et al. (2004) Am. J. Pathol. 165, 1799-1807). Signal intensities were extracted from the scanned images using BeadStudio ver.3 software (Illumina). Poor quality profiles were removed based on the proportion of gene probes with a “present” signal (% P-call) less than 25% as previously described (Hoshida, Y., et al. (2008) N. Engl. J. Med. 359, 1995-2004). Remaining profiles were normalized using the cubic spline algorithm (Workman, C., et al. (2002) Genome Biol. 3, research0048.1-research0048.16) implemented in the Illuminallormalizer module of the GenePattern software package available on the world wide web at the Broad Institute website. Only gene probes with a minimal 3-fold differential expression and absolute difference >500 units across the samples were included after applying floor and ceiling values of 100 and 40,000 units, respectively.

### E. Gene-Expression-Based Outcome Prediction

Gene-expression-based outcome prediction was performed using the nearest template prediction (NTP) method (Hoshida. Y., et al. (2008) N. Engl. J. Med. 359, 1995-2004; Workman, C., et al. (2002) Genome Biol. 3, research0048.1-research0048.16) as implemented in the NearestTemplatePrediction module of the GenePattern analysis toolkit available on the world wide web at the Broad Institute website. A prediction of poor outcome was made based on a false discovery rate (FDR) (Reiner, A., et al. (2003) Bioinformatics 19, 368-375)<0.05. Samples predicted as having “poor” outcome were compared to the rest of the samples.

### Example 8

### Fine Needle Biopsy Expression Profiling

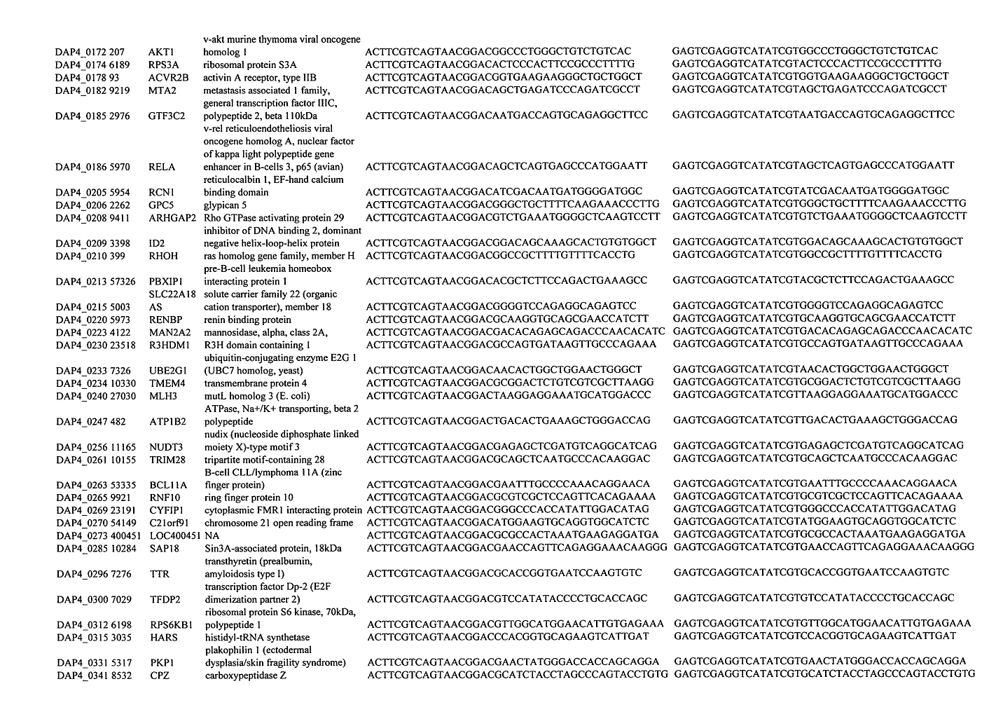

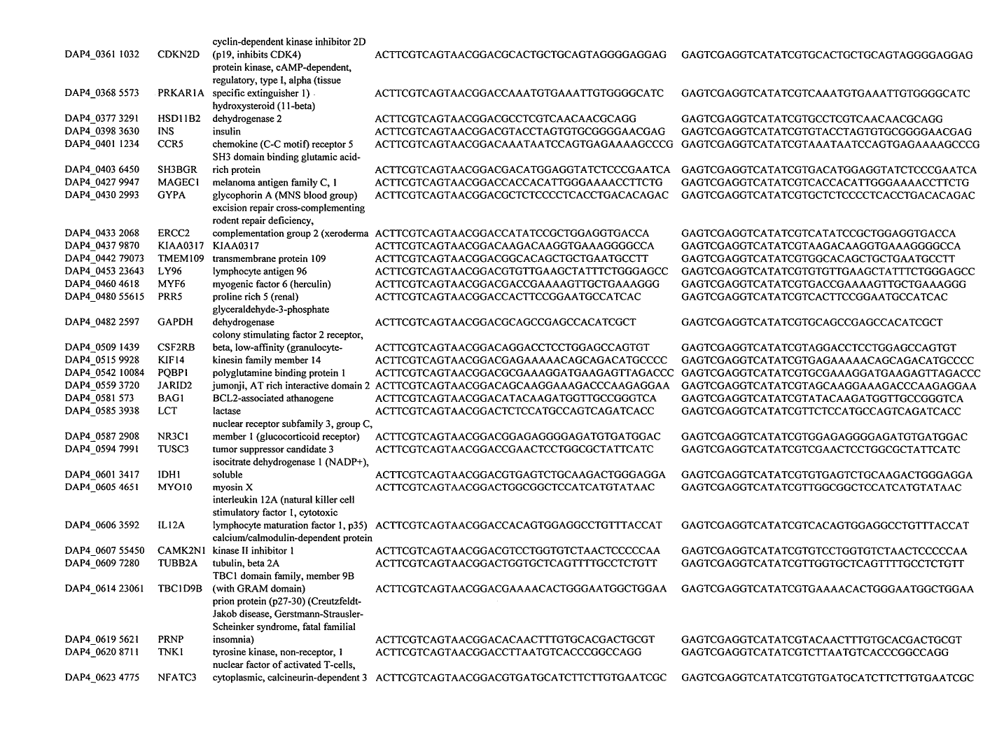

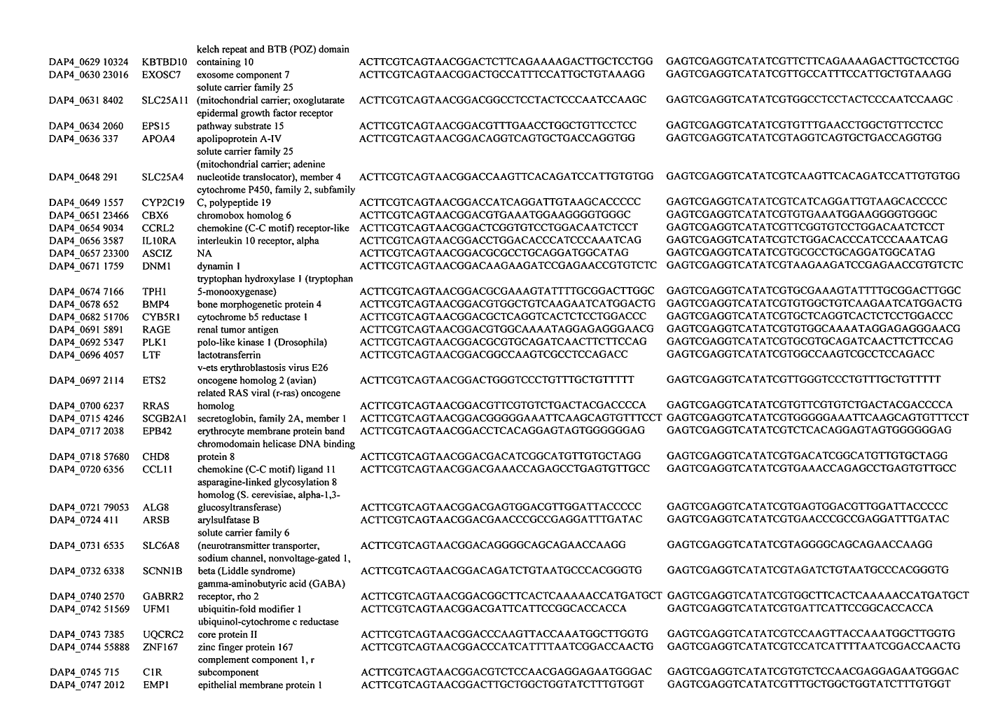

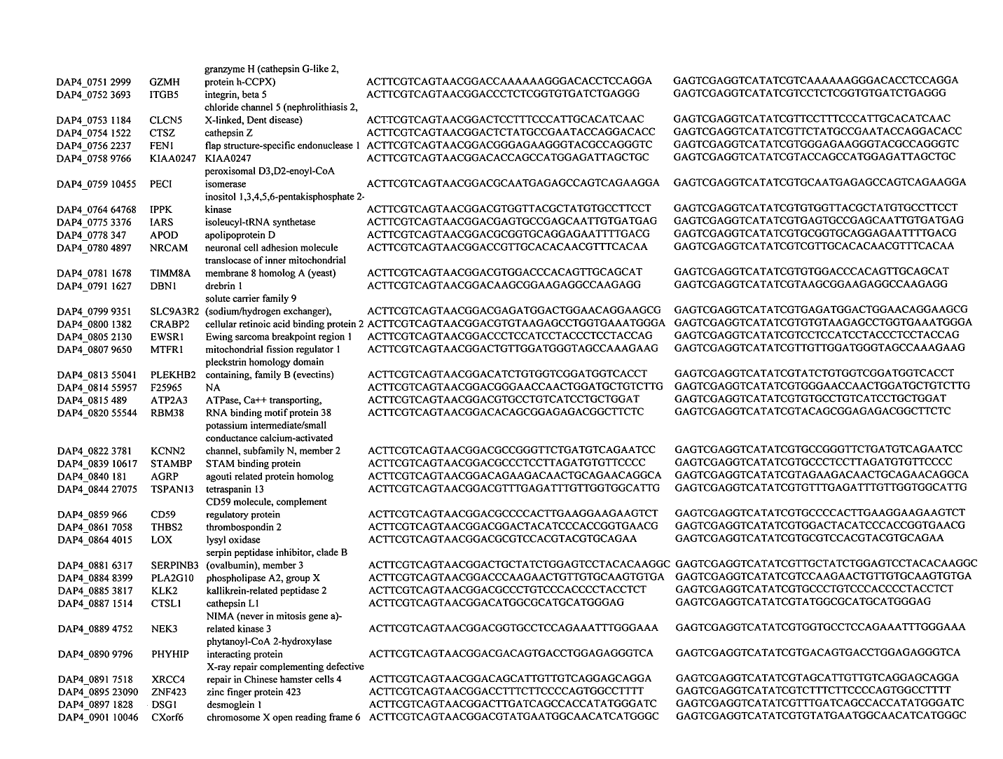

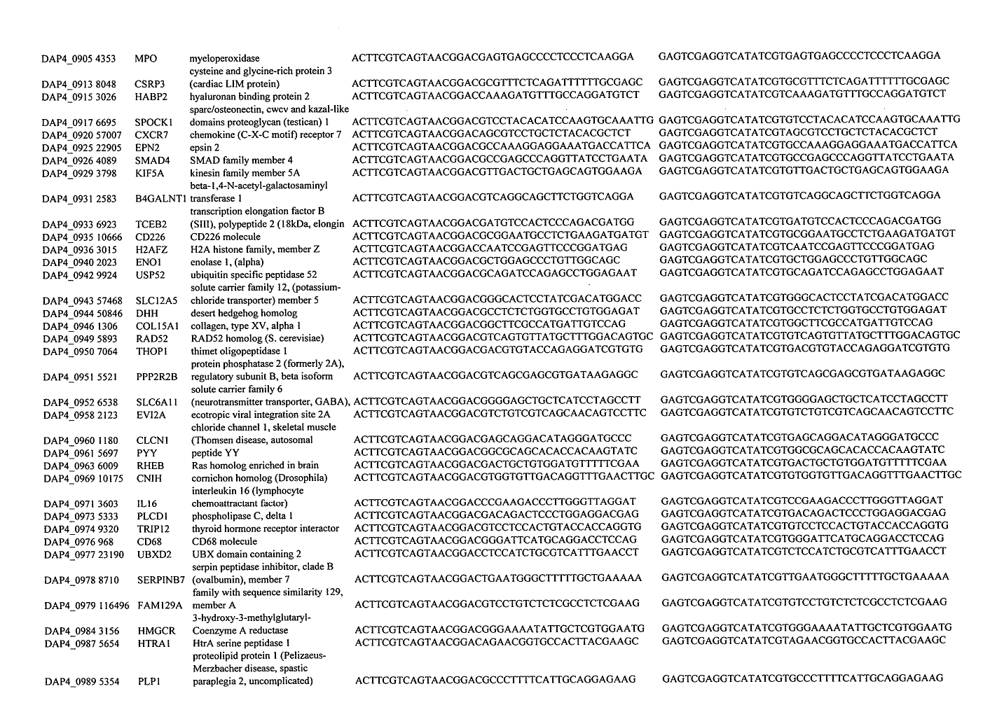

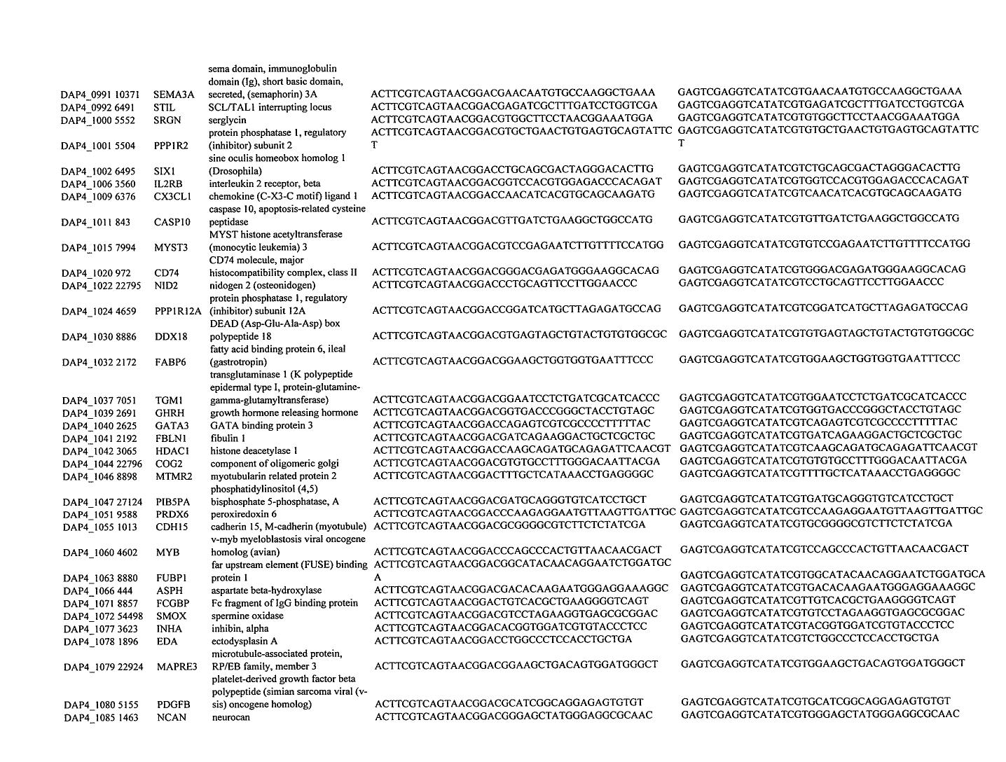

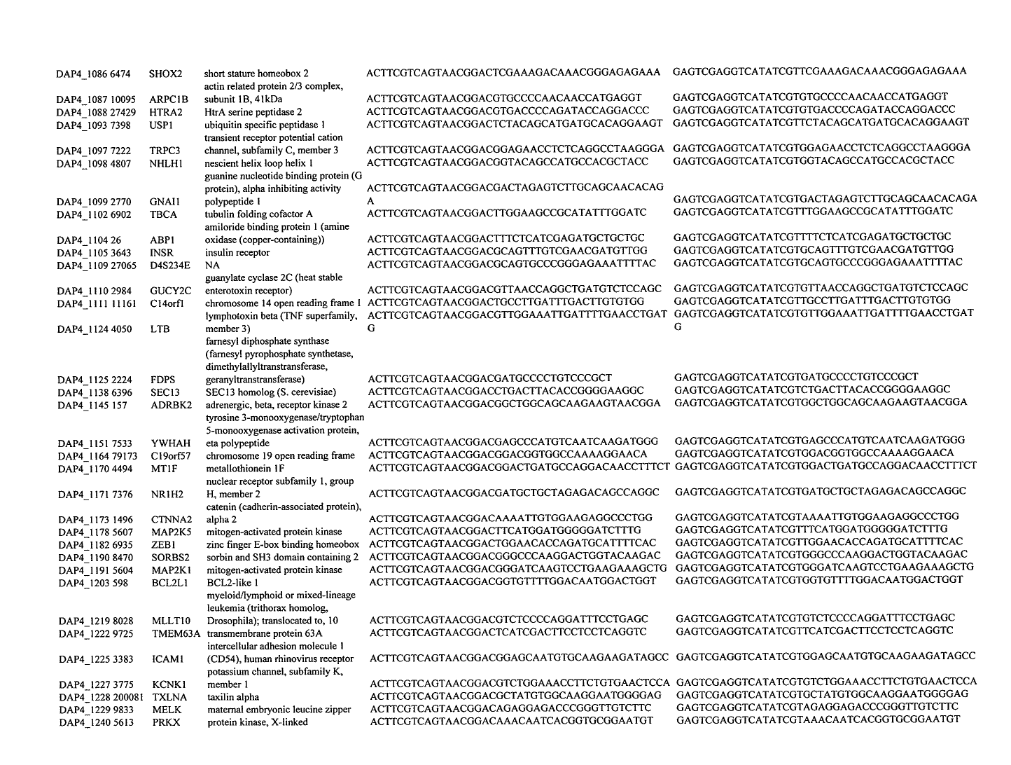

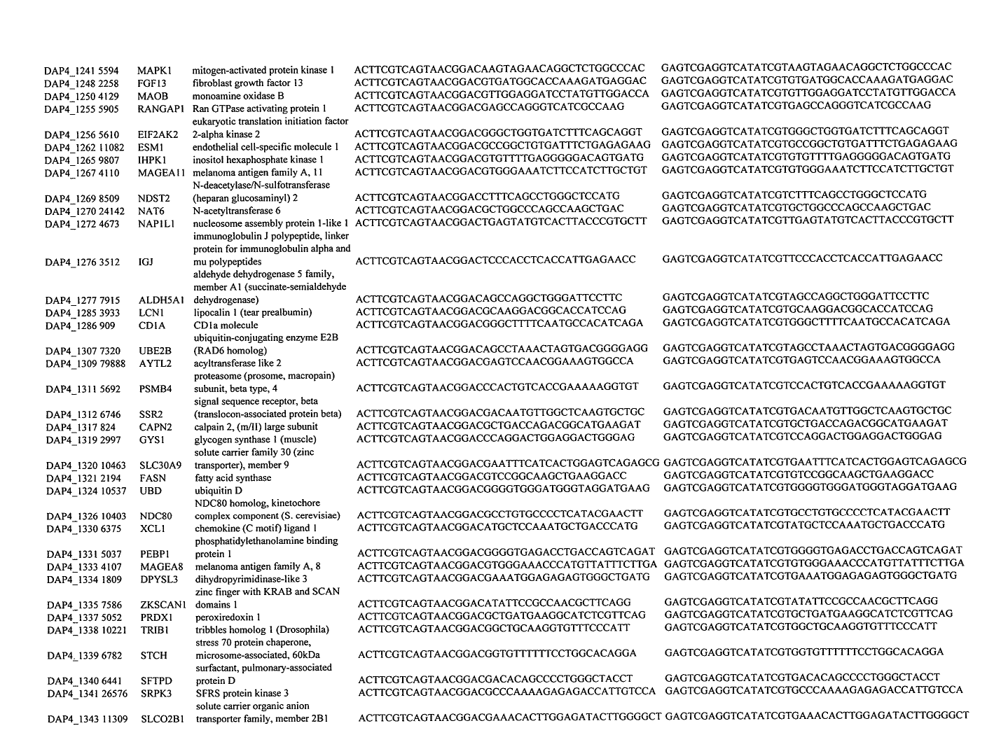

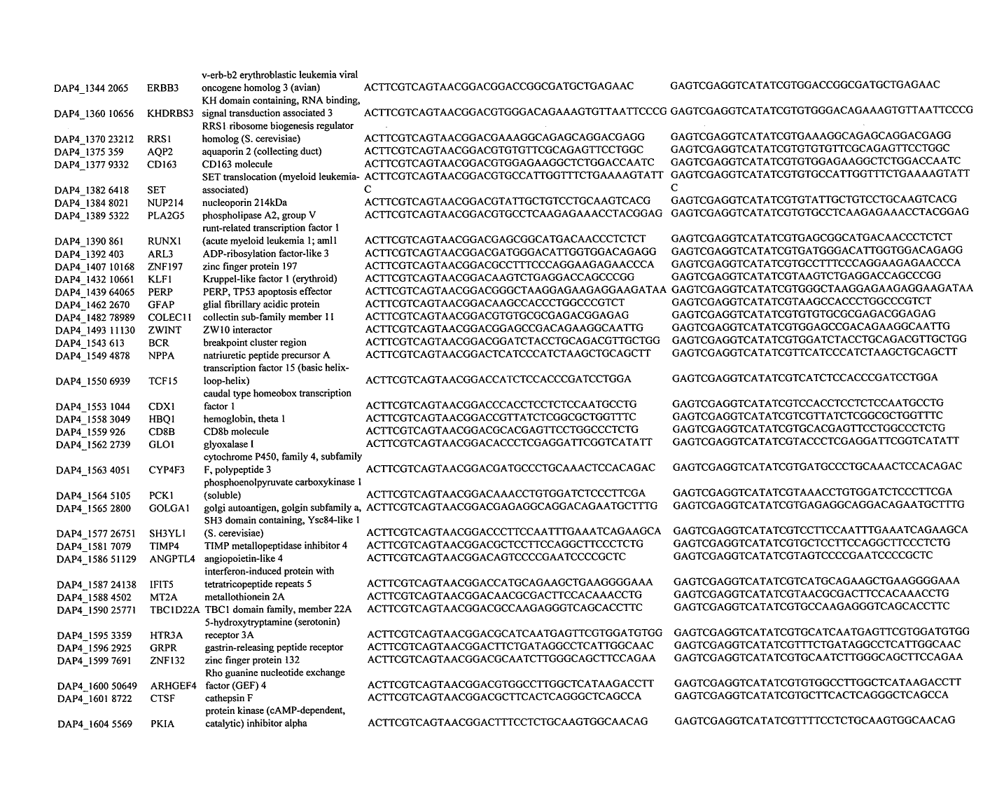

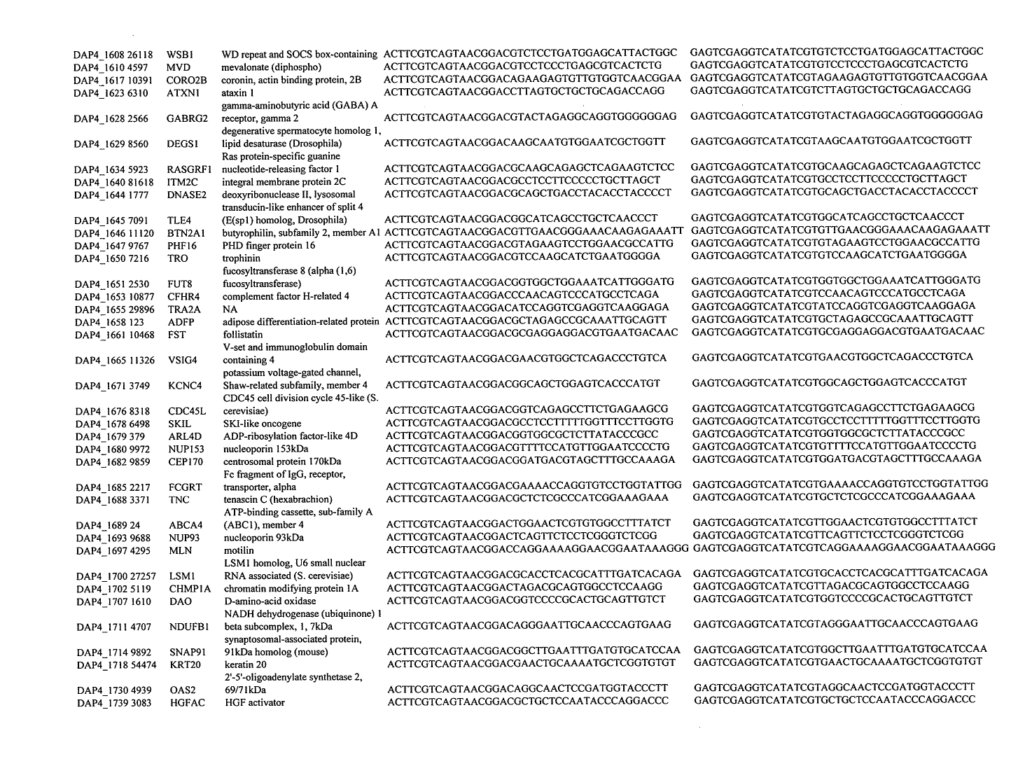

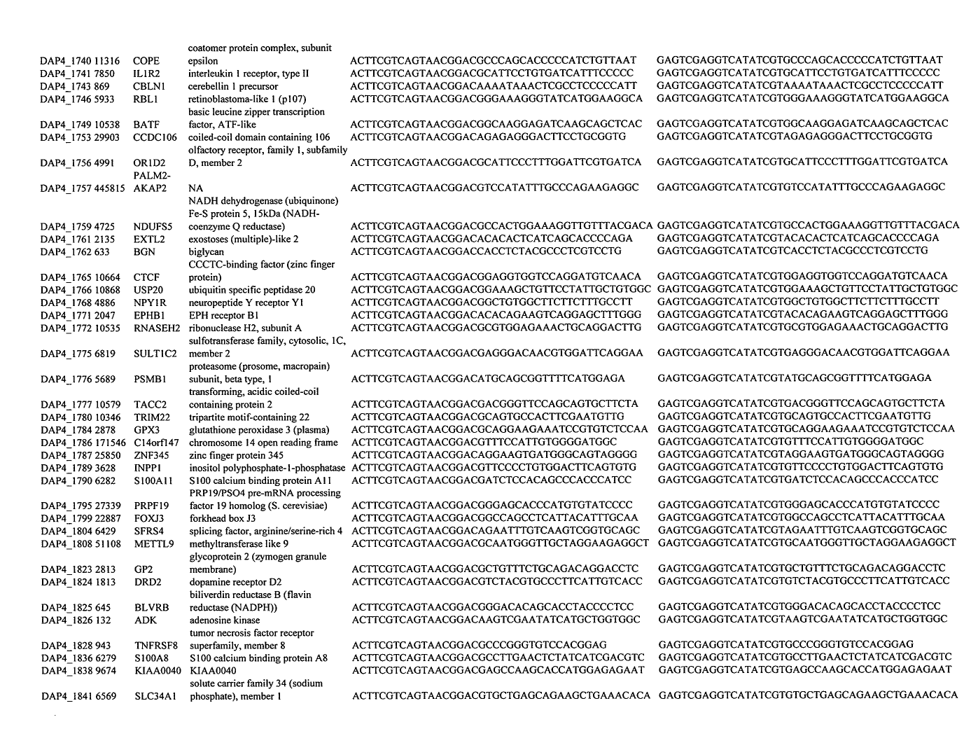

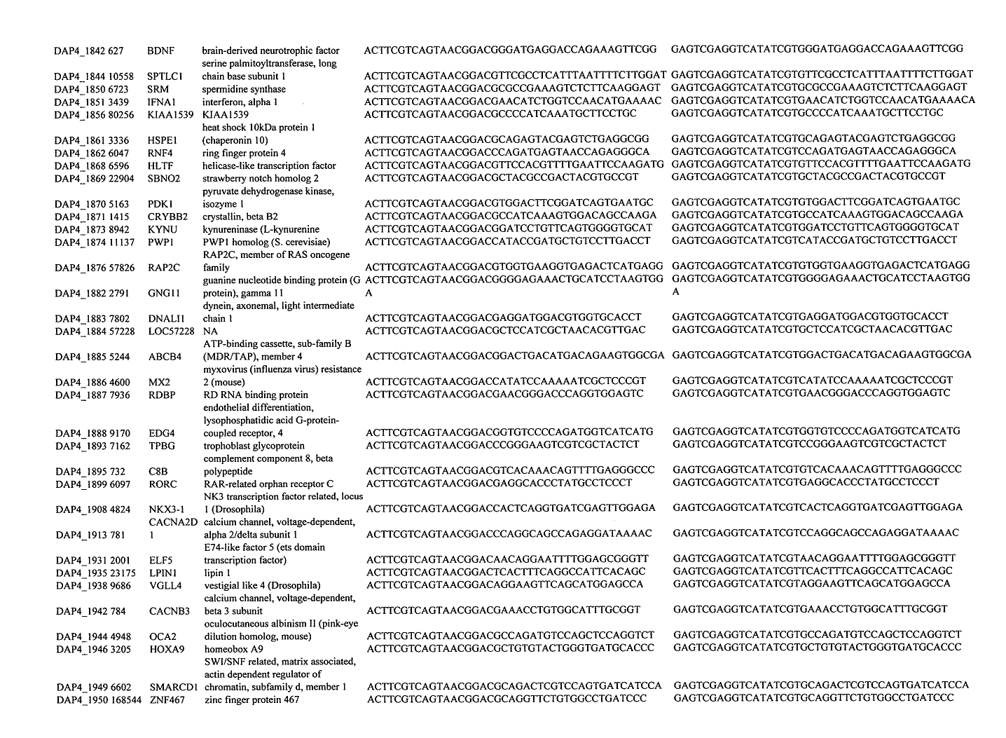

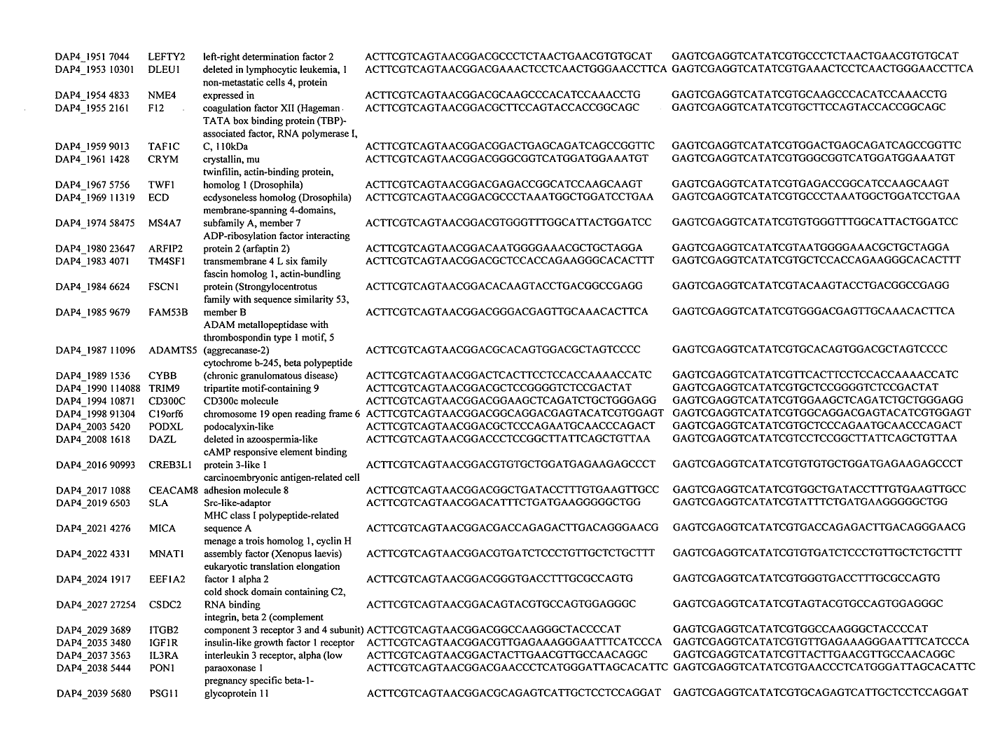

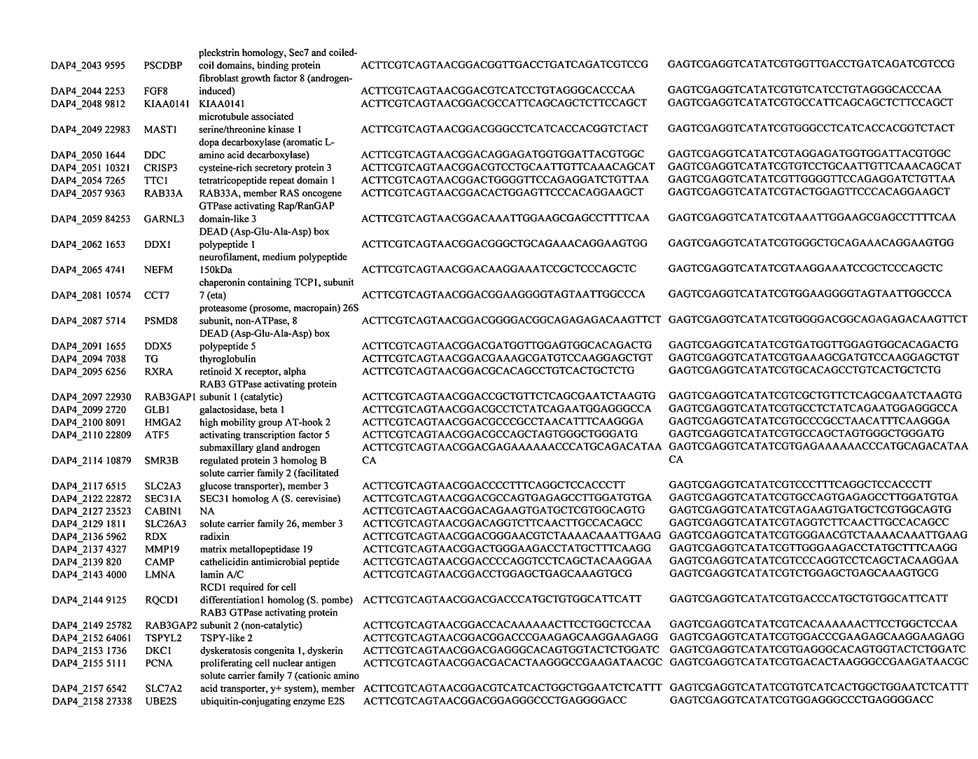

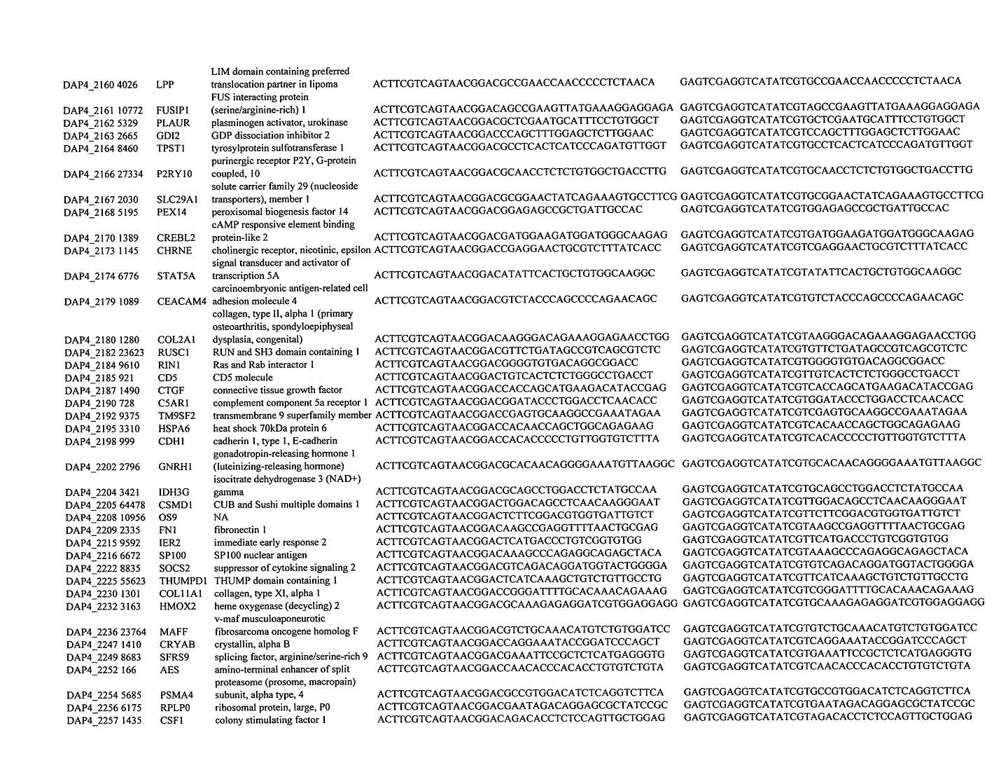

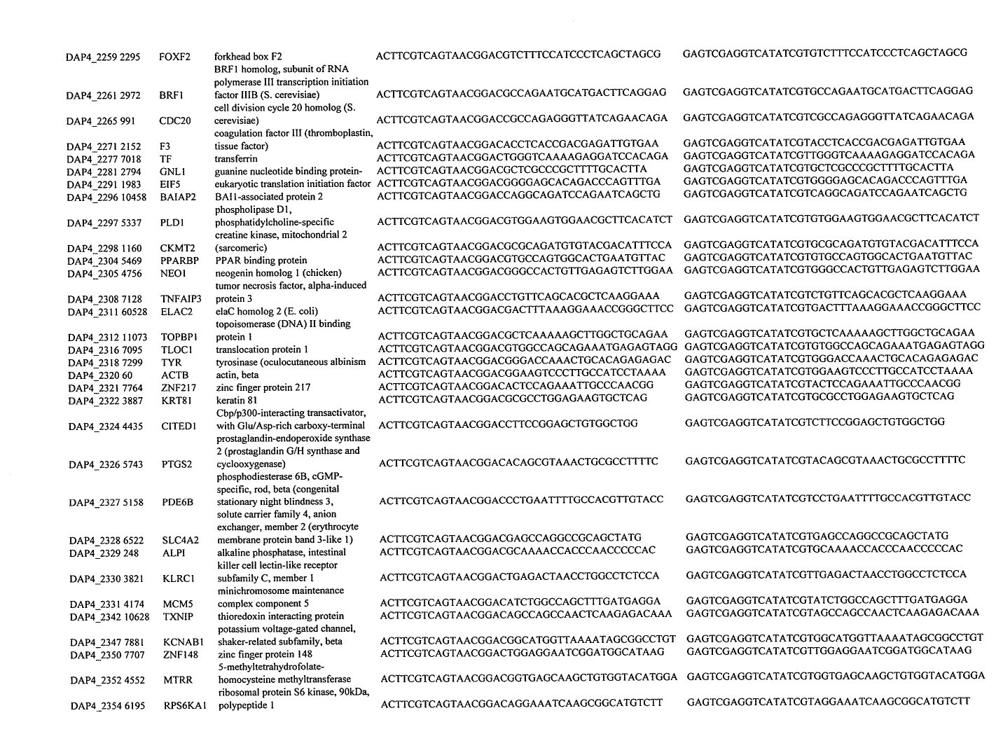

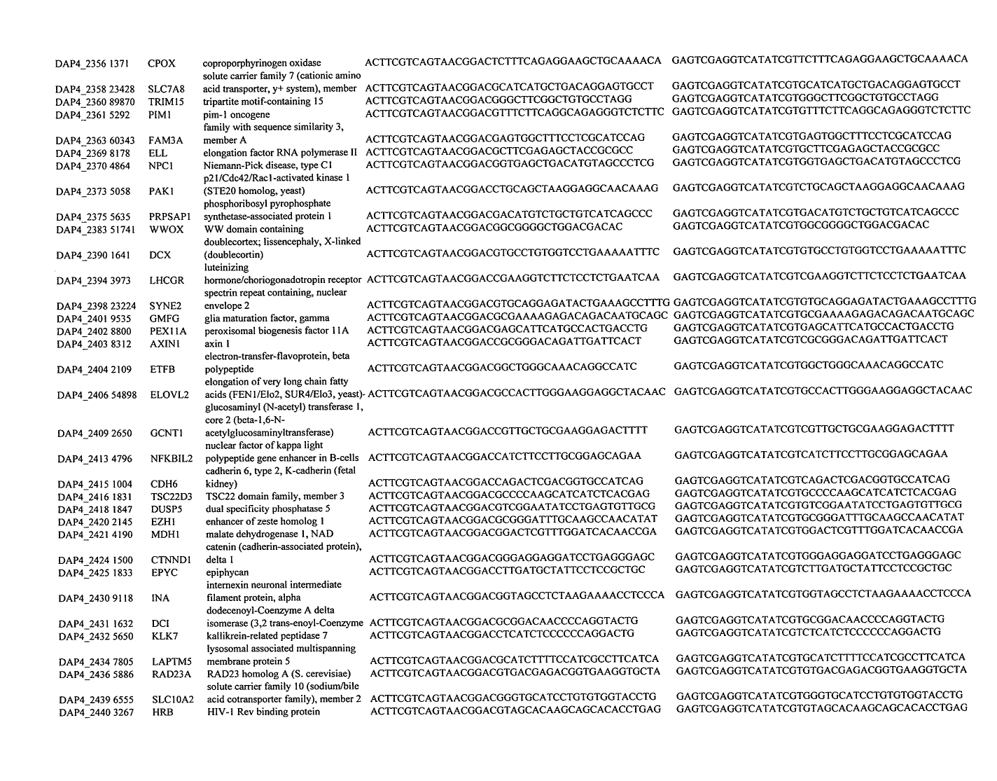

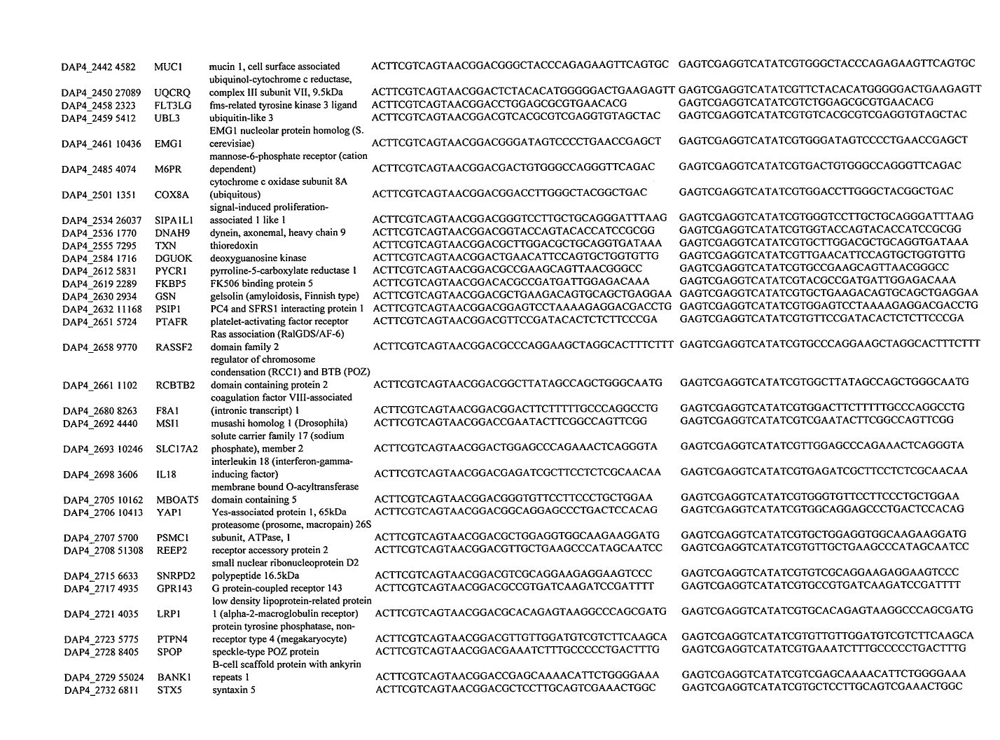

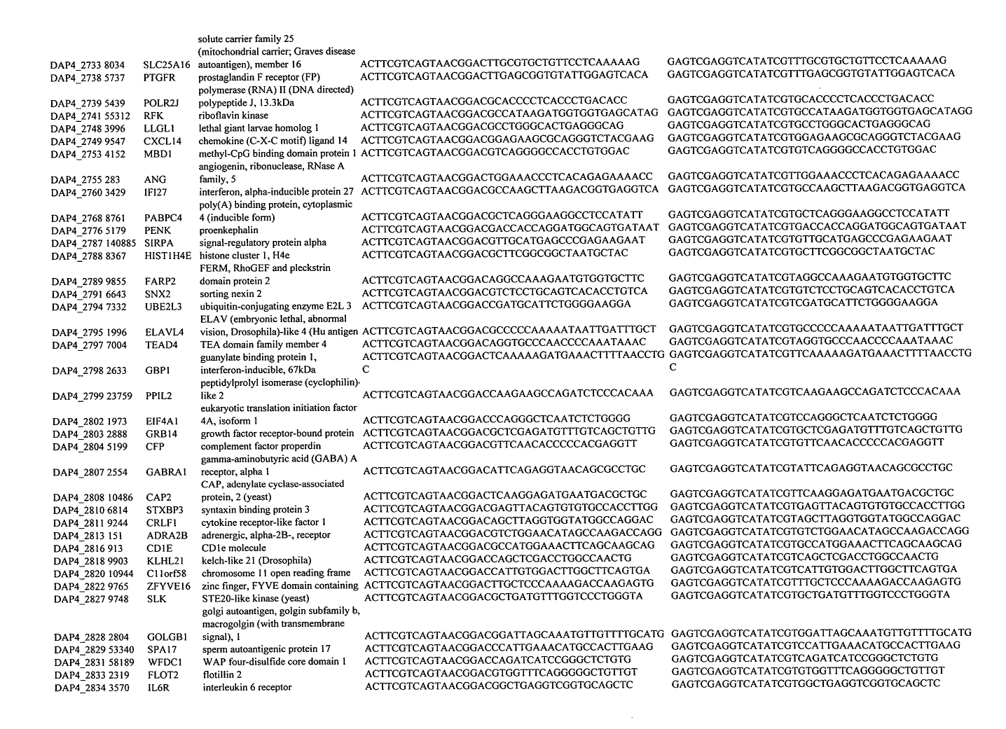

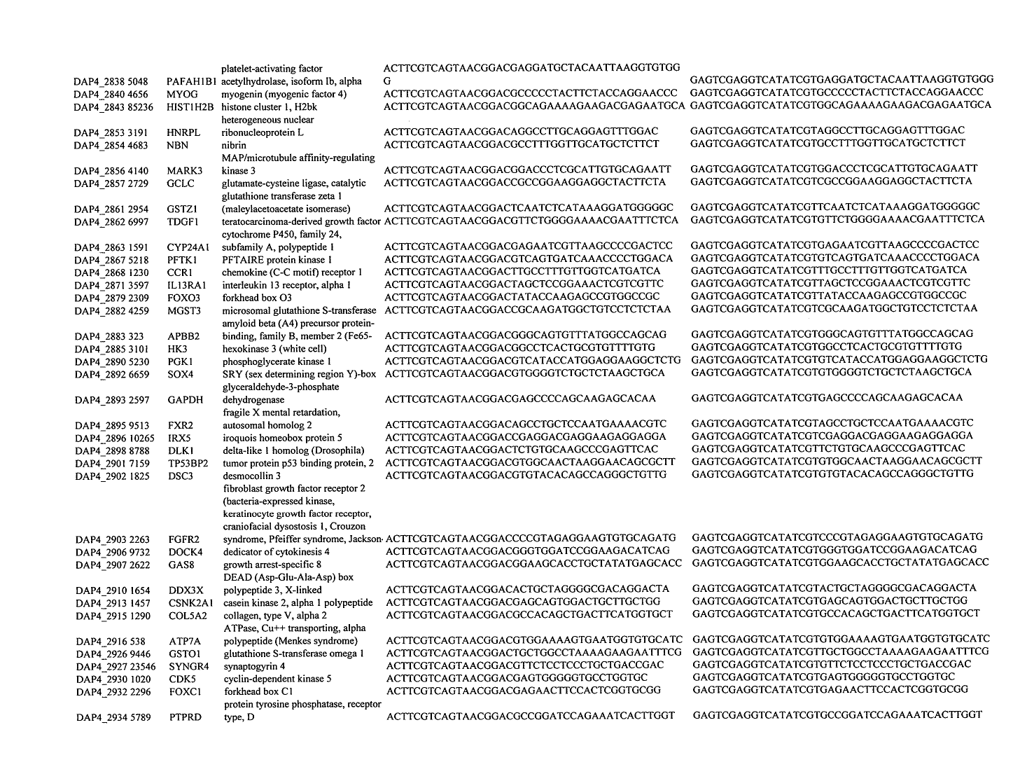

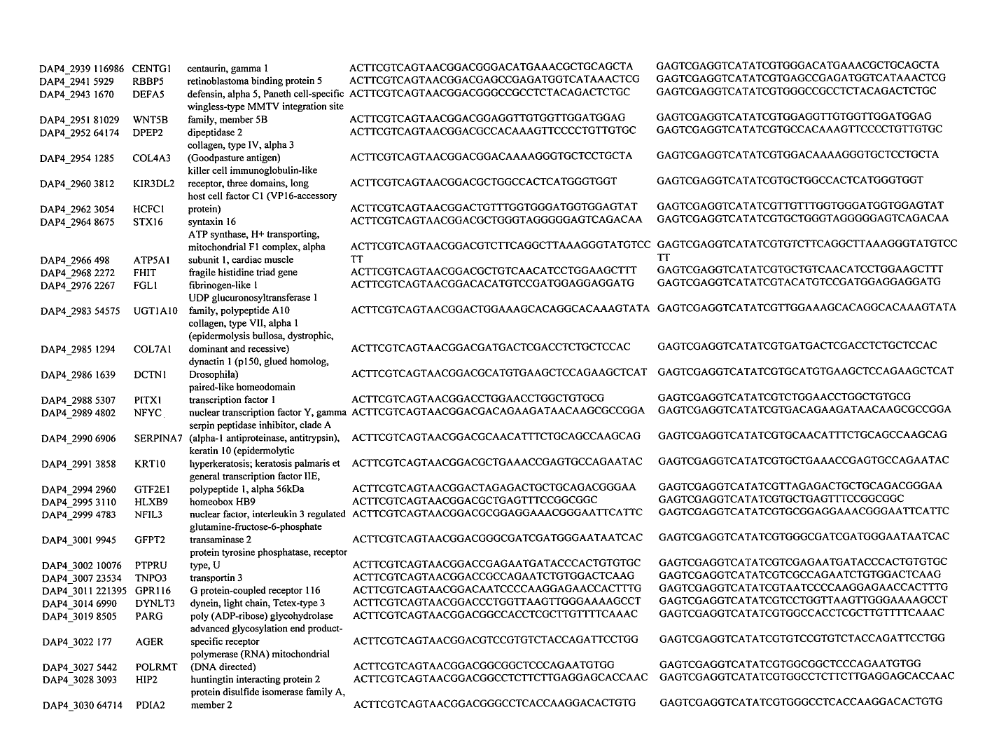

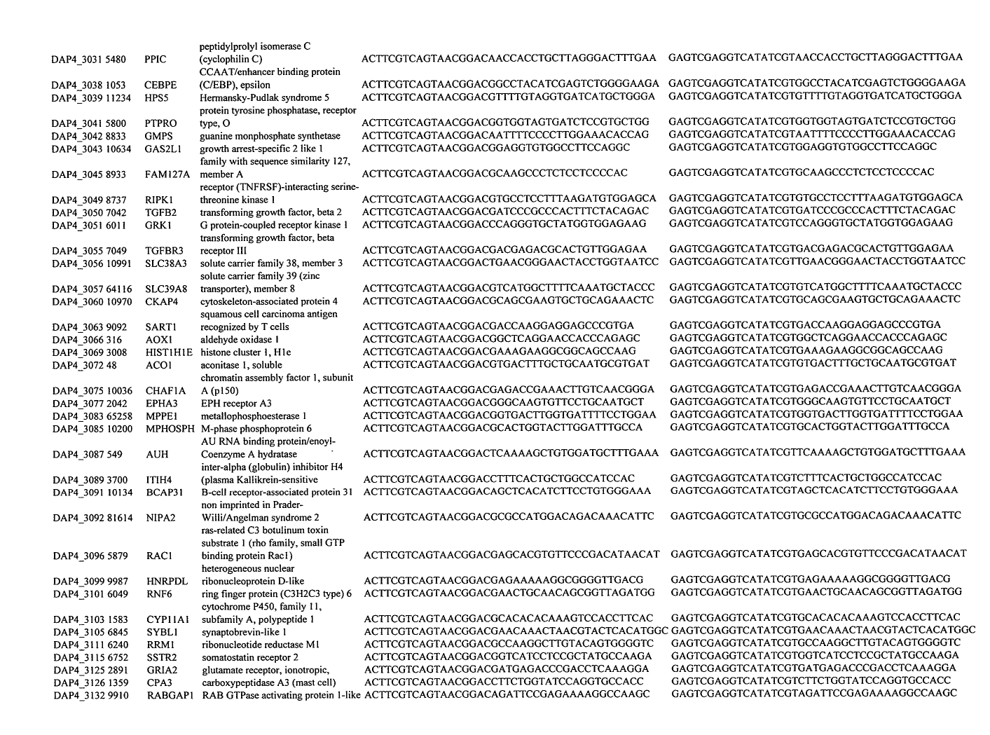

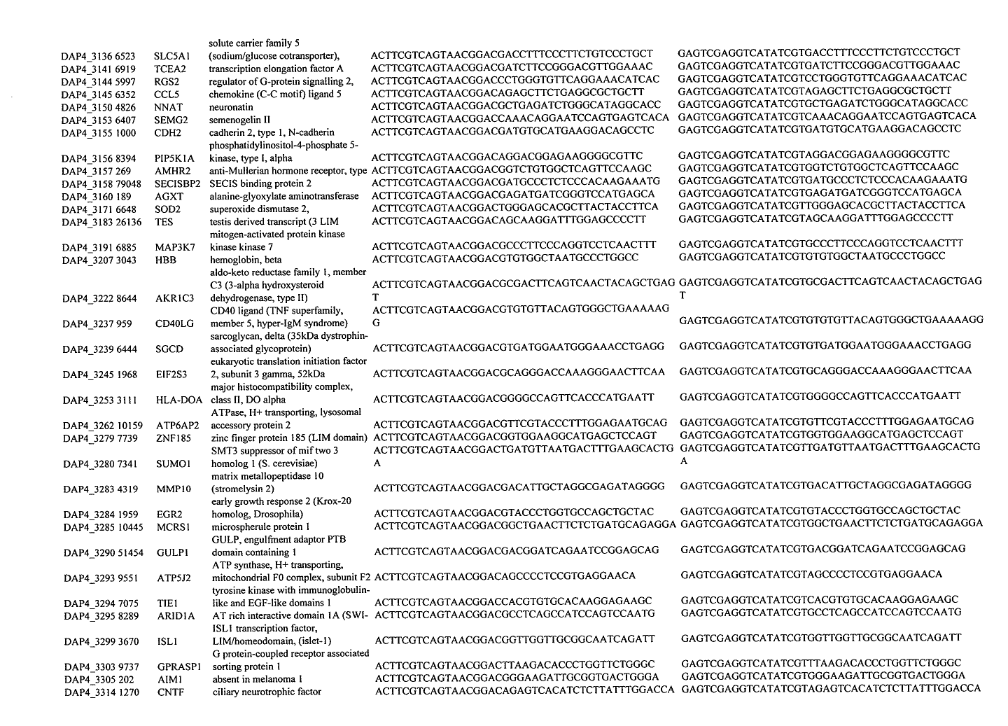

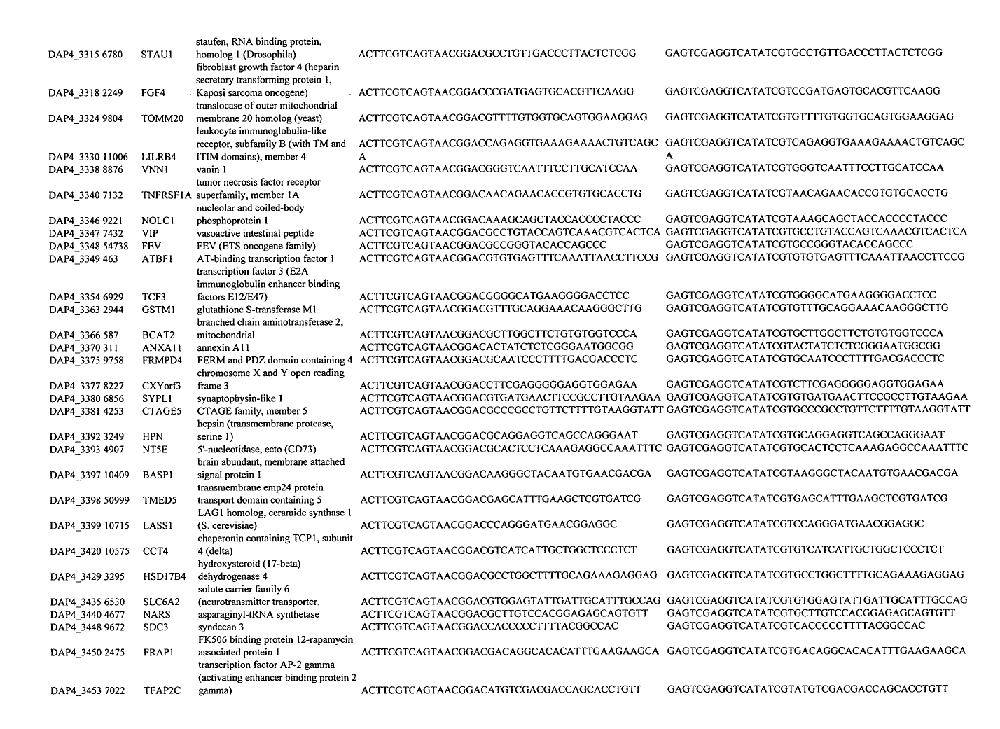

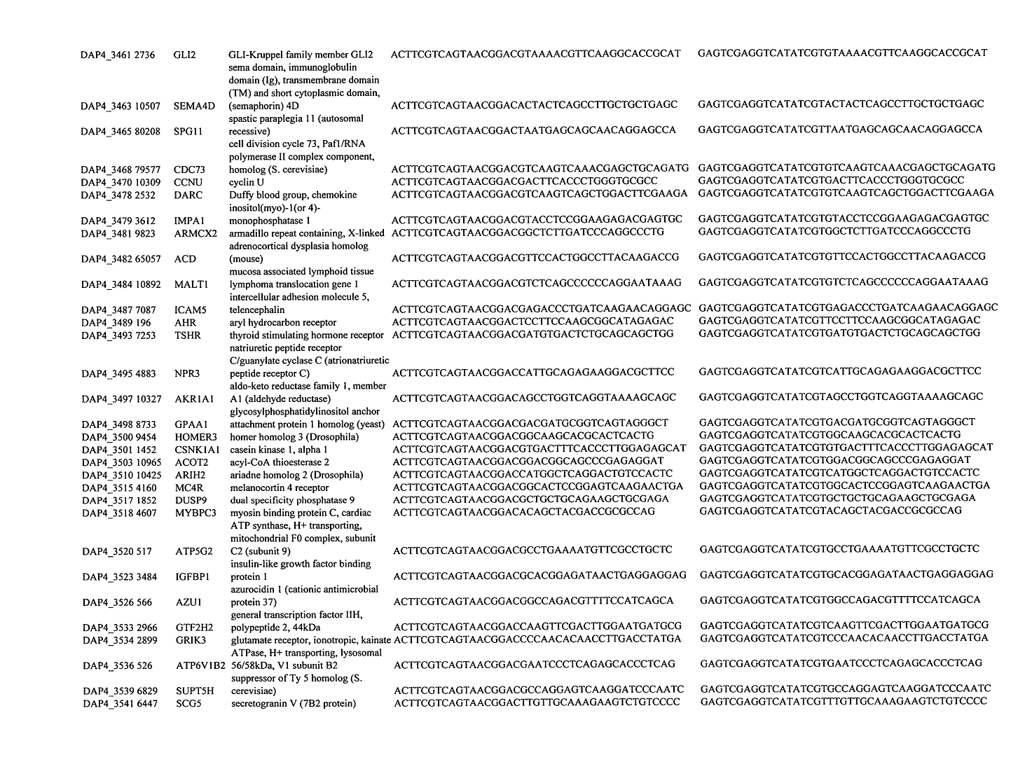

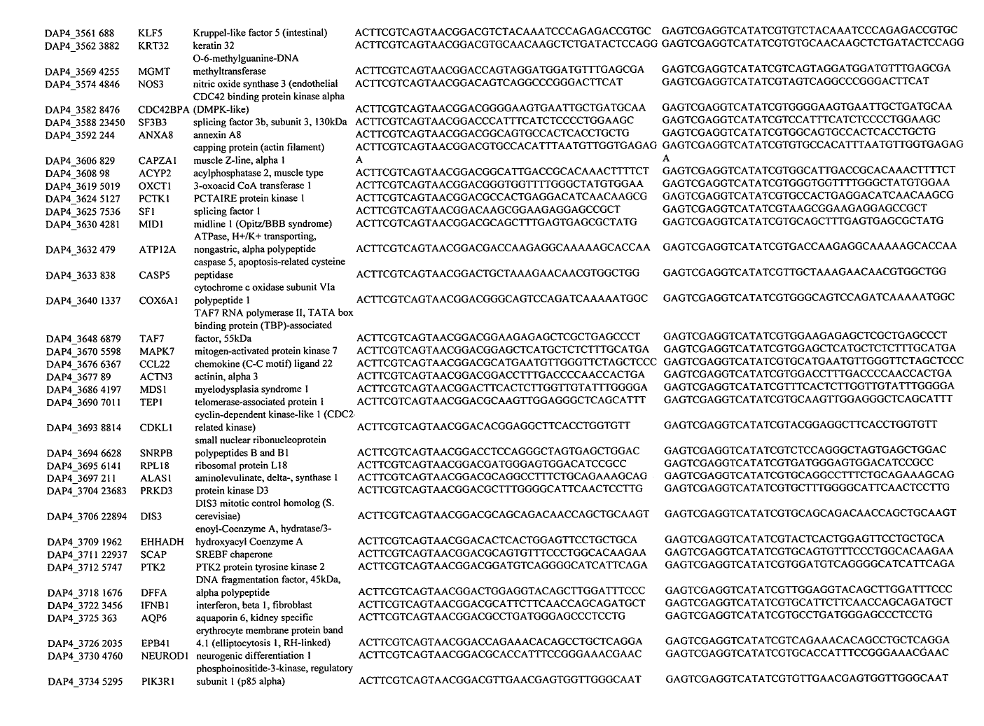

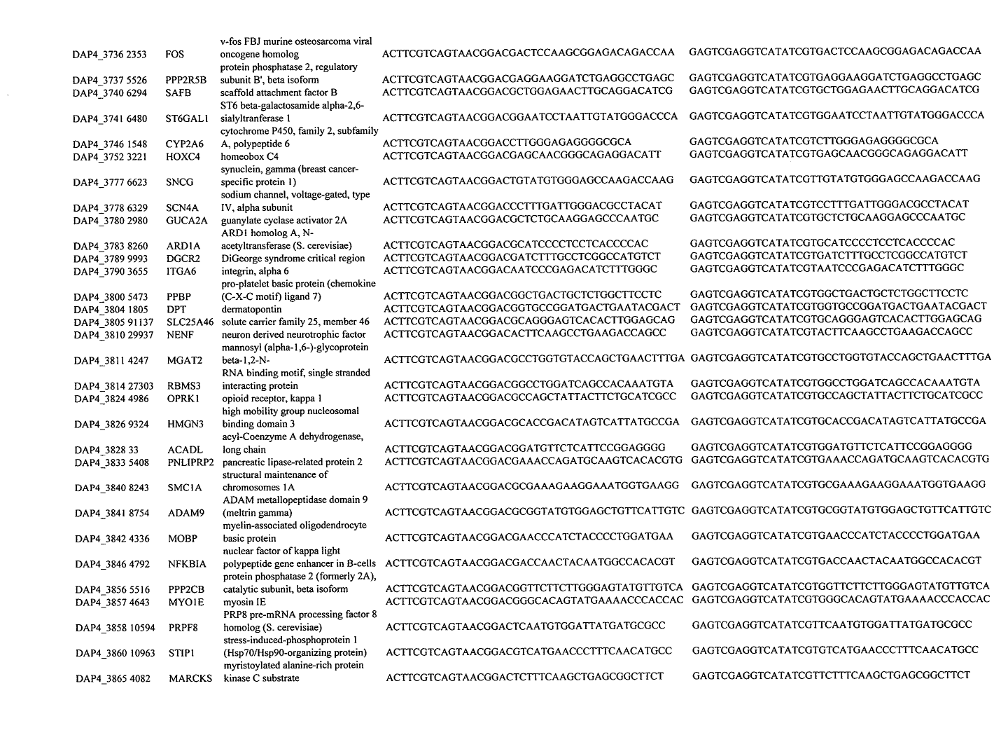

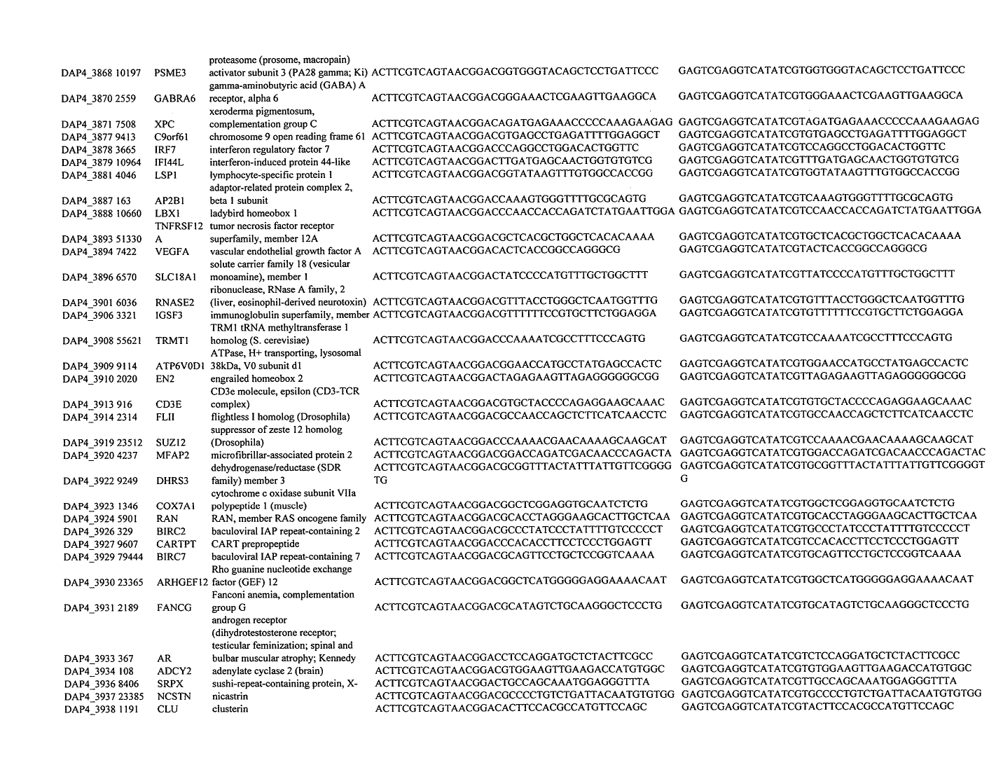

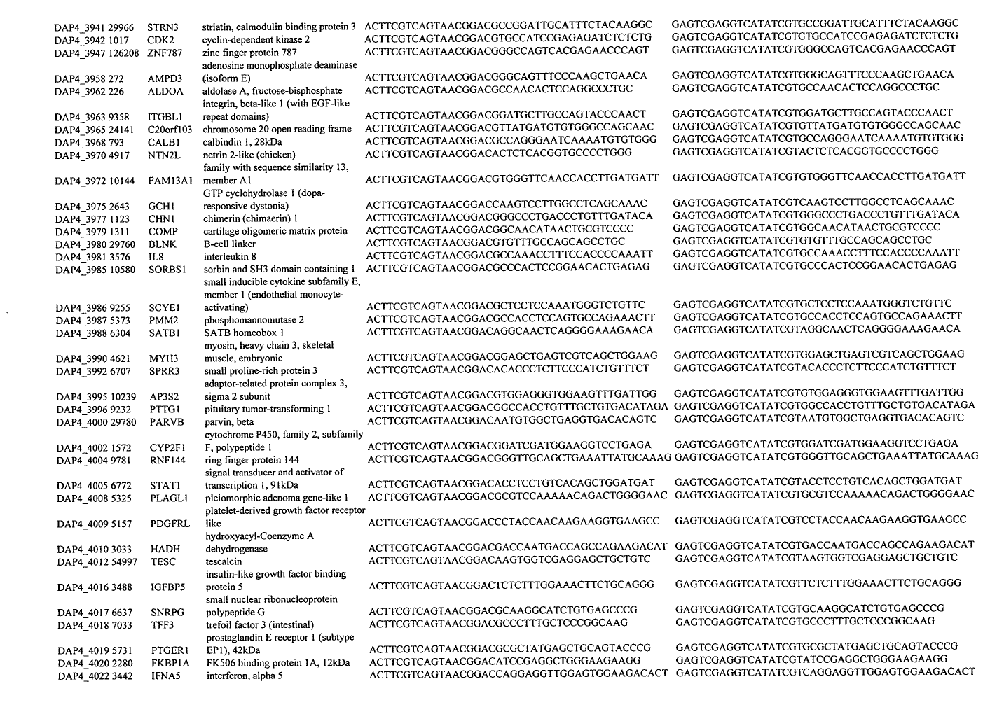

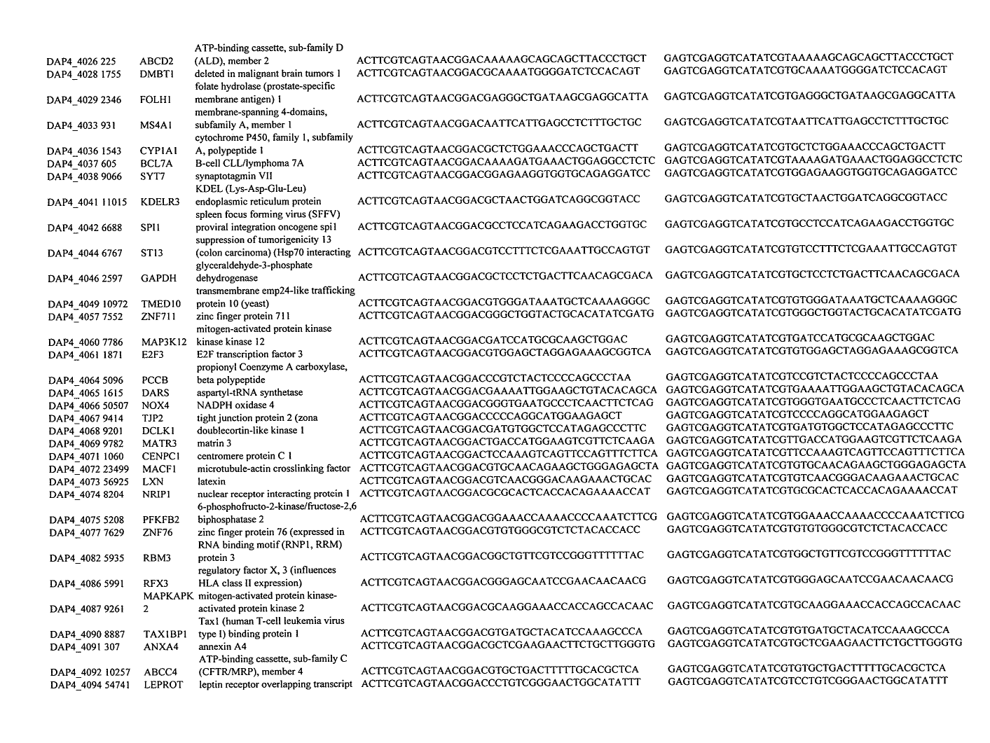

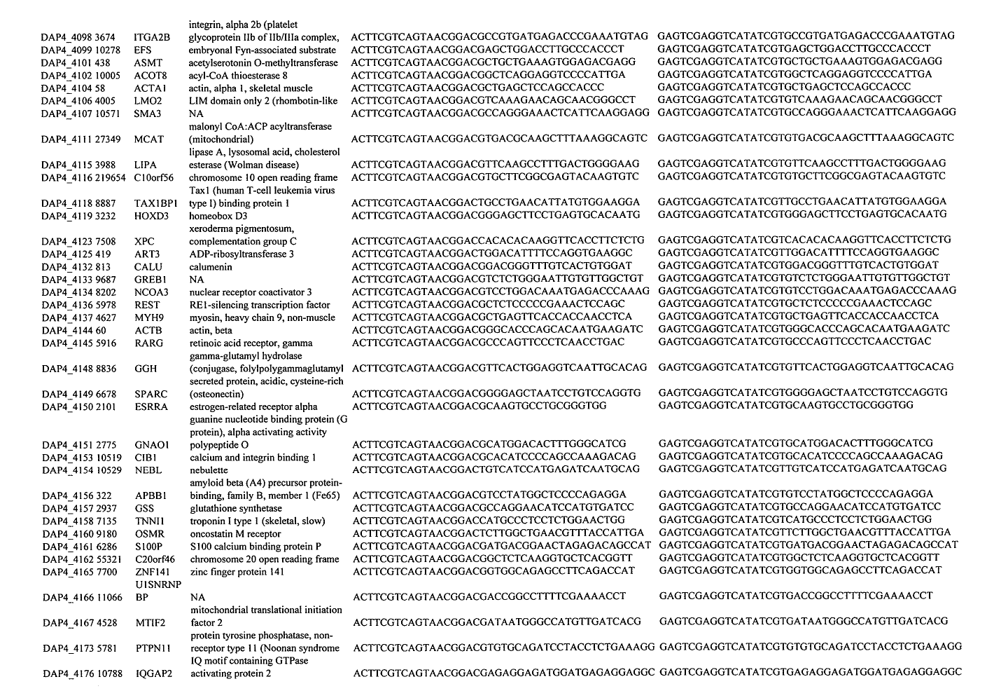

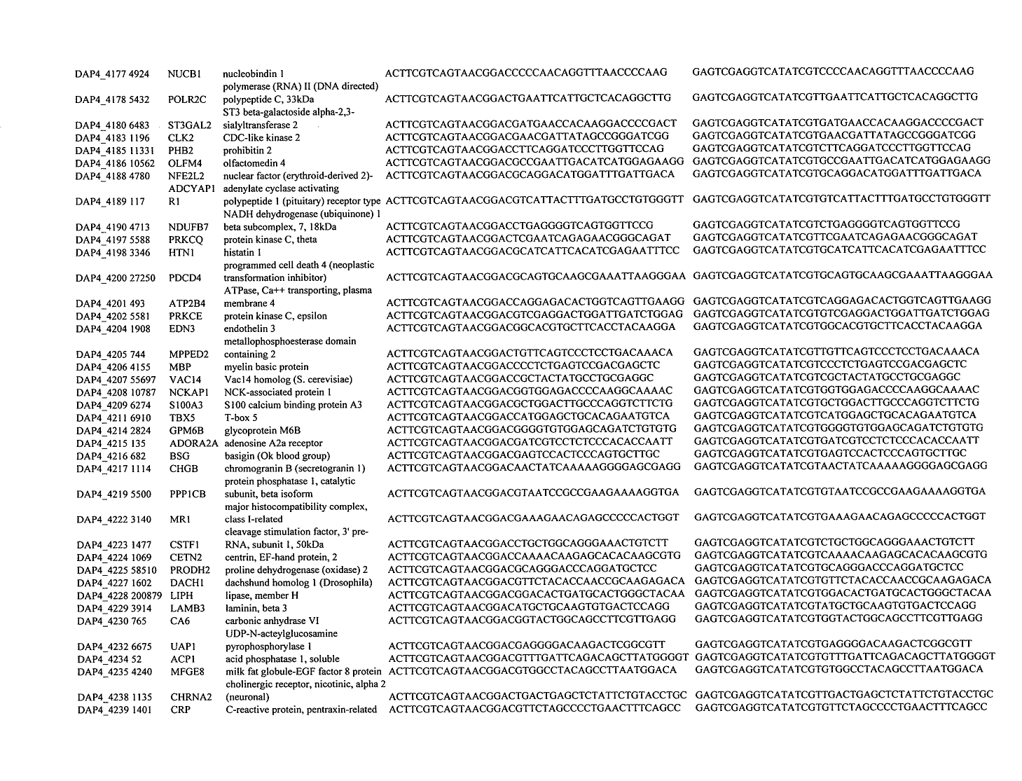

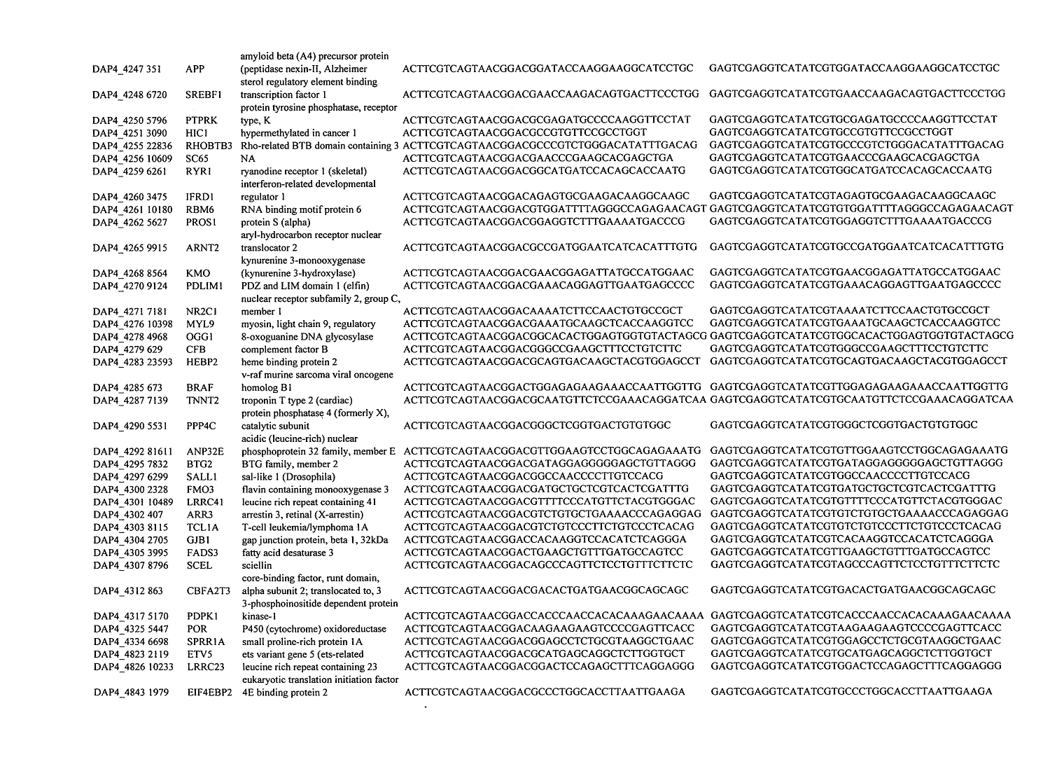

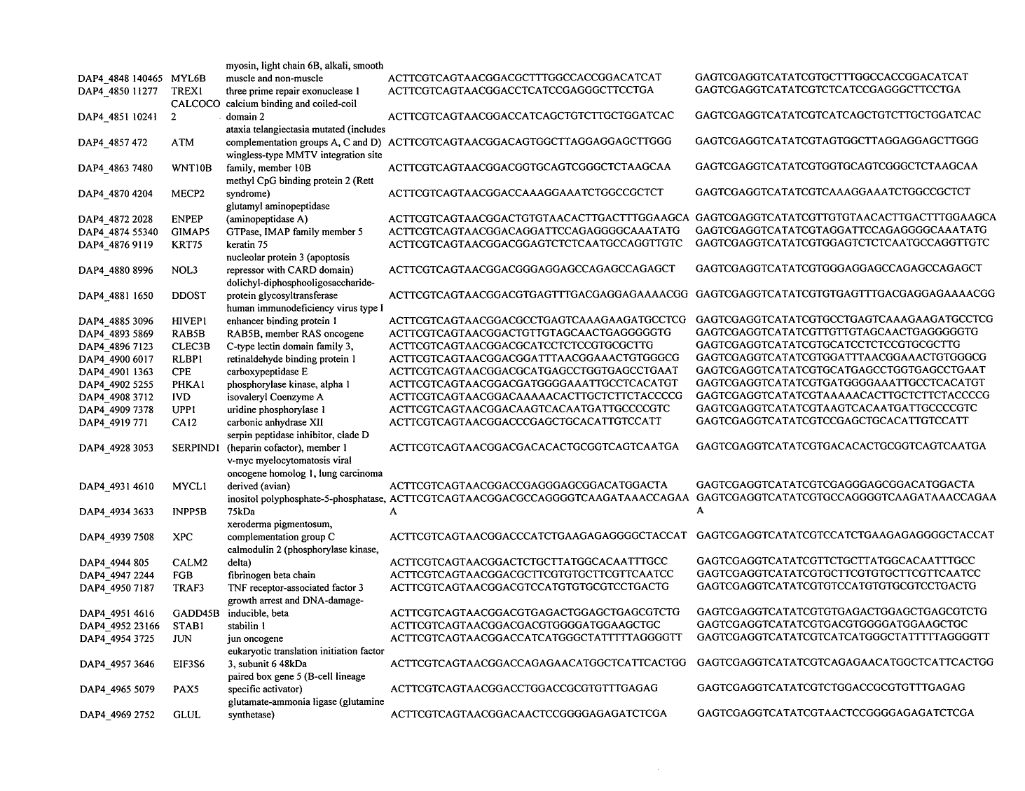

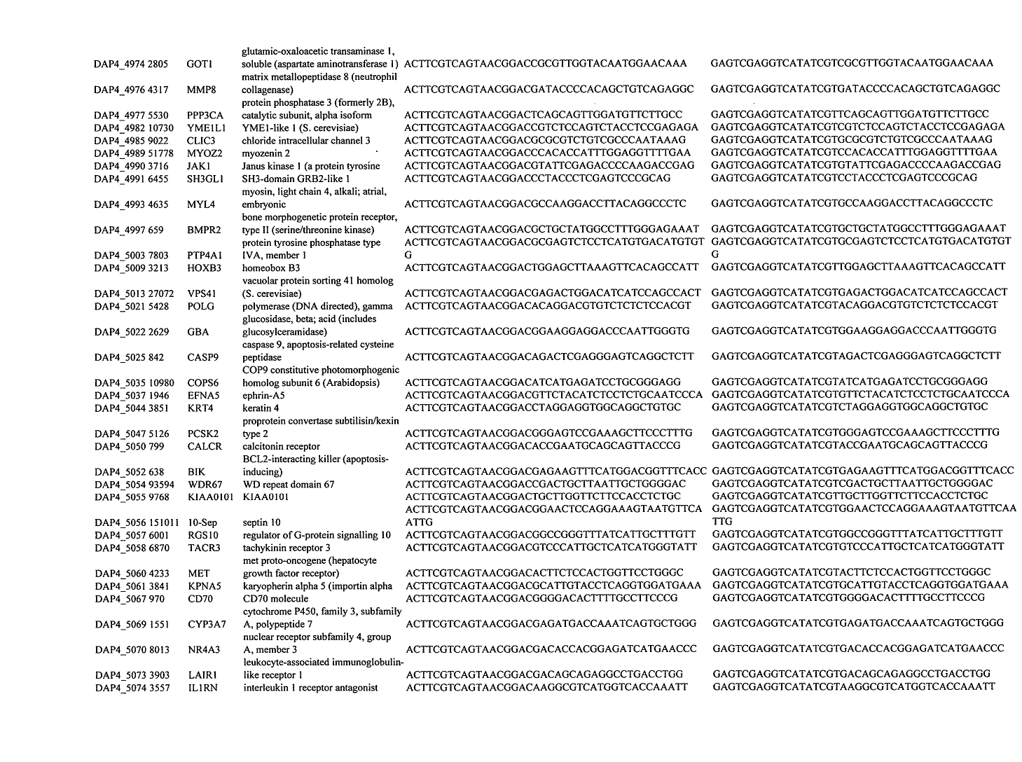

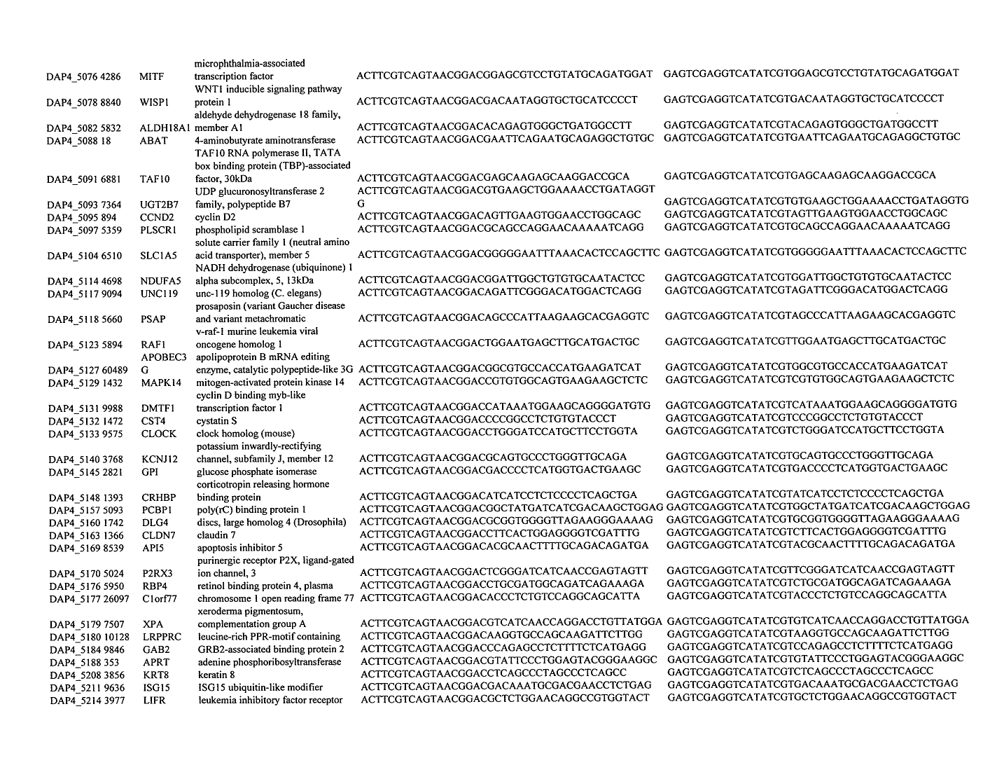

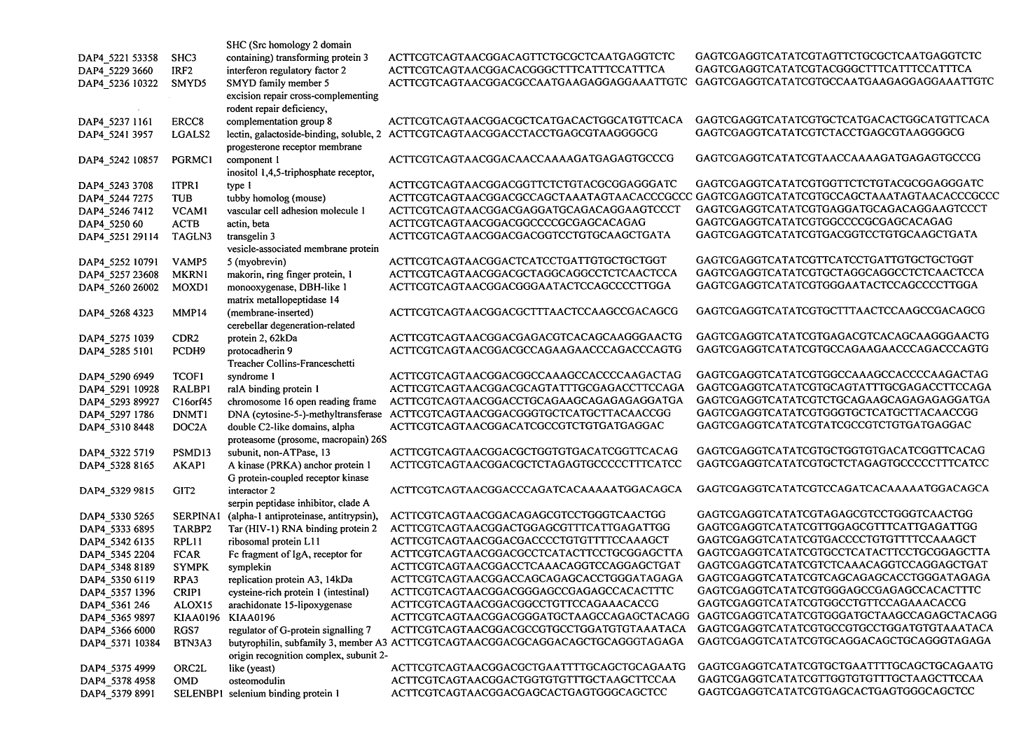

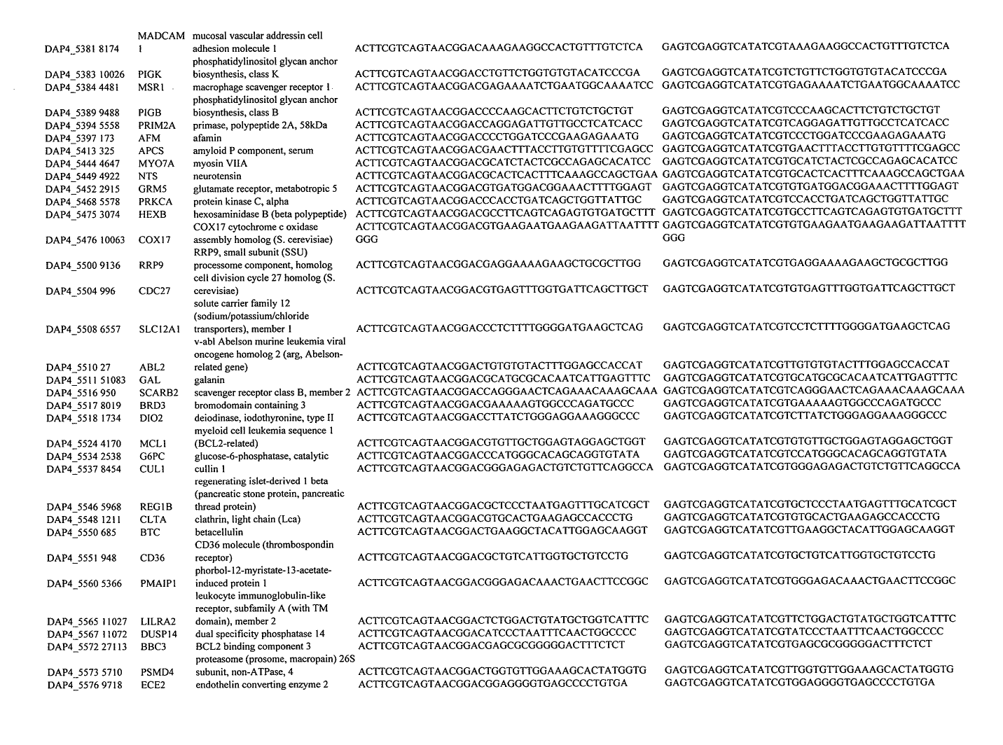

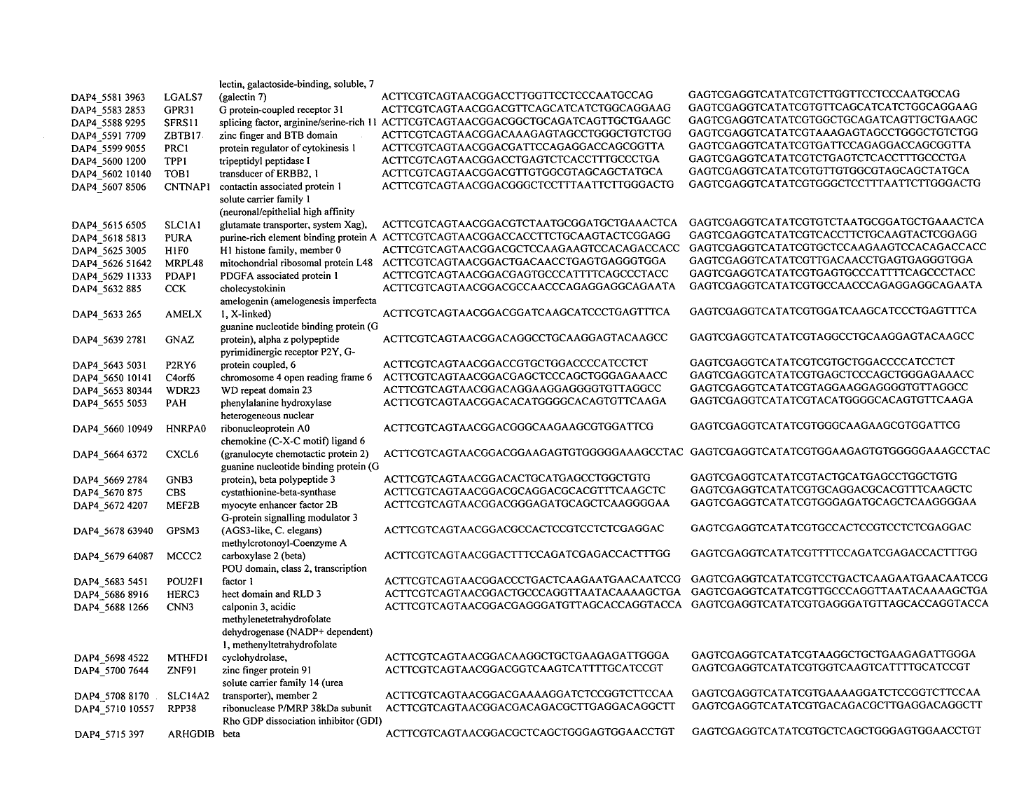

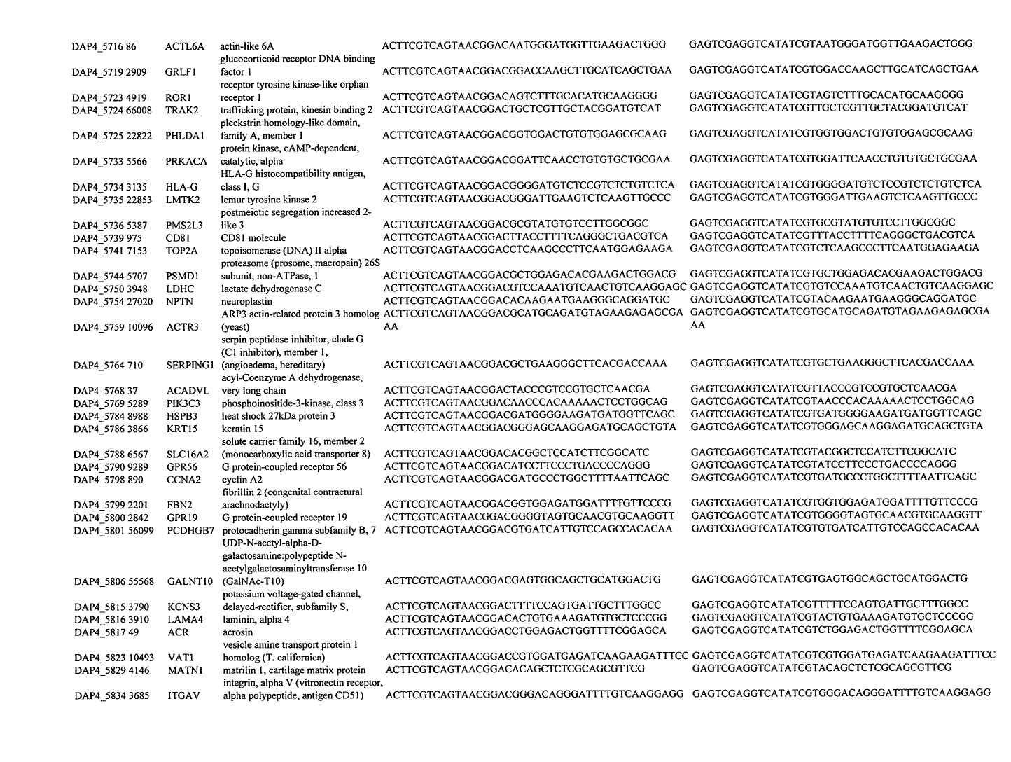

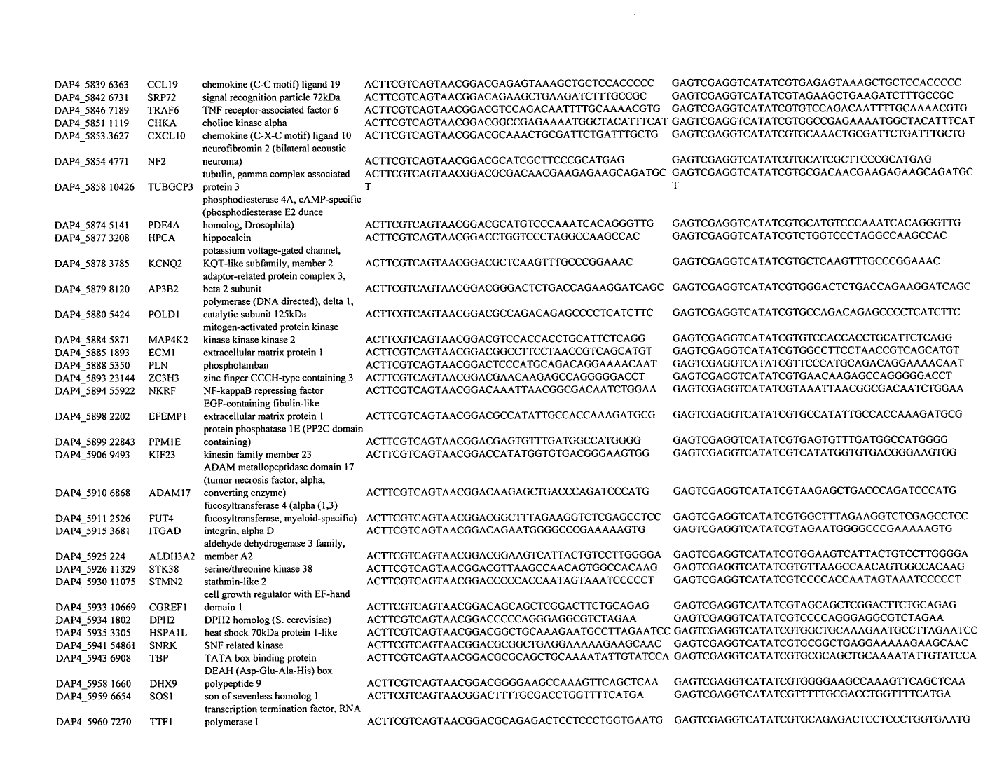

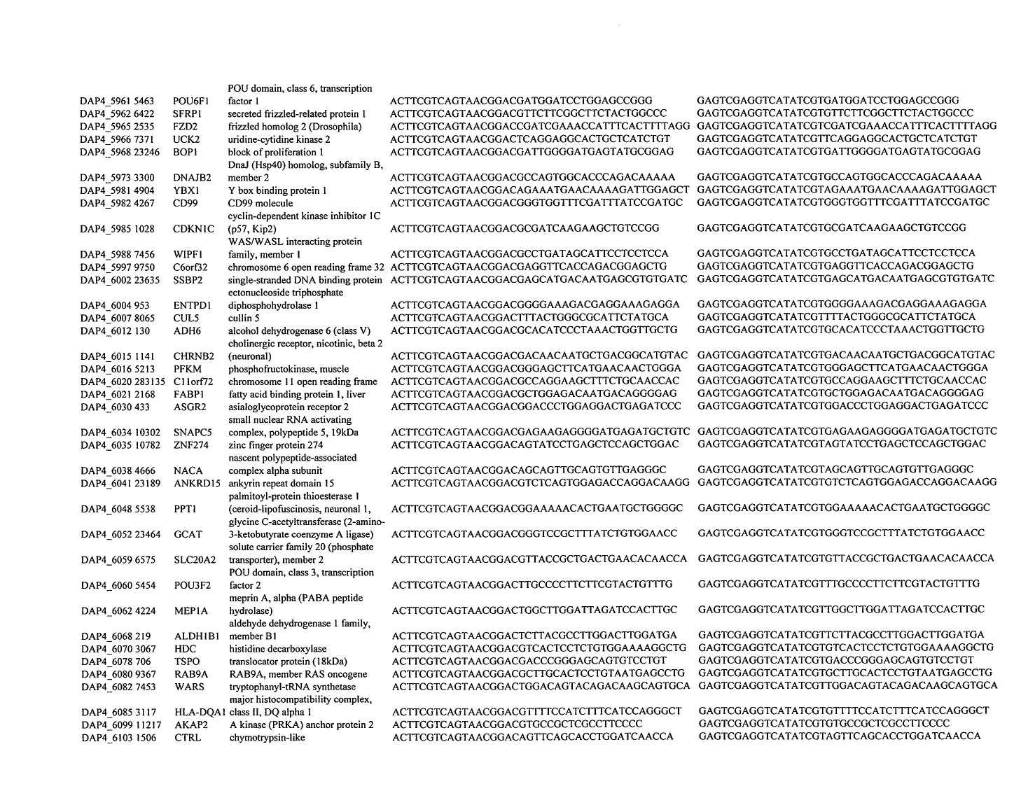

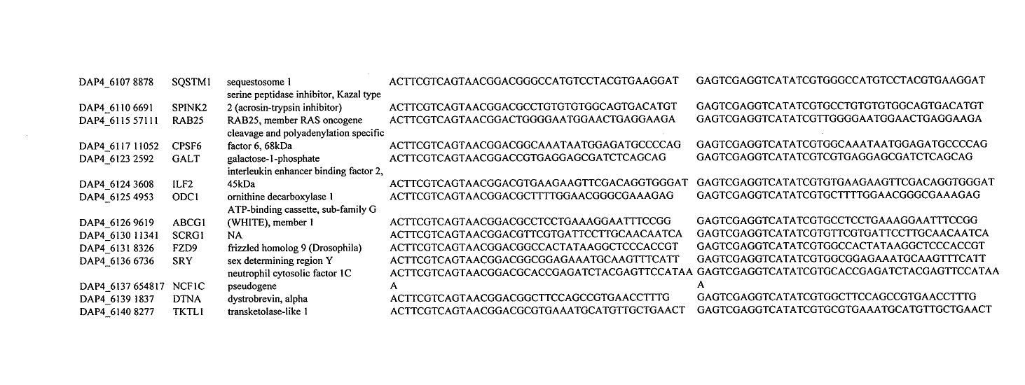

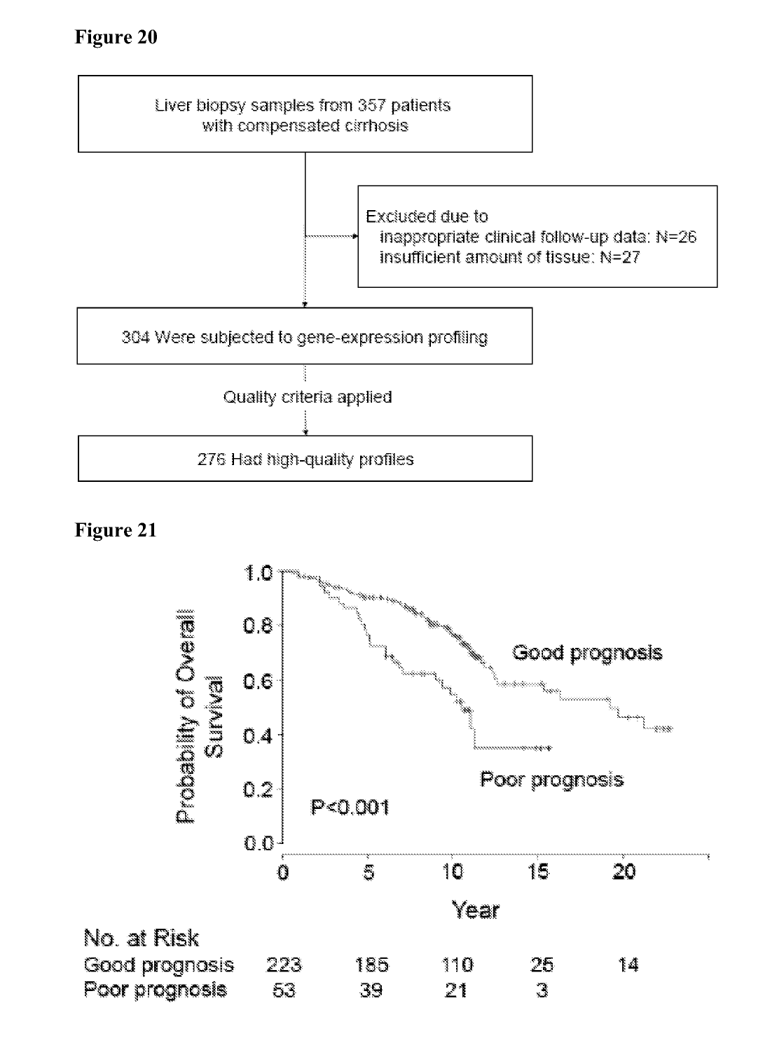

Because the standard approach to assessing cirrhosis in the clinical setting involves fine needle biopsies followed by formalin fixation, assessing the feasibility of performing genome-wide expression profiling on such small samples (typically 10 mm×1 mm pieces of tissue) was first determined. For expression profiling, the DASL assay was used, which was previously shown to allow the profiling of the expression of ˜6,000 transcripts in large formalin-fixed paraffin-embedded specimens obtained from surgical resection. Here, the ability of DASL to profile expression of all ˜24,000 genes in the human genome in fixed, fine needle biopsy specimens was tested. Of 357 patients enrolled in the study, 331 (93%) had sufficient clinical follow-up, and were therefore considered for expression profiling (FIG. 20). Of those, 304 (92%) had formalin-fixed paraffin-embedded tissue blocks available for study, and these were subjected to expression profiling. Quality control criteria established prior to this study were applied to these data, and 276/304 (91%) yielded high quality genome-wide expression profiles. This result was remarkable because of (1) the tiny size of the specimens, (2) the age of the archived specimens (up to 23 years old), and (3) the fact that the samples were not collected with expression profiling as a primary goal.

### Example 9

### Patient Characteristics

Table 14 summarizes clinical characteristics of the 276 patients analyzed. As described below, the patients were representative of the clinical course of patients with cirrhosis. Nearly all of the patients (270/276, 98%) presented with preservation of liver function as reflected in their being classified as Child-Pugh class A. Two hundred forty nine patients (90%) had evidence of hepatitis C virus infection. Ninety patients (33%) died during the follow-up period: 31 patients died of hepatocellular carcinoma, 34 patients died of liver failure, and the remaining 21 patients died of other or unknown causes. Eighty-eight patients (29%) had episodes of hepatic decompensation (presence of ascites and/or gastrointestinal bleeding) and 81 patients (29%) developed hepatocellular carcinoma. The annual incidence of hepatocellular carcinoma was 3%, consistent with prior studies of hepatocellular carcinoma incidence in patients with cirrhosis (Llovet J. M., et al. (2003) Lancet 362, 1907-1917). To further validate the representative nature of the study cohort, the relationship between well-established clinical variables and outcome (including platelet count, age, presence of varices and serum albumin) was examined. Univariate analysis showed that these factors, as expected, held modest prognostic value (Table 15), indicating that the study cohort was comparable to other populations of patients with cirrhosis.

### Example 10

### Validation of Survival Signature

It was next determined whether survival among patients with cirrhosis could be predicted based on gene expression. For this purpose, a 186-gene signature that was developed as a predictor of survival among patients with hepatocellular carcinoma following surgical resection of their primary tumors was used (Hoshida, Y., et al. (2008) N. Engl. J. Med. 359, 1995-2004). Importantly, the survival signature was applied to the cirrhosis cohort without modification, thereby precluding any over-optimization of the signature for the present dataset. Using this survival signature, 53 patients (19%) were classified as having the poor-prognosis signature, and this was statistically significantly associated with survival (P<0.001) (FIG. 21). The signature also showed significant association with hepatic decompensation (P=0.002) and marginal association with hepatocellular carcinoma development (P=0.08) (Table 15). These results indicate that the 186-gene signature, previously defined as a predictor of survival following primary tumor resection, is also predictive of outcome in patients with cirrhosis.

### Example 11

### Multivariate Analysis

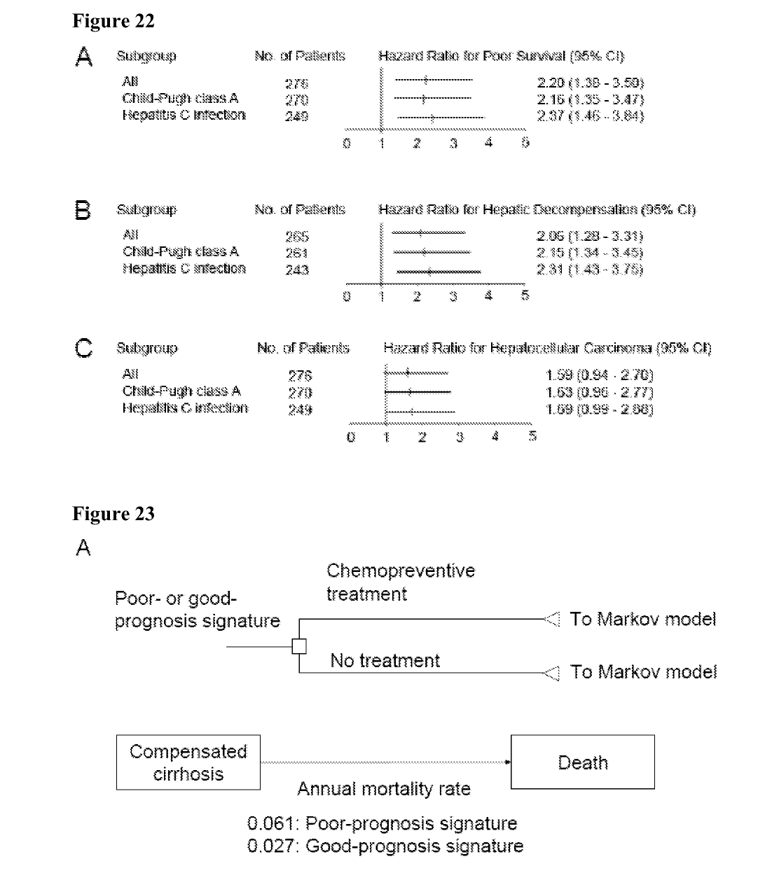

The value of the signature was further evaluated in the context of clinical variables generally found to be associated with outcome. Multivariate analyses showed that the survival signature showed significant association with survival (P<0.001) and hepatic decompensation (P=0.003) and a trend of association with hepatocellular carcinoma development (P=0.09) together with serum bilirubin and platelet count (Table 16). Prespecified subgroup analyses showed that the association with survival remained significant in both the subgroup of 270 patients of Child-Pugh class A (P=0.001) and the subgroup of 249 patient infected with hepatitis C virus (P<0.001) (FIG. 22 and Table 17). Even though the survival signature was originally trained for cancer-specific death, a significant association with non-cancer-specific death (P=0.004) was also observed (Table 18). These results show that the gene expression signature has outcome-predictive value above and beyond existing clinical parameters.

### Example 12

### Molecular Pathways Associated with Clinical Outcome

Molecular pathways associated with survival and hepatocellular carcinoma development were interrogated using Gene Set Enrichment Analysis (GSEA) (Subramanian, A., et al. (2005) Proc. Natl. Acad. Sci. U.S.A. 102, 15545-15550). First, genes on the microarray were rank-ordered according to the correlation to time-to-outcome calculated using the Cox score as previously described (Hoshida, Y., et al. (2008) N. Engl. J. Med. 359, 1995-2004). Subsequently, enrichment of two categories of annotated gene sets were evaluated on the rank-ordered gene list: target genes of experimental perturbation (473 sets) and literature-based curated pathway gene sets (150 sets) collected in our molecular signature database (MSigDB database available on the world wide web at the Broad Institute website). Poor prognosis-correlated gene expression is enriched in interferon- and inflammation-related pathways as well as matrix metalloproteinase pathway genes (which may contribute to liver fibrosis), while good prognosis is enriched in genes representing metabolic pathways involved in normal liver function (Table 19). These observations are consistent with the results for the 186-gene signature (Hoshida, Y., et al. (2008) N. Engl. J. Med. 359, 1995-2004).

### Example 13

### Multivariate Cox Regression Modeling

In the univariate Cox regression analysis, the following clinical variables showed relatively moderate association with survival: age (P=0.03), esophageal/gastric varices (P=0.006), and albumin (P=0.006) (Table 15). These variables were separately evaluated in the multivariate model together with the 186-gene signature, bilirubin, and platelet count. In summary, none of them remained to be significant in the multivariate models (Table 20).

More specifically, protein synthesis-related variables like albumin and prothrombin time were assumed not to be informative because the vast majority of the patients showed well-reserved protein synthesis capability. In fact, the numbers of patients with albumin <3.5 g/dL and prothrombin time (international normalized ratio)<1.7, both are the cut-off to discriminate the mildest Child-Pugh stage (Pugh, R. N., et al. (1973) Br. J. Surg. 60, 646-649), are only 10 (4%) and 0 (0%), respectively. The information of portal hypertension may be more sensitively captured by platelet count compared to presence of esophageal/gastric varices in the cohort of early-stage cirrhosis.

### Example 14

### Assessment of Other Prognostic Variables

The following prognostic factors were not included in the main analysis because the data were available in a limited number of patients. The association with outcome was evaluated together with the 186-gene signature, bilirubin, and platelet count in the multivariate modeling.

### Model for End-Stage Liver Disease (MELD) Score

MELD score is a survival predictor to discriminate patients with advanced cirrhosis, and has been used to prioritize patients indicated for liver transplantation (Kamath, P. S., et al. (2007) Hepatology 45, 797-805). The variables used to calculate MELD score, prothrombin time, serum creatinine, and bilirubin, were available for 179 patients in the cohort. MELD score above 6 showed association with survival (P=0.01) in univariate analysis, although it did not remain to be significant in the multivariate analysis (P=0.42) (Table 21). It was assumed that the score was less informative in the absence of patients with advanced cirrhosis. In fact, only 5 patients (3%) had the score above 10, which was reported to be predictive of poor survival (Bruno, S., et al. (2009) Am. J. Gastroenterol. 104, 1147-1158).

### Antiviral Therapy-Related Variables and Hepatitis C Virus Genotype

Among 249 patients infected with hepatitis C virus, 116 patients have a history of interferon treatment. Among 104 patients with information of treatment response, 22 patients showed sustained virological response (SVR), i.e., clearance of the virus. Despite the small sample size, SVR, but not history of interferon therapy, was associated with good outcome consistent with the current consensus (Bruno, S., et al. (2009) Am. J. Gastroenterol. 104, 1147-1158) (Table 22). Hepatitis C virus genotype 1b was also associated with outcome as previously described (Bruno, S., et al. (2009) Am. J. Gastroenterol. 104, 1147-1158). However, the genotype, also known as the predictor of poor response to interferon (Martinot-Peignoux, M., et al. (1995) Hepatology 22, 1050-1056), showed significant association with sustained virological response (10% response in genotype 1b and 35% response in other genotypes, P=0.003, Fisher's exact test). In a subset of 120 hepatitis C-infected patients who did not receive interferon therapy, genotype 1b showed a trend of association with survival (P=0.08), although the analysis might be underpowered.

### Death Unrelated to Hepatocellular Carcinoma Progression

Because the 186-gene survival signature was originally trained in hepatocellular carcinoma patients, specific association with non-cancer-related death was assessed by treating cancer-related deaths as censored observations in the multivariate Cox regression modeling. The survival signature remained to show significant association with survival (P=0.004) (Table 17), indicating that the signature also captures the risk of death unrelated to cancer itself.

### Each Component of Hepatic Decompensation Event: Ascites and Gastrointestinal Bleeding

To interrogate whether any variable correlated with a more specific decompensation event, the association of the 186-gene signature was also analyzed with either of the presence of ascites or gastrointestinal bleeding (Table 23). The survival signature showed significant association with the presence of ascites. The presence of esophageal/gastric varices was the only variable associated with gastrointestinal bleeding.

### Hepatocellular Carcinoma Development According to Baveno IV Staging of Cirrhosis

It was determined whether subgroups of patients identified with significant association for hepatocellular carcinoma developed. Application of the recently proposed Baveno IV prognostic staging system for cirrhosis (de Franchis, R. et al. (2005) J. Hepatol. 43, 167-76) classified patients into either of stage 1 (N=204) or stage 2 (N=65) according to absence or presence of esophageal/gastric varices (which was not independently associated with survival). A stronger association of the signature with hepatocellular carcinoma development in Baveno IV stage 2 patients was observed (Table 24), suggesting that the signature is particularly applicable in predicting the first hepatocellular carcinoma development in this subgroup of patients.

### Example 15

### Gene Expression in Cirrhotic Tissues and Association with Hepatocellular Carcinoma Development

The 186-gene signature showed moderate association with the first hepatocellular carcinoma development. To evaluate whether there is any gene expression pattern in cirrhotic tissues more strongly associated with hepatocellular carcinoma development compared to the 186-gene signature, a standard leave-one-out cross validation procedure was conducted as previously described (Hoshida, Y., et al. (2008) N. Engl. J. Med. 359, 1995-2004). Briefly, a single sample was left out one-by-one and an outcome-correlated signature was selected from the remaining samples (selecting marker genes based on permutation test P-value less than 0.005). A prediction label was assigned to the left-out sample using the NTP algorithm. The log-rank test showed no association between the prediction and hepatocellular carcinoma development (P=0.24), indicating no better transcriptional information in the dataset of cirrhosis.

### Example 16

### Survival Benefit of Chemopreventive Therapy According to Signature-Based Prediction

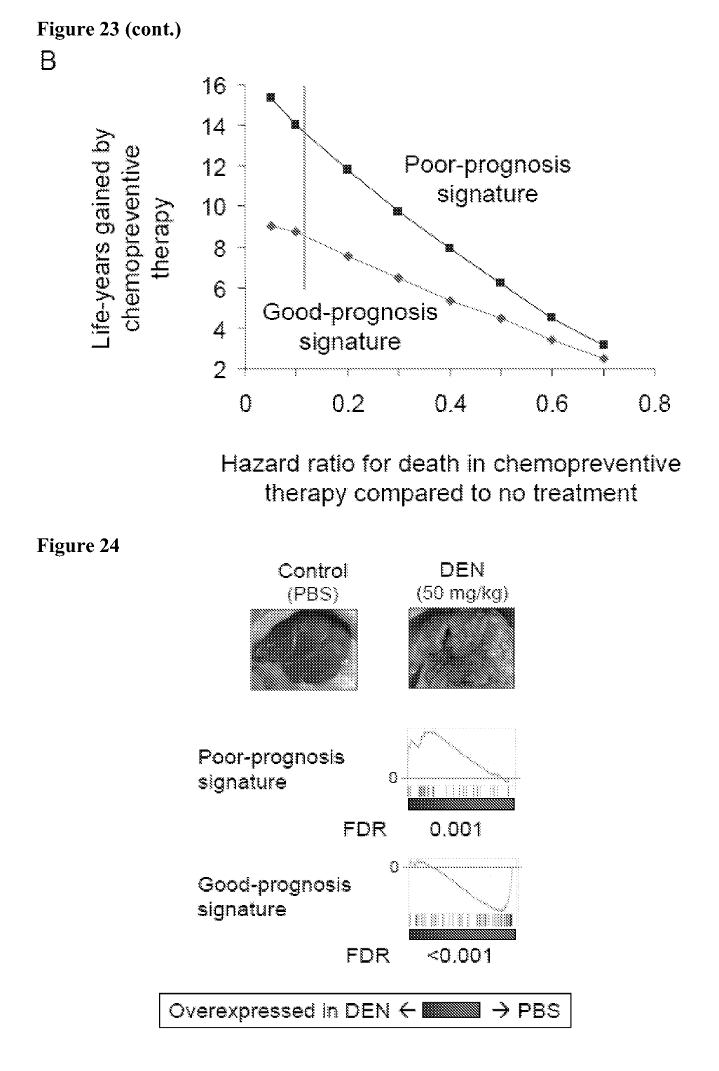

Survival benefit of signature-based patient selection in chemopreventive therapy for cirrhotic complication was estimated based on a simple Markov model (FIG. 23). The transition probabilities in the model were calculated by averaging annual mortality rates at 5, 10, and 15 years in FIG. 21 with the use of the declining exponential approximation of life expectancy (DEALE) (Beck, J. R., et al. (1982) Am. J. Med. 73, 889-97). Life years gained by the therapy were computed according to therapeutic effect, represented by hazard ratio compared to no therapy, ranging from 0.05 (marked effect) to 0.7 (mild effect) (FIG. 23). In the model, a conservative assumption that treatment effect, i.e., hazard ratio for the therapy, was the same between patients with poor-prognosis and good-prognosis signatures was used. The vertical line in the graph indicates the hazard ratio of 0.135 for death reported in a randomized clinical trial of interferon therapy for hepatitis C-related cirrhosis (Nishiguchi, S. et al. (2001) Lancet 357, 196-197.). The analysis was performed using TreeAge Pro ver.1.0.2 software (TreeAge Software).

Accordingly, testing of the signature on 276 patients followed in Milan for up to 23 years showed that indeed the signature was associated with hepatocellular carcinoma development. More striking, however, was the surprising observation that the signature was predictive of survival in this cohort due to all cirrhosis-related causes—not just liver cancer. Specifically, death from liver failure was predicted by the signature, despite the fact that the signature was originally developed to predict hepatocellular carcinoma-associated survival (Hoshida, Y., et al. (2008) N. Engl. J. Med. 359, 1995-2004). These observations are consistent with the idea of a “field effect” in which liver injury (most commonly as a result of hepatitis B or C infection) predisposes to not only liver cancer, but also worsening cirrhosis leading to portal hypertension, loss of synthetic liver function resulting in metabolic disturbances, coagulopathies, and ultimately death. This close relationship between liver cancer and cirrhosis-related liver failure is similarly reflected by the Child-Pugh classification system which was initially developed as a predictor of survival in patients with bleeding esophageal varices, but which turned out to also predict a number of cirrhosis-related clinical endpoints including hepatocellular carcinoma (D'Amico, G., et al. (2006) J. Hepatol. 44, 217-231).

Interestingly, the signature was more highly associated with liver failure than with development of hepatocellular carcinoma (P=0.003 vs. P=0.09). This is likely due, at least in part, to the difficulty in detecting asymptomatic hepatocellular carcinoma nodules (generally detected by ultrasound monitoring, which is highly operator-dependent). In contrast, liver failure is accompanied by obvious clinical parameters such as bleeding diatheses, jaundice and patients feeling ill. As improved radiographic monitoring for hepatocellular carcinoma becomes available, the signature's association with tumor development may increase.

The survival signature was an independent predictor of outcome in the study, predicting survival above and beyond existing clinical prognostic staging systems such as Child-Pugh classification and the Model for End Stage Liver Disease (MELD) score (amath, P. S., et al. (2007) Hepatology 45, 797-805). Notably, essentially all of the study patients were Child-Pugh class A at the time of analysis, reflective of the early stage at which most patients are diagnosed in major metropolitan areas. Child-Pugh staging accordingly offers limited clinical value in that setting. In addition, the clinical parameters comprising existing prognostic scoring systems (e.g., serum albumin concentration, degree of abdominal ascites) can be highly affected by medical intervention (e.g., albumin supplementation, paracentesis, diuretic use, etc), thereby making those clinical features inaccurate measures of liver function.

Of particular clinical relevance is our demonstration that genome-wide expression profiling can be performed on fine needle liver biopsies that are obtained during the routine clinical care of patients with cirrhosis. Examples 1-7 demonstrate that such profiling was possible from large, surgical resection specimens, but the feasibility of needle biopsy profiling suggests that the measurement of the survival signature and other such signatures could be implemented in a routine clinical setting.

It is believed that patients harboring the poor-prognosis vs. good-prognosis signature would differentially benefit from either enhanced surveillance or therapeutic intervention (e.g., chemopreventive strategies such as interferon). The potential public health impact of a test that identifies high-risk patients with a disease as common as cirrhosis cannot be overemphasized. Resources could be focused on those most likely to benefit, and toxicity could be spared for those patients with a low probability of cirrhosis-related morbidity or mortality. For example, a simple Markov model based on prioritizing patients for interferon therapy based on their survival signature suggests that on average an additional 5 life-years of therapeutic benefit would be realized in treating patients with the poor prognosis signature compared to the good prognosis signature (FIGS. 23A and 23B). The signature thus has the potential to enrich for patient populations most likely to show benefit from new experimental interventions for cirrhosis, thus allowing for smaller, more cost-effective clinical trial strategies. Accordingly, it is believed that the measurement of this signature should become a key component of all future clinical trials assessing the natural history or therapeutic interventions in patients with cirrhosis.

### Example 17

### Gene Signature-Based Monitoring of Cancer-Preventive Effect of Erlotinib

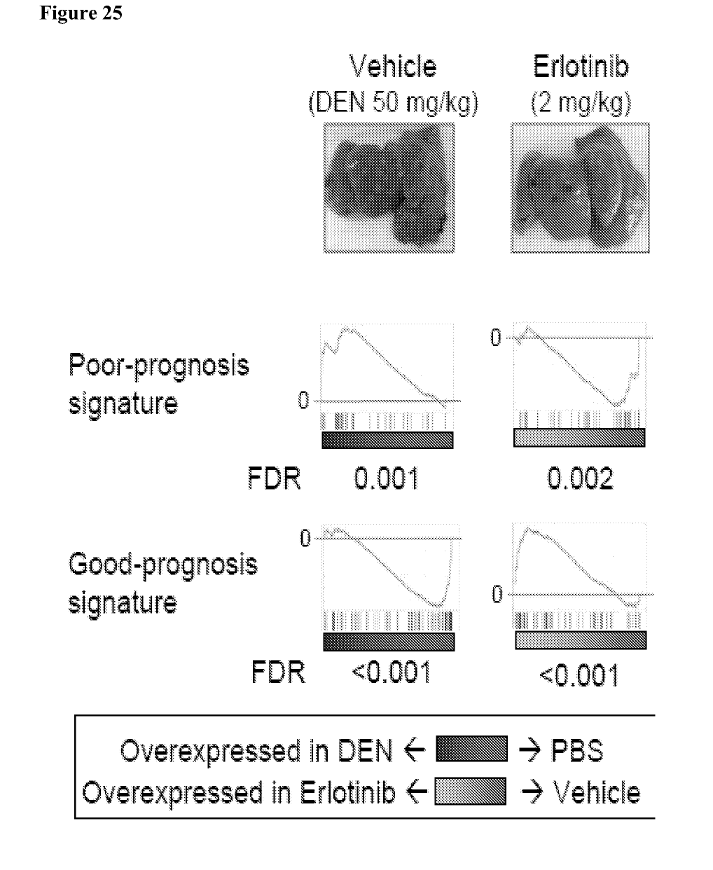

The 186-gene signature described in Examples 1-16 was further assessed for applicability to the monitoring of the liver cancer-preventive effects of erlotinib. Two groups of rats (n=6 for each group) were analyzed. One group was treated with diethylnitrosamine (DEN, 50 mg/kg), representing a rat model of liver cirrhosis and cancer by producing liver cirrhosis and cancer. The other group was a control group treated with phosphate buffered saline (PBS). The cirrhotic rats were treated with erlotinib (2 mg/kg) and compared with control group treated with vehicle based upon expression status of the 186-gene signature as evaluated using RatRef-12 DNA microarrays (Illumina). The livers of DEN-treated rats showed histologically established liver cirrhosis associated with impaired liver function consistent with human cirrhosis. The poor-prognosis signature was induced in the rat cirrhosis with statistical significance (FIG. 24). The expression pattern of the signature associated with erlotinib treatment was shifted toward direction of good prognosis with statistical significance (FIG. 25). Accordingly, it is believed that the 186-gene signature can be used to monitor the liver cancer-preventive effect of erlotinib. In addition, measurement of the signature in the rat cirrhosis model may be used to screen drugs preventing liver cancer development.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Insitute for Genomic Research (TIGR) on the world wide web and/or the National Center for Biotechnology Information (NCBI) on the world wide web.

### EQUIVALENTS

Those skilled in the art will recognize or be able to ascertain using no more than routine experimentation, many equivalents to the specific embodiments of the invention described herein. Such equivalents are intended to be encompassed by the claims herein.

TABLE 1

| | | Hazard | 95% confidence interval | | |
|---|---|---|---|---|---|
| Variable | Category | ratio | low | high | p-value |
| Age | ≧60 | 0.75 | 0.37 | 1.49 | 0.40 |
| Sex (male) | male | 0.41 | 0.14 | 1.16 | 0.09 |
| HBV | | 0.61 | 0.23 | 1.57 | 0.30 |
| HCV | | 2.18 | 0.84 | 5.69 | 0.11 |
| Alcohol | | 2.60 | 0.79 | 8.59 | 0.12 |
| BCLC stage | B (vs. 0/A) | 1.45 | 0.44 | 4.77 | 0.54 |
| | A/B (vs. 0) | 1.90 | 0.86 | 4.20 | 0.11 |
| Tumor diameter (cm) | ≧3 cm | 1.21 | 0.60 | 2.43 | 0.60 |
| Tumor differentiation | Moderate (vs. Well) | 0.84 | 0.39 | 1.85 | 0.67 |
| | Poor (vs. Well) | 0.67 | 0.23 | 2.02 | 0.48 |
| Vascular invasion | | 2.11 | 0.50 | 8.94 | 0.31 |
| Cirrhosis | | 1.90 | 0.78 | 4.58 | 0.16 |
| AFP (ng/mL) | ≧100 | 0.95 | 0.47 | 1.94 | 0.89 |
| Platelet count (×10⁹/L) | <10.0 | 1.68 | 0.86 | 3.28 | 0.13 |

HB: hepatitis B, HCV: hepatitis C virus, AFP: alpha-fetoprotein

### Table 2

TABLE 2A
Genes correlated with poor survival

| Probe ID | GeneID | Gene symbol | Description | Cox score |
|---|---|---|---|---|
| DAP1_5052 | 2488 | FSHB | follicle stimulating hormone, beta polypeptide | 4.80 |
| DAP1_0153 | 6456 | SH3GL2 | SH3-domain GRB2-like 2 | 4.21 |
| DAP1_2390 | 23029 | RBM34 | RNA binding motif protein 34 | 4.19 |
| DAP3_3833 | 23397 | NCAPH | non-SMC condensin I complex, subunit H | 4.02 |
| DAP1_0623 | 1950 | EGF | epidermal growth factor (beta-urogastrone) | 3.97 |
| DAP1_5926 | 7204 | TRIO | triple functional domain (PTPRF interacting) | 3.90 |
| DAP3_3842 | 1293 | COL6A3 | collagen, type VI, alpha 3 | 3.87 |
| DAP1_0171 | 3983 | ABLIM1 | actin binding LIM protein 1 | 3.86 |
| DAP3_0607 | 3680 | ITGA9 | integrin, alpha 9 | 3.81 |
| DAP4_5449 | 4922 | NTS | neurotensin | 3.78 |
| DAP3_1324 | 5055 | SERPINB2 | serpin peptidase inhibitor, clade B (ovalbumin), member 2 | 3.69 |
| DAP3_1228 | 4316 | MMP7 | matrix metallopeptidase 7 (matrilysin, uterine) | 3.59 |
| DAP3_4010 | 5593 | PRKG2 | protein kinase, cGMP-dependent, type II | 3.44 |
| DAP4_1888 | 9270 | EDG4 | endothelial differentiation, lysophosphatidic acid G-protein-coupled | 3.40 |
| DAP3_0208 | 4843 | NOS2A | nitric oxide synthase 2A (inducible, hepatocytes) | 3.33 |
| DAP1_4004 | 2043 | EPHA4 | EPH receptor A4 | 3.25 |
| DAP4_2216 | 5572 | SP100 | SP100 nuclear antigen | 3.19 |
| DAP2_0010 | 2325 | FMO1 | flavin containing monooxygenase 1 | 3.04 |
| DAP3_2729 | 2877 | GPX2 | glutathione peroxidase 2 (gastrointestinal) | 3.02 |
| DAP3_5508 | 496 | ATP4B | ATPase, H+/K+ exchanging, beta polypeptide | 2.99 |
| DAP1_5176 | 8870 | IER3 | immediate early response 3 | 2.98 |
| DAP4_5988 | 7456 | WIPF1 | WAS/WASL interacting protein family, member 1 | 2.98 |
| DAP1_3877 | 3489 | IGFBP6 | insulin-like growth factor binding protein 6 | 2.93 |
| DAP1_0897 | 1501 | CTNND2 | catenin (cadherin-associated protein), delta 2 (neural plakophilin-related | 2.92 |
| | | | arm-repeat protein) | |
| DAP3_5371 | 2200 | FBN1 | fibrillin 1 | 2.91 |
| DAP4_5022 | 2629 | GBA | glucosidase, beta; acid (includes glucosylceramidase) | 2.85 |
| DAP1_4874 | 22858 | ICK | intestinal cell (MAK-like) kinase | 2.83 |
| DAP1_3085 | 10523 | CHERP | calcium homeostasis endoplasmic reticulum protein | 2.81 |
| DAP3_3881 | 9734 | HDAC9 | histone deacetylase 9 | 2.81 |
| DAP3_1658 | 51406 | NOL7 | nucleolar protein 7, 27 kDa | 2.80 |
| DAP3_0609 | 8826 | IQGAP1 | IQ motif containing GTPase activating protein 1 | 2.79 |
| DAP3_3158 | 120 | ADD3 | adducin 3 (gamma) | 2.79 |
| DAP3_3933 | 306 | ANXA3 | annexin A3 | 2.78 |
| DAP2_5915 | 10362 | HMG20B | high-mobility group 20B | 2.76 |
| DAP1_0174 | 6558 | SLC12A2 | solute carrier family 12 (sodium/potassium/chloride transporters), member | 2.75 |
| DAP2_3448 | 1282 | COL4A1 | collagen, type IV, alpha 1 | 2.75 |
| DAP4_3126 | 1359 | CPA3 | carboxypeptidase A3 (mast cell) | 2.74 |
| DAP3_1093 | 3855 | KRT7 | keratin 7 | 2.74 |
| DAP1_1741 | 5271 | SERPINB8 | serpin peptidase inhibitor, clade B (ovalbumin), member 8 | 2.69 |
| DAP3_1042 | 4791 | NFKB2 | nuclear factor of kappa light polypeptide gene enhancer in B-cells 2 | 2.67 |
| DAP3_5816 | 165 | AEBP1 | AE binding protein 1 | 2.67 |
| DAP3_3879 | 7041 | TGFB1I1 | transforming growth factor beta 1 induced transcript 1 | 2.66 |
| DAP1_0509 | 2013 | EMP2 | epithelial membrane protein 2 | 2.63 |
| DAP2_3497 | 596 | BCL2 | B-cell CLL/lymphoma 2 | 2.63 |
| DAP3_2152 | 5691 | PSMB9 | proteasome (prosome, macropain) subunit, beta type, 9 (large | 2.59 |
| | | | multifunctional peptidase 2) | |
| DAP3_6062 | 10097 | ACTR2 | ARP2 actin-related protein 2 homolog (yeast) | 2.59 |
| DAP1_6137 | 780 | DDR1 | discoidin domain receptor family, member 1 | 2.58 |
| DAP2_3913 | 6541 | SLC7A1 | solute carrier family 7 (cationic amino acid transporter, y+ system), | 2.56 |
| DAP4_2003 | 5420 | PODXL | podocalyxin-like | 2.56 |
| DAP1_5750 | 1307 | COL16A1 | collagen, type XVI, alpha 1 | 2.55 |
| DAP1_3284 | 10437 | IFI30 | interferon, gamma-inducible protein 30 | 2.55 |
| DAP3_1596 | 9852 | EPM2AIP1 | EPM2A (laforin) interacting protein 1 | 2.55 |
| DAP3_1678 | 301 | ANXA1 | annexin A1 | 2.53 |
| DAP3_4123 | 6366 | CCL21 | chemokine (C-C motif) ligand 21 | 2.47 |
| DAP3_1610 | 22856 | CHSY1 | carbohydrate (chondroitin) synthase 1 | 2.45 |
| DAP1_4020 | 162 | AP1B1 | adaptor-related protein complex 1, beta 1 subunit | 2.45 |
| DAP4_2797 | 7004 | TEAD4 | TEA domain family member 4 | 2.39 |
| DAP4_2406 | 54898 | ELOVL2 | elongation of very long chain fatty acids (FEN1/Elo2, SUR4/Elo3, yeast)- | 2.39 |
| DAP1_0054 | 6925 | TCF4 | transcription factor 4 | 2.38 |
| DAP3_1020 | 9819 | TSC22D2 | TSC22 domain family, member 2 | 2.38 |
| DAP4_2418 | 1847 | DUSP5 | dual specificity phosphatase 5 | 2.36 |
| DAP3_5242 | 8030 | CCDC6 | coiled-coil domain containing 6 | 2.36 |
| DAP3_0973 | 962 | CD48 | CD48 molecule | 2.35 |
| DAP1_0901 | 10188 | TNK2 | tyrosine kinase, non-receptor, 2 | 2.35 |
| DAP3_1032 | 1601 | DAB2 | disabled homolog 2, mitogen-responsive phosphoprotein (Drosophila) | 2.35 |
| DAP2_3941 | 4017 | LOXL2 | lysyl oxidase-like 2 | 2.34 |
| DAP3_2205 | 6035 | RNASE1 | ribonuclease, RNase A family, 1 (pancreatic) | 2.34 |
| DAP4_2160 | 4026 | LPP | LIM domain containing preferred translocation partner in lipoma | 2.33 |
| DAP3_0038 | 7852 | CXCR4 | chemokine (C-X-C motif) receptor 4 | 2.33 |
| DAP3_1608 | 6586 | SLIT3 | slit homolog 3 (Drosophila) | 2.31 |
| DAP3_0744 | 13259 | FILIP1L | filamin A interacting protein 1-like | 2.25 |
| DAP4_5839 | 6363 | CCL19 | chemokine (C-C motif) ligand 19 | 2.23 |
| DAP3_5744 | 11214 | AKAP13 | A kinase (PRKA) anchor protein 13 | 2.23 |

TABLE 2B
Genes correlated with good survival

| Probe ID | GeneID | Gene symbol | Description | Cox score |
|---|---|---|---|---|
| DAP3_4190 | 223 | ALDH9A1 | aldehyde dehydrogenase 9 family, member A1 | −3.34 |
| DAP4_0296 | 7276 | TTR | transthyretin (prealbumin, amyloidosis type I) | −3.27 |
| DAP1_5588 | 6018 | RLF | rearranged L-myc fusion | −3.23 |
| DAP4_3479 | 3612 | IMPA1 | inositol(myo)-1(or 4)-monophosphatase 1 | −3.22 |
| DAP3_2208 | 5207 | PFKFB1 | 6-phosphofructo-2-kinase/fructose-2,6-biphosphatase 1 | −3.22 |
| DAP3_1951 | 6296 | ACSM3 | acyl-CoA synthetase medium-chain family member 3 | −3.21 |
| DAP4_2813 | 151 | ADRA2B | adrenergic, alpha-2B-, receptor | −3.19 |
| DAP1_3979 | 5771 | PTPN2 | protein tyrosine phosphatase, non-receptor type 2 | −3.12 |
| DAP3_1558 | 5691 | PSMB3 | proteasome (prosome, macropain) subunit, beta type, 3 | −3.09 |
| DAP3_2216 | 5502 | PPP1R1A | protein phosphatase 1, regulatory (inhibitor) subunit 1A | −3.07 |
| DAP3_0210 | 27346 | TMEM97 | transmembrane protein 97 | −3.06 |
| DAP2_4247 | 5313 | PKLR | pyruvate kinase, liver and RBC | −3.01 |
| DAP3_2434 | 9252 | RPS6KA5 | ribosomal protein S6 kinase, 90 kDa, polypeptide 5 | −3.00 |
| DAP1_0453 | 1528 | CYB5A | cytochrome b5 type A (microsomal) | −2.96 |
| DAP4_3541 | 6447 | SCG5 | secretogranin V (7B2 protein) | −2.93 |
| DAP1_1650 | 25828 | TXN2 | thioredoxin 2 | −2.90 |
| DAP2_1608 | 5340 | PLG | plasminogen | −2.88 |
| DAP3_2733 | 6309 | SC5DL | sterol-C5-desaturase (ERG3 delta-5-desaturase homolog, S. cerevisiae)- | −2.87 |
| DAP4_3933 | 367 | AR | androgen receptor (dihydrotestosterone receptor; testicular feminization; | −2.84 |
| | | | spinal and bulbar muscular atrophy; Kennedy disease) | |
| DAP3_5880 | 3479 | IGF1 | insulin-like growth factor 1 (somatomedin C) | −2.84 |
| DAP1_1983 | 8802 | SUCLG1 | succinate-CoA ligase, GDP-forming, alpha subunit | −2.84 |
| DAP3_5885 | 23498 | HAAO | 3-hydroxyanthranilate 3,4-dioxygenase | −2.83 |
| DAP2_6048 | 735 | C9 | complement component 9 | −2.83 |
| DAP4_1959 | 9013 | TAF1C | TATA box binding protein (TBP)-associated factor, RNA polymerase I, | −2.82 |
| | | | C, 110 kDa | |
| DAP4_2356 | 1371 | CPOX | coproporphyrinogen oxidase | −2.82 |
| DAP4_5179 | 7507 | XPA | xeroderma pigmentosum, complementation group A | −2.82 |
| DAP4_0915 | 3026 | HABP2 | hyaluronan binding protein 2 | −2.81 |
| DAP3_3625 | 2690 | GHR | growth hormone receptor | −2.77 |
| DAP4_1564 | 5105 | PCK1 | phosphoenolpyruvate carboxykinase 1 (soluble) | −2.76 |
| DAP2_1588 | 6718 | AKR1D1 | aldo-keto reductase family 1, member D1 (delta 4-3-ketosteroid-5-beta- | −2.76 |
| DAP3_1407 | 128 | ADH5 | alcohol dehydrogenase 5 (class III), chi polypeptide | −2.75 |
| DAP3_5846 | 16 | AARS | alanyl-tRNA synthetase | −2.70 |
| DAP4_1895 | 732 | C8B | complement component 8, beta polypeptide | −2.69 |
| DAP1_2114 | 51237 | MGC29506 | NA | −2.67 |
| DAP4_3262 | 10159 | ATP6AP2 | ATPase, H+ transporting, lysosomal accessory protein 2 | −2.67 |
| DAP4_2906 | 9732 | DOCK4 | dedicator of cytokinesis 4 | −2.66 |
| DAP4_4262 | 5627 | PROS1 | protein S (alpha) | −2.66 |
| DAP4_5591 | 7709 | ZBTB17 | zinc finger and BTB domain containing 17 | −2.65 |
| DAP1_2989 | 1603 | DAD1 | defender against cell death 1 | −2.65 |
| DAP4_0781 | 1678 | TIMM8A | translocase of inner mitochondrial membrane 8 homolog A (yeast) | −2.65 |
| DAP3_5291 | 3155 | HMGCL | 3-hydroxymethyl-3-methylglutaryl-Coenzyme A lyase | −2.65 |
| | | | (hydroxymethylglutaricaciduria) | |
| DAP3_4919 | 725 | C4BPB | complement component 4 binding protein, beta | −2.62 |
| DAP4_5846 | 7189 | TRAF6 | TNF receptor-associated factor 6 | −2.62 |
| DAP1_0147 | 1967 | EIF2B1 | eukaryotic translation initiation factor 2B, subunit 1 alpha, 26 kDa | −2.61 |
| DAP1_0559 | 3990 | LIPC | lipase, hepatic | −2.60 |
| DAP4_5383 | 10026 | PIGK | phosphatidylinositol glycan anchor biosynthesis, class K | −2.60 |
| DAP4_5653 | 80344 | WDR23 | WD repeat domain 23 | −2.59 |
| DAP4_0010 | 5982 | RFC2 | replication factor C (activator 1) 2, 40 kDa | −2.58 |
| DAP4_5452 | 2915 | GRM5 | glutamate receptor, metabotropic 5 | −2.56 |
| DAP3_1646 | 6391 | SDHC | succinate dehydrogenase complex, subunit C, integral membrane protein, | −2.55 |
| DAP3_2354 | 2073 | ERCC5 | excision repair cross-complementing rodent repair deficiency, | −2.54 |
| | | | complementation group 5 (xeroderma pigmentosum, complementation | |
| | | | group G (Cockayne syndrome)) | |
| DAP1_2179 | 2158 | F9 | coagulation factor IX (plasma thromboplastic component, Christmas | −2.54 |
| | | | disease, hemophilia B) | |
| DAP2_2062 | 157567 | ANKRD46 | ankyrin repeat domain 46 | −2.54 |
| DAP3_2994 | 417 | ART1 | ADP-ribosyltransferase 1 | −2.54 |
| DAP3_1761 | 1486 | CTBS | chitobiase, di-N-acetyl- | −2.54 |
| DAP3_3022 | 2542 | SLC37A4 | solute carrier family 37 (glucose-6-phosphate transporter), member 4 | −2.53 |
| DAP4_3697 | 211 | ALAS1 | aminolevulinate, delta-, synthase 1 | −2.53 |
| DAP4_5013 | 27072 | VPS41 | vacuolar protein sorting 41 homolog (S. cerevisiae) | −2.51 |
| DAP3_1312 | 2642 | GCGR | glucagon receptor | −2.51 |
| DAP1_5069 | 10694 | CCT8 | chaperonin containing TCP1, subunit 8 (theta) | −2.51 |
| DAP1_0656 | 25874 | BRP44 | brain protein 44 | −2.50 |
| DAP1_5381 | 2868 | GRK4 | G protein-coupled receptor kinase 4 | −2.50 |
| DAP4_1861 | 3336 | HSPE1 | heat shock 10 kDa protein 1 (chaperonin 10) | −2.50 |
| DAP2_5258 | 79731 | NARS2 | asparaginyl-tRNA synthetase 2, mitochondrial (putative) | −2.49 |
| DAP1_5672 | 667 | DST | dystonin | −2.49 |
| DAP1_5518 | 27032 | ATP2C1 | ATPase, Ca++ transporting, type 2C, member 1 | −2.48 |
| DAP4_3497 | 10327 | AKR1A1 | aldo-keto reductase family 1, member A1 (aldehyde reductase) | −2.48 |
| DAP1_1085 | 2010 | EMD | emerin (Emery-Dreifuss muscular dystrophy) | −2.47 |
| DAP4_5050 | 799 | CALCR | calcitonin receptor | −2.45 |
| DAP3_4223 | 22839 | DLGAP4 | discs, large (Drosophila) homolog-associated protein 4 | −2.45 |
| DAP4_3111 | 6240 | RRM1 | ribonucleotide reductase M1 polypeptide | −2.44 |
| DAP4_3810 | 29937 | NENF | neuron derived neurotrophic factor | −2.44 |
| DAP1_3440 | 29887 | SNX10 | sorting nexin 10 | −2.44 |
| DAP3_5257 | 5372 | PMM1 | phosphomannomutase 1 | −2.44 |
| DAP1_5842 | 6999 | TDO2 | tryptophan 2,3-dioxygenase | −2.43 |
| DAP4_3363 | 2944 | GSTM1 | glutathione S-transferase M1 | −2.43 |
| DAP1_5123 | 6721 | SREBF2 | sterol regulatory element binding transcription factor 2 | −2.42 |
| DAP4_0140 | 26469 | PTPN18 | protein tyrosine phosphatase, non-receptor type 18 (brain-derived) | −2.42 |
| DAP3_1623 | 27163 | ASAHL | N-acylsphingosine amidohydrolase (acid ceramidase)-like | −2.41 |
| DAP2_4928 | 5336 | PLCG2 | phospholipase C, gamma 2 (phosphatidylinositol-specific) | −2.41 |
| DAP3_5959 | 3760 | KCNJ3 | potassium inwardly-rectifying channel, subfamily II, member 3 | −2.40 |
| DAP3_1753 | 5833 | PCYT2 | phosphate cytidylyltransferase 2, ethanolamine | −2.40 |
| DAP4_4304 | 2705 | GJB1 | gap junction protein, beta 1, 32 kDa | −2.39 |
| DAP3_5067 | 7108 | TM7SF2 | transmembrane 7 superfamily member 2 | −2.39 |
| DAP4_5379 | 8991 | SELENBP1 | selenium binding protein 1 | −2.38 |
| DAP4_3066 | 316 | AOX1 | aldehyde oxidase 1 | −2.37 |
| DAP3_2882 | 10444 | ZER1 | zer-1 homolog (C. elegans) | −2.37 |
| DAP4_6012 | 130 | ADH6 | alcohol dehydrogenase 6 (class V) | −2.36 |
| DAP3_5076 | 2956 | MSH6 | mutS homolog 6 (E. coli) | −2.36 |
| DAP2_3569 | 8671 | SLC4A4 | solute carrier family 4, sodium bicarbonate cotransporter, member 4 | −2.34 |
| DAP3_3988 | 9097 | USP14 | ubiquitin specific peptidase 14 (tRNA-guanine transglycosylase) | −2.34 |
| DAP3_6123 | 727 | C5 | complement component 5 | −2.32 |
| DAP4_0949 | 5893 | RAD52 | RAD52 homolog (S. cerevisiae) | −2.32 |
| DAP4_0979 | 116496 | FAM129A | family with sequence similarity 129, member A | −2.31 |
| DAP4_2296 | 10458 | BAIAP2 | BAI1-associated protein 2 | −2.31 |
| DAP1_1550 | 6744 | SSFA2 | sperm specific antigen 2 | −2.30 |
| DAP2_6140 | 5446 | PON3 | paraoxonase 3 | −2.30 |
| DAP3_2198 | 2646 | GCKR | glucokinase (hexokinase 4) regulator | −2.30 |
| DAP3_3783 | 1385 | CREB1 | cAMP responsive element binding protein 1 | −2.30 |
| DAP3_3049 | 23316 | CUTL2 | cut-like 2 (Drosophila) | −2.29 |
| DAP1_5546 | 6427 | SFRS2 | splicing factor, arginine/serine-rich 2 | −2.28 |
| DAP4_0984 | 3156 | HMGCR | 3-hydroxy-3-methylglutaryl-Coenzyme A reductase | −2.28 |
| DAP3_5468 | 2677 | GGCX | gamma-glutamyl carboxylase | −2.27 |
| DAP2_5898 | 1555 | CYP2B6 | cytochrome P450, family 2, subfamily B, polypeptide 6 | −2.26 |
| DAP4_3279 | 7739 | ZNF185 | zinc finger protein 185 (LIM domain) | −2.25 |
| DAP3_1562 | 378 | ARF4 | ADP-ribosylation factor 4 | −2.23 |
| DAP4_3503 | 10965 | ACOT2 | acyl-CoA thioesterase 2 | −2.22 |
| DAP3_0889 | 513 | ATP5D | ATP synthase, H+ transporting, mitochondrial F1 complex, delta subunit | −2.22 |
| DAP2_4148 | 1369 | CPN1 | carboxypeptidase N, polypeptide 1 | −2.20 |
| DAP2_1935 | 5331 | PLCB3 | phospholipase C, beta 3 (phosphatidylinositol-specific) | −2.20 |
| DAP3_2137 | 3642 | INSM1 | insulinoma-associated 1 | −2.18 |
| DAP4_3027 | 5442 | POLRMT | polymerase (RNA) mitochondrial (DNA directed) | −2.14 |
| DAP3_5700 | 11145 | HRASLS3 | HRAS-like suppressor 3 | −2.13 |

TABLE 3

| | | |
|---|---|---|
| # genes | NES | FDR |

(a) Gene sets correlated with poor survival

| | | | |
|---|---|---|---|
| Experimental perturbation gene set | | | |
| IFNA_HCMV_6HRS_UP | 56 | 2.30 | 0.000 |
| CROONQUIST_IL6_STROMA_UP | 34 | 2.19 | 0.003 |
| SANA_IFNG_ENDOTHELIAL_UP | 30 | 1.98 | 0.034 |
| IFN_ALPHA_UP | 30 | 1.96 | 0.029 |
| SANA_TNFA_ENDOTHELIAL_UP | 46 | 1.96 | 0.024 |
| RADAEVA_IFNA_UP | 29 | 1.96 | 0.020 |
| ADIP_HUMAN_DN | 18 | 1.95 | 0.018 |
| IFNA_UV-CMV_COMMON_HCMV_6HRS_UP | 33 | 1.94 | 0.019 |
| O6BG_RESIST_MEDULLOBLASTOMA_DN | 24 | 1.90 | 0.025 |
| HADDAD_HSC_CD10_UP | 165 | 1.86 | 0.034 |
| TGFBETA_ALL_UP | 50 | 1.85 | 0.035 |
| ZUCCHI_EPITHELIAL_DN | 34 | 1.83 | 0.038 |
| BRG1_ALAB_DN | 18 | 1.83 | 0.036 |
| CROONQUIST_RAS_STROMA_DN | 18 | 1.81 | 0.041 |
| HINATA_NFKB_UP | 91 | 1.71 | 0.087 |
| Litrature-based pathway gene set | | | |
| INFLAMMATORY_RESPONSE_PATHWAY | 25 | 1.90 | 0.023 |

(b) Gene sets correlated with good survival

| | | | |
|---|---|---|---|
| Experimental perturbation gene set | | | |
| FETAL_LIVER_VS_ADULT_LIVER_GNF2 | 44 | −2.20 | 0.002 |
| Litrature-based pathway gene set | | | |
| ANDROGEN_AND_ESTROGEN_METABOLISM | 21 | −2.17 | 0.001 |
| FATTY_ACID_METABOLISM | 57 | −2.15 | 0.001 |
| TRYPTOPHAN_METABOLISM | 44 | −2.08 | 0.002 |
| BILE_ACID_BIOSYNTHESIS | 17 | −2.02 | 0.004 |
| ELECTRON_TRANSPORT_CHAIN | 48 | −2.01 | 0.003 |
| VALINE_LEUCINE_AND_ISOLEUCINE_DEGRADATION | 23 | −1.99 | 0.003 |
| INOSITOL_PHOSPHATE_METABOLISM | 19 | −1.90 | 0.007 |
| BUTANOATE_METABOLISM | 20 | −1.89 | 0.006 |
| BETA_ALANINE_METABOLISM | 21 | −1.81 | 0.014 |
| PYRUVATE_METABOLISM | 26 | −1.75 | 0.023 |
| GLYCINE_SERINE_AND_THREONINE_METABOLISM | 23 | −1.74 | 0.023 |
| GAMMA_HEXACHLOROCYCLOHEXANE_DEGRADATION | 25 | −1.71 | 0.028 |
| GLYCEROLIPID_METABOLISM | 27 | −1.70 | 0.028 |

NES: normalized enrichment score, FDR: false discovery rate

TABLE 4

Inflammation

| Prediction | None | Mild | Moderate | Severe |
|---|---|---|---|---|
| Poor survival | 2 | 11 | 9 | 5 |
| Good survival | 4 | 17 | 22 | 11 |

Fisher's exact test, p = 0.89
Scored according to Batts K, Ludwig J, Am J Surg Pathol 19: 1409, 1995

TABLE 5

| | |
|---|---|
| Hazard | 95% CI |

| | | | | |
|---|---|---|---|---|
| Variable | ratio | low | high | p-value |

Early recurrence

| | | | | |
|---|---|---|---|---|
| Multimodularity | 1.95 | 1.13 | 3.37 | 0.02 |
| Vascular invasion | 1.72 | 1.11 | 2.66 | 0.02 |
| AFP >100 ng/mL | 1.94 | 1.21 | 3.12 | 0.006 |

Late recurrence

| | | | | |
|---|---|---|---|---|
| Multimodularity | 2.10 | 0.73 | 6.06 | 0.17 |
| Vascular invasion | 0.85 | 0.38 | 1.92 | 0.70 |
| AFP >100 ng/mL | 0.45 | 0.17 | 1.19 | 0.11 |

Survival

| | | | | |
|---|---|---|---|---|
| Multimodularity | 1.66 | 0.77 | 3.59 | 0.19 |
| Vascular invasion | 2.05 | 1.12 | 3.76 | 0.02 |
| AFP >100 ng/mL | 2.10 | 1.08 | 4.07 | 0.03 |

AFP: alpha-fetoprotein

TABLE 6

| | |
|---|---|
| Hazard | 95% CI |

| | | | | |
|---|---|---|---|---|
| Variable | ratio | low | high | p-value |

Late recurrence (longer follow-up patients, n = 168)

| | | | | |
|---|---|---|---|---|
| Late recurrence signature | 2.94 | 1.39 | 6.20 | 0.005 |

Late recurrence (BCLC ≦ A, n = 204)

| | | | | |
|---|---|---|---|---|
| Late recurrence signature | 2.97 | 1.37 | 6.45 | 0.006 |

Survival (BCLC ≦ A, n = 204)

| | | | | |
|---|---|---|---|---|
| Survival signature | 1.93 | 0.87 | 4.28 | 0.10 |
| AFP >100 ng/mL | 2.30 | 1.04 | 5.05 | 0.04 |
| Vascular invasion | 1.80 | 0.84 | 3.88 | 0.13 |

Survival (longer follow-up patients, BCLC ≦ A, n = 154)

| | | | | |
|---|---|---|---|---|
| Survival signature | 2.04 | 0.91 | 4.59 | 0.08 |
| AFP >100 ng/mL | 2.13 | 0.95 | 4.76 | 0.07 |
| Vascular invasion | 2.01 | 0.92 | 4.36 | 0.08 |

AFP: alpha-fetoprotein

TABLE 7
A
Clonality analysis of paired primary and recurrent HCC

| | “Heterozygote in recurrence”/ | | “Homozygote in recurrence”/ | | Primary tumor | Recurrent tumor |
|---|---|---|---|---|---|---|
| Case ID | “Homozygote in primary” | | “Heterozygote in primary” | | subtype* | subtype* |
| hcc_018 | 26% | (562/2167) | 25% | (268/1086) | S2 | S1 |
| hcc_044 | 6% | (178/2887) | 13% | (156/1200) | S1 | S3 |
| hcc_082 | 1% | (31/3244) | 10% | (123/1205) | S3 | S2 |
| hcc_075 | 9% | (218/2548) | 15% | (171/1168) | S2 | S1 |
| hcc_101 | 8% | (168/2063) | 32% | (310/967) | S2 | S2 |

| | | | | |
|---|---|---|---|---|
| hcc_104 | — | — | S3 | S1 |

B
Clonality analysis of paired primary and recurrent/metastatic non-HCC tumors

| | | |
|---|---|---|
| | “Heterozygote in recurrence”/ | “Homozygote in recurrence”/ |
| Cancer type | “Homozygote in primary” | “Heterozygote in primary” |

| | | | | |
|---|---|---|---|---|
| Endometrial 1 | 0.1% | (19/40897) | 0.8% | (124/15033) |
| Endometrial 2 | 1.9% | (1621/83369) | 0.6% | (160/24907) |
| Endometrial 3 | 0.2% | (198/82423) | 0.9% | (229/26372) |
| Ovarian 1 | 1.9% | (1686/90069) | 1.7% | (300/17747) |
| Ovarian 2 | 1.3% | (1182/91547) | 3.3% | (549/16621) |
| Ovarian 3 | 0.3% | (121/44828) | 2.2% | (218/10075) |
| Ovarian 4 | 0.2% | (103/43429) | 4.5% | (441/9781) |
| Renal 1 | 1.6% | (682/42699) | 0.1% | (13/13701) |
| Renal 2 | 1.9% | (796/42753) | 0.1% | (9/12720) |
| Renal 3 | 0.1% | (55/42518) | 1.2% | (157/13154) |
| DLBCL 1 | 1.1% | (6503/605724) | 2.8% | (6001/216597) |
| DLBCL 2 | 0.9% | (5361/598243) | 2.2% | (5089/228143) |
| DLBCL 3 | 0.4% | (2216/625092) | 0.6% | (1402/239662) |
| DLBCL 4 | 1.0% | (6377/618375) | 2.0% | (4525/222075) |
| DLBCL 5 | 1.8% | (10689/582552) | 2.5% | (5697/232207) |
| DLBCL 6 | 0.6% | (3773/600753) | 6.1% | (13368/219845) |

HCV: Hepatitis C virus, HBV: Hepatitis B virus
*Molecular subtypes of HCC defined by a meta-analysis of published frozen sample-based microarray datasets (Hoshida et al. Manuscripts in preparation).
“Heterozygote in recurrence”/“Homozygote in primary” in adjacent non-tumor liver tissues of hcc_082, hcc_075, and Hela cells were 0.1% (4/3759), 0.6% (20/3275), and 0.3% (10/3701), respectively.
“Homozygote in recurrence”/“Heterozygote in primary” in adjacent non-tumor liver tissues of hcc_082, hcc_075, and Hela cells were 0.6% (11/1869), 2.3% (38/1676), and 0.7% (8/1216), respectively.
DLBCL: diffuse large B-cell lymphoma
Endometrial, ovarian, and renal cancers were profiles on Afymetrix 500k SNP array.
DLBCL samples were profiled on Affymetrix SNP 6.0 array.

TABLE 8

| Tissue type | Disease | # samples | Reference |
|---|---|---|---|
| Brain | Glioblastoma | 50 | Cancer Res 63; 1602, 2003 |
| | Medulloblastoma | 60 | Nature 415;436, 2002 |
| | Medulloblastoma | 23 | Nature Genet 29; 143, 2001 |
| Breast | Breast cancer | 73 | Unpublished |
| | Breast cancer | 49 | PNAS 98; 11462, 2001, |
| | | | Lancet 361; 1590, 2003 |
| | Breast cancer | 40 | Unpublished |
| Lung | Lung cancer | 62 | PNAS 98; 13790, 2001 |
| | Lung cancer | 86 | Nature Med 8; 816, 2002 |
| Stomach | Gastric cancer | 30 | Cancer Res 62; 233, 2003 |
| Liver | Hepatocellular carcinoma | 49 | Cancer Res 64; 7263, 2004 |
| | Hepatocellular carcinoma | 60 | Lancet 361; 923, 2003 |
| Ovary | Ovarian cancer | 113 | Unpublished |
| Prostate | Prostate cancer | 102 | Cacer Cell 1; 203, 2002 |
| | Prostate cancer | 120 | Unpublished |
| | Prostate cancer | 80 | Unpublished |
| Hematopoetic | Diffuse large B-cell lymphoma | 176 | Blood 105; 1851, 2005 |
| | Diffuse large B-cell lymphoma | 210 | Blood 102; 3871, 2003 |
| | Acute myeloid/ | 52 | Unpublished |
| | lymphoblastic leukemia | | |
| | Mixed-lineage leukemia | 72 | Nature Genet 30; 41, 2002 |
| Skin | Melanoma | 115 | Unpublished |
| Astrocyte | Astrocytoma | 13 | Cancer Res 63; 1865, 2003 |
| Cancer & normal | Cancer & normal tissues | 280 | PNAS 98; 15149, 2001 |
| tissues* | | | |
| Cancer tissues* | Primary & metastatic cancers | 76 | Nature Genet 33; 49, 2003 |
| Normal tissues* | Normal tissues | 158 | PNAS 04; 101; 6062 |
| | | 2149 | |

*Panel of multiple tissue types

TABLE 9

| | | | Fold change in fresh RNA | | | | | |
|---|---|---|---|---|---|---|---|---|
| | All genes | | >2-fold | | >5-fold | | >10-fold | |
| # genes DHL4 > Hela in fresh RNA | 3156 | | 811 | | 185 | | 93 | |
| # genes with concordant change in FFPE RNA | 2282 | (72%) | 687 | (85%) | 180 | (97%) | 91 | (98%) |
| # genes with discordant change in FFPE RNA | 874 | (28%) | 124 | (15%) | 5 | (3%) | 2 | (2%) |
| # genes DHL4 < Hela in fresh RNA | 2988 | | 1056 | | 339 | | 138 | |
| # genes with concordant change in FFPE RNA | 2135 | (71%) | 905 | (86%) | 321 | (95%) | 137 | (99%) |
| # genes with discordant change in FFPE RNA | 853 | (29%) | 151 | (14%) | 18 | (5%) | 1 | (1%) |

TABLE 10

| | |
|---|---|
| Prediction | Outcome |

| algorithm | Survival | Recurrence |
|---|---|---|
| CART | 40% | 21% |
| k-NN, 1 neighbor | 41% | 18% |
| k-NN, 3 neighbors | 43% | 18% |
| k-NN, 5 neighbors | 31% | 19% |
| k-NN, 7 neighbors | 36% | 19% |
| WV, 10 markers | 38% | 41% |
| WV, 50 markers | 48% | 45% |
| WV, 100 markers | 49% | 30% |
| SVM | 43% | 23% |

CART: classification and regression trees,
k-NN: k-nearest neighbor,
WV: weighted voting,
SVM: support vector machine

TABLE 11

| | Training Set | | Validation Set | | |
|---|---|---|---|---|---|
| Characteristic | (N = 82) | | (N = 225) | | P Value |
| Age - yr | | | | | <0.001 |
| Median | 59 | | 66 | | |
| Interquartile range | 52-64 | | 57-71 | | |
| Male sex - no. (%) | 64 | (78) | 173 | (77) | 0.88 |
| HCV infection - no. (%) | 60 | (73) | 104 | (48) | <0.001 |
| HBV infection - no. (%) | 17 | (21) | 61 | (29) | 0.25 |
| Alcohol use - no. (%) | 3 | (4) | 19 | (9) | 0.22 |
| Tumer diameter - cm | | | | | <0.001 |
| Median | 2.2 | | 3.5 | | |
| Interquartile range | 1.7-3.2 | | 2.3-5.5 | | |
| Histopathologic grade - | | | | | |
| no. (%)† | | | | | |
| Well differentiated | 18 | (22) | 34 | (26) | 0.68 |
| Moderately differentiated | 49 | (60) | 80 | (60) | |
| Poorly differentiated | 15 | (18) | 19 | (14) | |
| Vascular invasion - no. (%) | 4 | (5) | 74 | (34) | <0.001 |
| BCLC stage - no. (%) | | | | | |
| O | 25 | (30) | 21 | (9) | 1.00† |
| A | 50 | (61) | 183 | (82) | <0.001‡ |
| B | 7 | (9) | 19 | (8) | |
| Child-Pugh class A - no. (%) | 72 | (88) | 204 | (97) | 0.52 |
| Alpha-fetoprotein >100 ng/ml - | 53 | (65) | 53 | (24) | 0.14 |
| no. (%) | | | | | |

| | | | |
|---|---|---|---|
| Median follow-up - yr | 7.8 | 2.2 | — |

*Some data were not available for all patients. The Barcelona Clinic Liver Cancer staging system (BCLC) ranks hepatocellular carcinoma in five stages, ranging from 0 (very early stage) to D (terminal stage). Histopathologic grade was defined according to the International Union Against Cancer (UICC). The Child-Pugh system classifies the severity of liver disease from A to C, with A representing the best liver function. HBV denotes hepatitis B virus, and HCV hepatitis C virus.
†P = 1.00 for the pairwise comparison of stages 0 and A with stage B.
‡P < 0.001 for the multiple comparison of stage 0, stage A, and stage B.

TABLE 12

| | Hazard Ratio | |
|---|---|---|
| Variable | (95% CI)* | P Value |
| Late-recurrence signature | 2.94 (1.39-6.20) | 0.005 |
| Overall survival | | |
| All 225 patients | | |
| Poor-prognosis signature | 2.08 (1.03-4.18) | 0.04 |
| Alpha-fetoprotein >100 ng/ml | 2.29 (1.14-4.61) | 0.02 |
| Vascular invasion | 2.01 (1.01-3.99) | 0.05 |
| 168 Patients with longer follow-up | | |
| Poor-prognosis signature | 2.56 (1.22-5.38) | 0.01 |
| Alpha-fetoprotein >100 ng/ml | 2.01 (0.94-4.26) | 0.07 |
| Vascular invasion | 2.20 (1.06-4.53) | 0.03 |

*The hazard ratio was for late recurrence in patients with the late-recurrence gene signature or for overall survival in patients with the poor-prognosis gene signature, as compared with those without the signature.

### Table 13

TABLE 13A
Genes correlated with higher late recurrence

| ProbeID | GeneID | Gene symbol | Description | Cox_score |
|---|---|---|---|---|
| DAP3_0162 | 2564 | GABRE | gamma-aminobutyric acid | 3.70 |
| | | | (GABA) A receptor, epsilon | |
| DAP1_5851 | 10875 | FGL2 | fibrinogen-like 2 | 3.64 |
| DAP3_2951 | 4060 | LUM | lumican | 3.57 |
| DAP3_2261 | 1012 | CDH13 | cadherin 13, H-cadherin (heart) | 3.28 |
| DAP3_2729 | 2877 | GPX2 | glutathione peroxidase 2 | 2.98 |
| | | | (gastrointestinal) | |
| DAP3_5744 | 11214 | AKAP13 | A kinase (PRKA) anchor | 2.97 |
| | | | protein 13 | |
| DAP3_0973 | 962 | CD48 | CD48 molecule | 2.94 |
| DAP4_3183 | 26136 | TES | testis derived transcript (3 LIM | 2.94 |
| | | | domains) | |
| DAP3_5985 | 915 | CD3D | CD3d molecule, delta (CD3- | 2.91 |
| | | | TCR complex) | |
| DAP1_2827 | 5196 | PF4 | platelet factor 4 (chemokine (C- | 2.91 |
| | | | X-C motif) ligand 4) | |
| DAP3_2439 | 2959 | GTF2B | general transcription factor IIB | 2.91 |
| DAP4_4005 | 6772 | STAT1 | signal transducer and activator | 2.89 |
| | | | of transcription 1, 91 kDa | |
| DAP4_5069 | 1551 | CYP3A7 | cytochrome P450, family 3, | 2.88 |
| | | | subfamily A, polypeptide 7 | |
| DAP4_3963 | 9358 | ITGBL1 | integrin, beta-like 1 (with EGF- | 2.86 |
| | | | like repeat domains) | |
| DAP4_2308 | 7128 | TNFAIP3 | tumor necrosis factor, alpha- | 2.86 |
| | | | induced protein 3 | |
| DAP1_0992 | 22913 | RALY | RNA binding protein, | 2.86 |
| | | | autoantigenic (hnRNP- | |
| | | | associated with lethal yellow | |
| | | | homolog (mouse)) | |
| DAP1_4954 | 3066 | HDAC2 | histone deacetylase 2 | 2.85 |
| DAP4_3045 | 8933 | FAM127A | family with sequence similarity | 2.83 |
| | | | 127, member A | |
| DAP3_1678 | 301 | ANXA1 | annexin A1 | 2.80 |
| DAP3_4082 | 6867 | TACC1 | transforming, acidic coiled-coil | 2.77 |
| | | | containing protein 1 | |
| DAP3_4302 | 7319 | UBE2A | ubiquitin-conjugating enzyme | 2.77 |
| | | | E2A (RAD6 homolog) | |
| DAP1_0368 | 4297 | MLL | myeloid/lymphoid or mixed- | 2.76 |
| | | | lineage leukemia (trithorax | |
| | | | homolog, Drosophila) | |
| DAP4_0063 | 9071 | CLDN10 | claudin 10 | 2.76 |
| DAP3_5935 | 1513 | CTSK | cathepsin K | 2.73 |
| DAP4_1577 | 26751 | SH3YL1 | SH3 domain containing, Ysc84- | 2.73 |
| | | | like 1 (S. cerevisiae) | |
| DAP4_2297 | 5337 | PLD1 | phospholipase D1, | 2.72 |
| | | | phosphatidylcholine-specific | |
| DAP1_0933 | 313 | AOAH | acyloxyacyl hydrolase | 2.70 |
| | | | (neutrophil) | |
| DAP3_6062 | 10097 | ACTR2 | ARP2 actin-related protein 2 | 2.70 |
| | | | homolog (yeast) | |
| DAP3_2534 | 56265 | CPXM1 | carboxypeptidase X (M14 | 2.70 |
| | | | family), member 1 | |
| DAP1_0174 | 6558 | SLC12A2 | solute carrier family 12 | 2.67 |
| | | | (sodium/potassium/chloride | |
| | | | transporters), member 2 | |
| DAP4_4219 | 5500 | PPP1CB | protein phosphatase 1, catalytic | 2.67 |
| | | | subunit, beta isoform | |
| DAP3_3294 | 4818 | NKG7 | natural killer cell group 7 | 2.63 |
| | | | sequence | |
| DAP1_3171 | 6546 | SLC8A1 | solute carrier family 8 | 2.63 |
| | | | (sodium/calcium exchanger), | |
| | | | member 1 | |
| DAP4_2902 | 1825 | DSC3 | desmocollin 3 | 2.62 |
| DAP3_2152 | 5698 | PSMB9 | proteasome (prosome, | 2.61 |
| | | | macropain) subunit, beta type, 9 | |
| | | | (large multifunctional | |
| | | | peptidase 2) | |
| DAP2_5968 | 3912 | LAMB1 | laminin, beta 1 | 2.61 |
| DAP4_2828 | 2804 | GOLGB1 | golgi autoantigen, golgin | 2.60 |
| | | | subfamily b, macrogolgin (with | |
| | | | transmembrane signal), 1 | |
| DAP1_3780 | 10125 | RASGRP1 | RAS guanyl releasing protein 1 | 2.59 |
| | | | (calcium and DAG-regulated) | |
| DAP4_1324 | 10537 | UBD | ubiquitin D | 2.59 |
| DAP4_4256 | 10609 | SC65 | NA | 2.58 |
| DAP4_5268 | 4323 | MMP14 | matrix metallopeptidase 14 | 2.56 |
| | | | (membrane-inserted) | |
| DAP3_4222 | 54476 | TRIAD3 | NA | 2.55 |
| DAP4_2155 | 5111 | PCNA | proliferating cell nuclear | 2.55 |
| | | | antigen | |
| DAP1_0054 | 6925 | TCF4 | transcription factor 4 | 2.50 |
| DAP3_2983 | 2487 | FRZB | frizzled-related protein | 2.50 |
| DAP4_5163 | 1366 | CLDN7 | claudin 7 | 2.49 |
| | | | POU domain, class 2, | |
| DAP1_3805 | 5452 | POU2F2 | transcription factor 2 | 2.49 |
| DAP4_3972 | 10144 | FAM13A1 | family with sequence similarity | 2.46 |
| | | | 13, member A1 | |
| DAP1_4144 | 4436 | MSH2 | mutS homolog 2, colon cancer, | 2.45 |
| | | | nonpolyposis type 1 (E. coli) | |
| DAP4_2198 | 999 | CDH1 | cadherin 1, type 1, E-cadherin | 2.42 |
| | | | (epithelial) | |
| DAP3_3561 | 28984 | C13orf15 | chromosome 13 open reading | 2.42 |
| | | | frame 15 | |
| DAP1_3132 | 114876 | OSBPL1A | oxysterol binding protein-like 1A | 2.41 |
| DAP4_0119 | 8343 | HIST1H2BF | histone cluster 1, H2bf | 2.39 |
| DAP3_3533 | 3960 | LGALS4 | lectin, galactoside-binding, | 2.38 |
| | | | soluble, 4 (galectin 4) | |
| DAP4_1020 | 972 | CD74 | CD74 molecule, major | 2.38 |
| | | | histocompatibility complex, | |
| | | | class II invariant chain | |
| DAP1_1697 | 4803 | NGFB | nerve growth factor, beta | 2.34 |
| | | | polypeptide | |
| DAP1_5943 | 6591 | SNAI2 | snail homolog 2 (Drosophila) | 2.34 |
| DAP3_3002 | 3304 | HSPA1B | heat shock 70 kDa protein 1B | 2.32 |
| DAP1_5371 | 55719 | C10orf6 | chromosome 10 open reading | 2.32 |
| | | | frame 6 | |
| DAP3_1595 | 10487 | CAP1 | CAP, adenylate cyclase- | 2.32 |
| | | | associated protein 1 (yeast) | |
| DAP3_0758 | 546 | ATRX | alpha thalassemia/mental | 2.31 |
| | | | retardation syndrome X-linked | |
| | | | (RAD54 homolog, | |
| | | | S. cerevisiae) | |
| DAP3_4297 | 9019 | MPZL1 | myelin protein zero-like 1 | 2.30 |

TABLE 13B
Genes correlated with lower late recurrence

| ProbeID | GeneID | Gene symbol | Description | Cox_score |
|---|---|---|---|---|
| DAP1_2192 | 8834 | TMEM11 | transmembrane protein 11 | −3.94 |
| DAP3_1753 | 5833 | PCYT2 | phosphate cytidylyltransferase 2, | −3.74 |
| | | | ethanolamine | |
| DAP3_3624 | 10400 | PEMT | phosphatidylethanolamine N- | −3.28 |
| | | | methyltransferase | |
| DAP1_4086 | 3931 | LCAT | lecithin-cholesterol acyltransferase | −3.21 |
| DAP3_2733 | 6309 | SC5DL | sterol-C5-desaturase (ERG3 delta- | −3.19 |
| | | | 5-desaturase homolog, | |
| | | | S. cerevisiae)-like | |
| DAP3_0230 | 4128 | MAOA | monoamine oxidase A | −3.12 |
| DAP4_2988 | 5307 | PITX1 | paired-like homeodomain | −3.11 |
| | | | transcription factor 1 | |
| DAP1_5275 | 4184 | SMCP | sperm mitochondria-associated | −3.11 |
| | | | cysteine-rich protein | |
| DAP3_2225 | 26227 | PHGDH | phosphoglycerate dehydrogenase | −3.07 |
| DAP1_1861 | 4086 | SMAD1 | SMAD family member 1 | −3.07 |
| DAP3_2216 | 5502 | PPP1R1A | protein phosphatase 1, regulatory | −3.02 |
| | | | (inhibitor) subunit 1A | |
| DAP3_0220 | 9816 | KIAA0133 | KIAA0133 | −3.02 |
| DAP1_3348 | 4675 | NAP1L3 | nucleosome assembly protein 1-like 3 | −2.99 |
| DAP3_2840 | 7392 | USF2 | upstream transcription factor 2, c- | −2.98 |
| | | | fos interacting | |
| DAP4_2813 | 151 | ADRA2B | adrenergic, alpha-2B-, receptor | −2.96 |
| DAP3_4874 | 671 | BPI | bactericidal/permeability- | −2.93 |
| | | | increasing protein | |
| DAP3_4090 | 7068 | THRB | thyroid hormone receptor, beta | −2.92 |
| | | | (erythroblastic leukemia viral (v- | |
| | | | erb-a) oncogene homolog 2, avian) | |
| DAP3_1558 | 5691 | PSMB3 | proteasome (prosome, macropain) | −2.91 |
| | | | subunit, beta type, 3 | |
| DAP1_1001 | 5617 | PRL | prolactin | −2.87 |
| DAP3_5885 | 23498 | HAAO | 3-hydroxyanthranilate 3,4- | −2.84 |
| | | | dioxygenase | |
| DAP1_0718 | 1015 | CDH17 | cadherin 17, LI cadherin (liver- | −2.76 |
| | | | intestine) | |
| DAP2_5009 | 9925 | ZBTB5 | zinc finger and BTB domain | −2.74 |
| | | | containing 5 | |
| DAP1_5877 | 839 | CASP6 | caspase 6, apoptosis-related | −2.70 |
| | | | cysteine peptidase | |
| DAP4_4067 | 9414 | TJP2 | tight junction protein 2 (zona | −2.68 |
| | | | occludens 2) | |
| DAP1_1550 | 6744 | SSFA2 | sperm specific antigen 2 | −2.67 |
| DAP1_1628 | 5375 | PMP2 | peripheral myelin protein 2 | −2.66 |
| DAP3_3153 | 148 | ADRA1A | adrenergic, alpha-1A-, receptor | −2.65 |
| DAP3_3988 | 9097 | USP14 | ubiquitin specific peptidase 14 | −2.65 |
| | | | (tRNA-guanine transglycosylase) | |
| DAP3_3049 | 23316 | CUTL2 | cut-like 2 (Drosophila) | −2.64 |
| DAP4_6060 | 5454 | POU3F2 | POU domain, class 3, transcription | −2.63 |
| | | | factor 2 | |
| DAP4_2394 | 3973 | LHCGR | luteinizing hormone/choriogonadotropin | −2.62 |
| | | | receptor | |
| DAP1_1164 | 7016 | TESK1 | testis-specific kinase 1 | −2.61 |
| DAP1_2966 | 53 | ACP2 | acid phosphatase 2, lysosomal | −2.60 |
| DAP4_2059 | 84253 | GARNL3 | GTPase activating Rap/RanGAP | −2.59 |
| | | | domain-like 3 | |
| DAP3_0654 | 22837 | COBLL1 | COBL-like 1 | −2.58 |
| DAP3_1360 | 6817 | SULT1A1 | sulfotransferase family, cytosolic, | −2.57 |
| | | | 1A, phenol-preferring, member 1 | |
| DAP1_4205 | 11005 | SPINK5 | serine peptidase inhibitor, Kazal | −2.57 |
| | | | type 5 | |
| DAP1_5104 | 5575 | PRKAR1B | protein kinase, cAMP-dependent, | −2.57 |
| | | | regulatory, type I, beta | |
| DAP1_3995 | 2230 | FDX1 | ferredoxin 1 | −2.56 |
| DAP4_4227 | 1602 | DACH1 | dachshund homolog 1 (Drosophila) | −2.55 |
| DAP3_1272 | 5498 | PPOX | protoporphyrinogen oxidase | −2.54 |
| DAP3_2137 | 3642 | INSM1 | insulinoma-associated 1 | −2.54 |
| DAP1_2021 | 23640 | HSPBP1 | NA | −2.53 |
| DAP2_2968 | 10402 | ST3GAL6 | ST3 beta-galactoside alpha-2,3- | −2.52 |
| | | | sialyltransferase 6 | |
| DAP3_0587 | 11338 | U2AF2 | U2 small nuclear RNA auxiliary | −2.51 |
| | | | factor 2 | |
| DAP1_0041 | 10912 | GADD45G | growth arrest and DNA-damage- | −2.51 |
| | | | inducible, gamma | |
| DAP2_1307 | 4311 | MME | membrane metallo-endopeptidase | −2.50 |
| DAP1_6126 | 6005 | RHAG | Rh-associated glycoprotein | −2.47 |
| DAP3_5257 | 5372 | PMM1 | phosphomannomutase 1 | −2.47 |
| DAP3_2882 | 10444 | ZER1 | zer-1 homolog (C. elegans) | −2.45 |
| DAP3_0889 | 513 | ATP5D | ATP synthase, H+ transporting, | −2.43 |
| | | | mitochondrial F1 complex, delta | |
| | | | subunit | |
| DAP1_4214 | 3010 | HIST1H1T | histone cluster 1, H1t | −2.43 |
| DAP1_2555 | 2692 | GHRHR | growth hormone releasing hormone | −2.42 |
| | | | receptor | |
| DAP2_5025 | 1138 | CHRNA5 | cholinergic receptor, nicotinic, | −2.42 |
| | | | alpha 5 | |
| DAP1_5708 | 23169 | SLC35D1 | solute carrier family 35 (UDP- | −2.41 |
| | | | glucuronic acid/UDP-N- | |
| | | | acetylgalactosamine dual | |
| | | | transporter), member D1 | |
| DAP3_0247 | 2742 | GLRA2 | glycine receptor, alpha 2 | −2.39 |
| DAP1_3075 | 320 | APBA1 | amyloid beta (A4) precursor | −2.39 |
| | | | protein-binding, family A, member | |
| | | | 1 (X11) | |
| DAP3_2198 | 2646 | GCKR | glucokinase (hexokinase 4) | −2.38 |
| | | | regulator | |
| DAP3_2994 | 417 | ART1 | ADP-ribosyltransferase 1 | −2.38 |
| DAP1_1661 | 8544 | PIR | pirin (iron-binding nuclear protein) | −2.36 |
| DAP3_4057 | 10202 | DHRS2 | dehydrogenase/reductase (SDR | −2.36 |
| | | | family) member 2 | |
| DAP3_0731 | 210 | ALAD | aminolevulinate, delta-, | −2.34 |
| | | | dehydratase | |
| DAP1_5013 | 2644 | GCHFR | GTP cyclohydrolase I feedback | −2.34 |
| | | | regulator | |
| DAP3_5285 | 2549 | GAB1 | GRB2-associated binding protein 1 | −2.32 |
| DAP1_1883 | 5167 | ENPP1 | ectonucleotide | −2.32 |
| | | | pyrophosphatase/phosphodiesterase 1 | |
| DAP3_1587 | 7010 | TEK | TEK tyrosine kinase, endothelial | −2.32 |
| | | | (venous malformations, multiple | |
| | | | cutaneous and mucosal) | |
| DAP4_1604 | 5569 | PKIA | protein kinase (cAMP-dependent, | −2.30 |
| | | | catalytic) inhibitor alpha | |
| DAP4_2424 | 1500 | CTNND1 | catenin (cadherin-associated | −2.28 |
| | | | protein), delta 1 | |
| DAP1_0130 | 978 | CDA | cytidine deaminase | −2.23 |
| DAP4_3842 | 4336 | MOBP | myelin-associated oligodendrocyte | −2.16 |
| | | | basic protein | |

TABLE 14
Characteristics of Patients at the Time of Enrollment

| Characteristics | N = 276 | |
|---|---|---|
| Age-yr | | |
| Median | 54 | |
| Interquartile range | 50-59 | |
| Male sex - no. (%) | 171 | (62%) |
| Etiology of cirrhosis | | |
| Hepatitis C virus infection - no. (%)* | 249 | (90%) |
| Hepatitis B virus infestion - no. (%) | 25 | (9%) |
| Alcohol use - no. (%) | 53 | (19%) |
| Esophageal/gastric varices - no. (%) | 65 | (24%) |
| Child-Pugh class A - no. (%) | 270 | (98%) |
| Bilirubin >1.0 mg/dL - no. (%) | 99 | (36%) |
| Albumin <4.0 g/dL - no. (%) | 64 | (24%) |
| Prothrombin time (international normalized | 113 | (49%) |
| ratio) >1.0 - no. (%) | | |
| Platelet count <100,000/mm³ - no. (%) | 116 | (42%) |
| Alanine amino transferase >100 IU - no. (%) | 148 | (54%) |
| Alpha-fetoprotein >20 ng/mL - no. (%) | 44 | (16%) |

The Child-Pugh system classifies the severity of liver damage from A to C. A representing the best liver function
*6 cases had co-infection with hepatitis B virus and 48 cases had alcohol abuse.

TABLE 15
Association of 186-Gene Survival Signature and Clinical Variables with
Clinical Outcome (Univariate Analysis)

| | | | | | Hepatocellular | |
|---|---|---|---|---|---|---|
| | Overall survival | | Hepatic decompensation | | carcinoma development | |
| Variable | Hazard Ratio (95% CI) | P Value | Hazard Ratio (95% CI) | P Value | Hazard Ratio (95% CI) | P Value |
| Poor-prognosis signature | 2.26 (1.42-3.59) | <0.001 | 2.09 (1.31-3.35) | 0.002 | 1.60 (0.94-2.71) | 0.08 |
| Age >50 yr | 1.78 (1.08-2.92) | 0.03 | 1.30 (0.60-2.10) | 0.29 | 1.50 (0.90-2.48) | 0.12 |
| Male sex | 0.92 (0.60-1.41) | 0.70 | 0.84 (0.55-1.28) | 0.42 | 1.35 (0.94-2.19) | 0.21 |
| Hepatitis C virus infection | 1.53 (0.75-3.12) | 0.24 | 0.96 (0.46-1.99) | 0.91 | 2.28 (0.97-5.38) | 0.05 |
| Hepatitis B virus infection | 0.66 (0.31-1.39) | 0.27 | 0.89 (0.41-1.93) | 0.77 | 0.51 (0.22-1.20) | 0.12 |
| Alcohol use | 0.87 (0.51-1.50) | 0.52 | 1.01 (0.50-1.70) | 0.96 | 1.03 (0.60-1.76) | 0.92 |
| Esophagealigastric varices | 1.89 (1.20-2.99) | 0.008 | 2.65 (1.71-4.11) | <0.001 | 1.62 (1.00-2.63) | 0.05 |
| Bilirubin >1.0 mg/dL | 2.30 (1.52-3.49) | <0.001 | 2.52 (1.66-3.84) | <0.001 | 1.85 (1.19-2.88) | 0.006 |
| Albumin <4.0 g/dL | 1.89 (1.20-2.97) | 0.006 | 1.71 (1.08-2.71) | 0.02 | 1.17 (0.69-1.98) | 0.55 |
| Prothrombin time | 0.91 (0.56-1.46) | 0.68 | 1.11 (0.72-1.73) | 0.53 | 0.98 (0.59-1.60) | 0.92 |
| (international normalized ratio) >1.0 | | | | | | |
| Platelet count <100,000/mm³ | 2.07 (1.35-3.18) | <0.001 | 1.83 (1.20-2.80) | 0.005 | 1.96 (1.25-3.07) | 0.004 |
| Alanine amino transferase >100 IU | 1.46 (0.95-2.23) | 0.08 | 1.01 (0.66-1.54) | 0.97 | 1.11 (0.71-1.71) | 0.65 |
| Alpha-fetoprotein >20 ng/mL | 1.52 (0.89-2.59) | 0.12 | 0.90 (0.49-1.66) | 0.75 | 1.26 (0.71-2.25) | 0.43 |

TABLE 16
Association of 186-Gene Survival Signature and Clinical
Variables with Clinical Outcome (Multivariate Analysis)

| | | |
|---|---|---|
| Variable | Hazard Ratio (95% CI) | P Value |

| | | | |
|---|---|---|---|
| Overall survival | | | |
| Poor-prognosis signature | 2.20 | (1.38-3.50) | <0.001 |
| Bilirubin >1.0 mg/dL | 1.96 | (1.28-3.01) | 0.002 |
| Platelet count <100,000/mm³ | 1.78 | (1.14-2.76) | 0.01 |
| Hepatic decompensation | | | |
| Poor-prognosis signature | 2.06 | (1.28-3.31) | 0.003 |
| Bilirubin >1.0 mg/dL | 2.25 | (1.46-3.47) | <0.001 |
| Platelet count <100,000/mm³ | 1.55 | (1.01-2.39) | 0.05 |
| Hepatocellular carcinoma | | | |
| development | | | |
| Poor-prognosis signature | 1.59 | (0.94-2.70) | 0.09 |
| Bilirubin >1.0 mg/dL | 1.61 | (1.02-2.54) | 0.04 |
| Platelet count <100,000/mm³ | 1.76 | (1.10-2.79) | 0.02 |

TABLE 17
Association of 186-Gene Signature Signature and Clinical Variables with Clinical Outcome
in Child-Pugh class A and Hepatitis C Infection (Multivariate Subgroup Analysis)

| | | | | | Hepatocellular | |
|---|---|---|---|---|---|---|
| | Overall survival | | Hepatic decompensation | | carcinoma development | |
| Variable | Hazard Ratio (95% CI) | P Value | Hazard Ratio (95% CI) | P Value | Hazard Ratio (95% CI) | P Value |

(A) Child-Pugh class A (N = 270)

| | | | | | | |
|---|---|---|---|---|---|---|
| Poor-prognosis signature | 2.16 (1.35-3.47) | 0.001 | 2.15 (1.34-3.45) | 0.002 | 1.63 (0.96-2.77) | 0.07 |
| Bilirubin >1.0 mg/dL | 2.02 (1.31-3.11) | 0.002 | 2.18 (1.41-3.36) | <0.001 | 1.70 (1.08-2.68) | 0.02 |
| Platelet count <100,000/mm³ | 1.86 (1.19-2.92) | 0.007 | 1.50 (0.98-2.32) | 0.07 | 1.88 (1.17-3.01) | 0.009 |

(B) Patients with hepatitis C infection (N = 249)

| | | | | | | |
|---|---|---|---|---|---|---|
| Poor-prognosis signature | 2.37 (1.46-3.84) | <0.001 | 2.31 (1.43-3.75) | <0.001 | 1.69 (0.99-2.88) | 0.06 |
| Bilirubin >1.0 mg/dL | 2.05 (1.30-3.22) | 0.002 | 2.10 (1.34-3.30) | 0.001 | 1.60 (1.00-2.58) | 0.05 |
| Platelet count <100,000/mm³ | 2.10 (1.29-3.42) | 0.003 | 1.58 (1.00-2.49) | 0.05 | 1.88 (1.14-3.08) | 0.01 |

TABLE 18
Association of 186-Gene Survival Signature Non-
cancer-related Death (Multivariate Analysis)

| Variable | Hazard Ratio (95% CI) | P Value |
|---|---|---|
| Poor-prognosis signature | 2.27 (1.29-3.98) | 0.004 |
| Bilirubin >1.0 mg/dL | 2.02 (1.19-3.43) | 0.009 |
| Platelet count <100,000/mm³ | 1.38 (0.80-2.38) | 0.24 |

TABLE 19
Gene sets associated with Clinical Outcome by Gene Set Enrichment Analysis (For details
of each gene set, click the name for the link to MSigDB gene set annotation page)

| | | |
|---|---|---|
| No. of | | |
| genes | NES | FDR |

(A) Gene sets correlated with high risk of hepatocellular
carcinoma development

| | | | |
|---|---|---|---|
| Experimental perturbation gene set | | | |
| RADAEVA IFNA UP | 31 | 1.83 | 0.165 |
| IFNA UV-CMV COMMON HCMV 6HRS UP | 31 | 1.78 | 0.172 |
| SANA IFNG ENDOTHELIAL UP | 44 | 1.78 | 0.135 |
| SANA TNFA ENDOTHELIAL UP | 49 | 1.77 | 0.121 |
| CROONQUIST RAS STROMA DN | 17 | 1.75 | 0.123 |
| CROONQUIST IL6 STROMA UP | 25 | 1.75 | 0.108 |
| NF9D UP | 18 | 1.73 | 0.118 |
| IL1 CORNEA UP | 35 | 1.73 | 0.108 |
| IFN ALPHA UP | 22 | 1.71 | 0.113 |
| IL6 FIBRO UP | 45 | 1.70 | 0.113 |
| CMV HCMV 6HRS UP | 22 | 1.65 | 0.161 |
| UVC LOW ALL DN | 30 | 1.63 | 0.190 |
| CMV_24HRS_DN | 43 | 1.62 | 0.192 |
| UV-CMV UNIQUE HCMV 6HRS UP | 96 | 1.60 | 0.202 |
| CMV UV-CMV COMMON HCMV 6HRS UP | 18 | 1.60 | 0.191 |
| CMV HCMV TIMECOURSE 12HRS UP | 22 | 1.58 | 0.213 |
| BLEO HUMAN LYMPH HIGH 24HRS UP | 74 | 1.57 | 0.225 |
| Litrature-based pathway gene set | | | |
| MATRIX METALLOPROTEINASES | 17 | 1.88 | 0.047 |
| APOPTOSIS KEGG | 33 | 1.70 | 0.146 |
| APOPTOSIS | 44 | 1.62 | 0.191 |
| STRIATED_MUSCLE_CONTRACTION | 22 | 1.62 | 0.147 |
| INOSITIOL PHOSPHATE METABOLISM | 15 | 1.61 | 0.121 |

(B) Gene sets correlated with low risk of hepatocellular
carcinoma developmemt

| | | | |
|---|---|---|---|
| Experimental perturbation gene set | | | |
| NI2 LONG DN | 20 | −1.75 | 0.232 |
| SCHUMACHER MYC UP | 34 | −1.72 | 0.200 |
| Litrature-based pathway gene set | | | |
| TRYPTOPHAN METABOLISM | 37 | −2.38 | <0.001 |
| GAMMA HEXACHLOROCYCLOHEXANE DEGRADATION | 21 | −2.17 | <0.001 |
| FATTY ACID METABOLISM | 59 | −2.15 | <0.001 |
| BETA ALANINE METABOLISM | 20 | −2.07 | 0.002 |
| VALINE_LEUCINE_AND_ISOLEUCINE_DEGRADATION | 28 | −2.02 | 0.003 |
| PROPANOATE METABOLISM | 22 | −1.79 | 0.029 |
| BILE ACID BIOSYNTHESIS | 22 | −1.77 | 0.031 |
| TYROSINE METABOLISM | 22 | −1.69 | 0.054 |
| BLOOD CLOTTING CASCADE | 17 | −1.63 | 0.075 |
| CITRATE_CYCLE_TCA_CYCLE | 16 | −1.60 | 0.082 |
| NUCLEAR RECEPTORS | 23 | −1.55 | 0.107 |
| BUTANOATE METABOLISM | 18 | −1.48 | 0.148 |
| PYRUVATE METABOLISM | 26 | −1.45 | 0.164 |
| PURINE METABOLISM | 77 | −1.44 | 0.171 |
| GLUTATHIONE_METABOLISM | 29 | −1.42 | 0.179 |
| ALANINE AND ASPARTATE METABOLISM | 17 | −1.37 | 0.223 |
| GLUTAMATE METABOLISM | 19 | −1.35 | 0.232 |

TABLE 20
Association of 186-Gene Survival Signature and Clinical
Variables with Overall Survival (Multivariate Analysis):
Models with age, esophageal/gastric varices, and albumin

| | | |
|---|---|---|
| Variable | Hazard Ratio (95% CI) | P Value |

(A) Model with Age

| | | |
|---|---|---|
| Age >50 yr | 1.55 (0.92-2.60) | 0.10 |
| Poor-prognosis signature | 2.06 (1.29-3.30) | 0.003 |
| Bilirubin >1.0 mg/dL | 1.94 (1.26-2.97) | 0.003 |
| Platelet count <100,000/mm³ | 1.78 (1.14-2.76) | 0.01 |

(B) Model with Esophageal/gastric varices

| | | |
|---|---|---|
| Esophageal/gastric varices | 1.32 (0.81-2.17) | 0.26 |
| Poor-prognosis signature | 2.01 (1.25-3.24) | 0.004 |
| Bilirubin >1.0 mg/dL | 1.94 (1.25-3.01) | 0.003 |
| Platelet count <100,000/mm³ | 1.68 (1.05-2.67) | 0.03 |

(C) Model with Albumin

| | | |
|---|---|---|
| Albumin <4.0 g/dL | 1.26 (0.78-2.04) | 0.35 |
| Poor-prognosis signature | 2.03 (1.25-3.29) | 0.004 |
| Bilirubin >1.0 mg/dL | 1.99 (1.28-3.10) | 0.002 |
| Platelet count <100,000/mm³ | 1.77 (1.11-2.82) | 0.02 |

TABLE 21
Association of 186-Gene Survival Signature and Clinical Variables
with Overall Survival (Multivariate Analysis): Model with MELD
(Model For End-Stage Liver Disease) score (N = 179)

| Variable | Hazard Ratio (95% CI) | P Value |
|---|---|---|
| MELD score >6 | 1.28 (0.71-2.31) | 0.42 |
| Poor-prognosis signature | 2.63 (1.46-4.77) | 0.001 |
| Bilirubin >1.0 mg/dL | 2.45 (1.32-4.53) | 0.004 |
| Platelet count <100,000/mm³ | 1.72 (0.92-3.20) | 0.09 |

TABLE 22
Association of 186-Gene Survival Signature and Hepatitis C-related Clinical Variables with Clinical Outcome
(Univariate and Multivariate Analysis)
(A) Univariate analysis

| | | | | | Hepatocellular | |
|---|---|---|---|---|---|---|
| | Overall survival | | Hepatic decompensation | | carcinoma development | |
| | Hazard | | Hazard | | Hazard | |
| Variable | Ratio (95% CI) | P Value | Ratio (95% CI) | P Value | Ratio (95% CI) | P Value |
| History of interferon treatment (N = 248) | 0.76 (0.48-1.18) | 0.23 | 0.73 (0.47-1.14) | 0.10 | 0.92 (0.58-1.47) | 0.73 |
| Sustained virological response to interferon | —* | 0.006** | 0.18 (0.04-0.77) | 0.02 | 0.21 (0.05-0.97) | 0.09 |
| (N = 248) | | | | | | |
| Hepatitis C virus genotype 1b (N = 234) | 2.32 (0.26-3.97) | 0.005 | 1.41 (0.88-2.27) | 0.18 | 1.40 (0.96-3.30) | 0.18 |

(B) Multivariate analysis

| Variable | Hazard Ratio (95% CI) | P Value |
|---|---|---|
| Overall survival (all patents, (N = 235) | | |
| Hepatitis C virus genotype 1b | 2.11 (9.25-3.57) | 0.005 |
| Poor-prognosis signature | 2.26 (1.34-3.80) | 0.002 |
| Bilirubin >1.0 mg/dL | 2.09 (2.29-3.38) | 0.003 |
| Platelet count <100,000/mm³ | 2.04 (1.22-3.39) | 0.005 |
| Overall survival (no interferon therapy, (N = 123) | | |
| Hepatitis C virus genotype 1b | 1.86 (0.93-3.75) | 0.08 |
| Poor-prognosis signature | 1.87 (0.93-3.73) | 0.08 |
| Bilirubin >1.0 mg/dL | 1.47 (0.77-2.81) | 0.24 |
| Platelet count <100,000/mm³ | 2.38 (1.23-4.84) | 0.04 |
| Hepatitis decompensation (N = 104) | | |
| Sustained virological response to interferon | 0.22 (0.05-0.94) | 0.04 |
| Poor-prognosis signature | 2.22 (1.00-4.94) | 0.05 |
| Bilirubin >1.0 mg/dL | 1.90 (0.94-3.55) | 0.08 |
| Platelet count <100,000/mm³ | 1.20 (0.99-2.48) | 0.82 |
| Hepatocellular carcinoma development (N = 104) | | |
| Sustained virological response to interferon | 0.27 (0.06-1.17) | 0.09 |
| Poor-prognosis signature | 2.89 (1.27-8.81) | 0.04 |
| Bilirubin >1.0 mg/dL | 1.82 (0.84-0.97) | 0.13 |
| Platelet count <100,000/mm³ | 1.21 (0.55-2.84) | 0.63 |

*Impossible to compute hazard because there was no events in patents with sustained virological response.
**Log-rant test.

TABLE 23
Association of 186-Gene Survival Signature and Clinical Variables
with Ascites or Gastrointestinal bleeding (Multivariate Analysis)

| | | |
|---|---|---|
| Variable | Hazard Ratio (95% CI) | P Value |

(A) Association with Ascites

| | | |
|---|---|---|
| Poor-prognosis signature | 2.48 (1.52-4.03) | <0.001 |
| Bilirubin >1.0 mg/dL | 2.65 (1.66-4.22) | <0.001 |
| Platelet count <100,000/mm³ | 1.14 (0.71-1.84) | 0.59 |
| Esophageal/gastric varices | 2.08 (1.27-3.40) | 0.003 |

(B) Association with Gastrointestinal bleeding

| | | |
|---|---|---|
| Esophageal/gastric varices | 2.39 (1.05-5.43) | 0.04 |

TABLE 24
Association of 186-Gene Survival Signature and Hepatocellular
Carcinoma Development according to Baveno IV stage
(Multivariate Subgroup Analysis)

| | | |
|---|---|---|
| Variable | Hazard Ratio (95% CI) | P Value |

(A) Baveno IV stage 1 (N = 204)

| | | |
|---|---|---|
| Poor-prognosis signature | 1.24 (0.62-2.48) | 0.53 |
| Bilirubin >1.0 mg/dL | 2.41 (1.42-4.09) | 0.001 |
| Platelet count <100,000/mm³ | 1.34 (0.76-2.36) | 0.31 |

(B) Baveno IV stage 2 (N = 65) Hepatocellular carcinoma development

| | | |
|---|---|---|
| Poor-prognosis signature | 3.00 (1.19-7.58) | 0.02 |
| Bilirubin >1.0 mg/dL | 0.43 (0.18-1.05) | 0.07 |
| Platelet count <100,000/mm³ | 3.33 (1.12-9.92) | 0.03 |

1. A method for determining if a subject is at risk for developing a hepatic disorder, the method comprising comparing:
a) the level of expression of a marker or a plurality of markers in a subject sample; and
b) the level of expression of the marker or plurality of markers in a control sample,
wherein the marker or plurality of markers are selected from the group consisting of the markers listed in Table 2A, Table 2B, Table 13A, Table 13B, and FIG. 19 and a significant difference between the level of expression of the marker or plurality of markers in the subject sample and the control sample is an indication that the subject is at risk for developing the hepatic disorder.

2. (canceled)

3. The method of claim 1, wherein the marker or plurality of markers have increased expression relative to a control.

4. The method of claim 1, wherein the marker or plurality of markers have decreased expression relative to a control.

5. The method of claim 1, wherein at least one marker has increased expression and at least one marker has decreased expression relative to a control.

6. The method of claim 1, wherein the hepatic disorder is liver cancer and/or cirrhosis.

7. The method of claim 1, wherein the liver cancer is a hepatocellular carcinoma.

8. The method of claim 1, wherein the marker or plurality of markers comprise a transcribed polynucleotide or portion thereof.

9. The method of claim 1, wherein the marker or plurality of markers corresponds to a protein.

10. The method of claim 1, wherein the level of expression of the marker or plurality of markers in the samples is assessed by detecting the presence of a marker protein in the samples.

11. The method of claim 10, wherein the presence of the marker protein is detected using a reagent which specifically binds with the protein.

12. The method of claim 11, wherein the reagent is selected from the group consisting of an antibody, an antibody derivative, and an antibody fragment.

13. The method of claim 1, wherein the level of expression of the marker or plurality of markers in the samples is assessed by detecting the presence in the sample of a transcribed polynucleotide or portion thereof, corresponding to a nucleic acid marker.

14. The method of claim 13, wherein the transcribed polynucleotide is an mRNA or a cDNA.

15. The method of claim 13, wherein detecting the transcribed polynucleotide comprises amplifying the transcribed polynucleotide.

16. The method of claim 1, wherein the level of expression of the marker or plurality of markers in the samples is assessed by detecting the presence in the sample of a transcribed polynucleotide which anneals with a nucleic acid marker or a portion thereof under stringent hybridization conditions.

17. The method of claim 1, wherein the level of expression of the marker or plurality of markers in the subject sample differs from the level of expression of the marker or plurality of markers in the control sample by a factor of at least about 2 or at least about 5.

18. The method of claim 1, wherein the level of expression of the marker or plurality of markers is determined using oligonucleotide microarrays.

19. The method of claim 1, wherein the level of expression of the marker or plurality of markers is determined using a complementary DNA-mediated annealing, selection, extension, and ligation assay.

20-34. (canceled)

35. A diagnostic array comprising:
a) a solid support; and
b) a plurality of diagnostic agents coupled to the solid support,
wherein each of the agents is used to assay the expression level of a marker or a plurality of markers is selected from the group consisting of the markers listed in Table 2A, Table 2B, Table 13A, Table 13B, and FIG. 19.

36-38. (canceled)

39. A kit for assessing the presence of cells having or indicative of a hepatic disorder, the kit comprising at least one nucleic acid probe wherein the probe or probes specifically bind with transcribed polynucleotides corresponding to a marker or a plurality of markers selected from the group consisting of the markers listed in Table 2A, Table 2B, Table 13A, Table 13B, and FIG. 19.

40-41. (canceled)
